SEQUENCE LISTING (841 sequences)

SEQ ID NO: 1 - protein, 10 aa, synthetic construct
  REGION 1..10: note = Description of Artificial Sequence: Synthetic peptide
GGSFSDYYWS

SEQ ID NO: 2 - protein, 10 aa, synthetic construct
  REGION 1..10: note = Description of Artificial Sequence: Synthetic peptide
GGSLSDYHWS

SEQ ID NO: 3 - protein, 10 aa, synthetic construct
  REGION 1..10: note = Description of Artificial Sequence: Synthetic peptide
GGSFSDYYWS

SEQ ID NO: 4 - protein, 10 aa, synthetic construct
  REGION 1..10: note = Description of Artificial Sequence: Synthetic peptide
GGSFNDYYWS

SEQ ID NO: 5 - protein, 10 aa, synthetic construct
  REGION 1..10: note = Description of Artificial Sequence: Synthetic peptide
GGSFSDYYWS

SEQ ID NO: 6 - protein, 10 aa, synthetic construct
  REGION 1..10: note = Description of Artificial Sequence: Synthetic peptide
GGSFSDYYWS

SEQ ID NO: 7 - protein, 10 aa, synthetic construct
  REGION 1..10: note = Description of Artificial Sequence: Synthetic peptide
GGSFSDYYWS

SEQ ID NO: 8 - protein, 10 aa, synthetic construct
  REGION 1..10: note = Description of Artificial Sequence: Synthetic peptide
GGSFSDYYWS

SEQ ID NO: 9 - protein, 10 aa, synthetic construct
  REGION 1..10: note = Description of Artificial Sequence: Synthetic peptide
GGSFSDYYWS

SEQ ID NO: 10 - protein, 10 aa, synthetic construct
  REGION 1..10: note = Description of Artificial Sequence: Synthetic peptide
GGSFSDYYWS

SEQ ID NO: 11 - protein, 10 aa, synthetic construct
  REGION 1..10: note = Description of Artificial Sequence: Synthetic peptide
GGSFSDYYWS

SEQ ID NO: 12 - protein, 19 aa, synthetic construct
  REGION 1..19: note = Description of Artificial Sequence: Synthetic peptide
EVNHRGSINYNNYNPSLKS

SEQ ID NO: 13 - protein, 16 aa, synthetic construct
  REGION 1..16: note = Description of Artificial Sequence: Synthetic peptide
EINHSGSTNYNPSLKS

SEQ ID NO: 14 - protein, 16 aa, synthetic construct
  REGION 1..16: note = Description of Artificial Sequence: Synthetic peptide
EINHSGSTNYNPSLKS

SEQ ID NO: 15 - protein, 16 aa, synthetic construct
  REGION 1..16: note = Description of Artificial Sequence: Synthetic peptide
EVNHSGSTSYNPSLKS

SEQ ID NO: 16 - protein, 16 aa, synthetic construct
  REGION 1..16: note = Description of Artificial Sequence: Synthetic peptide
EINHSGSTNYNPSLKS

SEQ ID NO: 17 - protein, 19 aa, synthetic construct
  REGION 1..19: note = Description of Artificial Sequence: Synthetic peptide
EVNHSGSINYNNYNPSLKS

SEQ ID NO: 18 - protein, 19 aa, synthetic construct
  REGION 1..19: note = Description of Artificial Sequence: Synthetic peptide
EVNHAGSINYNNYNPSLKS

SEQ ID NO: 19 - protein, 16 aa, synthetic construct
  REGION 1..16: note = Description of Artificial Sequence: Synthetic peptide
EVNHRGSINYNPSLKS

SEQ ID NO: 20 - protein, 19 aa, synthetic construct
  REGION 1..19: note = Description of Artificial Sequence: Synthetic peptide
EVNHRGSINYNNYNPSLKS

SEQ ID NO: 21 - protein, 19 aa, synthetic construct
  REGION 1..19: note = Description of Artificial Sequence: Synthetic peptide
EVNHRGSINYNNYNPSLKS

SEQ ID NO: 22 - protein, 19 aa, synthetic construct
  REGION 1..19: note = Description of Artificial Sequence: Synthetic peptide
EVNHRGSINYNNYNPSLKS

SEQ ID NO: 23 - protein, 21 aa, synthetic construct
  REGION 1..21: note = Description of Artificial Sequence: Synthetic peptide
AKPLRPHCTNGVCYSGDAFDI

SEQ ID NO: 24 - protein, 21 aa, synthetic construct
  REGION 1..21: note = Description of Artificial Sequence: Synthetic peptide
AKPFRPHCTNGVCHSGDAFDI

SEQ ID NO: 25 - protein, 21 aa, synthetic construct
  REGION 1..21: note = Description of Artificial Sequence: Synthetic peptide
AKPFRPHCTNGVCYSGDAFDI

SEQ ID NO: 26 - protein, 21 aa, synthetic construct
  REGION 1..21: note = Description of Artificial Sequence: Synthetic peptide
AKPFRPHCTNGVCYSGDAFDI

SEQ ID NO: 27 - protein, 21 aa, synthetic construct
  REGION 1..21: note = Description of Artificial Sequence: Synthetic peptide
AKPFRPHCTNGVCYSGDAFDI

SEQ ID NO: 28 - protein, 21 aa, synthetic construct
  REGION 1..21: note = Description of Artificial Sequence: Synthetic peptide
AKPLRPHCTNGVCYSGDAFDI

SEQ ID NO: 29 - protein, 21 aa, synthetic construct
  REGION 1..21: note = Description of Artificial Sequence: Synthetic peptide
AKPLRPHCTNGVCYSGDAFDI

SEQ ID NO: 30 - protein, 21 aa, synthetic construct
  REGION 1..21: note = Description of Artificial Sequence: Synthetic peptide
AKPLRPHCTNGVCYSGDAFDI

SEQ ID NO: 31 - protein, 21 aa, synthetic construct
  REGION 1..21: note = Description of Artificial Sequence: Synthetic peptide
AKPLRPHCTNGVCYSGDAFDI

SEQ ID NO: 32 - protein, 21 aa, synthetic construct
  REGION 1..21: note = Description of Artificial Sequence: Synthetic peptide
AKPLRPHCTNGVCYSGDAFDI

SEQ ID NO: 33 - protein, 21 aa, synthetic construct
  REGION 1..21: note = Description of Artificial Sequence: Synthetic peptide
AKPLRPHCTNAVCYSGDAFDI

SEQ ID NO: 34 - protein, 11 aa, synthetic construct
  REGION 1..11: note = Description of Artificial Sequence: Synthetic peptide
GGNNIGSKNVH

SEQ ID NO: 35 - protein, 11 aa, synthetic construct
  REGION 1..11: note = Description of Artificial Sequence: Synthetic peptide
GGNNIGSKNVH

SEQ ID NO: 36 - protein, 11 aa, synthetic construct
  REGION 1..11: note = Description of Artificial Sequence: Synthetic peptide
GGNNIGTKNVH

SEQ ID NO: 37 - protein, 11 aa, synthetic construct
  REGION 1..11: note = Description of Artificial Sequence: Synthetic peptide
GGNNIGSKNVH

SEQ ID NO: 38 - protein, 11 aa, synthetic construct
  REGION 1..11: note = Description of Artificial Sequence: Synthetic peptide
GGNNIGSKNVH

SEQ ID NO: 39 - protein, 11 aa, synthetic construct
  REGION 1..11: note = Description of Artificial Sequence: Synthetic peptide
GGNNIGSKNVH

SEQ ID NO: 40 - protein, 11 aa, synthetic construct
  REGION 1..11: note = Description of Artificial Sequence: Synthetic peptide
GGNNIGSKNVH

SEQ ID NO: 41 - protein, 11 aa, synthetic construct
  REGION 1..11: note = Description of Artificial Sequence: Synthetic peptide
GGNNIGSKNVH

SEQ ID NO: 42 - protein, 11 aa, synthetic construct
  REGION 1..11: note = Description of Artificial Sequence: Synthetic peptide
GGNNIGSKNVH

SEQ ID NO: 43 - protein, 11 aa, synthetic construct
  REGION 1..11: note = Description of Artificial Sequence: Synthetic peptide
GGNNIGSKNVH

SEQ ID NO: 44 - protein, 11 aa, synthetic construct
  REGION 1..11: note = Description of Artificial Sequence: Synthetic peptide
GGNNIGSKNVH

SEQ ID NO: 45 - protein, 7 aa, synthetic construct
  REGION 1..7: note = Description of Artificial Sequence: Synthetic peptide
DDSDRPS

SEQ ID NO: 46 - protein, 7 aa, synthetic construct
  REGION 1..7: note = Description of Artificial Sequence: Synthetic peptide
DDSDRPS

SEQ ID NO: 47 - protein, 7 aa, synthetic construct
  REGION 1..7: note = Description of Artificial Sequence: Synthetic peptide
DDSDRPS

SEQ ID NO: 48 - protein, 7 aa, synthetic construct
  REGION 1..7: note = Description of Artificial Sequence: Synthetic peptide
DDSDRPS

SEQ ID NO: 49 - protein, 7 aa, synthetic construct
  REGION 1..7: note = Description of Artificial Sequence: Synthetic peptide
DDSDRPS

SEQ ID NO: 50 - protein, 7 aa, synthetic construct
  REGION 1..7: note = Description of Artificial Sequence: Synthetic peptide
DDSDRPS

SEQ ID NO: 51 - protein, 7 aa, synthetic construct
  REGION 1..7: note = Description of Artificial Sequence: Synthetic peptide
DDSDRPS

SEQ ID NO: 52 - protein, 7 aa, synthetic construct
  REGION 1..7: note = Description of Artificial Sequence: Synthetic peptide
DDSDRPS

SEQ ID NO: 53 - protein, 7 aa, synthetic construct
  REGION 1..7: note = Description of Artificial Sequence: Synthetic peptide
DDSDRPS

SEQ ID NO: 54 - protein, 7 aa, synthetic construct
  REGION 1..7: note = Description of Artificial Sequence: Synthetic peptide
DDSDRPS

SEQ ID NO: 55 - protein, 7 aa, synthetic construct
  REGION 1..7: note = Description of Artificial Sequence: Synthetic peptide
DDSDRPS

SEQ ID NO: 56 - protein, 11 aa, synthetic construct
  REGION 1..11: note = Description of Artificial Sequence: Synthetic peptide
QVWDSSSDHLV

SEQ ID NO: 57 - protein, 11 aa, synthetic construct
  REGION 1..11: note = Description of Artificial Sequence: Synthetic peptide
QVWDSSSDHLV

SEQ ID NO: 58 - protein, 11 aa, synthetic construct
  REGION 1..11: note = Description of Artificial Sequence: Synthetic peptide
QVWDSSSDHLV

SEQ ID NO: 59 - protein, 11 aa, synthetic construct
  REGION 1..11: note = Description of Artificial Sequence: Synthetic peptide
QVWDSSSDHLV

SEQ ID NO: 60 - protein, 11 aa, synthetic construct
  REGION 1..11: note = Description of Artificial Sequence: Synthetic peptide
QVWDSSSDHLV

SEQ ID NO: 61 - protein, 11 aa, synthetic construct
  REGION 1..11: note = Description of Artificial Sequence: Synthetic peptide
QVWDSSSDHLV

SEQ ID NO: 62 - protein, 11 aa, synthetic construct
  REGION 1..11: note = Description of Artificial Sequence: Synthetic peptide
QVWDSSSDHLV

SEQ ID NO: 63 - protein, 11 aa, synthetic construct
  REGION 1..11: note = Description of Artificial Sequence: Synthetic peptide
QVWDSSSDHLV

SEQ ID NO: 64 - protein, 11 aa, synthetic construct
  REGION 1..11: note = Description of Artificial Sequence: Synthetic peptide
QVWDSSSDHLV

SEQ ID NO: 65 - protein, 11 aa, synthetic construct
  REGION 1..11: note = Description of Artificial Sequence: Synthetic peptide
QVWDSSSDHLV

SEQ ID NO: 66 - protein, 11 aa, synthetic construct
  REGION 1..11: note = Description of Artificial Sequence: Synthetic peptide
QVWDSSSDHLV

SEQ ID NO: 67 - protein, 130 aa, synthetic construct [sequence omitted]

SEQ ID NO: 68 - protein, 127 aa, synthetic construct
  REGION 1..127: note = Description of Artificial Sequence: Synthetic polypeptide
QVQLQQWGAGLLKPSETLSLTCAVYGGSLSDYHWSWIRQPPGKGLEWIGEINHSGSTNYNPSLKSRVTISVDTSKNQFSLKLSSVSAADTAVYYCAKPFRPHCTNGVCHSGDAFDIWGQGTMATVSS

SEQ ID NO: 69 - protein, 127 aa, synthetic construct
  REGION 1..127: note = Description of Artificial Sequence: Synthetic polypeptide
QVQLQQWGAGLLKPSETLSLTCAVYGGSFSDYYWSWIRQPPGKGLEWIGEINHSGSTNYNPSLKSRVTISVDPSKNQFSLKLTSVTAADTAVYYCAKPFRPHCTNGVCYSGDAFDIWGQGTMVTVSS

SEQ ID NO: 70 - protein, 127 aa, synthetic construct
  REGION 1..127: note = Description of Artificial Sequence: Synthetic polypeptide
QVQLQQWGAGLLKPSETLSLTCAVYGGSFNDYYWSWIRQPPGKGLEWIGEVNHSGSTSYNPSLKSRVTISVDTSKNQFSLKLSSVTAADTAVYYCAKPFRPHCTNGVCYSGDAFDIWGQGTMVTVSS

SEQ ID NO: 71 - protein, 127 aa, synthetic construct; identical to SEQ ID NO: 69
  REGION 1..127: note = Description of Artificial Sequence: Synthetic polypeptide

SEQ ID NO: 72 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEVNHSGSINYNNYNPSLKSRVTISVDPSKNQFSLKLTSVTAADTAVYYCAKPLRPHCTNGVCYSGDAFDIWGQGTMVTVAS

SEQ ID NO: 73 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEVNHAGSINYNNYNPSLKSRVTISVDPSKNQFSLKLTSVTAADTAVYYCAKPLRPHCTNGVCYSGDAFDIWGQGTMVTVAS

SEQ ID NO: 74 - protein, 127 aa, synthetic construct
  REGION 1..127: note = Description of Artificial Sequence: Synthetic polypeptide
QVQLQQWGAGLLKPSETLSLTCAVYGGSFSDYYWSWIRQPPGKGLEWIGEVNHRGSINYNPSLKSRVTISVDPSKNQFSLKLTSVTAADTAVYYCAKPLRPHCTNGVCYSGDAFDIWGQGTMVTVAS

SEQ ID NO: 75 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEVNHRGSINYNNYNPSLKSRVTISVDTSKNQFSLKLTSVTAADTAVYYCAKPLRPHCTNGVCYSGDAFDIWGQGTMVTVAS

SEQ ID NO: 76 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEVNHRGSINYNNYNPSLKSRVTISVDASKNQFSLKLTSVTAADTAVYYCAKPLRPHCTNGVCYSGDAFDIWGQGTMVTVAS

SEQ ID NO: 77 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEVNHRGSINYNNYNPSLKSRVTISVDPSKNQFSLKLTSVTAADTAVYYCAKPLRPHCTNAVCYSGDAFDIWGQGTMVTVAS

SEQ ID NO: 78 - protein, 108 aa, synthetic construct [sequence omitted]

SEQ ID NO: 79 - protein, 108 aa, synthetic construct [sequence omitted]

SEQ ID NO: 80 - protein, 108 aa, synthetic construct
  REGION 1..108: note = Description of Artificial Sequence: Synthetic polypeptide
SYVLTQPPSVSVAPGQTARITCGGNNIGTKNVHWYQQKPGQAPVLVVYDDSDRPSGIPEQFSGSNSGNTATLTISRVEAGDEADYYCQVWDSSSDHLVFGGGTKLTVL

SEQ ID NO: 81 - protein, 108 aa, synthetic construct
  REGION 1..108: note = Description of Artificial Sequence: Synthetic polypeptide
SYVLTQPPSVSVAPGQTARITCGGNNIGSKNVHWYQQKPGQAPALVVYDDSDRPSGIPEQFSGSNSGNTATLTISRVEAGDEADYYCQVWDSSSDHLVFGGGTKLTVL

SEQ ID NO: 82 - protein, 108 aa, synthetic construct [sequence omitted]

SEQ ID NO: 83 - protein, 108 aa, synthetic construct [sequence omitted]

SEQ ID NO: 84 - protein, 108 aa, synthetic construct [sequence omitted]

SEQ ID NO: 85 - protein, 108 aa, synthetic construct [sequence omitted]

SEQ ID NO: 86 - protein, 108 aa, synthetic construct [sequence omitted]

SEQ ID NO: 87 - protein, 108 aa, synthetic construct [sequence omitted]

SEQ ID NO: 88 - protein, 108 aa, synthetic construct [sequence omitted]

SEQ ID NO: 89 - protein, 459 aa, synthetic construct
  REGION 1..459: note = Description of Artificial Sequence: Synthetic polypeptide
QVQLQQWGAGLLKPSETLSLTCAVYGGSFSDYYWSWIRQPPGKGLEWIGEVNHRGSINYNNYNPSLKSRVTISVDPSKNQFSLKLTSVTAADTAVYYCAKPLRPHCTNGVCYSGDAFDIWGQGTMVTVASAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 90 - protein, 214 aa, synthetic construct
  REGION 1..214: note = Description of Artificial Sequence: Synthetic polypeptide
SYVLTQPPSVSVAPGQTARITCGGNNIGSKNVHWYQQKPGQAPVLVVYDDSDRPSGIPEQFSGSNSGNTATLTISRVEAGDEADYYCQVWDSSSDHLVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 91 - protein, 15 aa, synthetic construct
  REGION 1..15: note = Description of Artificial Sequence: Synthetic peptide
GGGGSGGGGSGGGGS

SEQ ID NO: 92 - protein, 212 aa, synthetic construct
  REGION 1..212: note = Description of Artificial Sequence: Synthetic polypeptide
ITCPPPMSVEHADIWVKSYSLYSRERYICNSGFKRKAGTSSLTECVLNKATNVAHWTTPSLKCIRDPALVHQRPAPPGGGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 93 - protein, 212 aa, synthetic construct
  REGION 1..212: note = Description of Artificial Sequence: Synthetic polypeptide
ITCPPPMSVEHADIWVKSYSLYSRERYICNSGFKRKAGTSSLTECVLNKATNVAHWTTPSLKCIRDPALVHQRPAPPGGGGSGGGGSGGGSGGGGSLQNWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 94 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 95 - protein, 98 aa, Homo sapiens
TEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGN

SEQ ID NO: 96 - protein, 24 aa, Homo sapiens
MELQAARACFALLWGCALAAAAAA

SEQ ID NO: 97 - protein, 175 aa, Homo sapiens
QGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYK

SEQ ID NO: 98 - protein, 62 aa, Homo sapiens
KCPELLQGLAHFPETIAGSDAPSLATVAGTCVDHAVVPPGGEEPRMHCAVDGEWLVPIGQCL

SEQ ID NO: 99 - protein, 65 aa, Homo sapiens
CQAGYEKVEDACQACSPGFFKFEASESPCLECPEHTLPSPEGATSCECEEGFFRAPQDPASMPCT

SEQ ID NO: 100 - protein, 110 aa, Homo sapiens
RPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQ

SEQ ID NO: 101 - protein, 510 aa, Homo sapiens
QGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGN

SEQ ID NO: 102 - protein, 524 aa, Homo sapiens
SRVYPANEVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESFTQVDIGDRIMKLNTEIRDVGPLSKKGFYLAFQDVGACIALVSVRVFYKKCPLTVRNLAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPSRIIGDGANS

SEQ ID NO: 103 - protein, 512 aa, synthetic construct
  REGION 1..512: note = Description of Artificial Sequence: Synthetic polypeptide
QGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGN

SEQ ID NO: 104 - protein, 520 aa, synthetic construct
  REGION 1..520: note = Description of Artificial Sequence: Synthetic polypeptide
QGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPSRIIGDGANS

SEQ ID NO: 105 - protein, 17 aa, synthetic construct
  REGION 1..17: note = Description of Artificial Sequence: Synthetic peptide
TSGGGGSGGGGSGGGGS

SEQ ID NO: 106 - protein, 154 aa, synthetic construct
  REGION 1..154: note = Description of Artificial Sequence: Synthetic polypeptide
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 107 - protein, 482 aa, synthetic construct
  REGION 1..482: note = Description of Artificial Sequence: Synthetic polypeptide
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGGSGGGGSGGQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGGSGGGGSGGQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 108 - protein, 466 aa, synthetic construct
  REGION 1..466: note = Description of Artificial Sequence: Synthetic polypeptide
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGGQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGGQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 109 - protein, 178 aa, synthetic construct
  REGION 1..178: note = Description of Artificial Sequence: Synthetic polypeptide
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 110 - protein, 554 aa, synthetic construct
  REGION 1..554: note = Description of Artificial Sequence: Synthetic polypeptide
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGSGGGGSGGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGSGGGGSGGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 111 - protein, 252 aa, synthetic construct
  REGION 1..252: note = Description of Artificial Sequence: Synthetic polypeptide
QVQLQQSGAELAKPGTSVKLSCKASGYTFTSYYIYWVKQRPGQGLEWIGNIWPGNGGTFYGEKFMGKATFTADTSSSTAYMLLGSLTPEDSAYYFCARRPDYSGDDYFDYWGQGVLVTVSSGGGGSGGGGSGGGGSGGGGSQVVLTQPKSVSTSLKSTVKLSCKLNSGNIGSYYVHWYQQHAGRSPTTMIYRDDKRPDGVPDRFSGSIDSSSNSAFLTINNVQTEDDAIYFCHSYDSTITPVFGGGTKLTVL

SEQ ID NO: 112 - protein, 252 aa, synthetic construct
  REGION 1..252: note = Description of Artificial Sequence: Synthetic polypeptide
QVVLTQPKSVSTSLKSTVKLSCKLNSGNIGSYYVHWYQQHAGRSPTTMIYRDDKRPDGVPDRFSGSIDSSSNSAFLTINNVQTEDDAIYFCHSYDSTITPVFGGGTKLTVLGGGGSGGGGSGGGGSGGGGSQVQLQQSGAELAKPGTSVKLSCKASGYTFTSYYIYWVKQRPGQGLEWIGNIWPGNGGTFYGEKFMGKATFTADTSSSTAYMLLGSLTPEDSAYYFCARRPDYSGDDYFDYWGQGVLVTVSS

SEQ ID NO: 113 - protein, 253 aa, synthetic construct
  REGION 1..253: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDSPFLLDDYYYYYYMDVWGKGTTVTVSSGGGGSGGGGSGGGGSGGGGSDIQMTQSPSSVSASVGDRVTITCRASQGISSWLAWYQQKPGKAPKLLIYAASSLQSGVPSRFSGSGSGTDFTLTISSLQPEDFATYYCQQGHLFPITFGGGTKVEIK

SEQ ID NO: 114 - protein, 253 aa, synthetic construct
  REGION 1..253: note = Description of Artificial Sequence: Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGHLFPITFGGGTKVEIKGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDSPFLLDDYYYYYYMDVWGKGTTVTVSS

SEQ ID NO: 115 - protein, 253 aa, synthetic construct
  REGION 1..253: note = Description of Artificial Sequence: Synthetic polypeptide
EVQLLESGGGLVQPGGSLRLSCAASGFTFRNYAMSWVRQAPGKGLEWVSAISGSGDTTYYADSVKGRFTISRDNSKNTLYLQMNSLRAEDTAVYYCAKDSPFLLDDYYYYYYMDVWGKGTTVTVSSGGGGSGGGGSGGGGSGGGGSDIQMTQSPSSVSASVGDRVTITCRASQGISSWLAWYQQKPGKAPKLLIYAASSLQSGVPSRFSGSGSGTDFTLTISSLQPEDFATYYCQQGHLFPITFGGGTKVEIK

SEQ ID NO: 116 - protein, 253 aa, synthetic construct
  REGION 1..253: note = Description of Artificial Sequence: Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGHLFPITFGGGTKVEIKGGGGSGGGGSGGGGSGGGGSEVQLLESGGGLVQPGGSLRLSCAASGFTFRNYAMSWVRQAPGKGLEWVSAISGSGDTTYYADSVKGRFTISRDNSKNTLYLQMNSLRAEDTAVYYCAKDSPFLLDDYYYYYYMDVWGKGTTVTVSS

SEQ ID NO: 117 - protein, 250 aa, synthetic construct
  REGION 1..250: note = Description of Artificial Sequence: Synthetic polypeptide
QVKLVQSGAALVKPGASVKMSCKASDYTFNDYWVSWVKQRHGESLEWIGEIYPNSGATNFNGKFRGKATLTVDNPTSTAYMELSRLTSEDSAIYYCTREYTRDWFAYWGQGTLVTVSSGGGGSGGGGSGGGGSGGGGSDVVLTQTPSILSATIGQSVSISCRSSQSLLDSDGNTYLYWFLQRPGQSPQRLIYLVSNLGSGVPNRFSGSGSGTDFTLKISGVEAEDLGIYYCMQPTHAPYTFGAGTKLELK

SEQ ID NO: 118 - protein, 250 aa, synthetic construct
  REGION 1..250: note = Description of Artificial Sequence: Synthetic polypeptide
DVVLTQTPSILSATIGQSVSISCRSSQSLLDSDGNTYLYWFLQRPGQSPQRLIYLVSNLGSGVPNRFSGSGSGTDFTLKISGVEAEDLGIYYCMQPTHAPYTFGAGTKLELKGGGGSGGGGSGGGGSGGGGSQVKLVQSGAALVKPGASVKMSCKASDYTFNDYWVSWVKQRHGESLEWIGEIYPNSGATNFNGKFRGKATLTVDNPTSTAYMELSRLTSEDSAIYYCTREYTRDWFAYWGQGTLVTVSS

SEQ ID NO: 119 - protein, 246 aa, synthetic construct
  REGION 1..246: note = Description of Artificial Sequence: Synthetic polypeptide
QVQLQQSGAEVIKPGASVKLSCKASGYTFSSYWMHWVRQAPGQGLEWIGEINPGNGHTNYNEKFKSRATLTGDTSTSTVYMELSSLRSEDTAVYYCARSFTTARAFAYWGQGTLVTVSSGGGGSGGGGSGGGGSGGGGSDIVMTQSPAFLSVTPGEKVTITCRASQTISDYLHWYQQKPDQAPKLLIKYASQSISGIPSRFSGSGSGTDFTFTISSLEAEDAATYYCQDGHSFPPTFGQGTKLEIK

SEQ ID NO: 120 - protein, 246 aa, synthetic construct
  REGION 1..246: note = Description of Artificial Sequence: Synthetic polypeptide
DIVMTQSPAFLSVTPGEKVTITCRASQTISDYLHWYQQKPDQAPKLLIKYASQSISGIPSRFSGSGSGTDFTFTISSLEAEDAATYYCQDGHSFPPTFGQGTKLEIKGGGGSGGGGSGGGGSGGGGSQVQLQQSGAEVIKPGASVKLSCKASGYTFSSYWMHWVRQAPGQGLEWIGEINPGNGHTNYNEKFKSRATLTGDTSTSTVYMELSSLRSEDTAVYYCARSFTTARAFAYWGQGTLVTVSS

SEQ ID NO: 121 - protein, 247 aa, synthetic construct
  REGION 1..247: note = Description of Artificial Sequence: Synthetic polypeptide
EVQLVESGGGLVQPGGSLRLSCAASGFTFSDYWMSWVRQAPGKGLEWVADIKNDGSYTNYAPSLTNRFTISRDNAKNSLYLQMNSLRAEDTAVYYCARELTGTWGQGTMVTVSSGGGGSGGGGSGGGGSGGGGSDIVMTQSPDSLAVSLGERATINCKSSQSLLSSGNQKNYLAWYQQKPGQPPKLLIYYASTRQSGVPDRFSGSGSGTDFTLTISSLQAEDVAVYYCLQYDRYPFTFGQGTKLEIK

SEQ ID NO: 122 - protein, 247 aa, synthetic construct
  REGION 1..247: note = Description of Artificial Sequence: Synthetic polypeptide
DIVMTQSPDSLAVSLGERATINCKSSQSLLSSGNQKNYLAWYQQKPGQPPKLLIYYASTRQSGVPDRFSGSGSGTDFTLTISSLQAEDVAVYYCLQYDRYPFTFGQGTKLEIKGGGGSGGGGSGGGGSGGGGSEVQLVESGGGLVQPGGSLRLSCAASGFTFSDYWMSWVRQAPGKGLEWVADIKNDGSYTNYAPSLTNRFTISRDNAKNSLYLQMNSLRAEDTAVYYCARELTGTWGQGTMVTVSS

SEQ ID NO: 123 - protein, 328 aa, synthetic construct
  REGION 1..328: note = Description of Artificial Sequence: Synthetic polypeptide
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 325 aa, synthetic construct
  REGION 1..325: note = Description of Artificial Sequence: Synthetic polypeptide
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 125 - protein, 328 aa, synthetic construct
  REGION 1..328: note = Description of Artificial Sequence: Synthetic polypeptide
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 322 aa, synthetic construct
  REGION 1..322: note = Description of Artificial Sequence: Synthetic polypeptide
KTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 127 - protein, 328 aa, synthetic construct
  REGION 1..328: note = Description of Artificial Sequence: Synthetic polypeptide
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 128 - protein, 328 aa, synthetic construct
  REGION 1..328: note = Description of Artificial Sequence: Synthetic polypeptide
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 325 aa, synthetic construct
  REGION 1..325: note = Description of Artificial Sequence: Synthetic polypeptide
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLG

SEQ ID NO: 130 - protein, 325 aa, synthetic construct
  REGION 1..325: note = Description of Artificial Sequence: Synthetic polypeptide
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSKLTVDKSRWQEGNVFSCSVMHEALHNHYTQKSLSLSLG

SEQ ID NO: 131 - protein, 328 aa, synthetic construct
  REGION 1..328: note = Description of Artificial Sequence: Synthetic polypeptide
STKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 328 aa, synthetic construct
  REGION 1..328: note = Description of Artificial Sequence: Synthetic polypeptide
STKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 133 - protein, 125 aa, synthetic construct
  REGION 1..125: note = Description of Artificial Sequence: Synthetic polypeptide
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 134 - protein, 109 aa, synthetic construct
  REGION 1..109: note = Description of Artificial Sequence: Synthetic polypeptide
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 135 - protein, 125 aa, synthetic construct
  REGION 1..125: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 136 - protein, 109 aa, synthetic construct
  REGION 1..109: note = Description of Artificial Sequence: Synthetic polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 137 - protein, 125 aa, synthetic construct
  REGION 1..125: note = Description of Artificial Sequence: Synthetic polypeptide
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSS

SEQ ID NO: 138 - protein, 109 aa, synthetic construct
  REGION 1..109: note = Description of Artificial Sequence: Synthetic polypeptide
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 139 - protein, 125 aa, synthetic construct
  REGION 1..125: note = Description of Artificial Sequence: Synthetic polypeptide
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 140 - protein, 109 aa, synthetic construct; identical to SEQ ID NO: 138
  REGION 1..109: note = Description of Artificial Sequence: Synthetic polypeptide

SEQ ID NO: 141 - protein, 125 aa, synthetic construct
  REGION 1..125: note = Description of Artificial Sequence: Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 142 - protein, 109 aa, synthetic construct
  REGION 1..109: note = Description of Artificial Sequence: Synthetic polypeptide
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 143 - protein, 125 aa, synthetic construct
  REGION 1..125: note = Description of Artificial Sequence: Synthetic polypeptide
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 144 - protein, 109 aa, synthetic construct; identical to SEQ ID NO: 142
  REGION 1..109: note = Description of Artificial Sequence: Synthetic polypeptide

SEQ ID NO: 145 - protein, 125 aa, synthetic construct
  REGION 1..125: note = Description of Artificial Sequence: Synthetic polypeptide
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSS

SEQ ID NO: 146 - protein, 109 aa, synthetic construct; identical to SEQ ID NO: 142
  REGION 1..109: note = Description of Artificial Sequence: Synthetic polypeptide

SEQ ID NO: 147 - protein, 125 aa, synthetic construct
  REGION 1..125: note = Description of Artificial Sequence: Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSS

SEQ ID NO: 148 - protein, 109 aa, synthetic construct; identical to SEQ ID NO: 142
  REGION 1..109: note = Description of Artificial Sequence: Synthetic polypeptide

SEQ ID NO: 149 - protein, 125 aa, synthetic construct
  REGION 1..125: note = Description of Artificial Sequence: Synthetic polypeptide
EVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCVRHGNFGNSYVSYFAYWGQGTTVTVSS

SEQ ID NO: 150 - protein, 110 aa, synthetic construct
  REGION 1..110: note = Description of Artificial Sequence: Synthetic polypeptide
EIVVTQSPATLSVSPGERATLSCRSSTGAVTESNYANWVQEKPGQAFRGLIGGANKRAPGVPARFSGSLSGDEATLTISSLQSEDFAVYYCALFYSNTWVFGQGTKLEIK

SEQ ID NO: 151 - protein, 125 aa, synthetic construct
  REGION 1..125: note = Description of Artificial Sequence: Synthetic polypeptide
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 152 - protein, 109 aa, synthetic construct
  REGION 1..109: note = Description of Artificial Sequence: Synthetic polypeptide
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 153 - protein, 119 aa, synthetic construct
  REGION 1..119: note = Description of Artificial Sequence: Synthetic polypeptide
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 154 - protein, 112 aa, synthetic construct
  REGION 1..112: note = Description of Artificial Sequence: Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 155 - protein, 119 aa, synthetic construct
  REGION 1..119: note = Description of Artificial Sequence: Synthetic polypeptide
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 156 - protein, 112 aa, synthetic construct
  REGION 1..112: note = Description of Artificial Sequence: Synthetic polypeptide
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 157 - protein, 119 aa, synthetic construct; identical to SEQ ID NO: 155
  REGION 1..119: note = Description of Artificial Sequence: Synthetic polypeptide

SEQ ID NO: 158 - protein, 112 aa, synthetic construct
  REGION 1..112: note = Description of Artificial Sequence: Synthetic polypeptide
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCTQSFILRTFGQGTKVEIK

SEQ ID NO: 159 - protein, 121 aa, synthetic construct
  REGION 1..121: note = Description of Artificial Sequence: Synthetic polypeptide
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSS

SEQ ID NO: 160 - protein, 106 aa, synthetic construct
  REGION 1..106: note = Description of Artificial Sequence: Synthetic polypeptide
DIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIK

SEQ ID NO: 161 - protein, 119 aa, synthetic construct [sequence omitted]

SEQ ID NO: 162 - protein, 106 aa, synthetic construct [sequence omitted]

SEQ ID NO: 163 - protein, 119 aa, synthetic construct
  REGION 1..119: note = Description of Artificial Sequence: Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 164 - protein, 106 aa, synthetic construct [sequence omitted]

SEQ ID NO: 165 - protein, 119 aa, synthetic construct
  REGION 1..119: note = Description of Artificial Sequence: Synthetic polypeptide
QVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSS

SEQ ID NO: 166 - protein, 106 aa, synthetic construct
  REGION 1..106: note = Description of Artificial Sequence: Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEIN

SEQ ID NO: 167 - protein, 119 aa, synthetic construct
  REGION 1..119: note = Description of Artificial Sequence: Synthetic polypeptide
QVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYSLDYWGQGTTLTVSS

SEQ ID NO: 168 - protein, 106 aa, synthetic construct; identical to SEQ ID NO: 166
  REGION 1..106: note = Description of Artificial Sequence: Synthetic polypeptide

SEQ ID NO: 169 - protein, 119 aa, synthetic construct
  REGION 1..119: note = Description of Artificial Sequence: Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLAVSS

SEQ ID NO: 170 - protein, 106 aa, synthetic construct
  REGION 1..106: note = Description of Artificial Sequence: Synthetic polypeptide
QIVLTCSPAIMSASPGEKVTMTCRASSSVSYMNWYQQKSGTSPKRWIYDTSKVASGVPYRFSGSGSGTSYSLTISSMEAEDAATYYCQQWSSNPLTFGSGTKLEIN

SEQ ID NO: 171 - protein, 119 aa, synthetic construct
  REGION 1..119: note = Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 172 - protein, 106 aa, synthetic construct
  REGION 1..106: note = Description of Artificial Sequence: Synthetic polypeptide
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 173 - protein, 119 aa, synthetic construct
  REGION 1..119: note = Description of Artificial Sequence: Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 174 - protein, 106 aa, synthetic construct
  REGION 1..106: note = Description of Artificial Sequence: Synthetic polypeptide
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 175 - protein, 119 aa, synthetic construct [sequence omitted]

SEQ ID NO: 176 - protein, 106 aa, synthetic construct [sequence omitted]

SEQ ID NO: 177 - protein, 119 aa, synthetic construct
  REGION 1..119: note = Description of Artificial Sequence: Synthetic polypeptide
EVQLVESGGGLVQPGGSLRLSCAASGYTFTRYTMHWVRQAPGKGLEWIGYINPSRGYTYYADSVKGRFTLSTDKSKNTAYLQMSSLRAEDTAVYYCARYYDDHYCLDYWGQGTLVTVSS

SEQ ID NO: 178 - protein, 106 aa, synthetic construct
  REGION 1..106: note = Description of Artificial Sequence: Synthetic polypeptide
DIQLTQSPSSLSASVGDRVTITCRASSSVSYVAWYQQKPGKAPKRWIYDTSKLYSGVPSRFSGSGSGTDYTLTISSLQPEDFATYYCQQWSSNPPTFGQGTKVEIK

SEQ ID NO: 179 - protein, 119 aa, synthetic construct
  REGION 1..119: note = Description of Artificial Sequence: Synthetic polypeptide
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSS

SEQ ID NO: 180 - protein, 106 aa, synthetic construct [sequence omitted]

SEQ ID NO: 181 - protein, 119 aa, synthetic construct
  REGION 1..119: note = Description of Artificial Sequence: Synthetic polypeptide
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSS

SEQ ID NO: 182 - protein, 106 aa, synthetic construct
  REGION 1..106: note = Description of Artificial Sequence: Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQIT

SEQ ID NO: 183 - protein, 119 aa, synthetic construct
  REGION 1..119: note = Description of Artificial Sequence: Synthetic polypeptide
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYCLDYWGQGTPVTVSS

SEQ ID NO: 184 - protein, 106 aa, synthetic construct; identical to SEQ ID NO: 182
  REGION 1..106: note = Description of Artificial Sequence: Synthetic polypeptide

SEQ ID NO: 185 - protein, 122 aa, synthetic construct
  REGION 1..122: note = Description of Artificial Sequence: Synthetic polypeptide
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 186 - protein, 107 aa, synthetic construct
  REGION 1..107: note = Description of Artificial Sequence: Synthetic polypeptide
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 187 - protein, 122 aa, synthetic construct [sequence omitted]

SEQ ID NO: 188 - protein, 107 aa, synthetic construct
  REGION 1..107: note = Description of Artificial Sequence: Synthetic polypeptide
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 189 - protein, 128 aa, synthetic construct
  REGION 1..128: note = Description of Artificial Sequence: Synthetic polypeptide
QLQLQESGPGLVKPSQTLSLTCSVSGGSIRSGGHYWSWIRQHPGKGLEWIGYIHHSGSTYYNPSLKSRVIISVDTSKNQFSLKLRSVTAADTAIYYCARWRHDIFTTYPYYYYGMDVWGQGTLVTVSS

SEQ ID NO: 190 - protein, 107 aa, synthetic construct
  REGION 1..107: note = Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 191 - protein, 128 aa, synthetic construct
  REGION 1..128: note = Description of Artificial Sequence: Synthetic polypeptide
QLQLQESGPGLVKPSQTLSLTCSVSGGSIRSGGHYWSWIRQHPGKGLEWIGYISYSGSTYYNPSLKSRVTISVDTSKNQFSLKLSSVTAADTAVYYCARWRHDILTAYPYYYYGMDVWGQGTLVTVSS

SEQ ID NO: 192 - protein, 107 aa, synthetic construct; identical to SEQ ID NO: 190
  REGION 1..107: note = Description of Artificial Sequence: Synthetic polypeptide

SEQ ID NO: 193 - protein, 123 aa, synthetic construct
  REGION 1..123: note = Description of Artificial Sequence: Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 194 - protein, 107 aa, synthetic construct; identical to SEQ ID NO: 190
  REGION 1..107: note = Description of Artificial Sequence: Synthetic polypeptide

SEQ ID NO: 195 - protein, 123 aa, synthetic construct
  REGION 1..123: note = Description of Artificial Sequence: Synthetic polypeptide
EVQLVESGGGLVQPGRSLRLSCAASGFTFDNYAMHWVRQAPGKGLEWVSGISWNSGSIGYADSVKGRFTISRDNAKNSLYLQMNSLRAEDTALYYCAKDSRGYGDYSRGGAYWGQGTLVTVSS

SEQ ID NO: 196 - protein, 107 aa, synthetic construct; identical to SEQ ID NO: 190
  REGION 1..107: note = Description of Artificial Sequence: Synthetic polypeptide

SEQ ID NO: 197 - protein, 124 aa, synthetic construct
  REGION 1..124: note = Description of Artificial Sequence: Synthetic polypeptide
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 198 - protein, 108 aa, synthetic construct
  REGION 1..108: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 199 - protein, 124 aa, synthetic construct
  REGION 1..124: note = Description of Artificial Sequence: Synthetic polypeptide
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 200 - protein, 108 aa, synthetic construct; identical to SEQ ID NO: 198
  REGION 1..108: note = Description of Artificial Sequence: Synthetic polypeptide

SEQ ID NO: 201 - protein, 10 aa, synthetic construct
  REGION 1..10: note = Description of Artificial Sequence: Synthetic peptide
GGSFSGYYWS

SEQ ID NO: 202 - protein, 10 aa, synthetic construct
  REGION 1..10: note = Description of Artificial Sequence: Synthetic peptide
GGSFSDYYWS

SEQ ID NO: 203 - protein, 10 aa, synthetic construct
  REGION 1..10: note = Description of Artificial Sequence: Synthetic peptide
GGSFSDYYWS

SEQ ID NO: 204 - protein, 10 aa, synthetic construct
  REGION 1..10: note = Description of Artificial Sequence: Synthetic peptide
GGSFSDYYWS

SEQ ID NO: 205 - protein, 10 aa, synthetic construct
  REGION 1..10: note = Description of Artificial Sequence: Synthetic peptide
GGSFSDYYWS

SEQ ID NO: 206 - protein, 10 aa, synthetic construct
  REGION 1..10: note = Description of Artificial Sequence: Synthetic peptide
GGSFSDYYWS

SEQ ID NO: 207 - protein, 10 aa, synthetic construct
  REGION 1..10: note = Description of Artificial Sequence: Synthetic peptide
GGSFSDYYWS

SEQ ID NO: 208 - protein, 10 aa, synthetic construct
  REGION 1..10: note = Description of Artificial Sequence: Synthetic peptide
GGSFSDYYWS

SEQ ID NO: 209 - protein, 10 aa, synthetic construct
  REGION 1..10: note = Description of Artificial Sequence: Synthetic peptide
GGSFSDYYWS

SEQ ID NO: 210 - protein, 10 aa, synthetic construct
  REGION 1..10: note = Description of Artificial Sequence: Synthetic peptide
GGSFSDYYWS

SEQ ID NO: 211 - protein, 10 aa, synthetic construct
  REGION 1..10: note = Description of Artificial Sequence: Synthetic peptide
GGSFSDYYWS

SEQ ID NO: 212 - protein, 10 aa, synthetic construct
  REGION 1..10: note = Description of Artificial Sequence: Synthetic peptide
GGSFSDYYWS

SEQ ID NO: 213 - protein, 10 aa, synthetic construct
  REGION 1..10: note = Description of Artificial Sequence: Synthetic peptide
GGSFSDYYWS

SEQ ID NO: 214 - protein, 10 aa, synthetic construct
  REGION 1..10: note = Description of Artificial Sequence: Synthetic peptide
GGSFSDYYWS

SEQ ID NO: 215 - protein, 10 aa, synthetic construct
  REGION 1..10: note = Description of Artificial Sequence: Synthetic peptide
GGSFSDYYWS

SEQ ID NO: 216 - protein, 10 aa, synthetic construct
  REGION 1..10: note = Description of Artificial Sequence: Synthetic peptide
GGSFSGYYWS

SEQ ID NO: 217 - protein, 10 aa, synthetic construct
  REGION 1..10: note = Description of Artificial Sequence: Synthetic peptide
GGSFSGYYWS

SEQ ID NO: 218 - protein, 10 aa, synthetic construct
  REGION 1..10: note = Description of Artificial Sequence: Synthetic peptide
GGSFSGYYWS

SEQ ID NO: 219 - protein, 10 aa, synthetic construct
  REGION 1..10: note = Description of Artificial Sequence: Synthetic peptide
GGSFSGYYWS

SEQ ID NO: 220 - protein, 10 aa, synthetic construct
  REGION 1..10: note = Description of Artificial Sequence: Synthetic peptide
GGSFSGYYWS

SEQ ID NO: 221 - protein, 10 aa, synthetic construct
  REGION 1..10: note = Description of Artificial Sequence: Synthetic peptide
GGSFSGYYWS

SEQ ID NO: 222 - protein, 10 aa, synthetic construct
  REGION 1..10: note = Description of Artificial Sequence: Synthetic peptide
GGSFSGYYWS

SEQ ID NO: 223 - protein, 10 aa, synthetic construct
  REGION 1..10: note = Description of Artificial Sequence: Synthetic peptide
GGSFSGYYWS

SEQ ID NO: 224 - protein, 10 aa, synthetic construct
  REGION 1..10: note = Description of Artificial Sequence: Synthetic peptide
GGSFSDYYWS

SEQ ID NO: 225 - protein, 10 aa, synthetic construct
  REGION 1..10: note = Description of Artificial Sequence: Synthetic peptide
GGSFSDYYWS

SEQ ID NO: 226 - protein, 10 aa, synthetic construct
  REGION 1..10: note = Description of Artificial Sequence: Synthetic peptide
GGSFSDYYWS

SEQ ID NO: 227 - protein, 10 aa, synthetic construct
  REGION 1..10: note = Description of Artificial Sequence: Synthetic peptide
GGSFSDYYWS

SEQ ID NO: 228 - protein, 10 aa, synthetic construct
  REGION 1..10: note = Description of Artificial Sequence: Synthetic peptide
GGSFSDYYWS

SEQ ID NO: 229 - protein, 10 aa, synthetic construct
  REGION 1..10: note = Description of Artificial Sequence: Synthetic peptide
GGSFSDYYWS

SEQ ID NO: 230 - protein, 10 aa, synthetic construct
  REGION 1..10: note = Description of Artificial Sequence: Synthetic peptide
GGSFSDYYWS

SEQ ID NO: 231 - protein, 10 aa, synthetic construct
  REGION 1..10: note = Description of Artificial Sequence: Synthetic peptide
GGSFSDYYWS

SEQ ID NO: 232 - protein, 10 aa, synthetic construct
  REGION 1..10: note = Description of Artificial Sequence: Synthetic peptide
GGSFSDYYWS

SEQ ID NO: 233 - protein, 10 aa, synthetic construct
  REGION 1..10: note = Description of Artificial Sequence: Synthetic peptide
GGSFSDYYWS

SEQ ID NO: 234 - protein, 10 aa, synthetic construct
  REGION 1..10: note = Description of Artificial Sequence: Synthetic peptide
GGSFSDYYWS

SEQ ID NO: 235 - protein, 10 aa, synthetic construct
  REGION 1..10: note = Description of Artificial Sequence: Synthetic peptide
GGSFSDYYWS

SEQ ID NO: 236 - protein, 10 aa, synthetic construct
  REGION 1..10: note = Description of Artificial Sequence: Synthetic peptide
GGSFSDYYWS

SEQ ID NO: 237 - protein, 10 aa, synthetic construct
  REGION 1..10: note = Description of Artificial Sequence: Synthetic peptide
GGSFSDYYWS

SEQ ID NO: 238 - protein, 10 aa, synthetic construct
  REGION 1..10: note = Description of Artificial Sequence: Synthetic peptide
GGSFSGYYWS

SEQ ID NO: 239 - protein, 10 aa, synthetic construct
  REGION 1..10: note = Description of Artificial Sequence: Synthetic peptide
GGSFSGYYWS

SEQ ID NO: 240 - protein, 10 aa, synthetic construct
  REGION 1..10: note = Description of Artificial Sequence: Synthetic peptide
GGSFSGYYWS

SEQ ID NO: 241 - protein, 10 aa, synthetic construct
  REGION 1..10: note = Description of Artificial Sequence: Synthetic peptide
GGSFSGYYWS

SEQ ID NO: 242 - protein, 10 aa, synthetic construct
  REGION 1..10: note = Description of Artificial Sequence: Synthetic peptide
GGSFSGYYWS

SEQ ID NO: 243 - protein, 10 aa, synthetic construct
  REGION 1..10: note = Description of Artificial Sequence: Synthetic peptide
GGSFSGYYWS

SEQ ID NO: 244 - protein, 10 aa, synthetic construct
  REGION 1..10: note = Description of Artificial Sequence: Synthetic peptide
GGSFSGYYWS

SEQ ID NO: 245 - protein, 10 aa, synthetic construct
  REGION 1..10: note = Description of Artificial Sequence: Synthetic peptide
GGSFSGYYWS

SEQ ID NO: 246 - protein, 10 aa, synthetic construct
  REGION 1..10: note = Description of Artificial Sequence: Synthetic peptide
GGSFSGYYWS

SEQ ID NO: 247 - protein, 10 aa, synthetic construct
  REGION 1..10: note = Description of Artificial Sequence: Synthetic peptide
GGSFSGYYWS

SEQ ID NO: 248 - protein, 10 aa, synthetic construct
  REGION 1..10: note = Description of Artificial Sequence: Synthetic peptide
GGSFSGYYWS

SEQ ID NO: 249 - protein, 10 aa, synthetic construct
  REGION 1..10: note = Description of Artificial Sequence: Synthetic peptide
GGSFSGYYWS

SEQ ID NO: 250 - protein, 10 aa, synthetic construct
  REGION 1..10: note = Description of Artificial Sequence: Synthetic peptide
GGSFSGYYWS

SEQ ID NO: 251 - protein, 10 aa, synthetic construct
  REGION 1..10: note = Description of Artificial Sequence: Synthetic peptide
GGSFSGYYWS

SEQ ID NO: 252 - protein, 10 aa, synthetic construct
  REGION 1..10: note = Description of Artificial Sequence: Synthetic peptide
GGSFSGYYWS

SEQ ID NO: 253 - protein, 10 aa, synthetic construct
  REGION 1..10: note = Description of Artificial Sequence: Synthetic peptide
GGSFSGYYWS

SEQ ID NO: 254 - protein, 10 aa, synthetic construct
  REGION 1..10: note = Description of Artificial Sequence: Synthetic peptide
GGSFSGYYWS

SEQ ID NO: 255 - protein, 10 aa, synthetic construct
  REGION 1..10: note = Description of Artificial Sequence: Synthetic peptide
GGSFSGYYWS

SEQ ID NO: 256 - protein, 10 aa, synthetic construct
  REGION 1..10: note = Description of Artificial Sequence: Synthetic peptide
GGSFSGYYWS

SEQ ID NO: 257 - protein, 10 aa, synthetic construct
  REGION 1..10: note = Description of Artificial Sequence: Synthetic peptide
GGSFSGYYWS

SEQ ID NO: 258 - protein, 10 aa, synthetic construct
  REGION 1..10: note = Description of Artificial Sequence: Synthetic peptide
GGSFSGYYWS

SEQ ID NO: 259 - protein, 10 aa, synthetic construct
  REGION 1..10: note = Description of Artificial Sequence: Synthetic peptide
GGSFSGYYWS

SEQ ID NO: 260 - protein, 10 aa, synthetic construct
  REGION 1..10: note = Description of Artificial Sequence: Synthetic peptide
GGSFSGYYWS

SEQ ID NO: 261 - protein, 10 aa, synthetic construct
  REGION 1..10: note = Description of Artificial Sequence: Synthetic peptide
GGSFSGYYWS

SEQ ID NO: 262 - protein, 10 aa, synthetic construct
  REGION 1..10: note = Description of Artificial Sequence: Synthetic peptide
GGSFSGYYWS

SEQ ID NO: 263 - protein, 10 aa, synthetic construct
  REGION 1..10: note = Description of Artificial Sequence: Synthetic peptide
GGSFSGYYWS

SEQ ID NO: 264 - protein, 10 aa, synthetic construct
  REGION 1..10: note = Description of Artificial Sequence: Synthetic peptide
GGSFSGYYWS

SEQ ID NO: 265 - protein, 10 aa, synthetic construct
  REGION 1..10: note = Description of Artificial Sequence: Synthetic peptide
GGSFSGYYWS

SEQ ID NO: 266 - protein, 19 aa, synthetic construct
  REGION 1..19: note = Description of Artificial Sequence: Synthetic peptide
EVNHRGSINYNNYNPSLKS

SEQ ID NO: 267 - protein, 19 aa, synthetic construct
  REGION 1..19: note = Description of Artificial Sequence: Synthetic peptide
EVNHRGSINYNNYNPSLKS

SEQ ID NO: 268 - protein, 19 aa, synthetic construct
  REGION 1..19: note = Description of Artificial Sequence: Synthetic peptide
EVNHRGSINYNNYNPSLKS

SEQ ID NO: 269 - protein, 19 aa, synthetic construct
  REGION 1..19: note = Description of Artificial Sequence: Synthetic peptide
EINHRGSINYNNYNPSLKS

SEQ ID NO: 270 - protein, 19 aa, synthetic construct
  REGION 1..19: note = Description of Artificial Sequence: Synthetic peptide
EVNHRGSINYNNYNPSLKS

SEQ ID NO: 271 - protein, 19 aa, synthetic construct
  REGION 1..19: note = Description of Artificial Sequence: Synthetic peptide
EVNHRGSINYNNYNPSLKS

SEQ ID NO: 272 - protein, 19 aa, synthetic construct
  REGION 1..19: note = Description of Artificial Sequence: Synthetic peptide
EVNHRGSINYNNYNPSLKS

SEQ ID NO: 273 - protein, 19 aa, synthetic construct
  REGION 1..19: note = Description of Artificial Sequence: Synthetic peptide
EINHRGSINYNNYNPSLKS

SEQ ID NO: 274 - protein, 19 aa, synthetic construct
  REGION 1..19: note = Description of Artificial Sequence: Synthetic peptide
EINHRGSINYNNYNPSLKS

SEQ ID NO: 275 - protein, 19 aa, synthetic construct
  REGION 1..19: note = Description of Artificial Sequence: Synthetic peptide
EVNHRGSINYNNYNPSLKS

SEQ ID NO: 276 - protein, 19 aa, synthetic construct
  REGION 1..19: note = Description of Artificial Sequence: Synthetic peptide
EINHRGSINYNNYNPSLKS

SEQ ID NO: 277 - protein, 19 aa, synthetic construct
  REGION 1..19: note = Description of Artificial Sequence: Synthetic peptide
EINHRGSINYNNYNPSLKS

SEQ ID NO: 278 - protein, 19 aa, synthetic construct
  REGION 1..19: note = Description of Artificial Sequence: Synthetic peptide
EINHRGSINYNNYNPSLKS

SEQ ID NO: 279 - protein, 19 aa, synthetic construct
  REGION 1..19: note = Description of Artificial Sequence: Synthetic peptide
EINHRGSINYNNYNPSLKS

SEQ ID NO: 280 - protein, 19 aa, synthetic construct
  REGION 1..19: note = Description of Artificial Sequence: Synthetic peptide
EINHRGSINYNNYNPSLKS

SEQ ID NO: 281 - protein, 19 aa, synthetic construct
  REGION 1..19: note = Description of Artificial Sequence: Synthetic peptide
EINHRGSINYNNYNPSLKS

SEQ ID NO: 282 - protein, 19 aa, synthetic construct
  REGION 1..19: note = Description of Artificial Sequence: Synthetic peptide
EINHRGSINYNNYNPSLKS

SEQ ID NO: 283 - protein, 19 aa, synthetic construct
  REGION 1..19: note = Description of Artificial Sequence: Synthetic peptide
EINHRGSINYNNYNPSLKS

SEQ ID NO: 284 - protein, 19 aa, synthetic construct
  REGION 1..19: note = Description of Artificial Sequence: Synthetic peptide
EINHRGSINYNNYNPSLKS

SEQ ID NO: 285 - protein, 19 aa, synthetic construct
  REGION 1..19: note = Description of Artificial Sequence: Synthetic peptide
EINHAGSINYNNYNPSLKS

SEQ ID NO: 286 - protein, 19 aa, synthetic construct
  REGION 1..19: note = Description of Artificial Sequence: Synthetic peptide
EINHAGSINYNNYNPSLKS

SEQ ID NO: 287 - protein, 19 aa, synthetic construct
  REGION 1..19: note = Description of Artificial Sequence: Synthetic peptide
EINHQGSINYNNYNPSLKS

SEQ ID NO: 288 - protein, 19 aa, synthetic construct
  REGION 1..19: note = Description of Artificial Sequence: Synthetic peptide
EINHQGSINYNNYNPSLKS

SEQ ID NO: 289 - protein, 19 aa, synthetic construct
  REGION 1..19: note = Description of Artificial Sequence: Synthetic peptide
EINHRGSINYNNYNPSLKS

SEQ ID NO: 290 - protein, 19 aa, synthetic construct
  REGION 1..19: note = Description of Artificial Sequence: Synthetic peptide
EINHRGSINYNNYNPSLKS

SEQ ID NO: 291 - protein, 19 aa, synthetic construct
  REGION 1..19: note = Description of Artificial Sequence: Synthetic peptide
EINHRGSINYNNYNPSLKS

SEQ ID NO: 292 - protein, 19 aa, synthetic construct
  REGION 1..19: note = Description of Artificial Sequence: Synthetic peptide
EINHRGSINYNNYNPSLKS

SEQ ID NO: 293 - protein, 19 aa, synthetic construct
  REGION 1..19: note = Description of Artificial Sequence: Synthetic peptide
EINHRGSINYNNYNPSLKS

SEQ ID NO: 294 - protein, 19 aa, synthetic construct
  REGION 1..19: note = Description of Artificial Sequence: Synthetic peptide
EINHRGSINYNNYNPSLKS

SEQ ID NO: 295 - protein, 19 aa, synthetic construct
  REGION 1..19: note = Description of Artificial Sequence: Synthetic peptide
EINHRGSINYNNYNPSLKS

SEQ ID NO: 296 - protein, 19 aa, synthetic construct
  REGION 1..19: note = Description of Artificial Sequence: Synthetic peptide
EINHRGSINYNNYNPSLKS

SEQ ID NO: 297 - protein, 19 aa, synthetic construct
  REGION 1..19: note = Description of Artificial Sequence: Synthetic peptide
EINHRGSINYNNYNPSLKS

SEQ ID NO: 298 - protein, 19 aa, synthetic construct
  REGION 1..19: note = Description of Artificial Sequence: Synthetic peptide
EINHRGSINYNNYNPSLKS

SEQ ID NO: 299 - protein, 19 aa, synthetic construct
  REGION 1..19: note = Description of Artificial Sequence: Synthetic peptide
EINHRGSINYNNYNPSLKS

SEQ ID NO: 300 - protein, 19 aa, synthetic construct
  REGION 1..19: note = Description of Artificial Sequence: Synthetic peptide
EINHRGSINYNNYNPSLKS

SEQ ID NO: 301 - protein, 19 aa, synthetic construct
  REGION 1..19: note = Description of Artificial Sequence: Synthetic peptide
EINHRGSINYNNYNPSLKS

SEQ ID NO: 302 - protein, 19 aa, synthetic construct
  REGION 1..19: note = Description of Artificial Sequence: Synthetic peptide
EINHRGSINYNNYNPSLKS

SEQ ID NO: 303 - protein, 19 aa, synthetic construct
  REGION 1..19: note = Description of Artificial Sequence: Synthetic peptide
EINHRGSINYNNYNPSLKS

SEQ ID NO: 304 - protein, 19 aa, synthetic construct
  REGION 1..19: note = Description of Artificial Sequence: Synthetic peptide
EINHRGSINYNNYNPSLKS

SEQ ID NO: 305 - protein, 19 aa, synthetic construct
  REGION 1..19: note = Description of Artificial Sequence: Synthetic peptide
EINHRGSINYNNYNPSLKS

SEQ ID NO: 306 - protein, 19 aa, synthetic construct
  REGION 1..19: note = Description of Artificial Sequence: Synthetic peptide
EINHRGSINYNNYNPSLKS

SEQ ID NO: 307 - protein, 19 aa, synthetic construct
  REGION 1..19: note = Description of Artificial Sequence: Synthetic peptide
EINHRGSINYNNYNPSLKS

SEQ ID NO: 308 - protein, 19 aa, synthetic construct
  REGION 1..19: note = Description of Artificial Sequence: Synthetic peptide
EINHRGSINYNNYNPSLKS

SEQ ID NO: 309 - protein, 19 aa, synthetic construct
  REGION 1..19: note = Description of Artificial Sequence: Synthetic peptide
EINHRGSINYNNYNPSLKS

SEQ ID NO: 310 - protein, 19 aa, synthetic construct
  REGION 1..19: note = Description of Artificial Sequence: Synthetic peptide
EINHRGSINYNNYNPSLKS

SEQ ID NO: 311 - protein, 19 aa, synthetic construct
  REGION 1..19: note = Description of Artificial Sequence: Synthetic peptide
EINHRGSINYNNYNPSLKS

SEQ ID NO: 312 - protein, 19 aa, synthetic construct
  REGION 1..19: note = Description of Artificial Sequence: Synthetic peptide
EINHRGSINYNNYNPSLKS

SEQ ID NO: 313 - protein, 19 aa, synthetic construct
  REGION 1..19: note = Description of Artificial Sequence: Synthetic peptide
EINHRGSINYNNYNPSLKS

SEQ ID NO: 314 - protein, 19 aa, synthetic construct
  REGION 1..19: note = Description of Artificial Sequence: Synthetic peptide
EINHRGSINYNNYNPSLKS

SEQ ID NO: 315 - protein, 19 aa, synthetic construct
  REGION 1..19: note = Description of Artificial Sequence: Synthetic peptide
EINHRGSINYNNYNPSLKS

SEQ ID NO: 316 - protein, 19 aa, synthetic construct
  REGION 1..19: note = Description of Artificial Sequence: Synthetic peptide
EINHRGSINYNNYNPSLKS

SEQ ID NO: 317 - protein, 19 aa, synthetic construct
  REGION 1..19: note = Description of Artificial Sequence: Synthetic peptide
EINHRGSINYNNYNPSLKS

SEQ ID NO: 318 - protein, 19 aa, synthetic construct
  REGION 1..19: note = Description of Artificial Sequence: Synthetic peptide
EINHRGSINYNNYNPSLKS

SEQ ID NO: 319 - protein, 19 aa, synthetic construct
  REGION 1..19: note = Description of Artificial Sequence: Synthetic peptide
EINHRGSINYNNYNPSLKS

SEQ ID NO: 320 - protein, 19 aa, synthetic construct
  REGION 1..19: note = Description of Artificial Sequence: Synthetic peptide
EINHRGSINYNNYNPSLKS

SEQ ID NO: 321 - protein, 19 aa, synthetic construct
  REGION 1..19: note = Description of Artificial Sequence: Synthetic peptide
EINHRGSINYNNYNPSLKS

SEQ ID NO: 322 - protein, 19 aa, synthetic construct
  REGION 1..19: note = Description of Artificial Sequence: Synthetic peptide
EINHRGSINYNNYNPSLKS

SEQ ID NO: 323 - protein, 19 aa, synthetic construct
  REGION 1..19: note = Description of Artificial Sequence: Synthetic peptide
EINHRGSINYNNYNPSLKS

SEQ ID NO: 324 - protein, 19 aa, synthetic construct
  REGION 1..19: note = Description of Artificial Sequence: Synthetic peptide
EINHRGSINYNNYNPSLKS

SEQ ID NO: 325 - protein, 19 aa, synthetic construct
  REGION 1..19: note = Description of Artificial Sequence: Synthetic peptide
EINHRGSINYNNYNPSLKS

SEQ ID NO: 326 - protein, 19 aa, synthetic construct
  REGION 1..19: note = Description of Artificial Sequence: Synthetic peptide
EINHRGSINYNNYNPSLKS

SEQ ID NO: 327 - protein, 19 aa, synthetic construct
  REGION 1..19: note = Description of Artificial Sequence: Synthetic peptide
EINHRGSINYNNYNPSLKS

SEQ ID NO: 328 - protein, 19 aa, synthetic construct
  REGION 1..19: note = Description of Artificial Sequence: Synthetic peptide
EINHRGSINYNNYNPSLKS

SEQ ID NO: 329 - protein, 19 aa, synthetic construct
  REGION 1..19: note = Description of Artificial Sequence: Synthetic peptide
EINHRGSINYNNYNPSLKS

SEQ ID NO: 330 - protein, 19 aa, synthetic construct
  REGION 1..19: note = Description of Artificial Sequence: Synthetic peptide
EINHRGSINYNNYNPSLKS

SEQ ID NO: 331 - protein, 21 aa, synthetic construct
  REGION 1..21: note = Description of Artificial Sequence: Synthetic peptide
AKPLRPHCTNGVCYSGDAFDI

SEQ ID NO: 332 - protein, 21 aa, synthetic construct
  REGION 1..21: note = Description of Artificial Sequence: Synthetic peptide
AKPLRPHCTNGVCYSGDAFDI

SEQ ID NO: 333 - protein, 21 aa, synthetic construct
  REGION 1..21: note = Description of Artificial Sequence: Synthetic peptide
AKPLRPHCTNGVCYSGDAFDI

SEQ ID NO: 334 - protein, 21 aa, synthetic construct
  REGION 1..21: note = Description of Artificial Sequence: Synthetic peptide
AKPLRPHCTNGVCYSGDAFDI

SEQ ID NO: 335 - protein, 21 aa, synthetic construct
  REGION 1..21: note = Description of Artificial Sequence: Synthetic peptide
AKPLRPHCTNGVCYSGDAFDI

SEQ ID NO: 336 - protein, 21 aa, synthetic construct
  REGION 1..21: note = Description of Artificial Sequence: Synthetic peptide
AKPLRPHCTNGVCYSGDAFDI

SEQ ID NO: 337 - protein, 21 aa, synthetic construct
  REGION 1..21: note = Description of Artificial Sequence: Synthetic peptide
AKPLRPHCTNGVCYSGDAFDI

SEQ ID NO: 338 - protein, 21 aa, synthetic construct
  REGION 1..21: note = Description of Artificial Sequence: Synthetic peptide
AKPLRPHCTNGVCYSGDAFDI

SEQ ID NO: 339 - protein, 21 aa, synthetic construct
  REGION 1..21: note = Description of Artificial Sequence: Synthetic peptide
AKPLRPHCTNGVCYSGDAFDI

SEQ ID NO: 340 - protein, 21 aa, synthetic construct
  REGION 1..21: note = Description of Artificial Sequence: Synthetic peptide
AKPLRPHCTNGVCYSGDAFDI

SEQ ID NO: 341 - protein, 21 aa, synthetic construct
  REGION 1..21: note = Description of Artificial Sequence: Synthetic peptide
AKPLRPHCTNGVCYSGDAFDI

SEQ ID NO: 342 - protein, 21 aa, synthetic construct
  REGION 1..21: note = Description of Artificial Sequence: Synthetic peptide
AKPLRPHCTNGVCYSGDAFDI

SEQ ID NO: 343 - protein, 21 aa, synthetic construct
  REGION 1..21: note = Description of Artificial Sequence: Synthetic peptide
AKPLRPHCTNGVCYSGDAFDI

SEQ ID NO: 344 - protein, 21 aa, synthetic construct
  REGION 1..21: note = Description of Artificial Sequence: Synthetic peptide
AKPLRPHCTNGVCYSGDAFDI

SEQ ID NO: 345 - protein, 21 aa, synthetic construct
  REGION 1..21: note = Description of Artificial Sequence: Synthetic peptide
AKPLRPHCTNGVCYSGDAFDI

SEQ ID NO: 346 - protein, 21 aa, synthetic construct
  REGION 1..21: note = Description of Artificial Sequence: Synthetic peptide
AKPLRPHCTNGVCYSGDAFDI

SEQ ID NO: 347 - protein, 21 aa, synthetic construct
  REGION 1..21: note = Description of Artificial Sequence: Synthetic peptide
AKPLRPHCTNGVCYSGDAFDI

SEQ ID NO: 348 - protein, 21 aa, synthetic construct
  REGION 1..21: note = Description of Artificial Sequence: Synthetic peptide
AKPLRPHCTNGVCYSGDAFDI

SEQ ID NO: 349 - protein, 21 aa, synthetic construct
  REGION 1..21: note = Description of Artificial Sequence: Synthetic peptide
AKPLRPHCTNGVCYSGDAFDI

SEQ ID NO: 350 - protein, 21 aa, synthetic construct
  REGION 1..21: note = Description of Artificial Sequence: Synthetic peptide
AKPLRPHCTNGVCYSGDAFDI

SEQ ID NO: 351 - protein, 21 aa, synthetic construct
  REGION 1..21: note = Description of Artificial Sequence: Synthetic peptide
AKPLRPHCTNGVCYSGDAFDI

SEQ ID NO: 352 - protein, 21 aa, synthetic construct
  REGION 1..21: note = Description of Artificial Sequence: Synthetic peptide
AKPLRPHCTNGVCYSGDAFDI

SEQ ID NO: 353 - protein, 21 aa, synthetic construct
  REGION 1..21: note = Description of Artificial Sequence: Synthetic peptide
AKPLRPHCTNGVCYSGDAFDI

SEQ ID NO: 354 - protein, 21 aa, synthetic construct
  REGION 1..21: note = Description of Artificial Sequence: Synthetic peptide
AKPLRPHCINGVCYSGDAFDI

SEQ ID NO: 355 - protein, 21 aa, synthetic construct
  REGION 1..21: note = Description of Artificial Sequence: Synthetic peptide
AKPLRPHCTNGVCYSGDAFDI

SEQ ID NO: 356 - protein, 21 aa, synthetic construct
  REGION 1..21: note = Description of Artificial Sequence: Synthetic peptide
AKPLRPHCTNGVCYSGDAFDI

SEQ ID NO: 357 - protein, 21 aa, synthetic construct
  REGION 1..21: note = Description of Artificial Sequence: Synthetic peptide
AKPLRPHCTNGVCYSGDAFDI

SEQ ID NO: 358 - protein, 21 aa, synthetic construct
  REGION 1..21: note = Description of Artificial Sequence: Synthetic peptide
AKPLRPHCINGVCYSGDAFDI

SEQ ID NO: 359 - protein, 21 aa, synthetic construct
  REGION 1..21: note = Description of Artificial Sequence: Synthetic peptide
AKPLRPHCTNGVCYSGDAFDI

SEQ ID NO: 360 - protein, 21 aa, synthetic construct
  REGION 1..21: note = Description of Artificial Sequence: Synthetic peptide
AKPLRPHCTNGVCYSGDAFDI

SEQ ID NO: 361 - protein, 21 aa, synthetic construct
  REGION 1..21: note = Description of Artificial Sequence: Synthetic peptide
AKPLRPHCINGVCYSGDAFDI

SEQ ID NO: 362 - protein, 21 aa, synthetic construct
  REGION 1..21: note = Description of Artificial Sequence: Synthetic peptide
AKPLRPHCTNGVCYSGDAFDI

SEQ ID NO: 363 - protein, 21 aa, synthetic construct
  REGION 1..21: note = Description of Artificial Sequence: Synthetic peptide
AKPLRPHCINGVCYSGDAFDI

SEQ ID NO: 364 - protein, 21 aa, synthetic construct
  REGION 1..21: note = Description of Artificial Sequence: Synthetic peptide
AKPLRPHCINGVCYSGDAFDI

SEQ ID NO: 365 - protein, 21 aa, synthetic construct
  REGION 1..21: note = Description of Artificial Sequence: Synthetic peptide
AKPLRPHCTNGVCYSGDAFDI

SEQ ID NO: 366 - protein, 21 aa, synthetic construct
  REGION 1..21: note = Description of Artificial Sequence: Synthetic peptide
AKPLRPHCTNGVCYSGDAFDI

SEQ ID NO: 367 - protein, 21 aa, synthetic construct
  REGION 1..21: note = Description of Artificial Sequence: Synthetic peptide
AKPLRPHCTNGVCYSGDAFDI

SEQ ID NO: 368 - protein, 21 aa, synthetic construct
  REGION 1..21: note = Description of Artificial Sequence: Synthetic peptide
AKPLRPHCINGVCYSGDAFDI

SEQ ID NO: 369 - protein, 21 aa, synthetic construct
  REGION 1..21: note = Description of Artificial Sequence: Synthetic peptide
AKPLRPHCTNGVCYSGDAFDI

SEQ ID NO: 370 - protein, 21 aa, synthetic construct
  REGION 1..21: note = Description of Artificial Sequence: Synthetic peptide
AKPLRPHCTNGVCYSGDAFDI

SEQ ID NO: 371 - protein, 21 aa, synthetic construct
  REGION 1..21: note = Description of Artificial Sequence: Synthetic peptide
AKPLRPHCTNGVCYSGDAFDI

SEQ ID NO: 372 - protein, 21 aa, synthetic construct
  REGION 1..21: note = Description of Artificial Sequence: Synthetic peptide
AKPLRPHCINGVCYSGDAFDI

SEQ ID NO: 373 - protein, 21 aa, synthetic construct
  REGION 1..21: note = Description of Artificial Sequence: Synthetic peptide
AKPLRPHCTNGVCYSGDAFDI

SEQ ID NO: 374 - protein, 21 aa, synthetic construct
  REGION 1..21: note = Description of Artificial Sequence: Synthetic peptide
AKPLRPHCTNGVCYSGDAFDI

SEQ ID NO: 375 - protein, 21 aa, synthetic construct
  REGION 1..21: note = Description of Artificial Sequence: Synthetic peptide
AKPLRPHCINGVCYSGDAFDI

SEQ ID NO: 376 - protein, 21 aa, synthetic construct
  REGION 1..21: note = Description of Artificial Sequence: Synthetic peptide
AKPLRPHCTNGVCYSGDAFDI

SEQ ID NO: 377 - protein, 21 aa, synthetic construct
  REGION 1..21: note = Description of Artificial Sequence: Synthetic peptide
AKPLRPHCINGVCYSGDAFDI

SEQ ID NO: 378 - protein, 21 aa, synthetic construct
  REGION 1..21: note = Description of Artificial Sequence: Synthetic peptide
AKPLRPHCINGVCYSGDAFDI

SEQ ID NO: 379 - protein, 21 aa, synthetic construct
  REGION 1..21: note = Description of Artificial Sequence: Synthetic peptide
AKPLRPHCTNGVCYSGDAFDI

SEQ ID NO: 380 - protein, 21 aa, synthetic construct
  REGION 1..21: note = Description of Artificial Sequence: Synthetic peptide
AKPLRPHCTNGVCYSGDAFDI

SEQ ID NO: 381 - protein, 21 aa, synthetic construct
  REGION 1..21: note = Description of Artificial Sequence: Synthetic peptide
AKPLRPHCTNGVCYSGDAFDI

SEQ ID NO: 382 - protein, 21 aa, synthetic construct
  REGION 1..21: note = Description of Artificial Sequence: Synthetic peptide
AKPLRPHCINGVCYSGDAFDI

SEQ ID NO: 383 - protein, 21 aa, synthetic construct
  REGION 1..21: note = Description of Artificial Sequence: Synthetic peptide
AKPLRPHCTNGVCYSGDAFDI

SEQ ID NO: 384 - protein, 21 aa, synthetic construct
  REGION 1..21: note = Description of Artificial Sequence: Synthetic peptide
AKPLRPHCTNGVCYSGDAFDI

SEQ ID NO: 385 - protein, 21 aa, synthetic construct
  REGION 1..21: note = Description of Artificial Sequence: Synthetic peptide
AKPLRPHCTNGVCYSGDAFDI

SEQ ID NO: 386 - protein, 21 aa, synthetic construct
  REGION 1..21: note = Description of Artificial Sequence: Synthetic peptide
AKPLRPHCINGVCYSGDAFDI

SEQ ID NO: 387 - protein, 21 aa, synthetic construct
  REGION 1..21: note = Description of Artificial Sequence: Synthetic peptide
AKPLRPHCTNGVCYSGDAFDI

SEQ ID NO: 388 - protein, 21 aa, synthetic construct
  REGION 1..21: note = Description of Artificial Sequence: Synthetic peptide
AKPLRPHCTNGVCYSGDAFDI

SEQ ID NO: 389 - protein, 21 aa, synthetic construct
  REGION 1..21: note = Description of Artificial Sequence: Synthetic peptide
AKPLRPHCINGVCYSGDAFDI

SEQ ID NO: 390 - protein, 21 aa, synthetic construct
  REGION 1..21: note = Description of Artificial Sequence: Synthetic peptide
AKPLRPHCTNGVCYSGDAFDI

SEQ ID NO: 391 - protein, 21 aa, synthetic construct
  REGION 1..21: note = Description of Artificial Sequence: Synthetic peptide
AKPLRPHCINGVCYSGDAFDI

SEQ ID NO: 392 - protein, 21 aa, synthetic construct
  REGION 1..21: note = Description of Artificial Sequence: Synthetic peptide
AKPLRPHCINGVCYSGDAFDI

SEQ ID NO: 393 - protein, 21 aa, synthetic construct
  REGION 1..21: note = Description of Artificial Sequence: Synthetic peptide
AKPLRPHCTNGVCYSGDAFDI

SEQ ID NO: 394 - protein, 21 aa, synthetic construct
  REGION 1..21: note = Description of Artificial Sequence: Synthetic peptide
AKPLRPHCTNGVCYSGDAFDI

SEQ ID NO: 395 - protein, 21 aa, synthetic construct
  REGION 1..21: note = Description of Artificial Sequence: Synthetic peptide
AKPLRPHCTNGVCYSGDAFDI

SEQ ID NO: 396 - protein, 11 aa, synthetic construct
  REGION 1..11: note = Description of Artificial Sequence: Synthetic peptide
GGNNIGSKNVH

SEQ ID NO: 397 - protein, 11 aa, synthetic construct
  REGION 1..11: note = Description of Artificial Sequence: Synthetic peptide
GGNNIGSKSVH

SEQ ID NO: 398 - protein, 11 aa, synthetic construct
  REGION 1..11: note = Description of Artificial Sequence: Synthetic peptide
GGNNIGSKNVH

SEQ ID NO: 399 - protein, 11 aa, synthetic construct
  REGION 1..11: note = Description of Artificial Sequence: Synthetic peptide
GGNNIGSKNVH

SEQ ID NO: 400 - protein, 11 aa, synthetic construct
  REGION 1..11: note = Description of Artificial Sequence: Synthetic peptide
GGNNIGSKNVH

SEQ ID NO: 401 - protein, 11 aa, synthetic construct
  REGION 1..11: note = Description of Artificial Sequence: Synthetic peptide
GGNNIGSKNVH

SEQ ID NO: 402 - protein, 11 aa, synthetic construct
  REGION 1..11: note = Description of Artificial Sequence: Synthetic peptide
GGNNIGSKNVH

SEQ ID NO: 403 - protein, 11 aa, synthetic construct
  REGION 1..11: note = Description of Artificial Sequence: Synthetic peptide
GGNNIGSKNVH

SEQ ID NO: 404 - protein, 11 aa, synthetic construct
  REGION 1..11: note = Description of Artificial Sequence: Synthetic peptide
GGNNIGSKNVH

SEQ ID NO: 405 - protein, 11 aa, synthetic construct
  REGION 1..11: note = Description of Artificial Sequence: Synthetic peptide
GGNNIGSKNVH

SEQ ID NO: 406 - protein, 11 aa, synthetic construct
  REGION 1..11: note = Description of Artificial Sequence: Synthetic peptide
GGNNIGSKNVH

SEQ ID NO: 407 - protein, 11 aa, synthetic construct
  REGION 1..11: note = Description of Artificial Sequence: Synthetic peptide
GGNNIGSKNVH

SEQ ID NO: 408 - protein, 11 aa, synthetic construct
  REGION 1..11: note = Description of Artificial Sequence: Synthetic peptide
GGNNIGSKNVH

SEQ ID NO: 409 - protein, 11 aa, synthetic construct
  REGION 1..11: note = Description of Artificial Sequence: Synthetic peptide
GGNNIGSKNVH

SEQ ID NO: 410 - protein, 11 aa, synthetic construct
  REGION 1..11: note = Description of Artificial Sequence: Synthetic peptide
GGNNIGSKNVH

SEQ ID NO: 411 - protein, 11 aa, synthetic construct
  REGION 1..11: note = Description of Artificial Sequence: Synthetic peptide
GGNNIGSKNVH

SEQ ID NO: 412 - protein, 11 aa, synthetic construct
  REGION 1..11: note = Description of Artificial Sequence: Synthetic peptide
GGNNIGSKNVH

SEQ ID NO: 413 - protein, 11 aa, synthetic construct
  REGION 1..11: note = Description of Artificial Sequence: Synthetic peptide
GGNNIGSKNVH

SEQ ID NO: 414 - protein, 11 aa, synthetic construct
  REGION 1..11: note = Description of Artificial Sequence: Synthetic peptide
GGNNIGSKNVH

SEQ ID NO: 415 - protein, 11 aa, synthetic construct
  REGION 1..11: note = Description of Artificial Sequence: Synthetic peptide
GGNNIGSKNVH

SEQ ID NO: 416 - protein, 11 aa, synthetic construct
  REGION 1..11: note = Description of Artificial Sequence: Synthetic peptide
GGNNIGSKNVH

SEQ ID NO: 417 - protein, 11 aa, synthetic construct
  REGION 1..11: note = Description of Artificial Sequence: Synthetic peptide
GGNNIGSKNVH

SEQ ID NO: 418 - protein, 11 aa, synthetic construct
  REGION 1..11: note = Description of Artificial Sequence: Synthetic peptide
GGNNIGSKNVH

SEQ ID NO: 419 - protein, 11 aa, synthetic construct
  REGION 1..11: note = Description of Artificial Sequence: Synthetic peptide
RGNNIGSKNVH

SEQ ID NO: 420 - protein, 11 aa, synthetic construct
  REGION 1..11: note = Description of Artificial Sequence: Synthetic peptide
RGNNIGYKNVH

SEQ ID NO: 421 - protein, 11 aa, synthetic construct
  REGION 1..11: note = Description of Artificial Sequence: Synthetic peptide
RGNNIGSMNVH

SEQ ID NO: 422 - protein, 11 aa, synthetic construct
  REGION 1..11: note = Description of Artificial Sequence: Synthetic peptide
RGNNIGSKNVH

SEQ ID NO: 423 - protein, 11 aa, synthetic construct
  REGION 1..11: note = Description of Artificial Sequence: Synthetic peptide
GGNNIGYKNVH

SEQ ID NO: 424 - protein, 11 aa, synthetic construct
  REGION 1..11: note = Description of Artificial Sequence: Synthetic peptide
GGNNIGYMNVH

SEQ ID NO: 425 - protein, 11 aa, synthetic construct
  REGION 1..11: note = Description of Artificial Sequence: Synthetic peptide
GGNNIGYKNVH

SEQ ID NO: 426 - protein, 11 aa, synthetic construct
  REGION 1..11: note = Description of Artificial Sequence: Synthetic peptide
GGNNIGSMNVH

SEQ ID NO: 427 - protein, 11 aa, synthetic construct
  REGION 1..11: note = Description of Artificial Sequence: Synthetic peptide
GGNNIGSMNVH

SEQ ID NO: 428 - protein, 11 aa, synthetic construct
  REGION 1..11: note = Description of Artificial Sequence: Synthetic peptide
GGNNIGSKNVH

SEQ ID NO: 429 - protein, 11 aa, synthetic construct
  REGION 1..11: note = Description of Artificial Sequence: Synthetic peptide
GGNNIGYKNVH

SEQ ID NO: 430 - protein, 11 aa, synthetic construct
  REGION 1..11: note = Description of Artificial Sequence: Synthetic peptide
GGNNIGSKNVH

SEQ ID NO: 431 - protein, 11 aa, synthetic construct
  REGION 1..11: note = Description of Artificial Sequence: Synthetic peptide
GGNNIGSKNVH

SEQ ID NO: 432 - protein, 11 aa, synthetic construct
  REGION 1..11: note = Description of Artificial Sequence: Synthetic peptide
GGNNIGYKNVH

SEQ ID NO: 433 - protein, 11 aa, synthetic construct
  REGION 1..11: note = Description of Artificial Sequence: Synthetic peptide
RGNNIGSKNVH

SEQ ID NO: 434 - protein, 11 aa, synthetic construct
  REGION 1..11: note = Description of Artificial Sequence: Synthetic peptide
RGNNIGYKNVH

SEQ ID NO: 435 - protein, 11 aa, synthetic construct
  REGION 1..11: note = Description of Artificial Sequence: Synthetic peptide
RGNNIGSMNVH

SEQ ID NO: 436 - protein, 11 aa, synthetic construct
  REGION 1..11: note = Description of Artificial Sequence: Synthetic peptide
RGNNIGSKNVH

SEQ ID NO: 437 - protein, 11 aa, synthetic construct
  REGION 1..11: note = Description of Artificial Sequence: Synthetic peptide
GGNNIGYKNVH

SEQ ID NO: 438 - protein, 11 aa, synthetic construct
  REGION 1..11: note = Description of Artificial Sequence: Synthetic peptide
GGNNIGYMNVH

SEQ ID NO: 439 - protein, 11 aa, synthetic construct
  REGION 1..11: note = Description of Artificial Sequence: Synthetic peptide
GGNNIGYKNVH

SEQ ID NO: 440 - protein, 11 aa, synthetic construct
  REGION 1..11: note = Description of Artificial Sequence: Synthetic peptide
GGNNIGSMNVH

SEQ ID NO: 441 - protein, 11 aa, synthetic construct
  REGION 1..11: note = Description of Artificial Sequence: Synthetic peptide
GGNNIGSMNVH

SEQ ID NO: 442 - protein, 11 aa, synthetic construct
  REGION 1..11: note = Description of Artificial Sequence: Synthetic peptide
GGNNIGSKNVH

SEQ ID NO: 443 - protein, 11 aa, synthetic construct
  REGION 1..11: note = Description of Artificial Sequence: Synthetic peptide
GGNNIGYKNVH

SEQ ID NO: 444 - protein, 11 aa, synthetic construct
  REGION 1..11: note = Description of Artificial Sequence: Synthetic peptide
GGNNIGSKNVH

SEQ ID NO: 445 - protein, 11 aa, synthetic construct
  REGION 1..11: note = Description of Artificial Sequence: Synthetic peptide
GGNNIGSKNVH

SEQ ID NO: 446 - protein, 11 aa, synthetic construct
  REGION 1..11: note = Description of Artificial Sequence: Synthetic peptide
GGNNIGYKNVH

SEQ ID NO: 447 - protein, 11 aa, synthetic construct
  REGION 1..11: note = Description of Artificial Sequence: Synthetic peptide
RGNNIGSKNVH

SEQ ID NO: 448 - protein, 11 aa, synthetic construct
  REGION 1..11: note = Description of Artificial Sequence: Synthetic peptide
RGNNIGYKNVH

SEQ ID NO: 449 - protein, 11 aa, synthetic construct
  REGION 1..11: note = Description of Artificial Sequence: Synthetic peptide
RGNNIGSMNVH

SEQ ID NO: 450 - protein, 11 aa, synthetic construct
  REGION 1..11: note = Description of Artificial Sequence: Synthetic peptide
RGNNIGSKNVH

SEQ ID NO: 451 - protein, 11 aa, synthetic construct
  REGION 1..11: note = Description of Artificial Sequence: Synthetic peptide
GGNNIGYKNVH

SEQ ID NO: 452 - protein, 11 aa, synthetic construct
  REGION 1..11: note = Description of Artificial Sequence: Synthetic peptide
GGNNIGYMNVH

SEQ ID NO: 453 - protein, 11 aa, synthetic construct
  REGION 1..11: note = Description of Artificial Sequence: Synthetic peptide
GGNNIGYKNVH

SEQ ID NO: 454 - protein, 11 aa, synthetic construct
  REGION 1..11: note = Description of Artificial Sequence: Synthetic peptide
GGNNIGSMNVH

SEQ ID NO: 455 - protein, 11 aa, synthetic construct
  REGION 1..11: note = Description of Artificial Sequence: Synthetic peptide
GGNNIGSMNVH

SEQ ID NO: 456 - protein, 11 aa, synthetic construct
  REGION 1..11: note = Description of Artificial Sequence: Synthetic peptide
GGNNIGSKNVH

SEQ ID NO: 457 - protein, 11 aa, synthetic construct
  REGION 1..11: note = Description of Artificial Sequence: Synthetic peptide
GGNNIGYKNVH

SEQ ID NO: 458 - protein, 11 aa, synthetic construct
  REGION 1..11: note = Description of Artificial Sequence: Synthetic peptide
GGNNIGSKNVH

SEQ ID NO: 459 - protein, 11 aa, synthetic construct
  REGION 1..11: note = Description of Artificial Sequence: Synthetic peptide
GGNNIGSKNVH

SEQ ID NO: 460 - protein, 11 aa, synthetic construct
  REGION 1..11: note = Description of Artificial Sequence: Synthetic peptide
GGNNIGYKNVH

SEQ ID NO: 461 - protein, 7 aa, synthetic construct
  REGION 1..7: note = Description of Artificial Sequence: Synthetic peptide
DDSDRPS

SEQ ID NO: 462 - protein, 7 aa, synthetic construct
  REGION 1..7: note = Description of Artificial Sequence: Synthetic peptide
DDSDRPS

SEQ ID NO: 463 - protein, 7 aa, synthetic construct
  REGION 1..7: note = Description of Artificial Sequence: Synthetic peptide
DDSDRPS

SEQ ID NO: 464 - protein, 7 aa, synthetic construct
  REGION 1..7: note = Description of Artificial Sequence: Synthetic peptide
DDSDRPS

SEQ ID NO: 465 - protein, 7 aa, synthetic construct
  REGION 1..7: note = Description of Artificial Sequence: Synthetic peptide
DDSDRPS

SEQ ID NO: 466 - protein, 7 aa, synthetic construct
  REGION 1..7: note = Description of Artificial Sequence: Synthetic peptide
DDSDRPS

SEQ ID NO: 467 - protein, 7 aa, synthetic construct
  REGION 1..7: note = Description of Artificial Sequence: Synthetic peptide
DDSDRPS

SEQ ID NO: 468 - protein, 7 aa, synthetic construct
  REGION 1..7: note = Description of Artificial Sequence: Synthetic peptide
DDSDRPS

SEQ ID NO: 469 - protein, 7 aa, synthetic construct
  REGION 1..7: note = Description of Artificial Sequence: Synthetic peptide
DDSDRPS

SEQ ID NO: 470 - protein, 7 aa, synthetic construct
  REGION 1..7: note = Description of Artificial Sequence: Synthetic peptide
DDSDRPS

SEQ ID NO: 471 - protein, 7 aa, synthetic construct
  REGION 1..7: note = Description of Artificial Sequence: Synthetic peptide
DDSDRPS

SEQ ID NO: 472 - protein, 7 aa, synthetic construct
  REGION 1..7: note = Description of Artificial Sequence: Synthetic peptide
DDSDRPS

SEQ ID NO: 473 - protein, 7 aa, synthetic construct
  REGION 1..7: note = Description of Artificial Sequence: Synthetic peptide
DDSDRPS

SEQ ID NO: 474 - protein, 7 aa, synthetic construct
  REGION 1..7: note = Description of Artificial Sequence: Synthetic peptide
DDSDRPS

SEQ ID NO: 475 - protein, 7 aa, synthetic construct
  REGION 1..7: note = Description of Artificial Sequence: Synthetic peptide
DDSDRPS

SEQ ID NO: 476 - protein, 7 aa, synthetic construct
  REGION 1..7: note = Description of Artificial Sequence: Synthetic peptide
DDSDRPS

SEQ ID NO: 477 - protein, 7 aa, synthetic construct
  REGION 1..7: note = Description of Artificial Sequence: Synthetic peptide
DDSDRPS

SEQ ID NO: 478 - protein, 7 aa, synthetic construct
  REGION 1..7: note = Description of Artificial Sequence: Synthetic peptide
DDSDRPS

SEQ ID NO: 479 - protein, 7 aa, synthetic construct
  REGION 1..7: note = Description of Artificial Sequence: Synthetic peptide
DDSDRPS

SEQ ID NO: 480 - protein, 7 aa, synthetic construct
  REGION 1..7: note = Description of Artificial Sequence: Synthetic peptide
DDSDRPS

SEQ ID NO: 481 - protein, 7 aa, synthetic construct
  REGION 1..7: note = Description of Artificial Sequence: Synthetic peptide
DDSDRPS

SEQ ID NO: 482 - protein, 7 aa, synthetic construct
  REGION 1..7: note = Description of Artificial Sequence: Synthetic peptide
DDSDRPS

SEQ ID NO: 483 - protein, 7 aa, synthetic construct
  REGION 1..7: note = Description of Artificial Sequence: Synthetic peptide
DDSDRPS

SEQ ID NO: 484 - protein, 7 aa, synthetic construct
  REGION 1..7: note = Description of Artificial Sequence: Synthetic peptide
DDSDRPS

SEQ ID NO: 485 - protein, 7 aa, synthetic construct
  REGION 1..7: note = Description of Artificial Sequence: Synthetic peptide
DDSDRPS

SEQ ID NO: 486 - protein, 7 aa, synthetic construct
  REGION 1..7: note = Description of Artificial Sequence: Synthetic peptide
DDSDRPS

SEQ ID NO: 487 - protein, 7 aa, synthetic construct
  REGION 1..7: note = Description of Artificial Sequence: Synthetic peptide
DDSDRPS

SEQ ID NO: 488 - protein, 7 aa, synthetic construct
  REGION 1..7: note = Description of Artificial Sequence: Synthetic peptide
DDSDRPS

SEQ ID NO: 489 - protein, 7 aa, synthetic construct
  REGION 1..7: note = Description of Artificial Sequence: Synthetic peptide
DDSDRPS

SEQ ID NO: 490 - protein, 7 aa, synthetic construct
  REGION 1..7: note = Description of Artificial Sequence: Synthetic peptide
DDSDRPS

SEQ ID NO: 491 - protein, 7 aa, synthetic construct
  REGION 1..7: note = Description of Artificial Sequence: Synthetic peptide
DDSDRPS

SEQ ID NO: 492 - protein, 7 aa, synthetic construct
  REGION 1..7: note = Description of Artificial Sequence: Synthetic peptide
DDSDRPS

SEQ ID NO: 493 - protein, 7 aa, synthetic construct
  REGION 1..7: note = Description of Artificial Sequence: Synthetic peptide
DDSDRPS

SEQ ID NO: 494 - protein, 7 aa, synthetic construct
  REGION 1..7: note = Description of Artificial Sequence: Synthetic peptide
DDSDRPS

SEQ ID NO: 495 - protein, 7 aa, synthetic construct
  REGION 1..7: note = Description of Artificial Sequence: Synthetic peptide
DDSDRPS

SEQ ID NO: 496 - protein, 7 aa, synthetic construct
  REGION 1..7: note = Description of Artificial Sequence: Synthetic peptide
DDSDRPS

SEQ ID NO: 497 - protein, 7 aa, synthetic construct
  REGION 1..7: note = Description of Artificial Sequence: Synthetic peptide
DDSDRPS

SEQ ID NO: 498 - protein, 7 aa, synthetic construct
  REGION 1..7: note = Description of Artificial Sequence: Synthetic peptide
DDSDRPS

SEQ ID NO: 499 - protein, 7 aa, synthetic construct
  REGION 1..7: note = Description of Artificial Sequence: Synthetic peptide
DDSDRPS

SEQ ID NO: 500 - protein, 7 aa, synthetic construct
  REGION 1..7: note = Description of Artificial Sequence: Synthetic peptide
DDSDRPS

SEQ ID NO: 501 - protein, 7 aa, synthetic construct
  REGION 1..7: note = Description of Artificial Sequence: Synthetic peptide
DDSDRPS

SEQ ID NO: 502 - protein, 7 aa, synthetic construct
  REGION 1..7: note = Description of Artificial Sequence: Synthetic peptide
DDSDRPS

SEQ ID NO: 503 - protein, 7 aa, synthetic construct
  REGION 1..7: note = Description of Artificial Sequence: Synthetic peptide
DDSDRPS

SEQ ID NO: 504 - protein, 7 aa, synthetic construct
  REGION 1..7: note = Description of Artificial Sequence: Synthetic peptide
DDSDRPS

SEQ ID NO: 505 - protein, 7 aa, synthetic construct
  REGION 1..7: note = Description of Artificial Sequence: Synthetic peptide
DDSDRPS

SEQ ID NO: 506 - protein, 7 aa, synthetic construct
  REGION 1..7: note = Description of Artificial Sequence: Synthetic peptide
DDSDRPS

SEQ ID NO: 507 - protein, 7 aa, synthetic construct
  REGION 1..7: note = Description of Artificial Sequence: Synthetic peptide
DDSDRPS

SEQ ID NO: 508 - protein, 7 aa, synthetic construct
  REGION 1..7: note = Description of Artificial Sequence: Synthetic peptide
DDSDRPS

SEQ ID NO: 509 - protein, 7 aa, synthetic construct
  REGION 1..7: note = Description of Artificial Sequence: Synthetic peptide
DDSDRPS

SEQ ID NO: 510 - protein, 7 aa, synthetic construct
  REGION 1..7: note = Description of Artificial Sequence: Synthetic peptide
DDSDRPS

SEQ ID NO: 511 - protein, 7 aa, synthetic construct
  REGION 1..7: note = Description of Artificial Sequence: Synthetic peptide
DDSDRPS

SEQ ID NO: 512 - protein, 7 aa, synthetic construct
  REGION 1..7: note = Description of Artificial Sequence: Synthetic peptide
DDSDRPS

SEQ ID NO: 513 - protein, 7 aa, synthetic construct
  REGION 1..7: note = Description of Artificial Sequence: Synthetic peptide
DDSDRPS

SEQ ID NO: 514 - protein, 7 aa, synthetic construct
  REGION 1..7: note = Description of Artificial Sequence: Synthetic peptide
DDSDRPS

SEQ ID NO: 515 - protein, 7 aa, synthetic construct
  REGION 1..7: note = Description of Artificial Sequence: Synthetic peptide
DDSDRPS

SEQ ID NO: 516 - protein, 7 aa, synthetic construct
  REGION 1..7: note = Description of Artificial Sequence: Synthetic peptide
DDSDRPS

SEQ ID NO: 517 - protein, 7 aa, synthetic construct
  REGION 1..7: note = Description of Artificial Sequence: Synthetic peptide
DDSDRPS

SEQ ID NO: 518 - protein, 7 aa, synthetic construct
  REGION 1..7: note = Description of Artificial Sequence: Synthetic peptide
DDSDRPS

SEQ ID NO: 519 - protein, 7 aa, synthetic construct
  REGION 1..7: note = Description of Artificial Sequence: Synthetic peptide
DDSDRPS

SEQ ID NO: 520 - protein, 7 aa, synthetic construct
  REGION 1..7: note = Description of Artificial Sequence: Synthetic peptide
DDSDRPS

SEQ ID NO: 521 - protein, 7 aa, synthetic construct
  REGION 1..7: note = Description of Artificial Sequence: Synthetic peptide
DDSDRPS

SEQ ID NO: 522 - protein, 7 aa, synthetic construct
  REGION 1..7: note = Description of Artificial Sequence: Synthetic peptide
DDSDRPS

SEQ ID NO: 523 - protein, 7 aa, synthetic construct
  REGION 1..7: note = Description of Artificial Sequence: Synthetic peptide
DDSDRPS

SEQ ID NO: 524 - protein, 7 aa, synthetic construct
  REGION 1..7: note = Description of Artificial Sequence: Synthetic peptide
DDSDRPS

SEQ ID NO: 525 - protein, 7 aa, synthetic construct
  REGION 1..7: note = Description of Artificial Sequence: Synthetic peptide
DDSDRPS

SEQ ID NO: 526 - protein, 11 aa, synthetic construct
  REGION 1..11: note = Description of Artificial Sequence: Synthetic peptide
QVWDSSSDHLV

SEQ ID NO: 527 - protein, 11 aa, synthetic construct
  REGION 1..11: note = Description of Artificial Sequence: Synthetic peptide
QVWDSSSDHLV

SEQ ID NO: 528 - protein, 11 aa, synthetic construct
  REGION 1..11: note = Description of Artificial Sequence: Synthetic peptide
QVWDSSSDHVV

SEQ ID NO: 529 - protein, 11 aa, synthetic construct
  REGION 1..11: note = Description of Artificial Sequence: Synthetic peptide
QVWDSSSDHLV

SEQ ID NO: 530 - protein, 11 aa, synthetic construct
  REGION 1..11: note = Description of Artificial Sequence: Synthetic peptide
QVWDSSSDHLV

SEQ ID NO: 531 - protein, 11 aa, synthetic construct
  REGION 1..11: note = Description of Artificial Sequence: Synthetic peptide
QVWDSSSDHLV

SEQ ID NO: 532 - protein, 11 aa, synthetic construct
  REGION 1..11: note = Description of Artificial Sequence: Synthetic peptide
QVWDSSSDHLV

SEQ ID NO: 533 - protein, 11 aa, synthetic construct
  REGION 1..11: note = Description of Artificial Sequence: Synthetic peptide
QVWDSSSDHLV

SEQ ID NO: 534 - protein, 11 aa, synthetic construct
  REGION 1..11: note = Description of Artificial Sequence: Synthetic peptide
QVWDSSSDHLV

SEQ ID NO: 535 - protein, 11 aa, synthetic construct
  REGION 1..11: note = Description of Artificial Sequence: Synthetic peptide
QVWDSSSDHLV

SEQ ID NO: 536 - protein, 11 aa, synthetic construct
  REGION 1..11: note = Description of Artificial Sequence: Synthetic peptide
QVWDSSSDHLV

SEQ ID NO: 537 - protein, 11 aa, synthetic construct
  REGION 1..11: note = Description of Artificial Sequence: Synthetic peptide
QVWDSSSDHLV

SEQ ID NO: 538 - protein, 11 aa, synthetic construct
  REGION 1..11: note = Description of Artificial Sequence: Synthetic peptide
QVWDSSSDHVV

SEQ ID NO: 539 - protein, 11 aa, synthetic construct
  REGION 1..11: note = Description of Artificial Sequence: Synthetic peptide
QVWDSSSDHLV

SEQ ID NO: 540 - protein, 11 aa, synthetic construct
  REGION 1..11: note = Description of Artificial Sequence: Synthetic peptide
QVWDSSSDHVV

SEQ ID NO: 541 - protein, 11 aa, synthetic construct
  REGION 1..11: note = Description of Artificial Sequence: Synthetic peptide
QVWDSSSDHLV

SEQ ID NO: 542 - protein, 11 aa, synthetic construct
  REGION 1..11: note = Description of Artificial Sequence: Synthetic peptide
QVWDSSSDHVV

SEQ ID NO: 543 - protein, 11 aa, synthetic construct
  REGION 1..11: note = Description of Artificial Sequence: Synthetic peptide
QVWDSSSDHLV

SEQ ID NO: 544 - protein, 11 aa, synthetic construct
  REGION 1..11: note = Description of Artificial Sequence: Synthetic peptide
QVWDSSSDHVV

SEQ ID NO: 545 - protein, 11 aa, synthetic construct
  REGION 1..11: note = Description of Artificial Sequence: Synthetic peptide
QVWDSSSDHVV

SEQ ID NO: 546 - protein, 11 aa, synthetic construct
  REGION 1..11: note = Description of Artificial Sequence: Synthetic peptide
QVWDSSSDHVV

SEQ ID NO: 547 - protein, 11 aa, synthetic construct
  REGION 1..11: note = Description of Artificial Sequence: Synthetic peptide
QVWDSSSDHVV

SEQ ID NO: 548 - protein, 11 aa, synthetic construct
  REGION 1..11: note = Description of Artificial Sequence: Synthetic peptide
QVWDSSSDHVV

SEQ ID NO: 549 - protein, 11 aa, synthetic construct
  REGION 1..11: note = Description of Artificial Sequence: Synthetic peptide
QVWDSSSDHVV

SEQ ID NO: 550 - protein, 11 aa, synthetic construct
  REGION 1..11: note = Description of Artificial Sequence: Synthetic peptide
QVWDSSSDHVV

SEQ ID NO: 551 - protein, 11 aa, synthetic construct
  REGION 1..11: note = Description of Artificial Sequence: Synthetic peptide
QVWDSSSDHVV

SEQ ID NO: 552 - protein, 11 aa, synthetic construct
  REGION 1..11: note = Description of Artificial Sequence: Synthetic peptide
QVWDHSSDHVV

SEQ ID NO: 553 - protein, 11 aa, synthetic construct
  REGION 1..11: note = Description of Artificial Sequence: Synthetic peptide
QVWDSSSDHVV

SEQ ID NO: 554 - protein, 11 aa, synthetic construct
  REGION 1..11: note = Description of Artificial Sequence: Synthetic peptide
QVWDSSSDHVV

SEQ ID NO: 555 - protein, 11 aa, synthetic construct
  REGION 1..11: note = Description of Artificial Sequence: Synthetic peptide
QVWDHSSDHVV

SEQ ID NO: 556 - protein, 11 aa, synthetic construct
  REGION 1..11: note = Description of Artificial Sequence: Synthetic peptide
QVWDSSSDHVV

SEQ ID NO: 557 - protein, 11 aa, synthetic construct
  REGION 1..11: note = Description of Artificial Sequence: Synthetic peptide
QVWDHSSDHVV

SEQ ID NO: 558 - protein, 11 aa, synthetic construct
  REGION 1..11: note = Description of Artificial Sequence: Synthetic peptide
QVWDHSSDHVV

SEQ ID NO: 559 - protein, 11 aa, synthetic construct
  REGION 1..11: note = Description of Artificial Sequence: Synthetic peptide
QVWDHSSDHVV

SEQ ID NO: 560 - protein, 11 aa, synthetic construct
  REGION 1..11: note = Description of Artificial Sequence: Synthetic peptide
QVWDSRSDHVV

SEQ ID NO: 561 - protein, 11 aa, synthetic construct
  REGION 1..11: note = Description of Artificial Sequence: Synthetic peptide
QVWDSESDHVV

SEQ ID NO: 562 - protein, 11 aa, synthetic construct
  REGION 1..11: note = Description of Artificial Sequence: Synthetic peptide
QVWDSESDHVV

SEQ ID NO: 563 - protein, 11 aa, synthetic construct
  REGION 1..11: note = Description of Artificial Sequence: Synthetic peptide
QVWDSSSDHVV

SEQ ID NO: 564 - protein, 11 aa, synthetic construct
  REGION 1..11: note = Description of Artificial Sequence: Synthetic peptide
QVWDSSSDHVV

SEQ ID NO: 565 - protein, 11 aa, synthetic construct
  REGION 1..11: note = Description of Artificial Sequence: Synthetic peptide
QVWDSSSDHVV

SEQ ID NO: 566 - protein, 11 aa, synthetic construct
  REGION 1..11: note = Description of Artificial Sequence: Synthetic peptide
QVWDHSSDHVV

SEQ ID NO: 567 - protein, 11 aa, synthetic construct
  REGION 1..11: note = Description of Artificial Sequence: Synthetic peptide
QVWDSSSDHVV

SEQ ID NO: 568 - protein, 11 aa, synthetic construct
  REGION 1..11: note = Description of Artificial Sequence: Synthetic peptide
QVWDSSSDHVV

SEQ ID NO: 569 - protein, 11 aa, synthetic construct
  REGION 1..11: note = Description of Artificial Sequence: Synthetic peptide
QVWDHSSDHVV

SEQ ID NO: 570 - protein, 11 aa, synthetic construct
  REGION 1..11: note = Description of Artificial Sequence: Synthetic peptide
QVWDSSSDHVV

SEQ ID NO: 571 - protein, 11 aa, synthetic construct
  REGION 1..11: note = Description of Artificial Sequence: Synthetic peptide
QVWDHSSDHVV

SEQ ID NO: 572 - protein, 11 aa, synthetic construct
  REGION 1..11: note = Description of Artificial Sequence: Synthetic peptide
QVWDHSSDHVV

SEQ ID NO: 573 - protein, 11 aa, synthetic construct
  REGION 1..11: note = Description of Artificial Sequence: Synthetic peptide
QVWDHSSDHVV

SEQ ID NO: 574 - protein, 11 aa, synthetic construct
  REGION 1..11: note = Description of Artificial Sequence: Synthetic peptide
QVWDSRSDHVV

SEQ ID NO: 575 - protein, 11 aa, synthetic construct
  REGION 1..11: note = Description of Artificial Sequence: Synthetic peptide
QVWDSESDHVV

SEQ ID NO: 576 - protein, 11 aa, synthetic construct
  REGION 1..11: note = Description of Artificial Sequence: Synthetic peptide
QVWDSESDHVV

SEQ ID NO: 577 - protein, 11 aa, synthetic construct
  REGION 1..11: note = Description of Artificial Sequence: Synthetic peptide
QVWDSSSDHVV

SEQ ID NO: 578 - protein, 11 aa, synthetic construct
  REGION 1..11: note = Description of Artificial Sequence: Synthetic peptide
QVWDSSSDHVV

SEQ ID NO: 579 - protein, 11 aa, synthetic construct
  REGION 1..11: note = Description of Artificial Sequence: Synthetic peptide
QVWDSSSDHVV

SEQ ID NO: 580 - protein, 11 aa, synthetic construct
  REGION 1..11: note = Description of Artificial Sequence: Synthetic peptide
QVWDHSSDHVV

SEQ ID NO: 581 - protein, 11 aa, synthetic construct
  REGION 1..11: note = Description of Artificial Sequence: Synthetic peptide
QVWDSSSDHVV

SEQ ID NO: 582 - protein, 11 aa, synthetic construct
  REGION 1..11: note = Description of Artificial Sequence: Synthetic peptide
QVWDSSSDHVV

SEQ ID NO: 583 - protein, 11 aa, synthetic construct
  REGION 1..11: note = Description of Artificial Sequence: Synthetic peptide
QVWDHSSDHVV

SEQ ID NO: 584 - protein, 11 aa, synthetic construct
  REGION 1..11: note = Description of Artificial Sequence: Synthetic peptide
QVWDSSSDHVV

SEQ ID NO: 585 - protein, 11 aa, synthetic construct
  REGION 1..11: note = Description of Artificial Sequence: Synthetic peptide
QVWDHSSDHVV

SEQ ID NO: 586 - protein, 11 aa, synthetic construct
  REGION 1..11: note = Description of Artificial Sequence: Synthetic peptide
QVWDHSSDHVV

SEQ ID NO: 587 - protein, 11 aa, synthetic construct
  REGION 1..11: note = Description of Artificial Sequence: Synthetic peptide
QVWDHSSDHVV

SEQ ID NO: 588 - protein, 11 aa, synthetic construct
  REGION 1..11: note = Description of Artificial Sequence: Synthetic peptide
QVWDSRSDHVV

SEQ ID NO: 589 - protein, 11 aa, synthetic construct
  REGION 1..11: note = Description of Artificial Sequence: Synthetic peptide
QVWDSESDHVV

SEQ ID NO: 590 - protein, 11 aa, synthetic construct
  REGION 1..11: note = Description of Artificial Sequence: Synthetic peptide
QVWDSESDHVV

SEQ ID NO: 591 - protein, 130 aa, synthetic construct
  REGION 1..130: note = Description of Artificial Sequence: Synthetic polypeptide
QVQLQQWGAGLLKPSETLSLTCAVYGGSFSGYYWSWIRQPPGKGLEWIGEVNHRGSINYNNYNPSLKSRVTISVDTSKNQFSLKLTSVTAADTAVYYCAKPLRPHCTNGVCYSGDAFDIWGQGTMVTVAS

SEQ ID NO: 592 - protein, 130 aa, synthetic construct; identical to SEQ ID NO: 75
  REGION 1..130: note = Description of Artificial Sequence: Synthetic polypeptide

SEQ ID NO: 593 - protein, 130 aa, synthetic construct; identical to SEQ ID NO: 75
  REGION 1..130: note = Description of Artificial Sequence: Synthetic polypeptide

SEQ ID NO: 594 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEINHRGSINYNNYNPSLKSRVTISVDTSKNQFSLKLTSVTAADTAVYYCAKPLRPHCTNGVCYSGDAFDIWGQGTMVTVAS

SEQ ID NO: 595 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEVNHRGSINYNNYNPSLKSRVTISVDTSKNQFSLKLSSVTAADTAVYYCAKPLRPHCTNGVCYSGDAFDIWGQGTMVTVAS

SEQ ID NO: 596 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEVNHRGSINYNNYNPSLKSRVTISVDTSKNQFSLKLTSVTAADTAVYYCAKPLRPHCTNGVCYSGDAFDIWGQGTMVTVSS

SEQ ID NO: 597 - protein, 130 aa, synthetic construct; identical to SEQ ID NO: 75
  REGION 1..130: note = Description of Artificial Sequence: Synthetic polypeptide

SEQ ID NO: 598 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEINHRGSINYNNYNPSLKSRVTISVDTSKNQFSLKLSSVTAADTAVYYCAKPLRPHCTNGVCYSGDAFDIWGQGTMVTVAS

SEQ ID NO: 599 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEINHRGSINYNNYNPSLKSRVTISVDTSKNQFSLKLTSVTAADTAVYYCAKPLRPHCTNGVCYSGDAFDIWGQGTMVTVSS

SEQ ID NO: 600 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEVNHRGSINYNNYNPSLKSRVTISVDTSKNQFSLKLSSVTAADTAVYYCAKPLRPHCTNGVCYSGDAFDIWGQGTMVTVSS

SEQ ID NO: 601 - protein, 130 aa, synthetic construct [sequence omitted]

SEQ ID NO: 602 - protein, 130 aa, synthetic construct [sequence omitted]

SEQ ID NO: 603 - protein, 130 aa, synthetic construct; identical to SEQ ID NO: 598
  REGION 1..130: note = Description of Artificial Sequence: Synthetic polypeptide

SEQ ID NO: 604 - protein, 130 aa, synthetic construct; identical to SEQ ID NO: 598
  REGION 1..130: note = Description of Artificial Sequence: Synthetic polypeptide

SEQ ID NO: 605 - protein, 130 aa, synthetic construct; identical to SEQ ID NO: 598
  REGION 1..130: note = Description of Artificial Sequence: Synthetic polypeptide

SEQ ID NO: 606 - protein, 130 aa, synthetic construct
  REGION 1..130: note = Description of Artificial Sequence: Synthetic polypeptide
QVQLQQWGAGLLKPSETLSLTCAVYGGSFSGYYWSWIRQPPGKGLEWIGEINHRGSINYNNYNPSLKSRVTISVDTSKNQFSLKLSSVTAADTAVYYCAKPLRPHCTNGVCYSGDAFDIWGQGTMVTVAS

SEQ ID NO: 607 - protein, 130 aa, synthetic construct; identical to SEQ ID NO: 606
  REGION 1..130: note = Description of Artificial Sequence: Synthetic polypeptide

SEQ ID NO: 608 - protein, 130 aa, synthetic construct; identical to SEQ ID NO: 606
  REGION 1..130: note = Description of Artificial Sequence: Synthetic polypeptide

SEQ ID NO: 609 - protein, 130 aa, synthetic construct; identical to SEQ ID NO: 606
  REGION 1..130: note = Description of Artificial Sequence: Synthetic polypeptide

SEQ ID NO: 610 - protein, 130 aa, synthetic construct
  REGION 1..130: note = Description of Artificial Sequence: Synthetic polypeptide
QVQLQQWGAGLLKPSETLSLTCAVYGGSFSGYYWSWIRQPPGKGLEWIGEINHAGSINYNNYNPSLKSRVTISVDTSKNQFSLKLSSVTAADTAVYYCAKPLRPHCTNGVCYSGDAFDIWGQGTMVTVAS

SEQ ID NO: 611 - protein, 130 aa, synthetic construct; identical to SEQ ID NO: 610
  REGION 1..130: note = Description of Artificial Sequence: Synthetic polypeptide

SEQ ID NO: 612 - protein, 130 aa, synthetic construct
  REGION 1..130: note = Description of Artificial Sequence: Synthetic polypeptide
QVQLQQWGAGLLKPSETLSLTCAVYGGSFSGYYWSWIRQPPGKGLEWIGEINHQGSINYNNYNPSLKSRVTISVDTSKNQFSLKLSSVTAADTAVYYCAKPLRPHCTNGVCYSGDAFDIWGQGTMVTVAS

SEQ ID NO: 613 - protein, 130 aa, synthetic construct; identical to SEQ ID NO: 612
  REGION 1..130: note = Description of Artificial Sequence: Synthetic polypeptide

SEQ ID NO: 614 - protein, 130 aa, synthetic construct
  REGION 1..130: note = Description of Artificial Sequence: Synthetic polypeptide
QVQLQQWGAGLLKPSETLSLTCAVYGGSFSDYYWSWIRQPPGKGLEWIGEINHRGSINYNNYNPSLKSRVTISVDTSKNQFSLKLSSVTAADTAVYYCAKPLRPHCINGVCYSGDAFDIWGQGTMVTVAS

SEQ ID NO: 615 - protein, 130 aa, synthetic construct; identical to SEQ ID NO: 598
  REGION 1..130: note = Description of Artificial Sequence: Synthetic polypeptide

SEQ ID NO: 616 - protein, 130 aa, synthetic construct; identical to SEQ ID NO: 598
  REGION 1..130: note = Description of Artificial Sequence: Synthetic polypeptide

SEQ ID NO: 617 - protein, 130 aa, synthetic construct; identical to SEQ ID NO: 598
  REGION 1..130: note = Description of Artificial Sequence: Synthetic polypeptide

SEQ ID NO: 618 - protein, 130 aa, synthetic construct; identical to SEQ ID NO: 614
  REGION 1..130: note = Description of Artificial Sequence: Synthetic polypeptide

SEQ ID NO: 619 - protein, 130 aa, synthetic construct; identical to SEQ ID NO: 598
  REGION 1..130: note = Description of Artificial Sequence: Synthetic polypeptide

SEQ ID NO: 620 - protein, 130 aa, synthetic construct; identical to SEQ ID NO: 598
  REGION 1..130: note = Description of Artificial Sequence: Synthetic polypeptide

SEQ ID NO: 621 - protein, 130 aa, synthetic construct; identical to SEQ ID NO: 614
  REGION 1..130: note = Description of Artificial Sequence: Synthetic polypeptide

SEQ ID NO: 622 - protein, 130 aa, synthetic construct; identical to SEQ ID NO: 598
  REGION 1..130: note = Description of Artificial Sequence: Synthetic polypeptide

SEQ ID NO: 623 - protein, 130 aa, synthetic construct; identical to SEQ ID NO: 614
  REGION 1..130: note = Description of Artificial Sequence: Synthetic polypeptide

SEQ ID NO: 624 - protein, 130 aa, synthetic construct; identical to SEQ ID NO: 614
  REGION 1..130: note = Description of Artificial Sequence: Synthetic polypeptide

SEQ ID NO: 625 - protein, 130 aa, synthetic construct; identical to SEQ ID NO: 598
  REGION 1..130: note = Description of Artificial Sequence: Synthetic polypeptide

SEQ ID NO: 626 - protein, 130 aa, synthetic construct; identical to SEQ ID NO: 598
  REGION 1..130: note = Description of Artificial Sequence: Synthetic polypeptide

SEQ ID NO: 627 - protein, 130 aa, synthetic construct; identical to SEQ ID NO: 598
  REGION 1..130: note = Description of Artificial Sequence: Synthetic polypeptide

SEQ ID NO: 628 - protein, 130 aa, synthetic construct
  REGION 1..130: note = Description of Artificial Sequence: Synthetic polypeptide
QVQLQQWGAGLLKPSETLSLTCAVYGGSFSGYYWSWIRQPPGKGLEWIGEINHRGSINYNNYNPSLKSRVTISVDTSKNQFSLKLSSVTAADTAVYYCAKPLRPHCINGVCYSGDAFDIWGQGTMVTVAS

SEQ ID NO: 629 - protein, 130 aa, synthetic construct; identical to SEQ ID NO: 606
  REGION 1..130: note = Description of Artificial Sequence: Synthetic polypeptide

SEQ ID NO: 630 - protein, 130 aa, synthetic construct; identical to SEQ ID NO: 606
  REGION 1..130: note = Description of Artificial Sequence: Synthetic polypeptide

SEQ ID NO: 631 - protein, 130 aa, synthetic construct; identical to SEQ ID NO: 606
  REGION 1..130: note = Description of Artificial Sequence: Synthetic polypeptide

SEQ ID NO: 632 - protein, 130 aa, synthetic construct; identical to SEQ ID NO: 628
  REGION 1..130: note = Description of Artificial Sequence: Synthetic polypeptide

SEQ ID NO: 633 - protein, 130 aa, synthetic construct; identical to SEQ ID NO: 606
  REGION 1..130: note = Description of Artificial Sequence: Synthetic polypeptide

SEQ ID NO: 634 - protein, 130 aa, synthetic construct; identical to SEQ ID NO: 606
  REGION 1..130: note = Description of Artificial Sequence: Synthetic polypeptide

SEQ ID NO: 635 - protein, 130 aa, synthetic construct; identical to SEQ ID NO: 628
  REGION 1..130: note = Description of Artificial Sequence: Synthetic polypeptide

SEQ ID NO: 636 - protein, 130 aa, synthetic construct; identical to SEQ ID NO: 606
  REGION 1..130: note = Description of Artificial Sequence: Synthetic polypeptide

SEQ ID NO: 637 - protein, 130 aa, synthetic construct; identical to SEQ ID NO: 628
  REGION 1..130: note = Description of Artificial Sequence: Synthetic polypeptide

SEQ ID NO: 638 - protein, 130 aa, synthetic construct; identical to SEQ ID NO: 628
  REGION 1..130: note = Description of Artificial Sequence: Synthetic polypeptide

SEQ ID NO: 639 - protein, 130 aa, synthetic construct; identical to SEQ ID NO: 606
  REGION 1..130: note = Description of Artificial Sequence: Synthetic polypeptide

SEQ ID NO: 640 - protein, 130 aa, synthetic construct; identical to SEQ ID NO: 606
  REGION 1..130: note = Description of Artificial Sequence: Synthetic polypeptide

SEQ ID NO: 641 - protein, 130 aa, synthetic construct; identical to SEQ ID NO: 606
  REGION 1..130: note = Description of Artificial Sequence: Synthetic polypeptide

SEQ ID NO: 642 - protein, 130 aa, synthetic construct; identical to SEQ ID NO: 628
  REGION 1..130: note = Description of Artificial Sequence: Synthetic polypeptide

SEQ ID NO: 643 - protein, 130 aa, synthetic construct; identical to SEQ ID NO: 606
  REGION 1..130: note = Description of Artificial Sequence: Synthetic polypeptide

SEQ ID NO: 644 - protein, 130 aa, synthetic construct; identical to SEQ ID NO: 606
  REGION 1..130: note = Description of Artificial Sequence: Synthetic polypeptide

SEQ ID NO: 645 - protein, 130 aa, synthetic construct; identical to SEQ ID NO: 606
  REGION 1..130: note = Description of Artificial Sequence: Synthetic polypeptide

SEQ ID NO: 646 - protein, 130 aa, synthetic construct; identical to SEQ ID NO: 628
  REGION 1..130: note = Description of Artificial Sequence: Synthetic polypeptide

SEQ ID NO: 647 - protein, 130 aa, synthetic construct; identical to SEQ ID NO: 606
  REGION 1..130: note = Description of Artificial Sequence: Synthetic polypeptide

SEQ ID NO: 648 - protein, 130 aa, synthetic construct; identical to SEQ ID NO: 606
  REGION 1..130: note = Description of Artificial Sequence: Synthetic polypeptide

SEQ ID NO: 649 - protein, 130 aa, synthetic construct; identical to SEQ ID NO: 628
  REGION 1..130: note = Description of Artificial Sequence: Synthetic polypeptide

SEQ ID NO: 650 - protein, 130 aa, synthetic construct; identical to SEQ ID NO: 606
  REGION 1..130: note = Description of Artificial Sequence: Synthetic polypeptide

SEQ ID NO: 651 - protein, 130 aa, synthetic construct; identical to SEQ ID NO: 628
  REGION 1..130: note = Description of Artificial Sequence: Synthetic polypeptide

SEQ ID NO: 652 - protein, 130 aa, synthetic construct; identical to SEQ ID NO: 628
  REGION 1..130: note = Description of Artificial Sequence: Synthetic polypeptide

SEQ ID NO: 653 - protein, 130 aa, synthetic construct; identical to SEQ ID NO: 606
  REGION 1..130: note = Description of Artificial Sequence: Synthetic polypeptide

SEQ ID NO: 654 - protein, 130 aa, synthetic construct; identical to SEQ ID NO: 606
  REGION 1..130: note = Description of Artificial Sequence: Synthetic polypeptide

SEQ ID NO: 655 - protein, 130 aa, synthetic construct; identical to SEQ ID NO: 606
  REGION 1..130: note = Description of Artificial Sequence: Synthetic polypeptide

SEQ ID NO: 656 - protein, 108 aa, synthetic construct [sequence omitted]

SEQ ID NO: 657 - protein, 108 aa, synthetic construct
  REGION 1..108: note = Description of Artificial Sequence: Synthetic polypeptide
SYVLTQPPSVSVAPGQTARITCGGNNIGSKSVHWYQQKPGQAPVLVVYDDSDRPSGIPEQFSGSNSGNTATLTISRVEAGDEADYYCQVWDSSSDHLVFGGGTKLTVL

SEQ ID NO: 658 - protein, 108 aa, synthetic construct
  REGION 1..108: note = Description of Artificial Sequence: Synthetic polypeptide
SYVLTQPPSVSVAPGQTARITCGGNNIGSKNVHWYQQKPGQAPVLVVYDDSDRPSGIPEQFSGSNSGNTATLTISRVEAGDEADYYCQVWDSSSDHVVFGGGTKLTVL

SEQ ID NO: 659 - protein, 108 aa, synthetic construct [sequence omitted]

SEQ ID NO: 660 - protein, 108 aa, synthetic construct [sequence omitted]

SEQ ID NO: 661 - protein, 108 aa, synthetic construct [sequence omitted]

SEQ ID NO: 662 - protein, 108 aa, synthetic construct
  REGION 1..108: note = Description of Artificial Sequence: Synthetic polypeptide
SYVLTQPPSVSVAPGQTARITCGGNNIGSKNVHWYQQKPGQAPVLVVYDDSDRPSGIPERFSGSNSGNTATLTISRVEAGDEADYYCQVWDSSSDHLVFGGGTKLTVL

SEQ ID NO: 663 - protein, 108 aa, synthetic construct [sequence omitted]

SEQ ID NO: 664 - protein, 108 aa, synthetic construct [sequence omitted]

SEQ ID NO: 665 - protein, 108 aa, synthetic construct [sequence omitted]

SEQ ID NO: 666 - protein, 108 aa, synthetic construct [sequence omitted]

SEQ ID NO: 667 - protein, 108 aa, synthetic construct; identical to SEQ ID NO: 662
  REGION 1..108: note = Description of Artificial Sequence: Synthetic polypeptide

SEQ ID NO: 668 - protein, 108 aa, synthetic construct; identical to SEQ ID NO: 658
  REGION 1..108: note = Description of Artificial Sequence: Synthetic polypeptide

SEQ ID NO: 669 - protein, 108 aa, synthetic construct; identical to SEQ ID NO: 662
  REGION 1..108: note = Description of Artificial Sequence: Synthetic polypeptide

SEQ ID NO: 670 - protein, 108 aa, synthetic construct
  REGION 1..108: note = Description of Artificial Sequence: Synthetic polypeptide
SYVLTQPPSVSVAPGQTARITCGGNNIGSKNVHWYQQKPGQAPVLVVYDDSDRPSGIPERFSGSNSGNTATLTISRVEAGDEADYYCQVWDSSSDHVVFGGGTKLTVL

SEQ ID NO: 671 - protein, 108 aa, synthetic construct [sequence omitted]

SEQ ID NO: 672 - protein, 108 aa, synthetic construct; identical to SEQ ID NO: 658
  REGION 1..108: note = Description of Artificial Sequence: Synthetic polypeptide

SEQ ID NO: 673 - protein, 108 aa, synthetic construct; identical to SEQ ID NO: 662
  REGION 1..108: note = Description of Artificial Sequence: Synthetic polypeptide

SEQ ID NO: 674 - protein, 108 aa, synthetic construct; identical to SEQ ID NO: 670
  REGION 1..108: note = Description of Artificial Sequence: Synthetic polypeptide

SEQ ID NO: 675 - protein, 108 aa, synthetic construct; identical to SEQ ID NO: 658
  REGION 1..108: note = Description of Artificial Sequence: Synthetic polypeptide

SEQ ID NO: 676 - protein, 108 aa, synthetic construct; identical to SEQ ID NO: 670
  REGION 1..108: note = Description of Artificial Sequence: Synthetic polypeptide

SEQ ID NO: 677 - protein, 108 aa, synthetic construct; identical to SEQ ID NO: 658
  REGION 1..108: note = Description of Artificial Sequence: Synthetic polypeptide

SEQ ID NO: 678 - protein, 108 aa, synthetic construct; identical to SEQ ID NO: 670
  REGION 1..108: note = Description of Artificial Sequence: Synthetic polypeptide

SEQ ID NO: 679 - protein, 108 aa, synthetic construct
  REGION 1..108: note = Description of Artificial Sequence: Synthetic polypeptide
SYVLTQPPSVSVAPGQTARITCRGNNIGSKNVHWYQQKPGQAPVLVVYDDSDRPSGIPEQFSGSNSGNTATLTISRVEAGDEADYYCQVWDSSSDHVVFGGGTKLTVL

SEQ ID NO: 680 - protein, 108 aa, synthetic construct
  REGION 1..108: note = Description of Artificial Sequence: Synthetic polypeptide
SYVLTQPPSVSVAPGQTARITCRGNNIGYKNVHWYQQKPGQAPVLVVYDDSDRPSGIPEQFSGSNSGNTATLTISRVEAGDEADYYCQVWDSSSDHVVFGGGTKLTVL

SEQ ID NO: 681 - protein, 108 aa, synthetic construct
  REGION 1..108: note = Description of Artificial Sequence: Synthetic polypeptide
SYVLTQPPSVSVAPGQTARITCRGNNIGSMNVHWYQQKPGQAPVLVVYDDSDRPSGIPEQFSGSNSGNTATLTISRVEAGDEADYYCQVWDSSSDHVVFGGGTKLTVL

SEQ ID NO: 682 - protein, 108 aa, synthetic construct
  REGION 1..108: note = Description of Artificial Sequence: Synthetic polypeptide
SYVLTQPPSVSVAPGQTARITCRGNNIGSKNVHWYQQKPGQAPVLVVYDDSDRPSGIPEQFSGSNSGNTATLTISRVEAGDEADYYCQVWDHSSDHVVFGGGTKLTVL

SEQ ID NO: 683 - protein, 108 aa, synthetic construct
  REGION 1..108: note = Description of Artificial Sequence: Synthetic polypeptide
SYVLTQPPSVSVAPGQTARITCGGNNIGYKNVHWYQQKPGQAPVLVVYDDSDRPSGIPEQFSGSNSGNTATLTISRVEAGDEADYYCQVWDSSSDHVVFGGGTKLTVL

SEQ ID NO: 684 - protein, 108 aa, synthetic construct
  REGION 1..108: note = Description of Artificial Sequence: Synthetic polypeptide
SYVLTQPPSVSVAPGQTARITCGGNNIGYMNVHWYQQKPGQAPVLVVYDDSDRPSGIPEQFSGSNSGNTATLTISRVEAGDEADYYCQVWDSSSDHVVFGGGTKLTVL

SEQ ID NO: 685 - protein, 108 aa, synthetic construct
  REGION 1..108: note = Description of Artificial Sequence: Synthetic polypeptide
SYVLTQPPSVSVAPGQTARITCGGNNIGYKNVHWYQQKPGQAPVLVVYDDSDRPSGIPEQFSGSNSGNTATLTISRVEAGDEADYYCQVWDHSSDHVVFGGGTKLTVL

SEQ ID NO: 686 - protein, 108 aa, synthetic construct
  REGION 1..108: note = Description of Artificial Sequence: Synthetic polypeptide
SYVLTQPPSVSVAPGQTARITCGGNNIGSMNVHWYQQKPGQAPVLVVYDDSDRPSGIPEQFSGSNSGNTATLTISRVEAGDEADYYCQVWDSSSDHVVFGGGTKLTVL

SEQ ID NO: 687 - protein, 108 aa, synthetic construct
  REGION 1..108: note = Description of Artificial Sequence: Synthetic polypeptide
SYVLTQPPSVSVAPGQTARITCGGNNIGSMNVHWYQQKPGQAPVLVVYDDSDRPSGIPEQFSGSNSGNTATLTISRVEAGDEADYYCQVWDHSSDHVVFGGGTKLTVL

SEQ ID NO: 688 - protein, 108 aa, synthetic construct
  REGION 1..108: note = Description of Artificial Sequence: Synthetic polypeptide
SYVLTQPPSVSVAPGQTARITCGGNNIGSKNVHWYQQKPGQAPVLVVYDDSDRPSGIPEQFSGSNSGNTATLTISRVEAGDEADYYCQVWDHSSDHVVFGGGTKLTVL

SEQ ID NO: 689 - protein, 108 aa, synthetic construct; identical to SEQ ID NO: 685
  REGION 1..108: note = Description of Artificial Sequence: Synthetic polypeptide

SEQ ID NO: 690 - protein, 108 aa, synthetic construct
  REGION 1..108: note = Description of Artificial Sequence: Synthetic polypeptide
SYVLTQPPSVSVAPGQTARITCGGNNIGSKNVHWYQQKPGQAPVLVVYDDSDRPSGIPEQFSGSNSGNTATLTISRVEAGDEADYYCQVWDSRSDHVVFGGGTKLTVL

SEQ ID NO: 691 - protein, 108 aa, synthetic construct
  REGION 1..108: note = Description of Artificial Sequence: Synthetic polypeptide
SYVLTQPPSVSVAPGQTARITCGGNNIGSKNVHWYQQKPGQAPVLVVYDDSDRPSGIPEQFSGSNSGNTATLTISRVEAGDEADYYCQVWDSESDHVVFGGGTKLTVL

SEQ ID NO: 692 - protein, 108 aa, synthetic construct
  REGION 1..108: note = Description of Artificial Sequence: Synthetic polypeptide
SYVLTQPPSVSVAPGQTARITCGGNNIGYKNVHWYQQKPGQAPVLVVYDDSDRPSGIPEQFSGSNSGNTATLTISRVEAGDEADYYCQVWDSESDHVVFGGGTKLTVL

SEQ ID NO: 693 - protein, 108 aa, synthetic construct; identical to SEQ ID NO: 679
  REGION 1..108: note = Description of Artificial Sequence: Synthetic polypeptide

SEQ ID NO: 694 - protein, 108 aa, synthetic construct; identical to SEQ ID NO: 680
  REGION 1..108: note = Description of Artificial Sequence: Synthetic polypeptide

SEQ ID NO: 695 - protein, 108 aa, synthetic construct; identical to SEQ ID NO: 681
  REGION 1..108: note = Description of Artificial Sequence: Synthetic polypeptide

SEQ ID NO: 696 - protein, 108 aa, synthetic construct; identical to SEQ ID NO: 682
  REGION 1..108: note = Description of Artificial Sequence: Synthetic polypeptide

SEQ ID NO: 697 - protein, 108 aa, synthetic construct; identical to SEQ ID NO: 683
  REGION 1..108: note = Description of Artificial Sequence: Synthetic polypeptide

SEQ ID NO: 698 - protein, 108 aa, synthetic construct; identical to SEQ ID NO: 684
  REGION 1..108: note = Description of Artificial Sequence: Synthetic polypeptide

SEQ ID NO: 699 - protein, 108 aa, synthetic construct; identical to SEQ ID NO: 685
  REGION 1..108: note = Description of Artificial Sequence: Synthetic polypeptide

SEQ ID NO: 700 - protein, 108 aa, synthetic construct; identical to SEQ ID NO: 686
  REGION 1..108: note = Description of Artificial Sequence: Synthetic polypeptide

SEQ ID NO: 701 - protein, 108 aa, synthetic construct; identical to SEQ ID NO: 687
  REGION 1..108: note = Description of Artificial Sequence: Synthetic polypeptide

SEQ ID NO: 702 - protein, 108 aa, synthetic construct; identical to SEQ ID NO: 688
  REGION 1..108: note = Description of Artificial Sequence: Synthetic polypeptide

SEQ ID NO: 703 - protein, 108 aa, synthetic construct; identical to SEQ ID NO: 685
  REGION 1..108: note = Description of Artificial Sequence: Synthetic polypeptide

SEQ ID NO: 704 - protein, 108 aa, synthetic construct; identical to SEQ ID NO: 690
  REGION 1..108: note = Description of Artificial Sequence: Synthetic polypeptide

SEQ ID NO: 705 - protein, 108 aa, synthetic construct; identical to SEQ ID NO: 691
  REGION 1..108: note = Description of Artificial Sequence: Synthetic polypeptide

SEQ ID NO: 706 - protein, 108 aa, synthetic construct; identical to SEQ ID NO: 692
  REGION 1..108: note = Description of Artificial Sequence: Synthetic polypeptide

SEQ ID NO: 707 - protein, 108 aa, synthetic construct
  REGION 1..108: note = Description of Artificial Sequence: Synthetic polypeptide
SYVLTQPPSVSVAPGQTARITCRGNNIGSKNVHWYQQKPGQAPVLVVYDDSDRPSGIPERFSGSNSGNTATLTISRVEAGDEADYYCQVWDSSSDHVVFGGGTKLTVL

SEQ ID NO: 708 - protein, 108 aa, synthetic construct
  REGION 1..108: note = Description of Artificial Sequence: Synthetic polypeptide
SYVLTQPPSVSVAPGQTARITCRGNNIGYKNVHWYQQKPGQAPVLVVYDDSDRPSGIPERFSGSNSGNTATLTISRVEAGDEADYYCQVWDSSSDHVVFGGGTKLTVL

SEQ ID NO: 709 - protein, 108 aa, synthetic construct
  REGION 1..108: note = Description of Artificial Sequence: Synthetic polypeptide
SYVLTQPPSVSVAPGQTARITCRGNNIGSMNVHWYQQKPGQAPVLVVYDDSDRPSGIPERFSGSNSGNTATLTISRVEAGDEADYYCQVWDSSSDHVVFGGGTKLTVL

SEQ ID NO: 710 - protein, 108 aa, synthetic construct
  REGION 1..108: note = Description of Artificial Sequence: Synthetic polypeptide
SYVLTQPPSVSVAPGQTARITCRGNNIGSKNVHWYQQKPGQAPVLVVYDDSDRPSGIPERFSGSNSGNTATLTISRVEAGDEADYYCQVWDHSSDHVVFGGGTKLTVL

SEQ ID NO: 711 - protein, 108 aa, synthetic construct
  REGION 1..108: note = Description of Artificial Sequence: Synthetic polypeptide
SYVLTQPPSVSVAPGQTARITCGGNNIGYKNVHWYQQKPGQAPVLVVYDDSDRPSGIPERFSGSNSGNTATLTISRVEAGDEADYYCQVWDSSSDHVVFGGGTKLTVL

SEQ ID NO: 712 - protein, 108 aa, synthetic construct
  REGION 1..108: note = Description of Artificial Sequence: Synthetic polypeptide
SYVLTQPPSVSVAPGQTARITCGGNNIGYMNVHWYQQKPGQAPVLVVYDDSDRPSGIPERFSGSNSGNTATLTISRVEAGDEADYYCQVWDSSSDHVVFGGGTKLTVL

SEQ ID NO: 713 - protein, 108 aa, synthetic construct
  REGION 1..108: note = Description of Artificial Sequence: Synthetic polypeptide
SYVLTQPPSVSVAPGQTARITCGGNNIGYKNVHWYQQKPGQAPVLVVYDDSDRPSGIPERFSGSNSGNTATLTISRVEAGDEADYYCQVWDHSSDHVVFGGGTKLTVL

SEQ ID NO: 714 - protein, 108 aa, synthetic construct
  REGION 1..108: note = Description of Artificial Sequence: Synthetic polypeptide
SYVLTQPPSVSVAPGQTARITCGGNNIGSMNVHWYQQKPGQAPVLVVYDDSDRPSGIPERFSGSNSGNTATLTISRVEAGDEADYYCQVWDSSSDHVVFGGGTKLTVL

SEQ ID NO: 715 - protein, 108 aa, synthetic construct
  REGION 1..108: note = Description of Artificial Sequence: Synthetic polypeptide
SYVLTQPPSVSVAPGQTARITCGGNNIGSMNVHWYQQKPGQAPVLVVYDDSDRPSGIPERFSGSNSGNTATLTISRVEAGDEADYYCQVWDHSSDHVVFGGGTKLTVL

SEQ ID NO: 716 - protein, 108 aa, synthetic construct
  REGION 1..108: note = Description of Artificial Sequence: Synthetic polypeptide
SYVLTQPPSVSVAPGQTARITCGGNNIGSKNVHWYQQKPGQAPVLVVYDDSDRPSGIPERFSGSNSGNTATLTISRVEAGDEADYYCQVWDHSSDHVVFGGGTKLTVL

SEQ ID NO: 717 - protein, 108 aa, synthetic construct; identical to SEQ ID NO: 713
  REGION 1..108: note = Description of Artificial Sequence: Synthetic polypeptide

SEQ ID NO: 718 - protein, 108 aa, synthetic construct
  REGION 1..108: note = Description of Artificial Sequence: Synthetic polypeptide
SYVLTQPPSVSVAPGQTARITCGGNNIGSKNVHWYQQKPGQAPVLVVYDDSDRPSGIPERFSGSNSGNTATLTISRVEAGDEADYYCQVWDSRSDHVVFGGGTKLTVL

SEQ ID NO: 719 - protein, 108 aa, synthetic construct
  REGION 1..108: note = Description of Artificial Sequence: Synthetic polypeptide
SYVLTQPPSVSVAPGQTARITCGGNNIGSKNVHWYQQKPGQAPVLVVYDDSDRPSGIPERFSGSNSGNTATLTISRVEAGDEADYYCQVWDSESDHVVFGGGTKLTVL

SEQ ID NO: 720 - protein, 108 aa, synthetic construct
  REGION 1..108: note = Description of Artificial Sequence: Synthetic polypeptide
SYVLTQPPSVSVAPGQTARITCGGNNIGYKNVHWYQQKPGQAPVLVVYDDSDRPSGIPERFSGSNSGNTATLTISRVEAGDEADYYCQVWDSESDHVVFGGGTKLTVL

SEQ ID NO: 721 - protein, 121 aa, synthetic construct
  REGION 1..121: note = Description of Artificial Sequence: Synthetic polypeptide
EVQLVQSGAEVKKPGSSVKVSCKASGYTFTSYYIYWVRQAPGQGLEWIGNIWPGNGGTFYGEKFMGRATFTADTSTSTAYMELSSLRSEDTAVYYCARRPDYSGDDYFDYWGQGTLVTVSS

SEQ ID NO: 722 - protein, 121 aa, synthetic construct
  REGION 1..121: note = Description of Artificial Sequence: Synthetic polypeptide
QVQLQQSGAELAKPGTSVKLSCKASGYTFTSYYIYWVKQRPGQGLEWIGNIWPGNGGTFYGEKFMGKATFTADTSSSTAYMLLGSLTPEDSAYYFCARRPDYSGDDYFDYWGQGVLVTVSS

SEQ ID NO: 723 - protein, 118 aa, synthetic construct
  REGION 1..118: note = Description of Artificial Sequence: Synthetic polypeptide
QVKLVQSGAALVKPGASVKMSCKASDYTFNDYWVSWVKQRHGESLEWIGEIYPNSGATNFNGKFRGKATLTVDNPTSTAYMELSRLTSEDSAIYYCTREYTRDWFAYWGQGTLVTVSS

SEQ ID NO: 724 - protein, 119 aa, synthetic construct
  REGION 1..119: note = Description of Artificial Sequence: Synthetic polypeptide
QVQLQQSGAEVIKPGASVKLSCKASGYTFSSYWMHWVRQAPGQGLEWIGEINPGNGHTNYNEKFKSRATLTGDTSTSTVYMELSSLRSEDTAVYYCARSFTTARAFAYWGQGTLVTVSS

SEQ ID NO: 725 - protein, 114 aa, synthetic construct
  REGION 1..114: note = Description of Artificial Sequence: Synthetic polypeptide
EVQLVESGGGLVQPGGSLRLSCAASGFTFSDYWMSWVRQAPGKGLEWVADIKNDGSYTNYAPSLTNRFTISRDNAKNSLYLQMNSLRAEDTAVYYCARELTGTWGQGTMVTVSS

SEQ ID NO: 726 - protein, 121 aa, synthetic construct [sequence omitted]

SEQ ID NO: 727 - protein, 126 aa, synthetic construct
  REGION 1..126: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDSPFLLDDYYYYYYMDVWGKGTTVTVSS

SEQ ID NO: 728 - protein, 126 aa, synthetic construct
  REGION 1..126: note = Description of Artificial Sequence: Synthetic polypeptide
EVQLLESGGGLVQPGGSLRLSCAASGFTFRNYAMSWVRQAPGKGLEWVSAISGSGDTTYYADSVKGRFTISRDNSKNTLYLQMNSLRAEDTAVYYCAKDSPFLLDDYYYYYYMDVWGKGTTVTVSS

SEQ ID NO: 729 - protein, 119 aa, synthetic construct
  REGION 1..119: note = Description of Artificial Sequence: Synthetic polypeptide
QVQLVQSGAEVKKPGSSVKVSCKASGGTFNSYAISWVRQAPGQGLEWMGGIIPGFGTANYAQKFQGRVTITADESTSTAYMELSSLRSEDTAVYYCARKNEEDGGFDHWGQGTLVTVSS

SEQ ID NO: 730 - protein, 118 aa, synthetic construct
  REGION 1..118: note = Description of Artificial Sequence: Synthetic polypeptide
QVQLVESGGGLVQPGGSLRLSCAASGFTFSDYYMHWVRQAPGKGLEWVSVISGSGSNTYYADSVKGRFTISRDNSKNTLYLQMNSLRAEDTAVYYCARLYAQFEGDFWGQGTLVTVSS

SEQ ID NO: 731 - protein, 116 aa, synthetic construct
  REGION 1..116: note = Description of Artificial Sequence: Synthetic polypeptide
QVQLVQSGAEVKKPGESLKISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 732 - protein, 116 aa, synthetic construct
  REGION 1..116: note = Description of Artificial Sequence: Synthetic polypeptide
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 733 - protein, 116 aa, synthetic construct; identical to SEQ ID NO: 731
  REGION 1..116: note = Description of Artificial Sequence: Synthetic polypeptide

SEQ ID NO: 734 - protein, 116 aa, synthetic construct; identical to SEQ ID NO: 732
  REGION 1..116: note = Description of Artificial Sequence: Synthetic polypeptide

SEQ ID NO: 735 - protein, 119 aa, synthetic construct
  REGION 1..119: note = Description of Artificial Sequence: Synthetic polypeptide
QVQLVQSGAEVKKPGASVKLSCKASGYTFSSYWMHWVRQAPGQGLEWIGEINPGNGHTNYNEKFKSRVTMTRDTSTSTAYMELSSLRSEDTAVYYCARSFKTARAFAYWGQGTLVTVSS

SEQ ID NO: 736 - protein, 122 aa, synthetic construct
  REGION 1..122: note = Description of Artificial Sequence: Synthetic polypeptide
EVQLVESGGGLVKPGGSLRLSCAASGFTFSTFTMNWVRQAPGKGLEWVSSISSKSTYIEYADSFKVRFTISRDNAKNSLYLQMNSLRAEDTAVYYCARYGAKNFLNWVFDYWGQGTLVTVSS

SEQ ID NO: 737 - protein, 116 aa, synthetic construct
  REGION 1..116: note = Description of Artificial Sequence: Synthetic polypeptide
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 738 - protein, 120 aa, synthetic construct
  REGION 1..120: note = Description of Artificial Sequence: Synthetic polypeptide
  VARIANT 1: note = X can be any amino acid
XVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREPGYYGSGLDYWGQGTLVTVSS

SEQ ID NO: 739 - protein, 123 aa, synthetic construct
  REGION 1..123: note = Description of Artificial Sequence: Synthetic polypeptide
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSS

SEQ ID NO: 740 - protein, 118 aa, synthetic construct
  REGION 1..118: note = Description of Artificial Sequence: Synthetic polypeptide
EVQLLESGGGLVQPGGSLRLSCAASGFNFGYSYMSWVRQAPGKGLEWVSSIGSTSSHTYYADSVKGRFTISRDNSKNTLYLQMNSLRAEDTAVYYCARVYSSPGIDYWGQGTLVTVSS

SEQ ID NO: 741 - protein, 119 aa, synthetic construct
  REGION 1..119: note = Description of Artificial Sequence: Synthetic polypeptide
QVQLVQSGAEVVKPGASVKLSCKASGYTFSSYWMHWVKQAPGQVLEWIGEINPGNGHTNYNEKFKSKATLTVDKSASTAYMELSSLRSEDTAVYYCARSFTTARAFAYWGQGTLVTVSS

SEQ ID NO: 742 - protein, 119 aa, synthetic construct
  REGION 1..119: note = Description of Artificial Sequence: Synthetic polypeptide
QVQLVQSGAEVKKPGASVKVSCKASGYTFSSYWMHWVRQAPGQRLEWMGEINPGNGHTNYSQKFQGRVTITVDKSASTAYMELSSLRSEDTAVYYCARSFTTARAFAYWGQGTLVTVSS

SEQ ID NO: 743 - protein, 111 aa, synthetic construct
  REGION 1..111: note = Description of Artificial Sequence: Synthetic polypeptide
NVMLTQPHSVSESPGKTVTISCKLNSGNIGSYYVHWYQQRPGSSPTTMIYRDDKRPDGVPDRFSGSIDSSSNSASLTISGLKTEDEADYYCHSYDSTITPVFGGGTKLTVL

SEQ ID NO: 744 - protein, 111 aa, synthetic construct
  REGION 1..111: note = Description of Artificial Sequence: Synthetic polypeptide
QVVLTQPKSVSTSLKSTVKLSCKLNSGNIGSYYVHWYQQHAGRSPTTMIYRDDKRPDGVPDRFSGSIDSSSNSAFLTINNVQTEDDAIYFCHSYDSTITPVFGGGTKLTVL

SEQ ID NO: 745 - protein, 112 aa, synthetic construct
  REGION 1..112: note = Description of Artificial Sequence: Synthetic polypeptide
DVVLTQTPSILSATIGQSVSISCRSSQSLLDSDGNTYLYWFLQRPGQSPQRLIYLVSNLGSGVPNRFSGSGSGTDFTLKISGVEAEDLGIYYCMQPTHAPYTFGAGTKLELK

SEQ ID NO: 746 - protein, 107 aa, synthetic construct
  REGION 1..107: note = Description of Artificial Sequence: Synthetic polypeptide
DIVMTQSPAFLSVTPGEKVTITCRASQTISDYLHWYQQKPDQAPKLLIKYASQSISGIPSRFSGSGSGTDFTFTISSLEAEDAATYYCQDGHSFPPTFGQGTKLEIK

SEQ ID NO: 747 - protein, 113 aa, synthetic construct
  REGION 1..113: note = Description of Artificial Sequence: Synthetic polypeptide
DIVMTQSPDSLAVSLGERATINCKSSQSLLSSGNQKNYLAWYQQKPGQPPKLLIYYASTRQSGVPDRFSGSGSGTDFTLTISSLQAEDVAVYYCLQYDRYPFTFGQGTKLEIK

SEQ ID NO: 748 - protein, 109 aa, synthetic construct [sequence omitted]

SEQ ID NO: 749 - protein, 107 aa, synthetic construct
  REGION 1..107: note = Description of Artificial Sequence: Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGHLFPITFGGGTKVEIK

SEQ ID NO: 750 - protein, 107 aa, synthetic construct; identical to SEQ ID NO: 749
  REGION 1..107: note = Description of Artificial Sequence: Synthetic polypeptide

SEQ ID NO: 751 - protein, 107 aa, synthetic construct
  REGION 1..107: note = Description of Artificial Sequence: Synthetic polypeptide
DIELTQPPSVSVAPGQTARISCSGDNLGDYYASWYQQKPGQAPVLVIYDDSNRPSGIPERFSGSNSGNTATLTISGTQAEDEADYYCQTWDGTLHFVFGGGTKLTVL

SEQ ID NO: 752 - protein, 107 aa, synthetic construct
  REGION 1..107: note = Description of Artificial Sequence: Synthetic polypeptide
DIELTQPPSVSVAPGQTARISCSGDNIGSKYVSWYQQKPGQAPVLVIYSDSERPSGIPERFSGSNSGNTATLTISGTQAEDEADYYCQSWDGSISRVFGGGTKLTVL

SEQ ID NO: 753 - protein, 108 aa, synthetic construct
  REGION 1..108: note = Description of Artificial Sequence: Synthetic polypeptide
DIELTQPPSVSVAPGQTARISCSGDNIGDQYAHWYQQKPGQAPVVVIYQDKNRPSGIPERFSGSNSGNTATLTISGTQAEDEADYYCATYTGFGSLAVFGGGTKLTVL

SEQ ID NO: 754 - protein, 108 aa, synthetic construct
  REGION 1..108: note = Description of Artificial Sequence: Synthetic polypeptide
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 755 - protein, 108 aa, synthetic construct
  REGION 1..108: note = Description of Artificial Sequence: Synthetic polypeptide
DIELTQPPSVSVAPGQTARISCSGDNIGDQYAHWYQQKPGQAPVVVIYQDKNRPSGIPERFSGSNSGNTATLTISGTQAEDEADYYCSTYTFVGFTTVFGGGTKLTVL

SEQ ID NO: 756 - protein, 108 aa, synthetic construct; identical to SEQ ID NO: 754
  REGION 1..108: note = Description of Artificial Sequence: Synthetic polypeptide

SEQ ID NO: 757 - protein, 107 aa, synthetic construct
  REGION 1..107: note = Description of Artificial Sequence: Synthetic polypeptide
DIVMTQSPAFLSVTPGEKVTITCRASQTISDYLHWYQQKPDQAPKLLIKYASQSISGIPSRFSGSGSGTDFTFTISSLEAEDAATYYCQDGHSWPPTFGQGTKLEIK

SEQ ID NO: 758 - protein, 110 aa, synthetic construct
  REGION 1..110: note = Description of Artificial Sequence: Synthetic polypeptide
QSALTQPPSASGSPGQTVTISCTGTRYDVGYYEYVSWYQQHPGKAPKLMIYETSKRLSGVPDRFSGSKSGNTASLTVSGLQAEDEADYYCSSYRYEHQVSFGGGTKLTVL

SEQ ID NO: 759 - protein, 107 aa, synthetic construct
  REGION 1..107: note = Description of Artificial Sequence: Synthetic polypeptide
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIR

SEQ ID NO: 760 - protein, 109 aa, synthetic construct
  REGION 1..109: note = Description of Artificial Sequence: Synthetic polypeptide
SYVLTQPPSVSVAPGETARITCGGDDIGDKRVHWYQKKPDQAPVLVVYEDRYRPSGIPERISGSNSGNTATLTLSRVEAGDEADYYCQVWDSSSDHPGVFGGGTQLIIL

SEQ ID NO: 761 - protein, 106 aa, synthetic construct
  REGION 1..106: note = Description of Artificial Sequence: Synthetic polypeptide
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIK

SEQ ID NO: 762 - protein, 108 aa, synthetic construct
  REGION 1..108: note = Description of Artificial Sequence: Synthetic polypeptide
DIQMTQSPSSLSASVGDRVTITCRASQSIGSTLNWYQQKPGKAPKLLIYGASSLQSGVPSRFSGSGSGTDFTLTISSLQPEDFATYYCQQYYTWVPFTFGQGTKLEIK

SEQ ID NO: 763 - protein, 107 aa, synthetic construct
  REGION 1..107: note = Description of Artificial Sequence: Synthetic polypeptide
DIVMTQSPATQSVSPGERVTLSCRASQTISDYLHWYQQKPGQSPRLLIKYASQSISGIPSRFSGSGSGSDFTLTISSVEPEDFGVYYCQDGHSFPPTFGGGTKLEIK

SEQ ID NO: 764 - protein, 107 aa, synthetic construct
  REGION 1..107: note = Description of Artificial Sequence: Synthetic polypeptide
DIVMTQSPPTLSLSPGERVTLSCRASQSISDYLHWYQQKPGQSPRLLIKYASQSISGIPARFSGSGSGTDFTLTISSLEPEDFAVYYCQDGHSFPPTFGGGTKVEIK

SEQ ID NO: 765 - protein, 267 aa, synthetic construct
  REGION 1..267: note = Description of Artificial Sequence: Synthetic polypeptide
QVQLQQWGAGLLKPSETLSLTCAVYGGSFSGYYWSWIRQPPGKGLEWIGEINHRGSINYNNYNPSLKSRVTISVDTSKNQFSLKLSSVTAADTAVYYCAKPLRPHCINGVCYSGDAFDIWGQGTMVTVASGGGGSGGGGSGGGGSGGGGSSYVLTQPPSVSVAPGQTARITCGGNNIGYKNVHWYQQKPGQAPVLVVYDDSDRPSGIPERFSGSNSGNTATLTISRVEAGDEADYYCQVWDHSSDHVVFGGGTKLTVLGGGHHHHHH

SEQ ID NO: 766 - protein, 267 aa, synthetic construct
  REGION 1..267: note = Description of Artificial Sequence: Synthetic polypeptide
SYVLTQPPSVSVAPGQTARITCGGNNIGYKNVHWYQQKPGQAPVLVVYDDSDRPSGIPERFSGSNSGNTATLTISRVEAGDEADYYCQVWDHSSDHVVFGGGTKLTVLGGGGSGGGGSGGGGSGGGGSQVQLQQWGAGLLKPSETLSLTCAVYGGSFSGYYWSWIRQPPGKGLEWIGEINHRGSINYNNYNPSLKSRVTISVDTSKNQFSLKLSSVTAADTAVYYCAKPLRPHCINGVCYSGDAFDIWGQGTMVTVASGGGHHHHHH

SEQ ID NO: 767 - protein, 262 aa, synthetic construct
  REGION 1..262: note = Description of Artificial Sequence: Synthetic polypeptide
QVQLQQWGAGLLKPSETLSLTCAVYGGSFSGYYWSWIRQPPGKGLEWIGEINHRGSINYNNYNPSLKSRVTISVDTSKNQFSLKLSSVTAADTAVYYCAKPLRPHCINGVCYSGDAFDIWGQGTMVTVASGGGGSGGGGSGGGGSSYVLTQPPSVSVAPGQTARITCGGNNIGYKNVHWYQQKPGQAPVLVVYDDSDRPSGIPERFSGSNSGNTATLTISRVEAGDEADYYCQVWDHSSDHVVFGGGTKLTVLGGGHHHHHH

SEQ ID NO: 768 - protein, 262 aa, synthetic construct
  REGION 1..262: note = Description of Artificial Sequence: Synthetic polypeptide
SYVLTQPPSVSVAPGQTARITCGGNNIGYKNVHWYQQKPGQAPVLVVYDDSDRPSGIPERFSGSNSGNTATLTISRVEAGDEADYYCQVWDHSSDHVVFGGGTKLTVLGGGGSGGGGSGGGGSQVQLQQWGAGLLKPSETLSLTCAVYGGSFSGYYWSWIRQPPGKGLEWIGEINHRGSINYNNYNPSLKSRVTISVDTSKNQFSLKLSSVTAADTAVYYCAKPLRPHCINGVCYSGDAFDIWGQGTMVTVASGGGHHHHHH

SEQ ID NO: 769 - protein, 10 aa, synthetic construct
  REGION 1..10: note = Description of Artificial Sequence: Synthetic peptide
  VARIANT 4: note = X can be F or L
  VARIANT 5: note = X can be S or N
  VARIANT 8: note = X can be Y or H
  VARIANT 6: note = X can be D or G
GGSXXXYXWS

SEQ ID NO: 770 - protein, 19 aa, synthetic construct
  REGION 1..19: note = Description of Artificial Sequence: Synthetic peptide
  VARIANT 2: note = X can be I or V
  VARIANT 5: note = X can be A, Q, R or S
  VARIANT 8: note = X can be I or T
  VARIANT 9: note = X can be N or S
EXNHXGSXXYNNYNPSLKS

SEQ ID NO: 771 - protein, 21 aa, synthetic construct
  REGION 1..21: note = Description of Artificial Sequence: Synthetic peptide
  VARIANT 4: note = X can be L or F
  VARIANT 9: note = X can be I or T
  VARIANT 14: note = X can be Y or S
AKPXRPHCXNGVCXSGDAFDI

SEQ ID NO: 772 - protein, 11 aa, synthetic construct
  REGION 1..11: note = Description of Artificial Sequence: Synthetic peptide
  VARIANT 1: note = X can be G or R
  VARIANT 7: note = X can be S , T or Y
  VARIANT 8: note = X can be K or M
  VARIANT 9: note = X can be N or S
XGNNIGXXXVH

SEQ ID NO: 773 - protein, 11 aa, synthetic construct
  REGION 1..11: note = Description of Artificial Sequence: Synthetic peptide
  VARIANT 5: note = X can be H or S
  VARIANT 6: note = X can be E, R or S
  VARIANT 10: note = X can be L or V
QVWDXXSDHXV

SEQ ID NO: 774 - protein, 16 aa, synthetic construct
  REGION 1..16: note = Description of Artificial Sequence: Synthetic peptide
  VARIANT 2: note = X can be I or V
  VARIANT 5: note = X can be A,Q, R or S
  VARIANT 8: note = X can be I or T
  VARIANT 9: note = X can be N or S
EXNHXGSXXYNPSLKS

SEQ ID NO: 775 - protein, 10 aa, synthetic construct
  REGION 1..10: note = Description of Artificial Sequence: Synthetic peptide
  VARIANT 4: note = X can be F or L
  VARIANT 5: note = X can be S or N
  VARIANT 8: note = X can be Y or H
GGSXXDYXWS

SEQ ID NO: 776 - protein, 16 aa, synthetic construct
  REGION 1..16: note = Description of Artificial Sequence: Synthetic peptide
  VARIANT 2: note = X can be I or V
  VARIANT 5: note = X can be R or S
  VARIANT 8: note = X can be I or T
  VARIANT 9: note = X can be N or S
EXNHXGSXXYNPSLKS

SEQ ID NO: 777 - protein, 19 aa, synthetic construct
  REGION 1..19: note = Description of Artificial Sequence: Synthetic peptide
  VARIANT 2: note = X can be I or V
  VARIANT 5: note = X can be R or S
  VARIANT 8: note = X can be I or T
  VARIANT 9: note = X can be N or S
EXNHXGSXXYNNYNPSLKS

SEQ ID NO: 778 - protein, 21 aa, synthetic construct
  REGION 1..21: note = Description of Artificial Sequence: Synthetic peptide
  VARIANT 4: note = X can be L or F
  VARIANT 14: note = X can be Y or S
AKPXRPHCTNGVCXSGDAFDI

SEQ ID NO: 779 - protein, 11 aa, synthetic construct
  REGION 1..11: note = Description of Artificial Sequence: Synthetic peptide
  VARIANT 7: note = X can be S or T
GGNNIGXKNVH

SEQ ID NO: 780 - protein, 11 aa, synthetic construct
  REGION 1..11: note = Description of Artificial Sequence: Synthetic peptide
  VARIANT 1: note = X can be G or R
  VARIANT 7: note = X can be S, T or Y
  VARIANT 8: note = X can be K or M
  VARIANT 9: note = X can be N or I
XGNNIGXXXVH

SEQ ID NO: 781 - protein, 10 aa, synthetic construct
  REGION 1..10: note = Description of Artificial Sequence: Synthetic peptide
GGSGGGGSGG

SEQ ID NO: 782 - protein, 20 aa, synthetic construct
  REGION 1..20: note = Description of Artificial Sequence: Synthetic peptide
GGGGSGGGGSGGGGSGGGGS

SEQ ID NO: 783 - protein, 13 aa, unidentified
  REGION 1..13: note = Description of Unknown: EphA2 sequence
TEPPKVRLEGRST

SEQ ID NO: 784 - protein, 5 aa, unidentified
  REGION 1..5: note = Description of Unknown: EphA2 sequence
KKGDS

SEQ ID NO: 785 - protein, 7 aa, unidentified
  REGION 1..7: note = Description of Unknown: EphA2 sequence
TYLVQVQ

SEQ ID NO: 786 - protein, 11 aa, unidentified
  REGION 1..11: note = Description of Unknown: EphA2 sequence
AGSKVHEFQTL

SEQ ID NO: 787 - protein, 16 aa, unidentified
  REGION 1..16: note = Description of Unknown: EphA1 sequence
AESLSGLSLRLVKKEP

SEQ ID NO: 788 - protein, 5 aa, unidentified
  REGION 1..5: note = Description of Unknown: EphA1 sequence
LNQDE

SEQ ID NO: 789 - protein, 7 aa, unidentified
  REGION 1..7: note = Description of Unknown: EphA1 sequence
TYIVRVR

SEQ ID NO: 790 - protein, 11 aa, unidentified
  REGION 1..11: note = Description of Unknown: EphA1 sequence
PFSPDHEFRTS

SEQ ID NO: 791 - protein, 16 aa, unidentified
  REGION 1..16: note = Description of Unknown: EphA3 sequence
AAPSPVLTIKKDRTSR

SEQ ID NO: 792 - protein, 5 aa, unidentified
  REGION 1..5: note = Description of Unknown: EphA3 sequence
KQEQE

SEQ ID NO: 793 - protein, 7 aa, unidentified
  REGION 1..7: note = Description of Unknown: EphA3 sequence
IYVFQIR

SEQ ID NO: 794 - protein, 11 aa, unidentified
  REGION 1..11: note = Description of Unknown: EphA3 sequence
TNSRKFEFETS

SEQ ID NO: 795 - protein, 16 aa, unidentified
  REGION 1..16: note = Description of Unknown: EphA4 sequence
AAPSSIALVQAKEVTR

SEQ ID NO: 796 - protein, 5 aa, unidentified
  REGION 1..5: note = Description of Unknown: EphA4 sequence
KDQNE

SEQ ID NO: 797 - protein, 7 aa, unidentified
  REGION 1..7: note = Description of Unknown: EphA4 sequence
SYVFHVR

SEQ ID NO: 798 - protein, 11 aa, unidentified
  REGION 1..11: note = Description of Unknown: EphA4 sequence
DFSEPLEVTTN

SEQ ID NO: 799 - protein, 16 aa, unidentified
  REGION 1..16: note = Description of Unknown: EphA5 sequence
AAPSPVTNVKKGKIAK

SEQ ID NO: 800 - protein, 4 aa, unidentified
  REGION 1..4: note = Description of Unknown: EphA5 sequence
KDQE

SEQ ID NO: 801 - protein, 7 aa, unidentified
  REGION 1..7: note = Description of Unknown: EphA5 sequence
VYVFQIR

SEQ ID NO: 802 - protein, 11 aa, unidentified
  REGION 1..11: note = Description of Unknown: EphA5 sequence
VFSRRFEFETT

SEQ ID NO: 803 - protein, 16 aa, unidentified
  REGION 1..16: note = Description of Unknown: EphA6 sequence
DAPSLIGMMRKDWASQ

SEQ ID NO: 804 - protein, 5 aa, unidentified
  REGION 1..5: note = Description of Unknown: EphA6 sequence
KEHEQ

SEQ ID NO: 805 - protein, 7 aa, unidentified
  REGION 1..7: note = Description of Unknown: EphA6 sequence
KYVFHIR

SEQ ID NO: 806 - protein, 11 aa, unidentified
  REGION 1..11: note = Description of Unknown: EphA6 sequence
GYSQKFEFETG

SEQ ID NO: 807 - protein, 16 aa, unidentified
  REGION 1..16: note = Description of Unknown: EphA7 sequence
AAPSQVSGVMKERVLQ

SEQ ID NO: 808 - protein, 5 aa, unidentified
  REGION 1..5: note = Description of Unknown: EphA7 sequence
KDQRE

SEQ ID NO: 809 - protein, 7 aa, unidentified
  REGION 1..7: note = Description of Unknown: EphA7 sequence
VYVFQIR

SEQ ID NO: 810 - protein, 11 aa, unidentified
  REGION 1..11: note = Description of Unknown: EphA7 sequence
NYSPRLDVATL

SEQ ID NO: 811 - protein, 16 aa, unidentified
  REGION 1..16: note = Description of Unknown: EphA8 sequence
AAPSQVVVIRQERAGQ

SEQ ID NO: 812 - protein, 5 aa, unidentified
  REGION 1..5: note = Description of Unknown: EphA8 sequence
KDKEM

SEQ ID NO: 813 - protein, 7 aa, unidentified
  REGION 1..7: note = Description of Unknown: EphA8 sequence
RYVFQVR

SEQ ID NO: 814 - protein, 11 aa, unidentified
  REGION 1..11: note = Description of Unknown: EphA8 sequence
RFSQAMEVETG

SEQ ID NO: 815 - protein, 16 aa, unidentified
  REGION 1..16: note = Description of Unknown: EphA10 sequence
GAPWEEGEIRRDRVEP

SEQ ID NO: 816 - protein, 5 aa, unidentified
  REGION 1..5: note = Description of Unknown: EphA10 sequence
KGQSE

SEQ ID NO: 817 - protein, 7 aa, unidentified
  REGION 1..7: note = Description of Unknown: EphA10 sequence
RYVFQIR

SEQ ID NO: 818 - protein, 11 aa, unidentified
  REGION 1..11: note = Description of Unknown: EphA10 sequence
SFNPSIEVQTL

SEQ ID NO: 819 - protein, 16 aa, unidentified
  REGION 1..16: note = Description of Unknown: EphB1 sequence
AAPSTVPIMHQVSATM

SEQ ID NO: 820 - protein, 5 aa, unidentified
  REGION 1..5: note = Description of Unknown: EphB1 sequence
KEHNE

SEQ ID NO: 821 - protein, 7 aa, unidentified
  REGION 1..7: note = Description of Unknown: EphB1 sequence
VYVVQVR

SEQ ID NO: 822 - protein, 11 aa, unidentified
  REGION 1..11: note = Description of Unknown: EphB1 sequence
KFSGKMCFQTL

SEQ ID NO: 823 - protein, 16 aa, unidentified
  REGION 1..16: note = Description of Unknown: EphB2 sequence
AAPSAVSIMHQVSRTV

SEQ ID NO: 824 - protein, 5 aa, unidentified
  REGION 1..5: note = Description of Unknown: EphB2 sequence
KELSE

SEQ ID NO: 825 - protein, 7 aa, unidentified
  REGION 1..7: note = Description of Unknown: EphB2 sequence
IYVFQVR

SEQ ID NO: 826 - protein, 11 aa, unidentified
  REGION 1..11: note = Description of Unknown: EphB2 sequence
RYSGKMYFQTM

SEQ ID NO: 827 - protein, 16 aa, unidentified
  REGION 1..16: note = Description of Unknown: EphB3 sequence
AAPSEVPTLRLHSSSG

SEQ ID NO: 828 - protein, 7 aa, Rattus sp.
SKVHEFQ

SEQ ID NO: 829 - protein, 7 aa, unidentified
  REGION 1..7: note = Description of Unknown: EphB3 sequence
RYVVQVR

SEQ ID NO: 830 - protein, 11 aa, unidentified
  REGION 1..11: note = Description of Unknown: EphB3 sequence
QYSRPAEFETT

SEQ ID NO: 831 - protein, 16 aa, unidentified
  REGION 1..16: note = Description of Unknown: EphB4 sequence
EVPPAVSDIRVTRSSP

SEQ ID NO: 832 - protein, 5 aa, unidentified
  REGION 1..5: note = Description of Unknown: EphB4 sequence
KGAEG

SEQ ID NO: 833 - protein, 7 aa, unidentified
  REGION 1..7: note = Description of Unknown: EphB4 sequence
SYLVQVR

SEQ ID NO: 834 - protein, 11 aa, unidentified
  REGION 1..11: note = Description of Unknown: EphB4 sequence
PFGQEHHSQTQ

SEQ ID NO: 835 - protein, 16 aa, unidentified
  REGION 1..16: note = Description of Unknown: EphB6 sequence
EVPSAVPVVHQVSRAS

SEQ ID NO: 836 - protein, 5 aa, unidentified
  REGION 1..5: note = Description of Unknown: EphB6 sequence
QAEDE

SEQ ID NO: 837 - protein, 7 aa, unidentified
  REGION 1..7: note = Description of Unknown: EphB6 sequence
IYGFQVR

SEQ ID NO: 838 - protein, 11 aa, unidentified
  REGION 1..11: note = Description of Unknown: EphB6 sequence
PYGGKVYFQTL

SEQ ID NO: 839 - protein, 7 aa, Homo sapiens
SKVHEFQ

SEQ ID NO: 840 - protein, 7 aa, Mus sp.
SKVHEFQ

SEQ ID NO: 841 - protein, 7 aa, Macaca sp.
SKVHEFQ

(19) United States
(12) Patent Application Publication
(10) Pub. No.: US 2025/0276077 A1
(43) Pub. Date: Sep. 4, 2025

(54) EPHA2 ANTIBODIES

(71) Applicant: Immunome, Inc., Bothell, WA (US)

(72) Inventor: Alexander Scholz, San Carlos, CA (US)

(21) Appl. No.: 18/854,466

(22) PCT Filed: Apr. 5, 2023
(86) PCT No.: PCT/US2023/065395

§ 371 Date: Oct. 4, 2024

Related U.S. Application Data
(60) Provisional application No. 63/362,476, filed on Apr. 5, 2022.
(60) Provisional application No. 63/374,797, filed on Sep. 7, 2022.

(51) Int. Cl. A61K 47/68; A61K 9/00; A61P 35/00; C07K 16/28

(52) U.S. Cl. CPC A61K 47/68031; A61K 9/0019; A61K 47/6879; A61K 47/6889; A61P 35/00; C07K 16/2809; C07K 16/2818; C07K 16/2866; C07K 16/2878

(57) ABSTRACT

Provided herein immunoconjugate comprising an antibody that binds to ephrin receptor A2 (EphA2) and a cytotoxic agent. The cytotoxic agent is an auristatin analogue, and the antibody is conjugated to the auristatin analogue. These immunoconjugates comprising EphA2 antibodies bind preferentially to tumor tissue compared to normal tissue, and they can be used in methods of inducing an immune response and methods of inhibiting tumor cell growth.

### RELATED APPLICATIONS

This application claims priority to U.S. Application No. 63/362,476, filed on Apr. 5, 2022, and U.S. Application No. 63/374,797, filed on Sep. 7, 2022. The entire content of each provisional application is herein incorporated by reference for all purposes.

### REFERENCE TO SEQUENCE LISTING

The Sequence Listing .xml file, identified as 097519-1374172-2901 PCT, is 11,32,370 bytes in size and was created on Mar. 28, 2023. The Sequence Listing is hereby incorporated by reference in its entirety.

### FIELD OF CANCER THERAPEUTICS

This application relates to therapeutic antibodies for the treatment of various cancers.

### BACKGROUND

EphA2 is a tyrosine kinase and belongs to the family of Ephrin receptors. Like other Ephrin receptors, it is a single-pass membrane protein with large extracellular N-termini. EphA2 is typically involved in cell-cell repulsion or adhesion processes. EphA2 is also known to promote angiogenesis. EphA2 is overexpressed in tumor tissues compared to normal adult tissues, indicating its potential application in cancer treatment. Although several EphA2 antibodies have been developed, some of which are being assessed in clinical trials, these antibodies have the shortcomings of high toxicity and insufficient efficacy. They are unable to meet the needs of cancer patients.

### BRIEF SUMMARY

Provided herein is an immunoconjugate comprising an antibody that binds to ephrin receptor A2 (EphA2) and a cytotoxic agent, wherein the cytotoxic agent is an auristatin analogue, and wherein the antibody is conjugated to the auristatin analogue. In some embodiments, the auristatin analogue has a formula of:

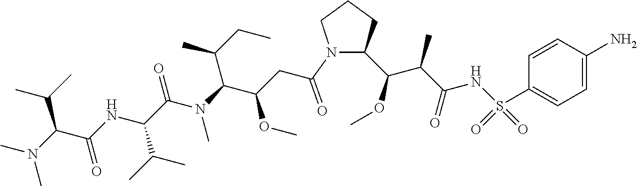 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C]

In some embodiments, the linker has a formula of

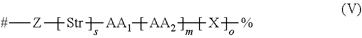

wherein:
Z is a linking group that joins the linker to a target group on the antibody;
Str is a stretcher:
AA₁ and AA₂ are each independently an amino acid, wherein AA₁-[AA₂]ᵣ forms a protease cleavage site;
X is a self-immolative group;
s is 0 or 1;
m is 1, 2, 3 or 4;
o is 0, 1 or 2;
# is the point of attachment to the antibody, and
% is the point of attachment to the auristatin analogue.

In some embodiments, the immunoconjugate has a formula of:

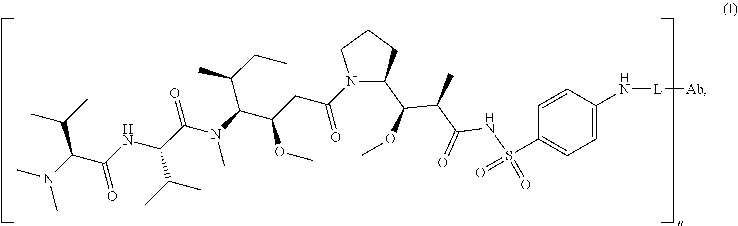

wherein: L is a cleavable linker; n is the drug-to-antibody ratio (DAR) and is an integer from 1 to 12, and Ab is the antibody.

In some embodiments, the immunoconjugate has a formula of:

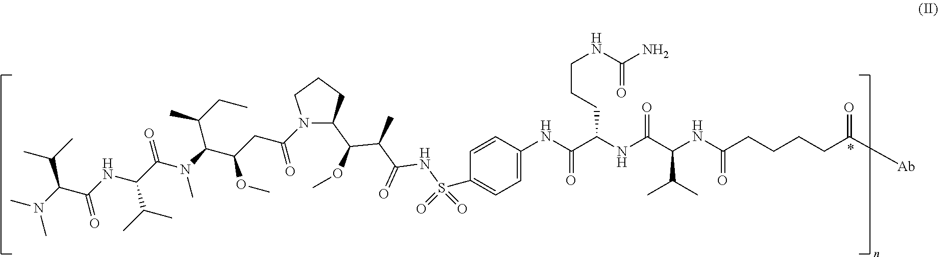

wherein n is the drug-to-antibody ratio (DAR) and is an integer from 1 to 12, and Ab is the antibody.

In some embodiments, the antibody of the immunoconjugate binds to an epitope of EphA2 located in the FN2 domain of EphA2, wherein the FN2 domain has the sequence of

(SEQ ID NO: 95)
TEPPKVRLEGRSTTSLSVSWSIPPPQQSRVWKYEVTYRKKGDSNSYNVR
RTEGFSVTLDDLAPDTTYLVQVQALTQEGQGAGSKVHEFQTLSPEGSG
N.

In some embodiments, the epitope comprises at least one, at least two, at least three, at least four, at least five, or at least six, at least seven, at least eight, or at least nine, or all ten of amino acid residues Leu444, Arg447, Lys476, Gln506, Ser519, Lys520, Val521, Glu523, Phe524, and Gln525 of SEQ ID NO: 94.

In some embodiments, the antibody of the immunoconjugate has one or more properties of: a) activates the ephrin A1-EphA2 signaling axis at least 95%, 90%, 85%, or 80% less than ephrin-A1 activates the ephrin A1-EphA2 signaling axis, b) binds preferentially to a tumor tissue than normal tissue, c) has an EC₅₀ for activation of the ephrinA1-EphA2 signaling axis that is at least 10-, 20-, 50-, 100-, 200-, 500-fold less potent than the ADCC EC₅₀ of the antibody; and/or d) has ADCP activity, and c) has ADCC activity.

In some embodiments, the antibody of the immunoconjugate disclosed herein binds to a tumor tissue preferentially compared to normal tissue.

In some embodiments, the antibody of the immunoconjugate comprises a heavy chain variable region comprising: an HCDR1 of any one of SEQ ID NOS:1-11 and 201-265 or a variant thereof in which 1, 2, 3, 4, or 5 amino acids are substituted relative to the sequence; an HCDR2 of any one of SEQ ID NOS:12-22 and 266-330 or a variant thereof in which 1, 2, 3, 4, or 5 amino acid is substituted relative to the sequence; and an HCDR3 of any one of SEQ ID NOS:23-33 and 331-395 or a variant thereof in which 1, 2, 3, 4, or 5 amino acids are substituted relative to the sequence; a light chain variable region comprising: an LCDR1 of any one of SEQ ID NOS:34-44 and 396-460 or a variant thereof in which 1, 2, 3, 4, or 5 amino acid is substituted relative to the sequence; an LCDR2 of any one of SEQ ID NOS:45-55 and 461-525 or a variant thereof in which 1, 2, or 3 amino acid is substituted relative to the sequence; and an LCDR3 of any one of SEQ ID NOS:56-66 and 526-590 or a variant thereof in which 1, 2, 3, 4, or 5 amino acid is substituted relative to the sequence.

In some embodiments, the antibody of the immunoconjugate comprises a heavy chain variable region comprising: an HCDR1 comprising a sequence (SEQ ID NO:1) or a variant HCDR1 in which 1, 2, 3, 4, or 5 amino acids are substituted relative to the sequence; an HCDR2 comprising a sequence (SEQ ID NO:12), or a variant HCDR2 in which 1, 2, 3, 4, or 5 amino acids substituted relative to the sequence; and an HCDR3 comprising a sequence (SEQ ID NO:23), or a variant HCDR3 in which 1, 2, 3, 4, or 5 amino acids are substituted relative to the sequence. The antibody further comprises a light chain variable region comprising: an LCDR1 comprising a sequence (SEQ ID NO:34) or a variant LCDR1 in which 1, 2, 3, 4, or 5 amino acids is substituted relative to the sequence; an LCDR2 comprising a sequence (SEQ ID NO:45), or variant LCDR2 in which 1, 2, or 3 amino acids is substituted relative to the sequence; and an LCDR3 comprising a sequence (SEQ ID NO:56), or a variant LCDR3 in which 1, 2, 3, 4, or 5 amino acids is substituted relative to the sequence.

In some embodiments, the antibody of the immunoconjugate comprises: (a) an HCDR1 sequence of GGSX₁X₂ X₃YX₄WS where X₁ is F or L, X₂ is S or N, X₃ is D or G, and X₄ is Y or H (SEQ ID NO: 769); (b) an HCDR2 sequence of EX₁NHX₂GSX₃X₄YNNYNPSLKS, where X₁ is I or V, X₂ is A, Q, R or S, X₃ is I or T, and X₄ is N or S (SEQ ID NO: 770); (c) an HCDR3 sequence of AKPX₁RPHC X₂NGVCX₃SGDAFDI, where X₁ is L or F, X₂ is I or T; and X₃ is Y or S (SEQ ID NO: 771); (d) an LCDR1 sequence of X₁GNNIGX₂ X₃ X₄VH, where X₁ is G or R, X₂ is S, T, or Y, X₃ is K or M, and X₄ is N or S (SEQ TD NO: 772); (e) an LCDR2 sequence of DDSDRPS (SEQ ID NO: 45); and (f) an LCDR3 sequence of QVWDX1 X2SDHX3V, where X1 is H or S, X2 is E, R, or S, and X3 is L or V (SEQ ID NO: 773).

In some embodiments, the antibody of the immunoconjugate comprises: (a) an HCDR1 sequence of GGSX₁X₂ X₃YX₄WS where X₁ is F or L, X₂ is S or N, and X₃ is D or G, and X₄ is Y or H (SEQ ID NO: 769); (b) an HCDR2 sequence of EX₁NHX₂GSX₃X₄YNPSLKS, where X₁ is I or V, X₂ is A, Q, R or S, X₃ is I or T, and X₄ is N or S (SEQ ID NO: 774); (c) an HCDR3 sequence of AKPX₁RPHCX₂NGVCX₃SGDAFDI, where X₁ is L or F, X₂ is I or T; and X₃ is Y or S (SEQ ID NO: 771); (d) an LCDR1 sequence of X₁GNNIGX₂ X₃ X₄VH, where X₁ is G or R, X₂ is S, T, or Y, X₃ is K or M, and X₄ is N or S (SEQ ID NO: 772); (e) an LCDR2 sequence of DDSDRPS (SEQ ID NO: 45); and (f) an LCDR3 sequence of QVWDX1 X2SDHX3V, where X1 is H or S, X2 is E, R, or S, and X3 is L or V (SEQ ID NO: 773).

In some embodiments, the antibody comprises: (a) an HCDR1 sequence of GGSX₁X₂DYX₃WS where X₁ is F or L, X₂ is S or N, and X₃ is Y or H (SEQ ID NO: 775); (b) an HCDR2 sequence of EX1NHX2 GS X₃ X₄YNPSLKS, where X₁ is I or V, X₂ is R or S, X₃ is I or T, and X₄ is N or S (SEQ ID NO: 775); or an HCDR2 sequence of EX₁NHX₂ GS X₃ X₄YNNYNPSLKS, where X₁ is I or V, X₂ is R or S, X₃ is I or T, and X₄ N or S(SEQ ID NO: 777); and (c) an HCDR3 sequence of AKP X₁RPHCTNGVCX2SGDAFDI, where X₁ is L or F and X₂ is Y or S (SEQ ID NO: 778); (d) an LCDR1 sequence of GGNNIGX₁KNVH, where X₁ is S or T (SEQ ID NO: 779); (e) an LCDR2 sequence DDSDRPS (SEQ ID NO: 45); and (f) an LCDR3 sequence QVWDSSSDHLV (SEQ ID NO: 56).

In some embodiments, the antibody of the immunoconjugate comprises a V_{H} comprising an amino acid sequence having at least 95% identity to a V_{H} in Table 8; and a V_{L} comprising an amino sequence having at 95% identity to a V_{L} in Table 8. In some embodiments, the V_{H} comprises an amino acid sequence having at least 95% identity to (SEQ ID NO: 67); and the V_{L} comprises an amino sequence having at 95% identity to (SEQ ID NO: 78). In some embodiments, the V_{H} comprises an amino acid sequence having at least 95% identity to a V_{H} of any one of SEQ ID NOS:67-77 and 591-655. The V_{L} comprises an amino sequence having at 95% identity to a V_{L} of any one of SEQ ID NOS:78-88 and 656-720.

In some embodiments, the antibody of the immunoconjugate comprises: a V_{H} region comprising amino acid sequence SEQ ID NO:67 and a V_{L} region comprising amino acid sequence SEQ ID NO:78; a V_{H} region comprising amino acid sequence SEQ ID NO:68 and a V_{L} region comprising amino acid sequence SEQ ID NO:79; a V_{H} region comprising amino acid sequence SEQ ID NO:69 and a VL region comprising amino acid sequence SEQ ID NO:80; a V_{H} region comprising amino acid sequence SEQ ID NO:70 and a VL region comprising amino acid sequence SEQ ID NO:81; a V_{H} region comprising amino acid sequence SEQ ID NO:71 and a V_{L} region comprising amino acid sequence SEQ TD NO:82; a V_{H} region comprising amino acid sequence SEQ TD NO:72 and a V_{L} region comprising amino acid sequence SEQ ID NO:83; a V_{H} region comprising amino acid sequence SEQ ID NO:73 and a V_{L} region comprising amino acid sequence SEQ ID NO:84; a V_{H} region comprising amino acid sequence SEQ ID NO:74 and a V_{L} region comprising amino acid sequence SEQ ID NO:85; a V_{H} region comprising amino acid sequence SEQ ID NO:75 and a V_{L} region comprising amino acid sequence SEQ ID NO:86; or a V_{H} region comprising amino acid sequence SEQ ID NO:76 and a V_{L} region comprising amino acid sequence SEQ ID NO:87; a V_{H} region comprising amino acid sequence SEQ ID NO:77 and a V_{L} region comprising amino acid sequence SEQ ID NO:88, a V_{H} region comprising amino acid sequence SEQ ID NO:652 and a V_{L} region comprising amino acid sequence SEQ ID NO:717, or a V_{H} region comprising amino acid sequence SEQ ID NO:607 and a V_{L} region comprising amino acid sequence SEQ ID NO:672.

In some embodiments, antibody of the immunoconjugate comprises: (1) a heavy chain variable (V_{H}) region and a light chain variable (V_{L}) region, wherein: (a) the V_{H} region has at least 70% identity to SEQ ID NO:67; and comprises a CDR1 of SEQ ID NO:1, or the CDR1 of SEQ ID NO:1 in which 1, 2, 3, 4, or 5 amino acids are substituted; a CDR2 of SEQ ID NO:12, or the CDR2 of SEQ ID NO:12 in which 1, 2, 3, 4, or 5 amino acids are substituted; a CDR3 of SEQ ID NO:23 or the CDR3 of SEQ ID NO:23 in which 1, 2, 3, 4, or 5 are substituted; and (b) the V_{L} region has at least 70% identity to SEQ ID NO: 78, and comprises a CDR1 of SEQ ID NO:34 or the CDR1 of SEQ ID NO:34 in which 1, 2, 3, 4, or 5 amino acids are substituted; a CDR2 of SEQ ID NO:45, or the CDR2 of SEQ ID NO:45 in which 1, 2, 3, 4, or 5 amino acids are substituted; a CDR3 of SEQ ID NO:56 or the CDR3 of SEQ ID NO:56 in which 1, 2, 3, 4, or 5 are substituted; (2) a heavy chain variable (V_{H}) region and a light chain variable (V_{L}) region, wherein: (a) the V_{H} region has at least 70% identity to SEQ ID NO:652; and comprises a CDR1 of SEQ ID NO:262, or the CDR1 of SEQ ID NO:262 in which 1, 2, 3, 4, or 5 amino acids are substituted; a CDR2 of SEQ ID NO:327, or the CDR2 of SEQ ID NO:327 in which 1, 2, 3, 4, or 5 amino acids are substituted; a CDR3 of SEQ ID NO:392 or the CDR3 of SEQ ID NO:392 in which 1, 2, 3, 4, or 5 are substituted; and (b) the VL region has at least 70% identity to SEQ ID NO: 717, and comprises a CDR1 of SEQ TD NO:457 or the CDR1 of SEQ TD NO:457 in which 1, 2, 3, 4, or 5 amino acids are substituted; a CDR2 of SEQ ID NO:522, or the CDR2 of SEQ ID NO:522 in which 1, 2, 3, 4, or 5 amino acids are substituted; a CDR3 of SEQ ID NO:587 or the CDR3 of SEQ ID NO:587 in which 1, 2, 3, 4, or 5 are substituted; or (3) a heavy chain variable (V_{H}) region and a light chain variable (V_{L}) region, wherein: (a) the V_{H} region has at least 70% identity to SEQ ID NO:607; and comprises a CDR1 of SEQ ID NO:217, or the CDR1 of SEQ ID NO:217 in which 1, 2, 3, 4, or 5 amino acids are substituted; a CDR2 of SEQ ID NO:282, or the CDR2 of SEQ ID NO:282 in which 1, 2, 3, 4, or 5 amino acids are substituted; a CDR3 of SEQ ID NO:347 or the CDR3 of SEQ ID NO:347 in which 1, 2, 3, 4, or 5 are substituted; and (b) the V_{L} region has at least 70% identity to SEQ ID NO: 672, and comprises a CDR1 of SEQ ID NO:412 or the CDR1 of SEQ ID NO:412 in which 1, 2, 3, 4, or 5 amino acids are substituted; a CDR2 of SEQ ID NO:477, or the CDR2 of SEQ ID NO:477 in which 1, 2, 3, 4, or 5 amino acids are substituted; a CDR3 of SEQ ID NO:542 or the CDR3 of SEQ ID NO:542 in which 1, 2, 3, 4, or 5 are substituted.

The immunoconjugate of any one of claims 1-8, wherein the antibody comprises: a heavy chain variable (VH) region and a light chain variable (VL) region, (i) wherein the VH region has at least 70% identity to SEQ ID NO:67 and comprises (a) an HCDR1 sequence of GGSX1X2DYX3WS where X1 is F or L, X2 is S or N, and X3 is Y or H (SEQ ID NO: 775); (b) an HCDR2 sequence of EX1NHX2 CS X3 X4YNPSLKS, where X1 is I or V, X2 is R or S, X3 is I or T, and X4 is N or S (SEQ ID NO: 776); or an HCDR2 sequence of EX1NHX2 GS X3 X4YNNYNPSLKS, where X1 is I or V, X2 is R or S, X3 is I or T, and X4 N or S (SEQ ID NO: 777); and (c) an HCDR3 sequence of AKP X1RPHCTNGVCX2SGDAFDI, where X1 is L or F and X2 is Y or S (SEQ ID NO: 778); and (ii) wherein the VL region has at least 70% identity to SEQ ID NO:78 and comprises (d) an LCDR1 sequence of GGNNIGX1KNVH, where X1 is S or T (SEQ ID NO: 779); (e) an LCDR2 sequence DDSDRPS (SEQ ID NO: 45); and (f) an LCDR3 sequence QVWDSSSDHLV (SEQ ID NO: 56).

In some embodiments, the antibody of the immunoconjugate comprises an HCDR1, HCDR2, HCDR3, LCDR1, LCDR2, and LCDR3 of an antibody designated as AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698, AB-010699, AB-010700, AB-010701, or AB-010702, or a variant thereof in which at least one, two, three, four, five, or all six of the CDRs contain 1 or 2 amino acid substitutions compared to the corresponding CDR.

In some embodiments, the antibody of the immunoconjugate comprises a heavy chain variable region and a light chain variable region of an antibody designated as AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698, AB-010699, AB-010700, AB-010701, or AB-010702, or a variant thereof, wherein the variant comprises a heavy chain variable region having a sequence that is at least 95% identical to that of the corresponding heavy chain variable region and a light chain variable region having a sequence that is at least 95% identical to the corresponding light chain variable region.

In some embodiments, at least 1 or 2 of the substitutions referenced above are conservative substitutions and at least 50% of the substitutions are conservative substitutions. In some embodiments, all the substitutions are conservative substitutions.

In some embodiments, the antibody of the immunoconjugate comprises a V_{H} region of any one of SEQ ID NO 67-77 and 591-655, and/or a V_{L} region of any one of SEQ ID NO: 78-88 and 656-720, or an antibody comprising a V_{H} region with at least 80% identity to any one of SEQ ID NO 67-77 and 591-655 and a V_{L} region having at least 80% identity to any one of SEQ ID NO: 78-88 and 656-720, with variations to the corresponding V_{H} or V_{L} regions present only in Framework regions.

In some embodiments, the antibody of the immunoconjugate comprises an HCDR1 of any one of SEQ ID NOS: 1-11, and 201-265, an HCDR2 of any one of SEQ ID NOS:12-22 and 266-330, an HCDR3 of any one of SEQ ID NOS:23-33 and 331-395, an LCDR1 of any one of SEQ ID NOS:34-44 and 396-460, an LCDR2 of any one of SEQ ID NOS:45-55 and 461-525, an LCDR3 of any one of SEQ ID NOS:56-66 and 526-590, wherein the FW regions in the V_{H} region are at least 80% identical to the FW regions present in the V_{H} region of SEQ ID NO 67-77 and 591-655, and wherein the FW regions in the V_{L} region are at least 80% identical to the FW regions present in the V_{L} region of SEQ ID NO: 78-88 and 656-720.

In some embodiments, the antibody of the immunoconjugate is a non-natural antibody.

In some embodiments, the antibody is a bispecific antibody comprising two antigen binding fragments, one binding to EphA2, and the other binds to a different antigen. In some embodiments, the different antigen is 4-1bb or CD3.

In some embodiment, the immunoconjugate comprising the antibody disclosed herein further comprises one or more of an IL-15 receptor agonist, a TGFβ trap, a TLR agonist, or a 4-1BB ligand (4-1BBL).

Also provided in this disclosure is a pharmaceutical composition comprising any of the immunoconjugates disclosed herein and a pharmaceutically acceptable carrier.

Also provided in this disclosure is a method of inducing an immune response and/or treating cancer, the method comprising administering any one of the immunoconjugates disclosed herein. In some embodiments, the immune response comprises an antibody-dependent cellular cytotoxicity (ADCC) and/or antibody dependent cellular phagocytosis (ADCP). In some embodiments, the antibody is administered intravenously.

Also provided in this disclosure is a method of treating a cancer patient having a tumor overexpressing EphA2, the method comprising administering any one of the immunoconjugates disclosed herein to the patient. In some embodiments, the cancer is a gastric cancer, ovarian cancer, soft tissue sarcoma, lung cancer, head and neck cancer, uterine cancer, breast cancer, colorectal cancer, esophageal cancer, stomach cancer, kidney cancer, melanoma, liver cancer, bladder cancer, or testicular cancer. In some embodiments, the cancer is non-small cell lung cancer, triple-negative breast cancer, colorectal cancer, ovarian cancer or melanoma. In some embodiments, the method further comprises administering chemotherapy and/or radiation therapy. In some embodiments, the method comprises administering an agent that targets an immunological checkpoint antigen. In some embodiments, the agent is a monoclonal antibody. In some embodiments, the monoclonal antibody blocks PD-1 ligand binding to PD-1. In some embodiments, the monoclonal antibody is an anti-PD-1 antibody.

### BRIEF DESCRIPTION OF THE DRAWINGS

FIG. 1 shows surface binding profile of AB-008873 in A549 cells and CT26 cells (in vitro and ex vivo).

FIG. 2A and FIG. 2B shows results of assessing the ADCC activity (FIG. 2A) and ADCP activity (FIG. 2B) of AB-008873 in vitro functional assays.

FIG. 3 shows the ADC activity of AB-008873 on in vitro cell lines.

FIG. 4 compares the binding activity and functional activity of AB-008873 and other EphA2 antibodies on mouse and human cells.

FIG. 5A illustrates a method of octet in tandem binning for epitope mapping. FIG. 5B illustrates the epitopes in the EphA2 domains recognized by EphA2 antibodies.

FIG. 6 shows the results from a yeast display that demonstrates the binding of AB-008873 to FN2 of EphA2.

FIG. 7 shows the design of an in vivo study to assess the effect of the AB-008873 treatment.

FIG. 8 shows that AB-008873 treatment increased T cells in the blood.

FIG. 9 shows the effects on tumor growth of treatment of AB-008873 on mice inoculated with CT26 cells.

FIG. 10 shows that AB-008873 treatment did not result in significant changes in TIL number.

FIG. 11 shows that AB-008873 treatment did not significantly change T & NK cell numbers in the TME.

FIG. 12 shows increased macrophage numbers in TME at Day 10 with 40 mg/kg AB-008873 treatment.

FIG. 13 shows representative CD3+ CD8+ staining of cytotoxic T cells.

FIG. 14 shows representative CD4+ FoxP3+ staining of regulatory T cells.

FIG. 15 shows representative F4/80+ iNOS+ staining on M1 macrophages.

FIG. 16 shows the effect of 40 mg/kg AB-00 8873 administered on tumor growth and body weight in mice inoculated with CT26 cells (murine colon carcinoma cells).

FIG. 17 shows flow cytometry analysis of total tumor-infiltrating leukocytes (TIL) and myeloid cells from tumors in mice inoculated with CT26 cells. The mice were treated with 40 mg/kg AB-008873 or PBS.

FIG. 18 shows flow cytometry analysis of lymphoid cells from tumors in mice inoculated with CT26 cells. The mice were treated with 40 mg/kg AB-008873 or PBS.

FIGS. 19A, 19B, 19C, 19D, and 19E show results of activation of 4-1BB by EphA2-4-1BB bispecific antibodies in tumor cell lines.

FIG. 20 shows the functional activity of AB-008873 sibling antibodies (antibodies that are derived from the same lineage as AB-008873)

FIG. 21A-C show the functional activity of engineered AB-008873 variants: flow binding, ADC activity, and ADCC activity, respectively.

FIG. 22 shows the reactivity of AB-010699 across a panel of human cancer samples.

FIG. 23 shows the binding sites of various commercial EphA2 antibodies.

FIG. 24 shows the reactivity of EphA2 antibodies across a panel of human cancer samples.

FIG. 25 shows immunofluorescence study results demonstrating a possible unique epitope of AB-008873 in gastric cancer.

FIG. 26 compares EphA2 antibodies in binding ovarian cancer and soft tissue sarcoma cells.

FIG. 27 compares AB-008873 with commercial anti-EphA2 antibodies in binding to ovarian cancer and soft tissue sarcoma cells.

FIG. 28 compares the binding pattern of EphA2 antibodies in tumor tissues from different uterine cancer donors.

FIG. 29 shows the binding pattern of EphA2 antibodies in tumor tissues from different head and neck cancer donors.

FIG. 30 shows the binding pattern of EphA2 antibodies in tumor tissues from different Non-small Cell Lung cancer (NSCLC) donors.

FIG. 31 shows the reactivity of EphA2 antibodies across a panel of additional human cancer samples.

FIG. 32 shows schematics of the formats of multivalent antibodies, including the format of the Fc regions.

FIG. 33 shows the binding of AB-008873, variants thereof, AB-010018 and commercial anti-EphA2 antibodies to normal kidney.

FIG. 34 shows the binding of AB-008873 and its variants, AB-010018, and a commercial anti-EphA2 antibody to a normal stomach.

FIG. 35 shows a comparison of the ADC activity of AB-008873 with AB-009815 (comprising an H76PT mutation relative to AB-008873). This mutation removes a medium-risk DP proteolytic cleavage site that could cause AB degradation in the serum, decreasing half-life after injection in mice/humans. The results showed that the ADC activity of AB-009815 is substantially similar to that of AB-008873 in H522, LoVo, A375, SKOV3, A549, and MDAMB231 cells.

FIG. 36 is a schematic illustration of AB-008873-IL15 fusions.

FIG. 37 shows that AB-008873-IL15 multiMabs treatment resulted in dose-dependent increases in human NK cell proliferation.

FIG. 38 shows the flow cytometry analysis of the binding of the AB-008873-IL15 multiMabs to tumor cells using flow cytometry.

FIG. 39A-D show alignments and CDR designations for various EphA2 antibodies. FIG. 39A-D discloses SEQ ID NOS 67-88, 67, 75, 598, 603, 607, 609, 652, 78, 86, 663, 668, 672, 674, and 717, respectively, in order of appearance.

FIG. 40 compares the ADCC activity of several of anti-EphA2 antibodies AB-010361 (“AB-010361-15”), AB-010681 (“AB-010681-2”), and AB-010685 (“AB-010681-2”) on A549 cells.

FIG. 41 shows the results of thermostability analysis of the anti-EphA2 antibodies AB-008873 and AB-010699.

FIG. 42 shows immunofluorescence staining results of AB-008873 variants as compared to AB-008873, and AB-010018.

FIG. 43A and FIG. 43B show the phosphorylation of Y588 and S897 on EphA2 by anti-EphA2 antibodies AB-008873, AB-010361. AB-010699, AB-010016, AB-010018, and AB-010019 in agonist (FIG. 43A) and antagonist (FIG. 43B) assay.

FIG. 44 shows identification of AB-008873 epitope residues by yeast display. The epitope residues identified by this method include Pro439, Lys441, Arg443, Leu444, Arg447, Lys476, Gly477, Leu504, Gln506, Ser519, Lys520, Val521, His522, Glu523, Phe524, and Gln525.

FIG. 45 shows the alignment of the epitope residues across the EphA/B family members. The results show that the residues are divergent across the EphA/B family and the epitope is conformational. FIG. 45 discloses SEQ ID NOS 783-827 and 829-838, respectively, in order of appearance.

FIG. 46 shows the alignment of the epitope residues across multiple species: human, cynomolgus monkey (“cyno”), mouse, and rat. FIG. 46 discloses SEQ ID NOS 839-841 and 828, respectively, in order of appearance.

FIG. 47 illustrates the co-crystallization configuration of the EphA2 protein and the AB-008873 antibody. It indicates that the HCDRs changes conformation upon binding to the EphA2 protein: the disulfide loop in HCDR3 shows substantial rotation as compared to the structure that is not in contact with EphA2.

FIG. 48 shows measurements of tumor growth in mice inoculated with CT26 cells upon treatment of the AB-008873-IL15.

FIG. 49 shows measurements of tumor growth in mice inoculated with CT26 cells upon treatment of a bispecific antibody comprising scFv of the anti-4-1BB antibody 9365 and AB-010361.

FIG. 50A compares the cytotoxicity of AB-008873, AB-010361, AB-010699, and AB-010018 on A549 cells. FIG. 50B compares the cytotoxicity of AB-010699, AB-010695, AB-010363, AB-010699, and Cetuximab on A549 cells.

FIG. 51 compares the cytotoxicity of AB-008873, AB-010361, and AB-010699 in an ADC assay.

FIG. 52 compares the cytotoxicity of AB-010361 (“AB-010361-15”), AB-010018 (“AB-010018-1”), and AB-010685 (“AB-010685-2”) on PC3-KILR cells in an ADCC assay.

FIG. 53 shows representative anti-CD3 bispecific antibody constructs.

FIG. 54 shows measurements of tumor growth of NSG-DKO mice carrying PC3 tumor cells upon treatment of a bispecific antibody comprised of AB-010361 and an anti-CD3 binding arm.

FIG. 55A-D show cytotoxicity results of antibody-drug conjugates, each comprising an EphA2 antibody, AB-010361 (IgG1), AB-010361 (IgG4), AB-10699 (IgG1), or AB-010671 (IgG1), directly conjugated to a ZymeLink Auristatin (ZLA) payload. The cytotoxicity on SKOV3, MDAM1B231, and PC3 cells are shown in FIG. 55A-C and EC₅₀ values are reported in FIG. 55D.

FIG. 56A-D show measurements of tumor growth of NPG mice carrying PC3 tumor cells upon treatment of antibody-drug conjugates each comprising an EphA2 antibody and a ZLA payload.

FIG. 57 shows endpoint tumor weight analysis of NPG mice carrying tumors established from PC3 cells upon treatment of ZLA-conjugated EphA2 antibodies.

FIG. 58A-58D show the body weight analysis of NPG mice that carrying tumors established from PC3 cells upon treatment of ZLA-conjugated EphA2 antibodies.

FIG. 59A shows cytotoxicity results on PC3 cells of AB-10699-IgG1, AB-10699-IgG4, AB-010671-IgG1, and AB-010671-IgG4 directly conjugated to a ZymeLink Auristatin (ZLA) payload. The IC₅₀ values of the study are reported in FIG. 59B. IgG1 and IgG4 isotype antibodies are included as controls.

FIG. 60A shows measurements of tumor growth of NPG mice carrying PC3 tumor cells measuring ˜150 mm³ upon administration of AB-10699-IgG1, AB-10699-IgG4, AB-010671-IgG1, and AB-010671-IgG4 directly conjugated to a ZymeLink Auristatin (ZLA). FIG. 60B shows the body weight of the mice over the course of the study. IgG1 and IgG4 isotype antibodies are included as controls.

FIG. 61A shows measurements of tumor growth of NPG mice carrying PC3 tumor cells measuring ˜400 mm³ upon administration of AB-10699-IgG1, AB-10699-IgG4, AB-010671-IgG1, and AB-010671-IgG4 directly conjugated to a ZymeLink Auristatin (ZLA). FIG. 61B shows the body weight of the mice over the course of the study. IgG1 and IgG4 isotype antibodies are included as controls.

### DETAILED DESCRIPTION

This application incorporates the entire disclosure of PCT publication WO 2022/187710 by reference for all purposes.

As used in herein, the singular forms “a,” “an,” and “the” include plural referents unless the content clearly dictates otherwise. Thus, for example, reference to “an antibody” optionally includes a combination of two or more such molecules, and the like.

The term “about,” as used herein, refers to the usual error range for the respective value readily known to the skilled person in this technical field, for example ±20%, ±10%, or ±5%, are within the intended meaning of the recited value.

As used herein, the term “antibody” means an isolated or recombinant binding agent that comprises the necessary variable region sequences to specifically bind an antigenic epitope. Therefore, an “antibody” as used herein is any form of antibody of any class or subclass or fragment thereof that exhibits the desired biological activity, e.g., binding a specific target antigen. Thus, it is used in the broadest sense and specifically covers a monoclonal antibody (including full-length monoclonal antibodies), human antibodies, chimeric antibodies, nanobodies, diabodies, multispecific antibodies (e.g., bispecific antibodies), and antibody fragments including but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)₂, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment with two antigen combining sites and is still capable of cross-linking antigen.

As used herein, the term “targets a tumor,” or “tumor-targeting,” with respect to an antibody, refers to an antibody that binds preferentially to a tumor tissue than normal tissue. In some embodiments, the normal tissue is the tissue that is adjacent to the tumor, referred to as tumor-adjacent tissue or TAT. In some embodiments, a tumor targeting antibody also decreases the rate of tumor growth, tumor size, invasion, and/or metastasis, via direct or indirect effects on tumor cells.

As used herein, “V-region” refers to an antibody variable region domain comprising the segments of Framework 1, CDR1, Framework 2, CDR2, and Framework 3, including CDR3 and Framework 4. The heavy chain V-region, V_{H}, is a consequence of rearrangement of a V-gene (HV), a D-gene (HD), and a J-gene (HJ), in what is termed V(D)J recombination during B-cell differentiation. The light chain V-region, V_{L}, is a consequence of rearrangement of a V-gene (LV) and a J-gene (LJ).

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3, numbered sequentially starting from the N-terminus, and are also identified by the chain in which the CDR is located. Thus, for example, a V_{H} CDR3 (HCDR3) is in the variable domain of the heavy chain of the antibody in which it is found, whereas a V_{L} CDR3 (LCDR3) is the CDR3 from the variable domain of the light chain of the antibody in which it is found. The term “CDR” is used interchangeably with “HVR” in this application when referring to CDR sequences.

The amino acid sequences of the CDRs and framework regions can be determined using various well-known definitions in the art, e.g., Kabat, Chothia, international ImMunoGeneTics database (IMGT), and AbM (see, e.g., Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human V_{H} segments J. Mol. Biol. 227, 799-817; Al-Lazikani et al., J. Mol. Biol. 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996). Reference to CDRs as determined by Kabat numbering are based, for example, on Kabat et al., Sequences of Proteins of Immunological Interest, 5^{th} Ed. Public Health Service, National Institute of Health, Bethesda, MD (1991)). Chothia CDRs are determined as defined by Chothia (see, e.g., Chothia and Lesk J. Mol. Biol. 196:901-917 (1987)).

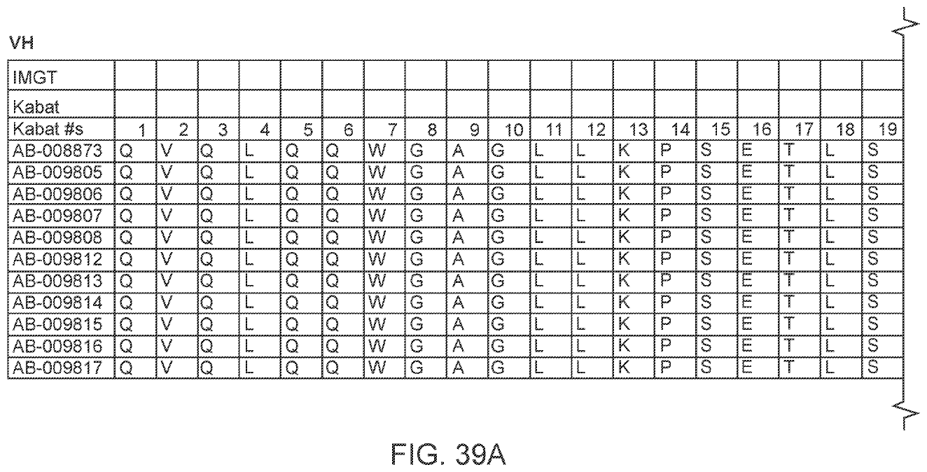

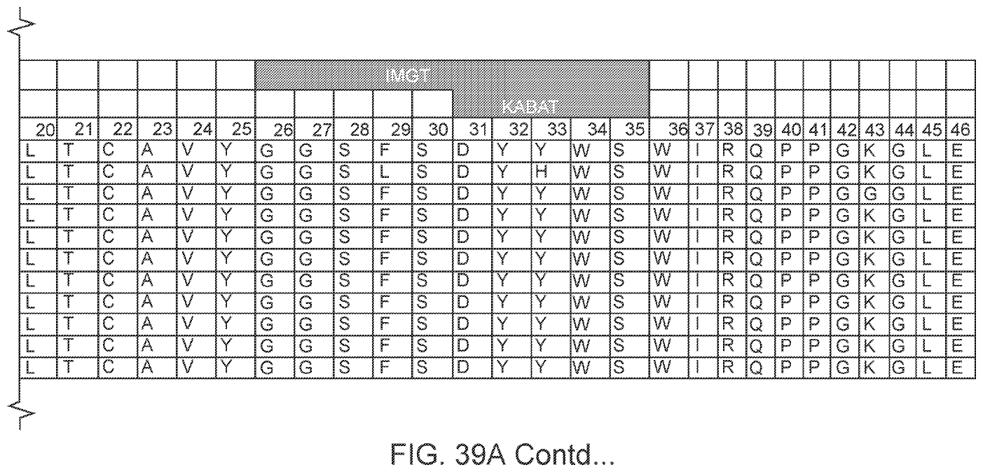

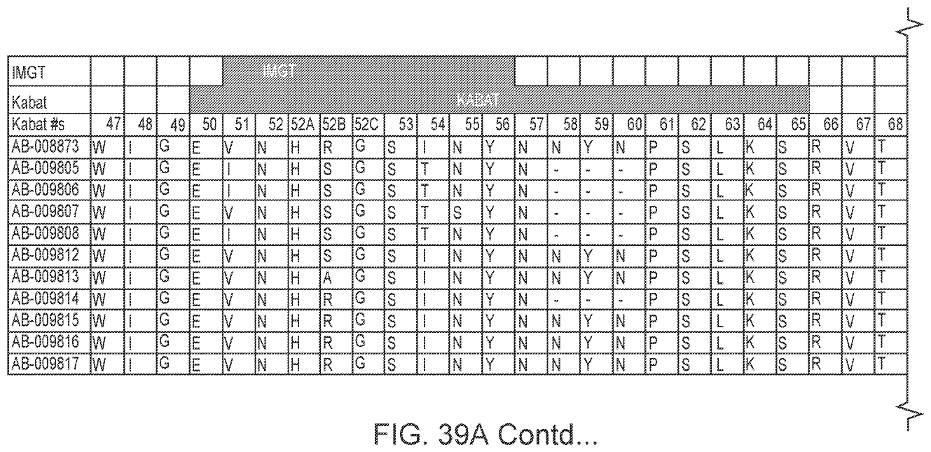

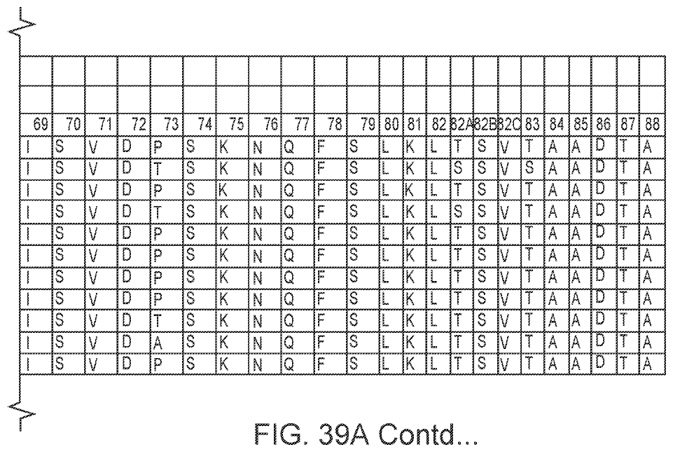

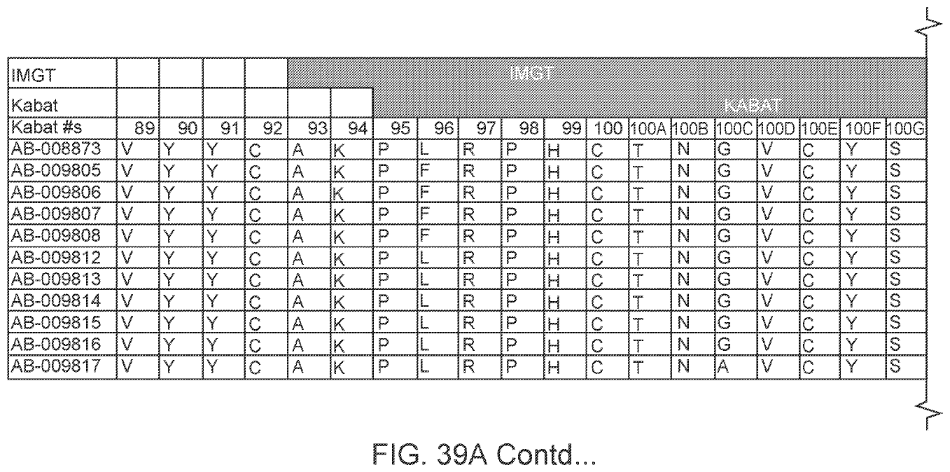

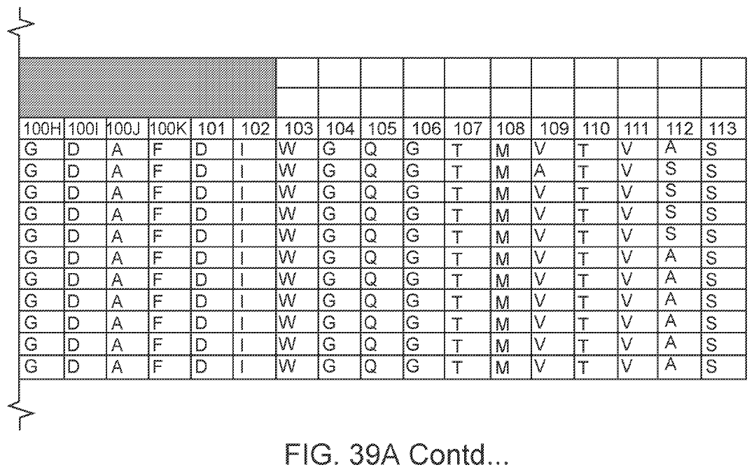

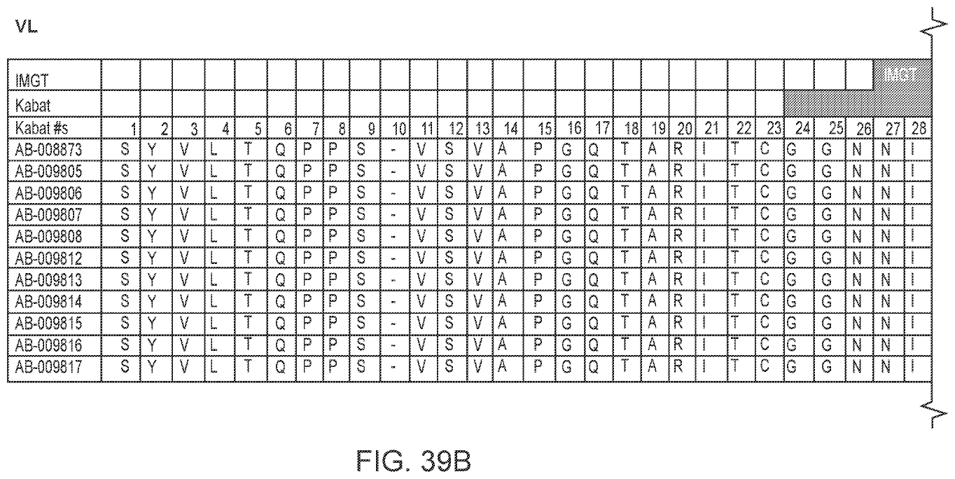

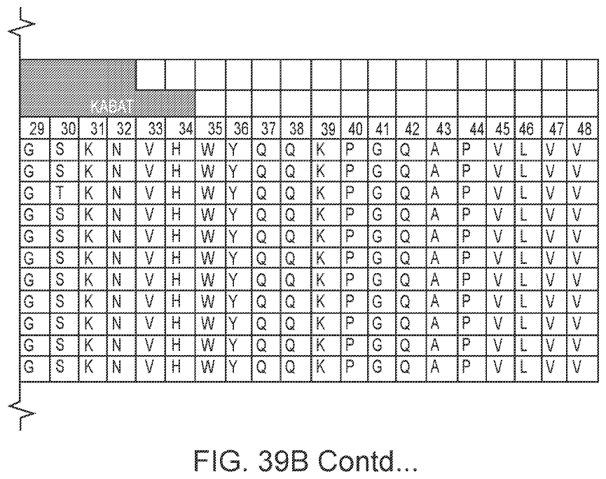

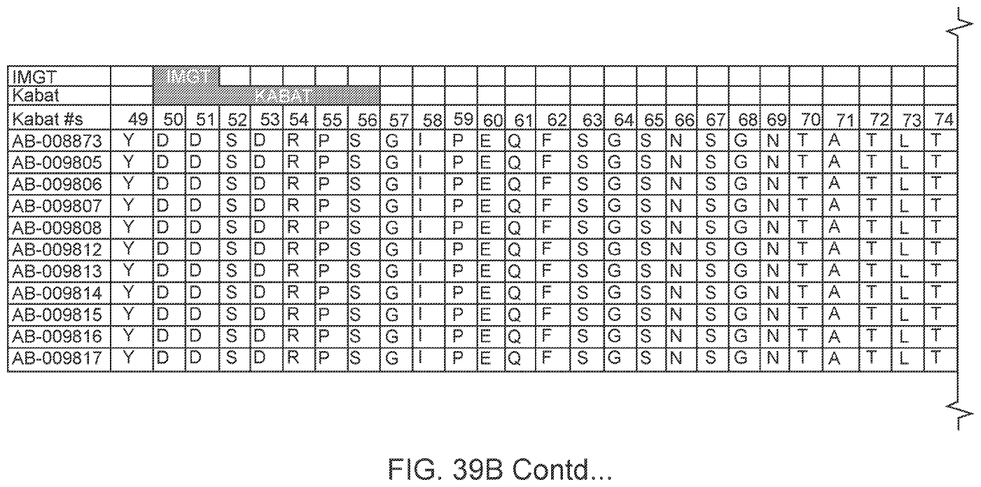

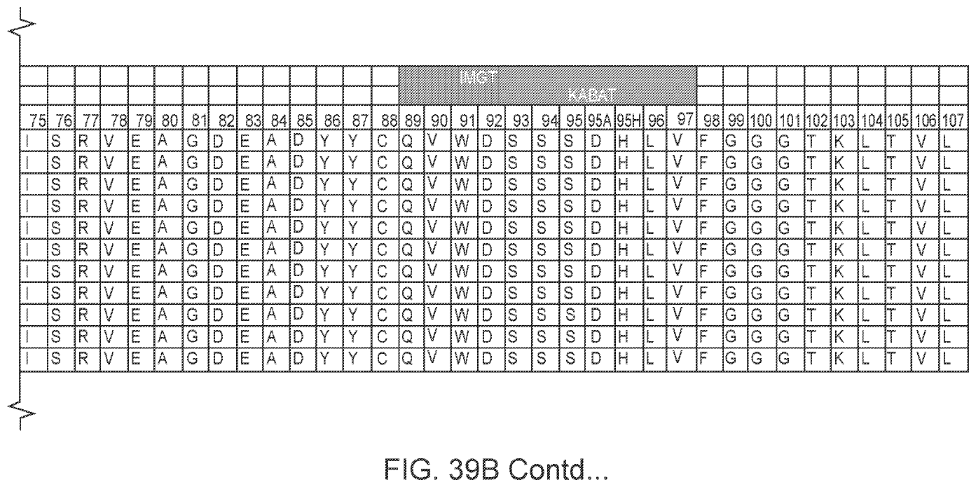

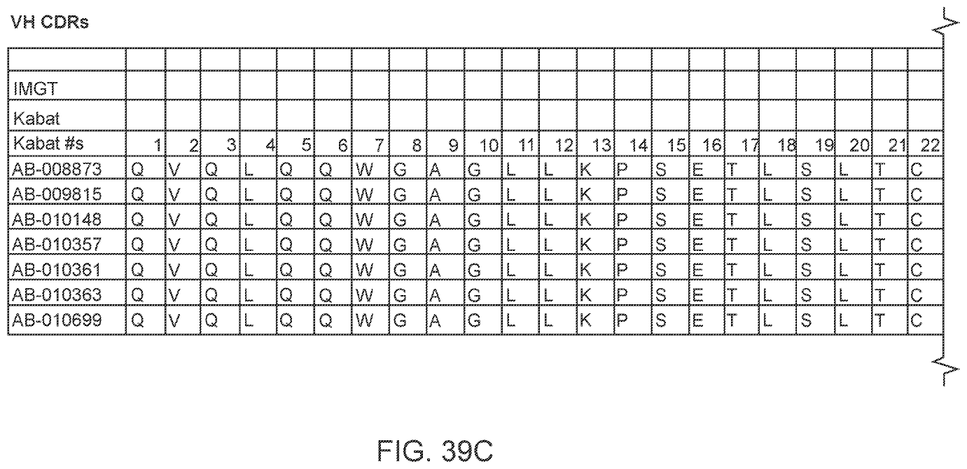

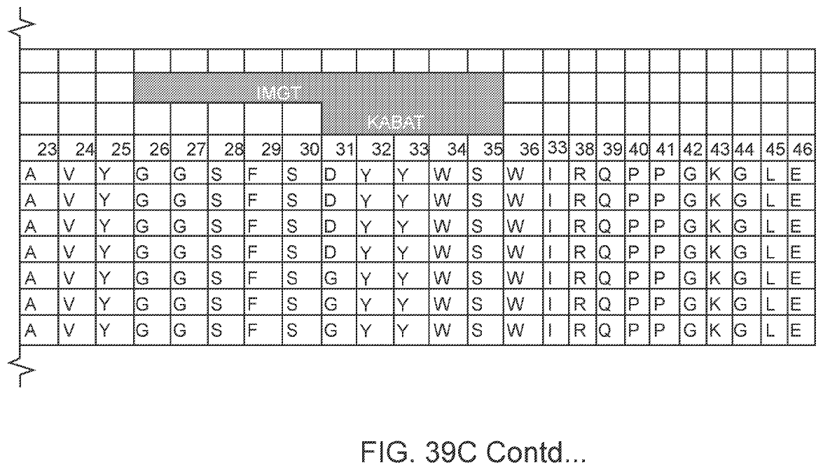

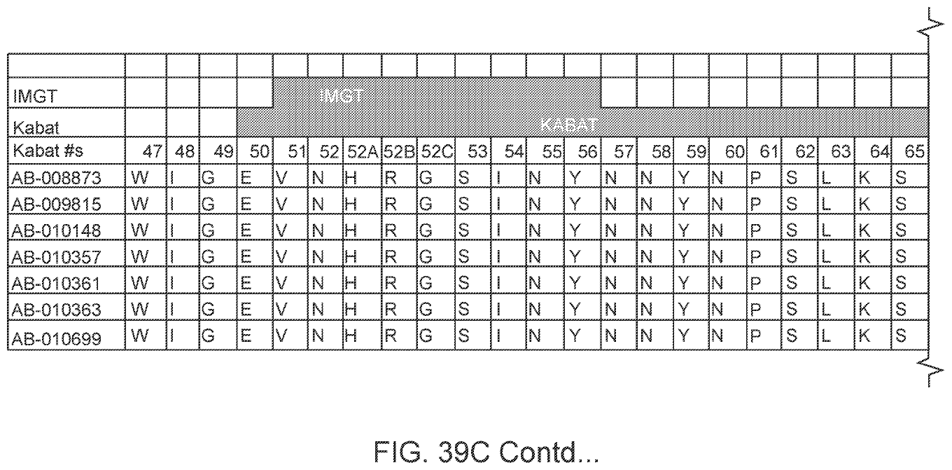

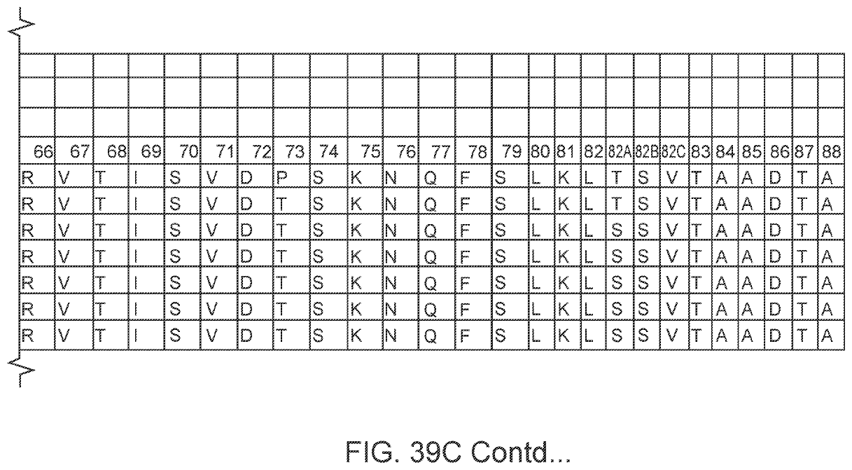

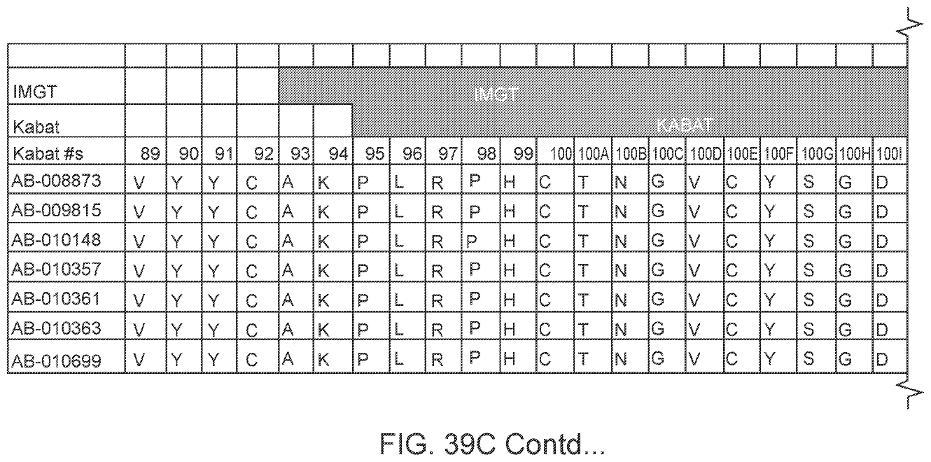

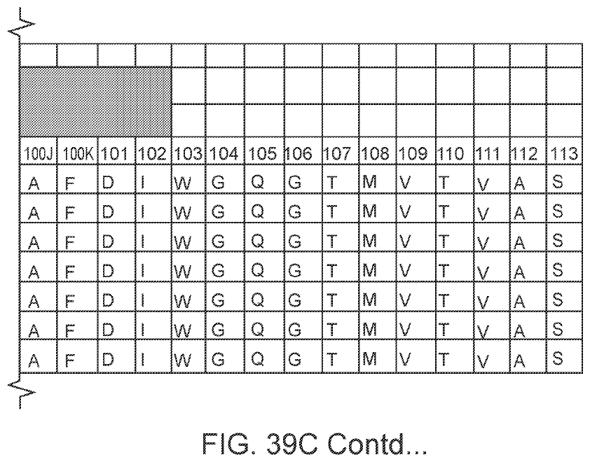

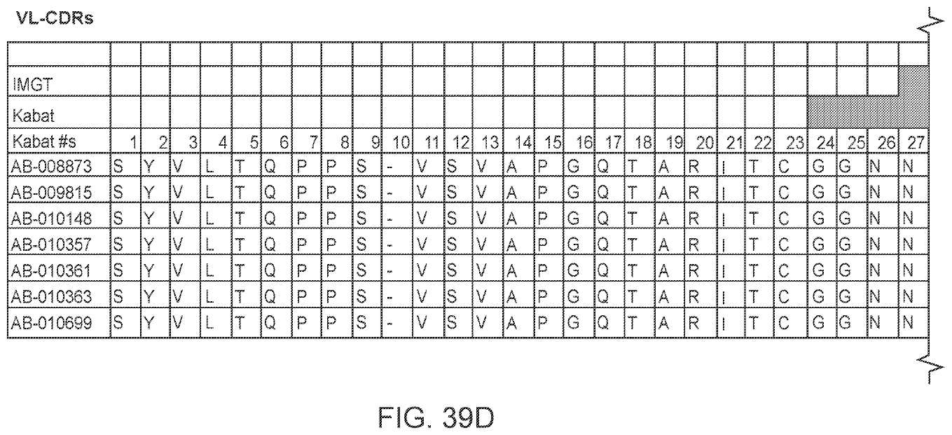

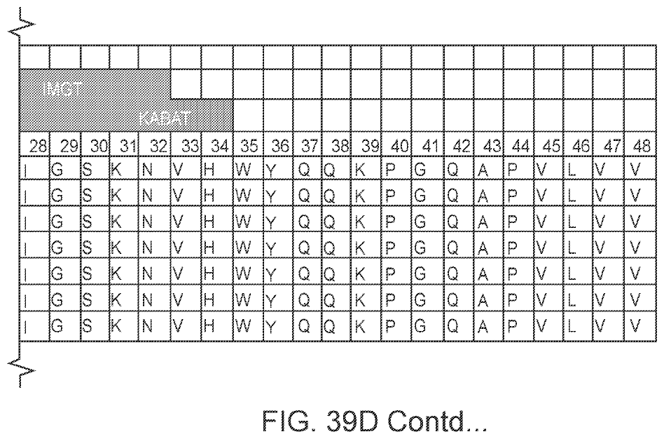

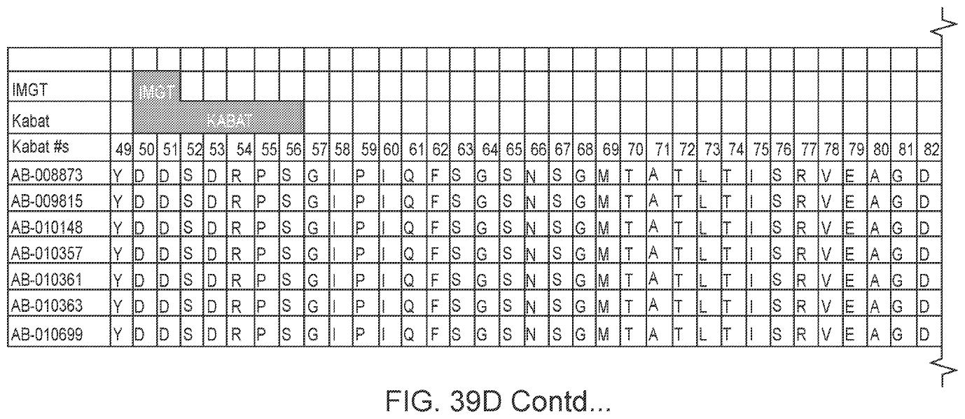

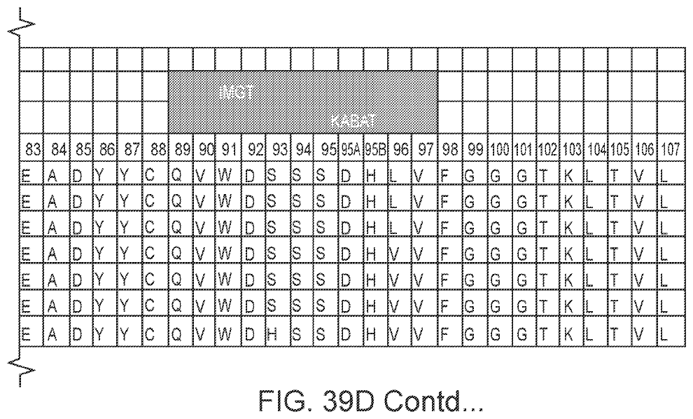

CDRs as shown in Tables 6 and 7 are defined by IMGT and Kabat. The V_{H} CDRs as listed in Table 6, are defined as follows: HCDR1 is defined by combining Kabat and IMGT; HCDR2 is defined by Kabat; and the HCDR3 is defined by IMGT. The V_{L} CDRs as listed in Table 7 are defined by Kabat. FIG. 39A-39B shows alignment of EphA2 antibody V_{H} and V_{L} sequences with CDRs designated by Kabat and IMGT. As known in the art, numbering and placement of the CDRs can differ depending on the numbering system employed. It is understood that disclosure of a variable heavy and/or variable light sequence includes the disclosure of the associated CDRs, regardless of the numbering system employed.

A “nonlinear epitope” or “conformational epitope” comprises noncontiguous polypeptides (or amino acids) within the antigenic protein to which an antibody specific to the epitope binds. A conformational epitope is typically formed by a three-dimensional interaction of amino acids in the epitope that may not necessarily be contained in a single stretch of amino acids.

An “Fc region” refers to the constant region of an antibody excluding the first constant region immunoglobulin domain. Thus, e.g., for human immunoglobulins, “Fc” refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ2. It is understood in the art that the boundaries of the Fc region may vary, however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or this region in the context of an antibody or antibody fragment. “Fc region” includes naturally occurring allelic variants of the Fc region as well as modified Fc regions, e.g., that are modified to modulate effector function or other properties such as pharmacokinetics, stability, or production properties of an antibody. Fc regions also include variants that do not exhibit alterations in biological function. For example, one or more amino acids can be deleted from the N-terminus or C-terminus of the Fc region of an immunoglobulin without substantial loss of biological function. Such variants can be selected according to general rules known in the art to have minimal effect on activity (see, e.g., Bowie et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibody (see, e.g., Angal et al., Mol Immunol 30:105-108, 1993).

An “EC₅₀” as used herein in the context of a binding or functional assay, refers to the half maximal effective concentration, which is the concentration of an antibody that induces a response (readouts can include but are not limited to fluorescence or luminescence signals) halfway between the baseline and maximum after a specified exposure time. In some embodiments, the “fold over EC₅₀” is determined by dividing the EC₅₀ of a reference antibody by the EC₅₀ of the test antibody.

The term “equilibrium dissociation constant” abbreviated (K_{D}), refers to the dissociation rate constant (k_{d}, time⁻¹) divided by the association rate constant (kₐ, time⁻¹ M⁻¹). Equilibrium dissociation constants can be measured using any method. Thus, in some embodiments antibodies of the present disclosure have a K_{D} of less than about 50 nM, typically less than about 25 nM, or less than 10 nM, e.g., less than about 5 nM or than about 1 nM and often less than about 10 nM as determined by surface plasmon resonance analysis using a biosensor system such as a Biacore® system performed at 37° C. In some embodiments, an antibody of the present disclosure has a K_{D} of less than 5×10⁻⁵ M, less than 10⁻⁵ M, less than 5×10⁻⁶M, less than 10⁻⁶M, less than 5×10⁻⁷ M, less than 10⁻⁷ M, less than 5×10⁻⁸M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower as measured as a bivalent antibody. In the context of the present disclosure, an “improved” K_{D} refers to a lower K_{D}. In some embodiments, an antibody of the present disclosure has a K_{D} of less than 5×10⁻⁵ M, less than 10⁻⁵ M, less than 5×10⁻⁶M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower as measured as a monovalent antibody, such as a monovalent Fab. In some embodiments, an EphA2 antibody of the present disclosure has K_{D} less than 100 pM, e.g., or less than 75 pM, e.g., in the range of 1 to 100 pM, when measured by surface plasmon resonance analysis using a biosensor system such as a Biacore® system performed at 37° C. In some embodiments, an EphA2 antibody of the present disclosure has K_{D} of greater than 100 pM, e.g., in the range of 100-1000 pM or 500-1000 pM when measured by surface plasmon resonance analysis using a biosensor system such as a Biacore® system performed at 37° C. The term “EphA2 antibody” is used interchangeably with “anti-EphA2 antibody” in this application.

The term “monovalent molecule” as used herein refers to a molecule with one antigen-binding site, e.g., a Fab or scFv.

The term “bivalent molecule” as used herein refers to a molecule with two antigen-binding sites. In some embodiments, a bivalent molecule of the present disclosure is a bivalent antibody or a bivalent fragment thereof. In some embodiments, a bivalent molecule of the present disclosure is a bivalent antibody. In some embodiments, a bivalent molecule of the present disclosure is an IgG. In general, monoclonal antibodies have a bivalent basic structure. IgG and IgE have only one bivalent unit, while IgA and IgM consist of multiple bivalent units (2 and 5, respectively) and thus have higher valencies. This bivalency increases the avidity of antibodies for antigens.

The terms “monovalent binding” or “monovalently binds to” as used herein refer to the binding of one antigen-binding site to its antigen.

The terms “bivalent binding” or “bivalently binds to” as used herein refer to the binding of both antigen-binding sites of a bivalent molecule to its antigen. In some embodiments, both antigen-binding sites of a bivalent molecule share the same antigen specificity.

The term “valency” as used herein refers to the number of different binding sites of an antibody for an antigen. A monovalent antibody comprises one binding site for an antigen. A bivalent antibody comprises two binding sites for the same antigen. A multivalent antibody comprises two or more binding sites for the same antigen. A trivalent antibody comprises three binding sites for the same antigen. A tetravalent antibody comprises four binding sites for the same antigen.

The term “avidity” as used herein in the context of antibody binding to an antigen refers to the combined binding strength of multiple binding sites of the antibody. Thus, “bivalent avidity” refers to the combined strength of two binding sites.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, e.g., the length of the two sequences, when compared and aligned for maximum correspondence over a comparison window or designated region. Alignment for purposes of determining percent amino acid sequence identity can be performed in various methods, including those using publicly available computer software such as BLAST. BLAST-2, ALIGN or Megalign (DNASTAR) software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, for purposes of this invention, BLAST 2.0 can be used with the default parameters to determine percent sequence identity.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. The polypeptide that is aligned to the reference sequence need not be the same length as the reference sequence.

A “conservative” substitution as used herein refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe. Tyr and Trp; (iv) nitrogen ring amino acids His and Trp; (v) aliphatic hydrophobic amino acids Ala, Val, Leu and Ile; (vi) hydrophobic sulfur-containing amino acids Met and Cys, which are not as hydrophobic as Val, Leu, and Ile; (vii) small polar uncharged amino acids Ser, Thr, Asp, and Asn (viii) small hydrophobic or neutral amino acids Gly, Ala, and Pro; (ix) amide-comprising amino acids Asn and Gln; and (xi) beta-branched amino acids Thr, Val, and Ile. Reference to the charge of an amino acid in this paragraph refers to the charge at pH 6-7.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, or combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors can direct the expression of nucleic acids to which they are operatively linked. Such vectors are referred to herein as “expression vectors”.

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy or light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Thus, a host cell can be a recombinant host cell, a primary transformed cell and progeny derived therefrom without regard to the number of passages.

A polypeptide “variant,” as the term is used herein, is a polypeptide that typically differs from one or more polypeptide sequences specifically disclosed herein in one or more substitutions, deletions, additions and/or insertions.

The term “cancer cell” or “tumor cell” as used herein refers to a neoplastic cell. The term includes cells from tumors that are benign as well as malignant. Neoplastic transformation is associated with phenotypic changes of the tumor cell relative to the cell type from which it is derived. The changes can include loss of contact inhibition, morphological changes, and unregulated cell growth,

“Inhibiting growth of a tumor” and “inhibiting growth of a cancer” as used interchangeably and refer to slowing growth and/or reducing the cancer cell burden of a patient who has cancer. “Inhibiting growth of a cancer” thus includes killing cancer cells, as well as decreasing the rate of tumor growth, tumor size, invasion, and/or metastasis by direct or indirect effects on tumor cells.

As used herein, “therapeutic agent” refers to an agent that will cure, or at least partially arrest the symptoms of a disease and complications associated with the disease when administered to a patient suffering from the disease in a therapeutically effective dose.

As used herein, the term “a tumor overexpressing EphA2”, or “a tumor that overexpresses EphA2”, or “a cancer overexpressing EphA2” or “a cancer that overexpresses EphA2”, refers to a tumor or cancer that expresses EphA2 or demonstrates EpA2 reactivity at a level that higher than the level of EphA2 expressed or EphA2 reactivity in normal tissue (e.g., tumor adjacent tissues or TAT). In certain embodiments, a tumor or cancer that overexpresses EphA2 is one that expresses EphA2 at a level that is at least 10%, at least 20%, at least 30%, at least 40%, at least 50%, at least 80%, or at 100% higher than the normal tissue (e.g., tumor adjacent tissues or TAT). In certain embodiments, a tumor or cancer that overexpresses EphA2 is one that demonstrates EphA2 reactivity at level that is at least 10%, at least 20%, at least 30%, at least 40%, at least 50%, at least 80%, or at 100% higher than the normal tissue (e.g., tumor adjacent tissues or TAT).

In some aspects, the disclosure additionally provides methods of identifying subjects who are candidates for treatment with an EphA2 antibody having tumor-targeting effects. Thus, in one embodiment, the invention provides a method of identifying a patient who can benefit from treatment with an EphA2 antibody of the present disclosure. In one embodiment, the patient has tumor that expresses EphA2. In one embodiment, the patient has tumor that overexpresses EphA2. In some embodiments, the tumor sample is from a primary tumor. In alternative embodiments, the tumor sample is a metastatic lesion. Binding of antibody to tumor cells through a binding interaction with the EphA2 can be measured using any assay, such as immunohistochemistry or flow cytometry. In some embodiments, binding of antibody to at least 0.2%, 0.5%, or 1%, or at least 5% or 10%, or at least 20%, 30%, or 50%, of the tumor cells in a sample may be used as a selection criterion for determining a patient to be treated with an EphA2 antibody as described herein. In other embodiments, analysis of components of the blood, e.g., circulating protein levels, is used to identify a patient whose tumor cells are overexpressing EphA2.

An EphA2 antibody disclosed herein can be used to treat several different cancers. In some embodiments, a cancer patient who can benefit from the treatment of the EphA2 antibody has a cancer expressing EphA2. In some embodiments, a cancer patient who can benefit from the treatment of the EphA2 antibody has a cancer overexpressing EphA2. In some embodiments, the cancer is a carcinoma or a sarcoma.

As used herein, the term “an antibody binds to an EphA2” or “an antibody binds to EphA2,” means that the antibody binds to EphA2 under permissible conditions (e.g., in a suitable buffer), and the detected signal resulted from the binding is at least 2-fold, at least 5-fold, at least 10 fold, at least 20 fold, at least 100 fold, at least 150 fold, or at least 200 fold above a reference level. In some embodiments, the reference level is a detected signal produced by contacting a control antibody with the EphA2, or by contacting the antibody with a control protein.

As used herein, the term “sibling antibodies” refer to antibodies derived from the same B-cell clonal lineage. In some embodiments, sibling antibodies share sequence and structural properties as well as epitope specificity.

As used herein, the term “convergent antibodies” refer to that antibodies derived from different B-cell lineages that exhibit similar sequence and structural properties.

Several anti-EphA2 comparator antibodies are referenced in this disclosure. AB-010018 is a mouse monoclonal antibody precursor of clinical candidate humanized DS-8895a (Daiichi Sankyo Company, Ltd.), as disclosed in U.S. Pat. No. 9,150,657, SEQ ID NO: 35 and 37, Hasegawa J. et al., Cancer Biol Ther. 2016 November; 17(11):1158-1167. doi: 10.1080/15384047.2016.1235663. Epub 2016 Sep. 21; Shitara K. et al., J Immunother Cancer, 2019 Aug. 14; 7(1):219. doi: 10.1186/s40425-019-0679-9)),

AB-010016 is an anti-EphA2 antibody generated based on clinical candidate Medi-547 (MedImmune, Inc.) It was generated using the V_{H} and V_{L} amino acid sequences from Protein Data Bank code 3SKJ, Peng, L., et. al, (2011) J Mol Biol 413: 390-405.

AB-010017 is an anti-EphA2 antibody generated based on clinical candidate MM-310 (Merrimack, Inc.) It was generated using V_{H} and V_{L} amino acid sequences from the scFv of SEQ ID NO: 40 in US20190070113A1; See also, Geddie et al., MAbs. 2017 January; 9(1): 58-67; Kamoun et al., Nat Biomed Eng. 2019 April; 3(4):264-280. doi: 10.1038/s41551-019-0385-4. Epub 2019 Apr. 5.)

### Overview

This application relates to tumor-targeting antibodies that bind to EphA2 and bind preferentially to tumor tissues than normal tissues. In some embodiments, the EphA2 antibody binds to one or more epitopes in the FN2 domain of EphA2. The EphA2 antibodies disclosed herein can target a tumor in a variety of ways, for example, it can mediate antibody-dependent cell-mediated cytotoxicity (ADCC) and/or ADCP to target and kill tumor cells. These antibodies also demonstrated significant cytotoxicity towards tumor cells and show therapeutic potential in treating cancers. Some of the EphA2 antibodies can target both mouse tumor and human tumor cells. The EphA2 antibodies are also useful in detecting tumors suitable for treatment in diagnostic applications.

As compared to existing clinical candidates and commercial EphA2 antibodies, EphA2 antibodies provided in this disclosure showed a higher binding affinity to various tumors and lower binding to normal tissues (e.g., tumor adjacent tissues) and exhibit differential binding profile on various cancer tumor types. Unlike existing clinical candidates and commercial EphA2 antibodies, the EphA2 antibodies disclosed herein exhibit reduced agonistic or antagonistic effects on the EphA2 receptor. These properties render them ideal for use as cancer therapeutics.

### EPHA2

Ephrin receptor A2 (EphA2) is expressed mainly in proliferating epithelial cells in adults. Under normal conditions, EphA2 interacts with membrane proteins, ephrin A1, on the neighboring cell and induce diverse signaling networks following cell-to-cell contact. For example, EphA2 may decrease in cell-extracellular matrix (ECM) attachment upon phosphorylation.

EphA2 is overexpressed in many solid tumors including melanoma, glioma, prostate cancer, breast cancer, ovarian cancer, lung cancer, colon cancer, esophageal cancer, gastric cancer cervical cancer, bladder cancer. EphA2 contributes to cell adhesion and angiogenesis, and its expression is also linked to increased malignancy and poor prognosis.

Human EphA2 has a sequence of

(SEQ ID NO: 94)
MELQAARACFALLWGCALAAAAAAQGKEVVLLDFAAAGGELGWLTHPYG
KGWDLMQNIMNDMPIYMYSVCNVMSGDQDNWLRTNWVYRGEAERIFIEL
KFTVRDCNSFPGGASSCKETFNLYYAESDLDYGTNFQKRLFTKIDTIAP
DEITVSSDFEARHVKLNVEERSVGPLTRKGFYLAFQDIGACVALLSVRV
YYKKCPELLQGLAHFPETIAGSDAPSLATVAGTCVDHAVVPPGGEEPRM
HCAVDGEWLVPIGQCLCQAGYEKVEDACQACSPGFFKFEASESPCLECP
EHTLPSPEGATSCECEEGFFRAPQDPASMPCTRPPSAPHYLTAVGMGAK
VELRWTPPQDSGGREDIVYSVTCEQCWPESGECGPCEASVRYSEPPHGL
TRTSVTVSDLEPHMNYTFTVEARNGVSGLVTSRSFRTASVSINQTEPPK
VRLEGRSTTSLSVSWSIPPPQQSRVWKYEVTYRKKGDSNSYNVRRTEGF
SVTLDDLAPDTTYLVQVQALTQEGQGAGSKVHEFQTLSPEGSGNLAVIG
GVAVGVVLLLVLAGVGFFIHRRRKNQRARQSPEDVYFSKSEQLKPLKTY
VDPHTYEDPNQAVLKFTTEIHPSCVTRQKVIGAGEFGEVYKGMLKTSSG
KKEVPVAIKTLKAGYTEKQRVDFLGEAGIMGQFSHHNIIRLEGVISKYK
PMMIITEYMENGALDKFLREKDGEFSVLQLVGMLRGIAAGMKYLANMNY
VHRDLAARNILVNSNLVCKVSDFGLSRVLEDDPEATYTTSGGKIPIRWT
APEAISYRKFTSASDVWSFGIVMWEVMTYGERPYWELSNHEVMKAINDG
FRLPTPMDCPSAIYQLMMQCWQQERARRPKFADIVSILDKLIRAPDSLK
TLADFDPRVSIRLPSTSGSEGVPFRTVSEWLESIKMQQYTEHFMAAGYT
AIEKVVQMTNDDIKRIGVRLPGHQKRIAYSLLGLKDQVNTVGIPI

Human EphA2 comprises: a signal peptide having the sequence of:

(SEQ ID NO: 96)
MELQAARACFALLWGCALAAAAAA;

a ligand binding domain having the sequence of

(SEQ ID NO: 97)
QGKEVVLLDFAAAGGELGWLTHPYGKGWDLMQNIMNDMPIYMYSVCNVM
SGDQDNWLRTNWVYRGEAERIFIELKFTVRDCNSFPGGASSCKETFNLY
YAESDLDYGTNFQKRLFTKIDTIAPDEITVSSDFEARHVKLNVEERSVG
PLTRKGFYLAFQDIGACVALLSVRVYYK;

a sushi domain having the sequence of

(SEQ ID NO: 98)
KCPELLQGLAHFPETIAGSDAPSLATVAGTCVDHAVVPPGGEEPRMHCA
VDGEWLVPIGQCL;

an EGF domain having the sequence of

(SEQ ID NO: 99)
CQAGYEKVEDACQACSPGFFKFEASESPCLECPEHTLPSPEGATSCECE
EGFFRAPQDPASMPCT;

an FN1 domain having the sequence of

(SEQ ID NO: 100)
RPPSAPHYLTAVGMGAKVELRWTPPQDSGGREDIVYSVTCEQCWPESGE
CGPCEASVRYSEPPHGLTRTSVTVSDLEPHMNYTFTVEARNGVSGLVTS
RSFRTASVSINQ;
and

an FN2 domain having the sequence of

(SEQ ID NO: 95)
TEPPKVRLEGRSTTSLSVSWSIPPPQQSRVWKYEVTYRKKGDSNSYNVR
RTEGFSVTLDDLAPDTTYLVQVQALTQEGQGAGSKVHEFQTLSPEGSGN

EPHA2 comprises an ectodomain, an intracellular region, and a single transmembrane helix. The ectodomain of EphA2, consisting of amino acid residues 23-546 using SEQ ID NO: 94 as a reference, comprises a ligand binding domain (LBD), which interacts with ephrin A1, a Sushi domain, an epidermal growth factor like domain, and two fibronectin type III domains (FN1 and FN2). The intracellular region comprises a tyrosine kinase domain and a sterile α-motif domain. The transmembrane helix of the protein is flanked by juxtamembrane linkers, which connects the ectodomain and the intracellular domain. A membrane-binding motif FN2, located within amino acid residues 437-534 of SEQ ID NO: 94, includes positively charged residues, which can recruit negatively charged lipids to the site of membrane-protein interaction. The interactions of FN2 with lipids stabilize the otherwise flexible EphA2 ectodomain in two main conformations relative to the membrane.

The EphA2 ectodomain is highly conserved across species. The ectodomain of human EphA2 has sequence identity of 90.8%, 90.4%, 98.8%, and 98.8% to mouse, rat, macaque, and cyno, respectively.

### EPHA2 Antibodies

For purpose of this disclosure, an EphA2 antibody may be any one of AB-008873, the siblings and variants thereof, as further described below.

AB-008873 was discovered in antibody repertoires generated by Immune Repertoire Capture® (IRC™) technology from plasmablast B cells isolated from a non-small cell lung cancer patient with an active anti-tumor immune response after treatment with the anti-PD-1 antibody OPDTVO® (nivolumab) (Bristol Myers Squibb). The IRC™ technology and its use in antibody discovery is well known and disclosed in, e.g., WO 2012148497A2, the entire content of which is herein incorporated by reference. AB-008873 aligns to its closest human germline genes (IGHV4-34*02, IGHD2-8*01, IGHJ3*02, IGLV3-21*02, and IGLJ2*01), and to its four known siblings AB-009805, AB-009806. AB-009807, and AB-009808. Structures of these antibodies are further disclosed below, e.g., in Tables 6-8.

### Variants

In some embodiments, variants of any of the EphA2 antibodies disclosed herein can be generated by introducing mutations to the heavy chain and/or light chain sequences. In some embodiments, the mutation(s) are introduced into one or more of the CDRs of an EphA2 antibody disclosed herein, e.g., AB-008873, AB-010148, or AB-010363. In some embodiments, the mutation(s) are introduced in the framework regions. In some embodiments, an EphA2 antibody provided herein comprises a V_{H} region of any one of SEQ ID NO 67-77 and 591-655, and/or a V_{L} region of any one of SEQ ID NO: 78-88 and 656-720, or an antibody comprising a V_{H} region with at least 80% identity to any one of SEQ ID NO 67-77 and 591-655 and a V_{L} region having at least 80% identity to any one of SEQ ID NO: 78-88 and 656-720, with variations to the corresponding V_{H} or V_{L} regions present only in Framework regions.

In some embodiments, EphA2 antibody provided herein comprises an HCDR1 of any one of SEQ ID NOS:1-11, and 201-265, an HCDR2 of any one of SEQ ID NOS:12-22 and 266-330, an HCDR3 of any one of SEQ ID NOS:23-33 and 331-395, an LCDR1 of any one of SEQ ID NOS:34-44 and 396-460, an LCDR2 of any one of SEQ ID NOS:45-55 and 461-525, an LCDR3 of any one of SEQ ID NOS:56-66 and 526-590; and the FW regions in the V_{H} region are at least 80% identical to the FW regions present in the V_{H} region of SEQ ID NO 67-77 and 591-655, and wherein the FW regions in the V_{L} region are at least 80% identical to the FW regions present in the V_{L} region of SEQ ID NO: 78-88 and 656-720

In some embodiments, a variant is engineered to be as much like self as possible to minimize immunogenicity. One approach to do so is to identify a close germline sequence and mutate one of the EphA2 antibodies at as many mismatched positions (also known as “germline deviations”) to the germline residue type as possible. In some embodiments, a variant (e.g., AB-009812) comprises a mutation H54RS relative to AB-008873 (heavy chain position 54 mutated from arginine to serine). In some embodiments, a variant (e.g., AB-009813) comprises a mutation H54RA relative to AB-008873 (heavy chain position 54 mutated from arginine to alanine). In some embodiments, a variant (e.g., AB-009814) comprises deletion of three residues, H61N-H62Y-H63N, relative to AB-008873. In some embodiments, a variant comprises one or more mutations selected from the group consisting of H54RS, H54RA, and deletions of three residues H61N-H62Y-H63N, relative to the sequence of the heavy chain variable region of AB-008873 (SEQ ID NO: 67). Variant antibodies AB-009812, AB-009813, and AB-009814 are described in Tables 1-3.

Additional variants antibodies may also be generated by introducing one or more mutations to any one of the AB-008873 and its siblings. In some embodiments, a variant comprises one or more mutations selected from the group consisting of H31DG, L31NS, L97LV, H51VI, H86TS, H129AS, and L60QR relative to AB-009815. Each using the Hv and Lv sequences of the AB-009815 (SEQ ID NO: 75 and 86) as references. For any the mutation disclosed in this application, the name indicates whether the mutation is a heavy chain or light chain, the position in the heavy chain or light chain of the mutation, the amino acid residue at the position before introduction of the mutation, and the amino acid at the position after introduction of the mutation. For example, H129AS, refers to that the alanine at heavy chain position 129 is mutated to a serine. Table 1 shows exemplary variants of AB-009815.

TABLE 1
Exemplary variants of AB-009815

| | Mutations relative to AB- |
|---|---|
| Antibody ID | 009815 |
| AB-010141 | H31DG |
| AB-010142 | L31NS |
| AB-010143 | L97LV |
| AB-010144 | H51VI |
| AB-010145 | H86TS |
| AB-010146 | H129AS |
| AB-010147 | L60QR |
| AB-010148 | H51VI; H86TS |
| AB-010149 | H51VI; H129AS |
| AB-010150 | H86TS; H129AS |
| AB-010151 | H51VI, H86TS, H129AS |
| AB-010152 | H51VI, H86TS, H129AS, |
| | L60QR |

Further mutations can be introduced to any one of the AB-008873 variants listed in Table 1. For example, one or more mutations are introduced to AB-0010148 and exemplary variants resulted from the further mutagenesis are shown in in Table 2, below.

TABLE 2
Exemplary AB-0010148 variants

| | Mutations relative to AB- |
|---|---|
| Antibody ID | 0010148 |
| AB-010357 | L97LV |
| AB-010358 | L60QR |
| AB-010359 | L97LV; L60QR |
| AB-010360 | H31DG |
| AB-010361 | H31DG; L97LV |
| AB-010362 | H31DG; L60QR |
| AB-010363 | H31DG; L60QR; L97LV |
| AB-010364 | H31DG; H54RA; L97LV |
| AB-010365 | H31DG; H54RA; L97LV; |
| | L60QR |
| AB-010366 | H31DG; H54RQ; L97LV |
| AB-010367 | H31DG; H54RQ; L97LV; |
| | L60QR |

Further mutations can be introduced to any one of the AB-0010148 variants listed in Table 2. For example, one or more mutations are introduced to AB-010357, AB-010361, AB-010363, and exemplary antibodies resulted from the further mutagenesis are shown in in Table 3, 4, 5, respectively.

TABLE 3
Exemplary variants of AB-010357

| Antibody ID | Mutations relative to AB-010357 |
|---|---|
| AB-010661 | H107TI; L23GR |
| AB-010662 | L23GR; L29SY |
| AB-010663 | L23GR; L30KM |
| AB-010664 | L23GR; L92SH |
| AB-010665 | H107T; L29SY |
| AB-010666 | L29SY; L30KM |
| AB-010667 | L29SY; L92SH |
| AB-010668 | H107TI; L30KM |
| AB-010669 | L30KM; L92SH |
| AB-010670 | H107TI; L92SH |
| AB-010671 | H107TI; L29SY; L92SH |
| AB-010672 | L93SR |
| AB-010673 | L93SE |
| AB-010674 | L29SY; L93SE |

TABLE 4
Exemplary variants of AB-010361

| Antibody ID | Mutations relative to AB-010361 |
|---|---|
| AB-010675 | H107TI; L23GR |
| AB-010676 | L23GR; L29SY |
| AB-010677 | L23GR; L30KM |
| AB-010678 | L23GR; L92SH |
| AB-010679 | H107TI; L29SY |
| AB-010680 | L29SY; L30KM |
| AB-010681 | L29SY; L92SH |
| AB-010682 | H107TI; L30KM |
| AB-010683 | L30KM; L92SH |
| AB-010684 | H107TI; L92SH |
| AB-010685 | H107TI; L29SY; L92SH |
| AB-010686 | L93SR |
| AB-010687 | L93SE |
| AB-010688 | L29SY; L93SE |

TABLE 5
shows exemplary variants of AB-010363

| Antibody ID | Mutations relative to AB-010363 |
|---|---|
| AB-010689 | H107TI; L23GR |
| AB-010690 | L23GR; L29SY |
| AB-010691 | L23GR; L30KM |
| AB-010692 | L23GR; L92SH |
| AB-010693 | H107TI; L29SY |
| AB-010694 | L29SY; L30KM |
| AB-010695 | L29SY; L92SH |
| AB-010696 | H107TI; L30KM |
| AB-010697 | L30KM; L92SH |
| AB-010698 | H107TI; L92SH |
| AB-010699 | H107TI; L29SY; L92SH |
| AB-010700 | L93SR |
| AB-010701 | L93SE |
| AB-010702 | L29SY; L93SE |

Methods of generating variants are further described in the section entitled “ENGINEERING VARIANTS” and Example 2 below.

### Structures of the EphA2 Antibodies

In some embodiments, an EphA2 antibody that binds to EphA2 and comprises a heavy chain variable region comprising: an HCDR1 comprising a sequence (SEQ ID NO:1), or a variant HCDR1 in which 1, 2, 3, 4, or 5 amino acids are substituted relative to the sequence; an HCDR2 comprising a sequence (SEQ ID NO:12), or a variant HCDR2 in which 1, 2, 3, 4, or 5 amino acid is substituted relative to the sequence; and an HCDR3 comprising a sequence (SEQ ID NO:23), or a variant HCDR3 in which 1, 2, 3, 4, or 5 amino acids are substituted relative to the sequence. In some embodiments, the antibody comprises a light chain variable region comprising: an LCDR1 comprising a sequence (SEQ ID NO:34), or a variant LCDR1 in which 1, 2, 3, 4, or 5 amino acid is substituted relative to the sequence; an LCDR2 comprising a sequence (SEQ ID NO:45), or variant LCDR2 in which 1, 2, or 3 amino acid is substituted relative to the sequence; and an LCDR3 comprising a sequence (SEQ ID NO:56), or a variant LCDR3 in which 1, 2, 3, 4, or 5 amino acid is substituted relative to the sequence.

In some embodiments, an antibody that binds to EphA2 comprises: a heavy chain variable region comprising: an HCDR1 of any one of SEQ ID NOS: 1-11 and 201-265 or a variant thereof in which 1, 2, 3, 4, or 5 amino acids are substituted relative to the sequence; an HCDR2 of any one of SEQ ID NOS: 12-22 and 266-330 or a variant thereof in which 1, 2, 3, 4, or 5 amino acid is substituted relative to the sequence; and an HCDR3 of any one of SEQ ID NOS: 23-33 and 331-395 or a variant thereof in which 1, 2, 3, 4, or 5 amino acids are substituted relative to the sequence. The antibody also comprises a light chain variable region comprising: an LCDR1 of any one of SEQ ID NOS: 34-44 and 396-460 or a variant thereof in which 1, 2, 3, 4, or 5 amino acid is substituted relative to the sequence; an LCDR2 of any one of SEQ ID NOS: 45-55 and 461-525 or a variant thereof in which 1, 2, or 3 amino acid is substituted relative to the sequence; and an LCDR3 of any one of SEQ ID NOS: 56-66 and 526-590 or a variant thereof in which 1, 2, 3, 4, or 5 amino acid is substituted relative to the sequence.

In some embodiments, an antibody that binds to EphA2 comprises (a) an HCDR1 sequence of GGSX₁X₂ X₃YX₄WS where X₁ is F or L, X₂ is S or N, and X₃ is D or G, and X₄ is Y or H (SEQ ID NO: 769); (b) an HCDR2 sequence of EX₁NHX₂GSX₃X₄YNNYNPSLKS, where X₁ is I or V, X₂ is A, Q, R or S, X₃ is I or T, and X₄ is N or S (SEQ ID NO: 770); and (c) an HCDR3 sequence of AKPX₁RPHC X₂NGVCX₃SGDAFDI, where X₁ is L or F, X₂ is I or T; and X₃ is Y or S(SEQ ID NO: 771). The antibody also comprises (d) an LCDR1 sequence of X₁GNNIGX₂ X₃ X₄VH, where X₁ is G or R, X₂ is S, T, or Y, X₃ is K or M, and X₄ is N or S (SEQ ID NO: 772); I an LCDR2 sequence of DDSDRPS (SEQ ID NO: 45); and (f) an LCDR3 sequence of QVWDX1 X2SDHX3V, where X1 is H or S, X2 is E, R, or S, and X3 is L or V (SEQ ID NO: 773).

In some embodiments, an antibody that binds to EphA2 comprises (a) an HCDR1 sequence of GGSX₁X₂ X₃YX₄WS where X₁ is F or L, X₂ is S or N, and X₃ is D or G, and X₄ is Y or H (SEQ ID NO: 769); (b) an HCDR2 sequence of EX₁NHX₂GSX₃X₄YNPSLKS, where X₁ is I or V, X₂ is A, Q, R or S, X₃ is I or T, and X₄ is N or S (SEQ ID NO: 774); or an HCDR2 sequence of EX₁NHX₂ GS X₃ X₄YNNYNPSLKS, where X₁ is I or V, X₂ is R or S, X₃ is I or T, and X₄ N or S (SEQ ID NO: 777); and (c) an HCDR3 sequence of AKPX₁RPHCX₂NGVCX₃SGDAFDI, where X₁ is L or F, X₂ is I or T; and X₃ is Y or S (SEQ ID NO: 771). The antibody also comprises (d) an LCDR1 sequence of X₁GNNIGX₂ X₃ X₄VH, where X₁ is G or R, X₂ is S, T, or Y, X₃ is K or M, and X₄ is N or I (SEQ ID NO: 780) (e) an LCDR2 sequence of DDSDRPS (SEQ ID NO: 45); and (f) an LCDR3 sequence of QVWDX1 X2SDHX3V, where X1 is H or S, X2 is E, R, or S, and X3 is L or V. (SEQ ID NO: 773).

In some embodiments, an antibody that binds to EphA2 comprises a V_{H} comprising an amino acid sequence having at least 95% identity to (SEQ ID NO: 67); and a V_{L} comprising an amino sequence having at 95% identity to (SEQ ID NO: 78).

In some embodiments, an antibody that binds to EphA2 has a V_{H} comprising an amino acid sequence having at least 95% identity to any one of SEQ ID NO: 67-77 and 591-655; and a V_{L} comprising an amino sequence having at 95% identity to any one of SEQ ID NOs: 78-88 and 656-720.

In some embodiments, an antibody that binds to EphA2 comprises a heavy chain variable (V_{H}) region and a light chain variable (V_{L}) region. The V_{H} region has at least 70% amino acid sequence identity to SEQ ID NO:67; and comprises a CDR1 of SEQ ID NO:1, or the CDR1 of SEQ ID NO:1 in which 1, 2, 3, 4, or 5 amino acids are substituted; a CDR2 of SEQ ID NO:83, or the CDR2 of SEQ ID NO:12 in which 1, 2, 3, 4, or 5 amino acids are substituted; a CDR3 of SEQ ID NO:23 or the CDR3 of SEQ ID NO:23 in which 1, 2, 3, 4, or 5 are substituted. The V_{L} region has at least 70% amino acid sequence identity to SEQ ID NO: 78, and comprises a CDR1 of SEQ ID NO:34 or the CDR1 of SEQ ID NO:34 in which 1, 2, 3, 4, or 5 amino acids are substituted; a CDR2 of SEQ ID NO:45, or the CDR2 of SEQ ID NO:45 in which 1, 2, 3, 4, or 5 amino acids are substituted; a CDR3 of SEQ ID NO:56 or the CDR3 of SEQ ID NO:56 in which 1, 2, 3, 4, or 5 are substituted.

In some embodiments, an antibody that binds to EphA2 comprises: a V_{H} region comprising amino acid sequence SEQ ID NO:67 and a V_{L} region comprising amino acid sequence SEQ ID NO:78; a V_{H} region comprising amino acid sequence SEQ ID NO:68 and a V_{L} region comprising amino acid sequence SEQ ID NO:79; a V_{H} region comprising amino acid sequence SEQ ID NO:69 and a V_{L} region comprising amino acid sequence SEQ ID NO:80; a V_{H} region comprising amino acid sequence SEQ ID NO:70 and a V_{L} region comprising amino acid sequence SEQ ID NO:81; a V_{H} region comprising amino acid sequence SEQ ID NO:71 and a V_{L} region comprising amino acid sequence SEQ ID NO:82; a V_{H} region comprising amino acid sequence SEQ ID NO:72 and a V_{L} region comprising amino acid sequence SEQ ID NO:83; a V_{H} region comprising amino acid sequence SEQ ID NO:73 and a V_{L} region comprising amino acid sequence SEQ ID NO:84; a V_{H} region comprising amino acid sequence SEQ ID NO:74 and a V_{L} region comprising amino acid sequence SEQ ID NO:85; a V_{H} region comprising amino acid sequence SEQ ID NO:75 and a V_{L} region comprising amino acid sequence SEQ ID NO:86; a V_{H} region comprising amino acid sequence SEQ TD NO:76 and a V_{L} region comprising amino acid sequence SEQ ID NO:87; or a V_{H} region comprising amino acid sequence SEQ ID NO:77 and a V_{L} region comprising amino acid sequence SEQ ID NO:88.

In some embodiments, an EphA2 antibody of the present disclosure has one, two, or three CDRs of a V_{L} sequence in Table 7. In some embodiments, the EphA2 antibody has at least one mutation and no more than 10, 20, 30, 40 or 50 mutations in the V_{L} amino acid sequences compared to a V_{L} sequence set forth in Table 8. In some embodiments, the V_{L} amino acid sequence may comprise 1, 2, 3, 4, 5, 6, 7, 8, 9, or 10 amino acid insertions or deletions compared to a V_{L} sequence set forth in Table 8. In some embodiments, the V_{L} amino acid sequence may comprise a deletion or insertion, e.g., a 1, 2, 3, 4, 5, 6, or 7 amino acid deletion or insertion, relative to a CDR sequence shown in Table 7. In some embodiments, the V_{L} region comprises a CDR1 having 1 or 2 substitutions in relative to a CDR1 sequence shown in Table 7. In some embodiments, a CDR1 has 3, 4, or 5 substitutions relative to a CDR1 sequence shown in Table 7. In some embodiments, the V_{L} region comprises a CDR2 with 1 or 2; or 1, 2, or 3; substitutions relative to the CDR2 sequence shown in Table 7. In some embodiments, the V_{L} region comprises a CDR3 with 1, 2, or 3; or 1, 2, 3, or 4; substitutions relative to a CDR3 sequence shown in Table 7. In some embodiments, an EphA2 antibody of the present disclosure comprises a CDR1, CDR2, and CDR3, each having at least 70% identity to a CDR1, CDR2, and CDR3 as shown in Table 7. In some embodiments, an EphA2 antibody of the present disclosure comprises a CDR1, CDR2, and CDR3, each having at least 80% identity to a CDR1, CDR2, and CDR3 as shown in Table 7. In some embodiments, an anti-tumor antibody of the present disclosure comprises a CDR1, CDR2, and CDR3 as shown in Table 7. In some embodiments, an EphA2 antibody of the present disclosure comprises HCDR1, HCDR2, HCDR3, LCDR1, LCDR2, and LCDR3 of an antibody designated as AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698, AB-010699, AB-010700, AB-010701, or AB-010702 in Table 7. In some embodiments, an EphA2 antibody of the present disclosure comprises a CDR1, CDR2, and CDR3 of the VL of an antibody designated as AB-010361 or AB-010699. Of these antibodies, AB-008873; AB-009805; AB-009806; AB-009807; AB-009808 are sibling antibodies as they are derived from the same lineage as AB-008873. See Example 1.

In some embodiments, the antibody comprises a heavy chain variable region and a light chain variable region of an antibody designated as AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698, AB-010699. AB-010700, AB-010701, or AB-010702, or a variant thereof, wherein the variant comprises a heavy chain variable region having a sequence that is at least 95% identical to that of the corresponding heavy chain variable region and a light chain variable region having a sequence that is at least 95% identical to the corresponding light chain variable region.

In some embodiments, an EphA2 antibody of the present disclosure has one, two, or three CDRs of a V_{H} sequence in Table 8. In some embodiments, the EphA2 antibody has at least one mutation and no more than 10, 20, 30, 40 or 50 mutations in the V_{H} amino acid sequences compared to a V_{H} sequence set forth in Table 8. In some embodiments, the V_{H} amino acid sequence may comprise 1, 2, 3, 4, 5, 6, 7, 8, 9, or 10 amino acid insertions or deletions compared to a V_{H} sequence set forth in Table 8. In some embodiments, the V_{H} amino acid sequence may comprise a deletion or insertion, e.g., a 1, 2, 3, 4, 5, 6, or 7 amino acid deletion or insertion, relative to a CDR sequence shown in Table 6. In some embodiments, the V_{H} region comprises a CDR1 having 1 or 2 substitutions in relative to a CDR1 sequence shown in Table 6. In some embodiments, a CDR1 has 3, 4, or 5 substitutions relative to a CDR1 sequence shown in Table 6. In some embodiments, the V_{H} region comprises a CDR2 with 1 or 2; or 1, 2, or 3; substitutions relative to the CDR2 sequence shown in Table 6. In some embodiments, the V_{H} region comprises a CDR3 with 1, 2, or 3; or 1, 2, 3, or 4; substitutions relative to a CDR3 sequence shown in Table 6. In some embodiments, an EphA2 antibody of the present disclosure comprises a CDR1, CDR2, and CDR3, each having at least 70% identity to a CDR1, CDR2, and CDR3 as shown in Table 6. In some embodiments, an EphA2 antibody of the present disclosure comprises a CDR1, CDR2, and CDR3, each having at least 80% identity to a CDR1, CDR2, and CDR3 as shown in Table 6. In some embodiments, an EphA2 antibody of the present disclosure comprises a CDR1, CDR2, and CDR3 as shown in Table 6. In some embodiments, an EphA2 antibody of the present disclosure comprises a CDR1, CDR2, and CDR3 of an antibody designated as AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698, AB-010699, AB-010700, AB-010701, or AB-010702 in Table 6. In some embodiments, an EphA2 antibody of the present disclosure comprises a CDR1, CDR2, and CDR3 of the VH of an antibody designated as AB-010361 and AB-010699.

Exemplary EphA2 antibodies include AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698, AB-010699. AB-010700, AB-010701, or AB-010702. In some embodiments, the EphA2 antibody is AB-010361 or AB-010699. These exemplary EphA2 antibodies have structures (HCDRs, LCDRs, V_{H} and/or V_{L} sequences) shown in Tables 6-8.

TABLE 6
Heavy chain CDRs

| Antibody ID | HCDR1 | HCDR2 | HCDR3 |
|---|---|---|---|
| AB-008873 | (SEQ ID NO: 1) | (SEQ ID NO: 12) | (SEQ ID NO: 23) |
| AB-009805 | (SEQ ID NO: 2) | (SEQ ID NO: 13) | (SEQ ID NO: 24) |
| AB-009806 | (SEQ ID NO: 3) | (SEQ ID NO: 14) | (SEQ ID NO: 25) |
| AB-009807 | (SEQ ID NO: 4) | (SEQ ID NO: 15) | (SEQ ID NO: 26) |
| AB-009808 | (SEQ ID NO: 5) | (SEQ ID NO: 16) | (SEQ ID NO: 27) |
| AB-009812 | (SEQ ID NO: 6) | (SEQ ID NO: 17) | (SEQ ID NO: 28) |
| AB-009813 | (SEQ ID NO: 7) | (SEQ ID NO: 18) | (SEQ ID NO: 29) |
| AB-009814 | (SEQ ID NO: 8) | (SEQ ID NO: 19) | (SEQ ID NO: 30) |
| AB-009815 | (SEQ ID NO: 9) | (SEQ ID NO: 20) | (SEQ ID NO: 31) |
| AB-009816 | (SEQ ID NO: 10) | (SEQ ID NO: 21) | (SEQ ID NO: 32) |
| AB-009817 | (SEQ ID NO: 11) | (SEQ ID NO: 22) | (SEQ ID NO: 33) |
| AB-010141 | (SEQ ID NO: 201) | (SEQ ID NO: 266) | (SEQ ID NO: 331) |
| AB-010142 | (SEQ ID NO: 202) | (SEQ ID NO: 267) | (SEQ ID NO: 332) |
| AB-010143 | (SEQ ID NO: 203) | (SEQ ID NO: 268) | (SEQ ID NO: 333) |
| AB-010144 | (SEQ ID NO: 204) | (SEQ ID NO: 269) | (SEQ ID NO: 334) |
| AB-010145 | (SEQ ID NO: 205) | (SEQ ID NO: 270) | (SEQ ID NO: 335) |
| AB-010146 | (SEQ ID NO: 206) | (SEQ ID NO: 271) | (SEQ ID NO: 336) |
| AB-010147 | (SEQ ID NO: 207) | (SEQ ID NO: 272) | (SEQ ID NO: 337) |
| AB-010148 | (SEQ ID NO: 208) | (SEQ ID NO: 273) | (SEQ ID NO: 338) |
| AB-010149 | (SEQ ID NO: 209) | (SEQ ID NO: 274) | (SEQ ID NO: 339) |
| AB-010150 | (SEQ ID NO: 210) | (SEQ ID NO: 275) | (SEQ ID NO: 340) |
| AB-010151 | (SEQ ID NO: 211) | (SEQ ID NO: 276) | (SEQ ID NO: 341) |
| AB-010152 | (SEQ ID NO: 212) | (SEQ ID NO: 277) | (SEQ ID NO: 342) |
| AB-010357 | (SEQ ID NO: 213) | (SEQ ID NO: 278) | (SEQ ID NO: 343) |
| AB-010358 | (SEQ ID NO: 214) | (SEQ ID NO: 279) | (SEQ ID NO: 344) |
| AB-010359 | (SEQ ID NO: 215) | (SEQ ID NO: 280) | (SEQ ID NO: 345) |
| AB-010360 | (SEQ ID NO: 216) | (SEQ ID NO: 281) | (SEQ ID NO: 346) |
| AB-010361 | (SEQ ID NO: 217) | (SEQ ID NO: 282) | (SEQ ID NO: 347) |
| AB-010362 | (SEQ ID NO: 218) | (SEQ ID NO: 283) | (SEQ ID NO: 348) |
| AB-010363 | (SEQ ID NO: 219) | (SEQ ID NO: 284) | (SEQ ID NO: 349) |
| AB-010364 | (SEQ ID NO: 220) | (SEQ ID NO: 285) | (SEQ ID NO: 350) |
| AB-010365 | (SEQ ID NO: 221) | (SEQ ID NO: 286) | (SEQ ID NO: 351) |
| AB-010366 | (SEQ ID NO: 222) | (SEQ ID NO: 287) | (SEQ ID NO: 352) |
| AB-010367 | (SEQ ID NO: 223) | (SEQ ID NO: 288) | (SEQ ID NO: 353) |
| AB-010661 | (SEQ ID NO: 224) | (SEQ ID NO: 289) | (SEQ ID NO: 354) |
| AB-010662 | (SEQ ID NO: 225) | (SEQ ID NO: 290) | (SEQ ID NO: 355) |
| AB-010663 | (SEQ ID NO: 226) | (SEQ ID NO: 291) | (SEQ ID NO: 356) |
| AB-010664 | (SEQ ID NO: 227) | (SEQ ID NO: 292) | (SEQ ID NO: 357) |
| AB-010665 | (SEQ ID NO: 228) | (SEQ ID NO: 293) | (SEQ ID NO: 358) |
| AB-010666 | (SEQ ID NO: 229) | (SEQ ID NO: 294) | (SEQ ID NO: 359) |
| AB-010667 | (SEQ ID NO: 230) | (SEQ ID NO: 295) | (SEQ ID NO: 360) |
| AB-010668 | (SEQ ID NO: 231) | (SEQ ID NO: 296) | (SEQ ID NO: 361) |
| AB-010669 | (SEQ ID NO: 232) | (SEQ ID NO: 297) | (SEQ ID NO: 362) |
| AB-010670 | (SEQ ID NO: 233) | (SEQ ID NO: 298) | (SEQ ID NO: 363) |
| AB-010671 | (SEQ ID NO: 234) | (SEQ ID NO: 299) | (SEQ ID NO: 364) |
| AB-010672 | (SEQ ID NO: 235) | (SEQ ID NO: 300) | (SEQ ID NO: 365) |
| AB-010673 | (SEQ ID NO: 236) | (SEQ ID NO: 301) | (SEQ ID NO: 366) |
| AB-010674 | (SEQ ID NO: 237) | (SEQ ID NO: 302) | (SEQ ID NO: 367) |
| AB-010675 | (SEQ ID NO: 238) | (SEQ ID NO: 303) | (SEQ ID NO: 368) |
| AB-010676 | (SEQ ID NO: 239) | (SEQ ID NO: 304) | (SEQ ID NO: 369) |
| AB-010677 | (SEQ ID NO: 240) | (SEQ ID NO: 305) | (SEQ ID NO: 370) |
| AB-010678 | (SEQ ID NO: 241) | (SEQ ID NO: 306) | (SEQ ID NO: 371) |
| AB-010679 | (SEQ ID NO: 242) | (SEQ ID NO: 307) | (SEQ ID NO: 372) |
| AB-010680 | (SEQ ID NO: 243) | (SEQ ID NO: 308) | (SEQ ID NO: 373) |
| AB-010681 | (SEQ ID NO: 244) | (SEQ ID NO: 309) | (SEQ ID NO: 374) |
| AB-010682 | (SEQ ID NO: 245) | (SEQ ID NO: 310) | (SEQ ID NO: 375) |
| AB-010683 | (SEQ ID NO: 246) | (SEQ ID NO: 311) | (SEQ ID NO: 376) |
| AB-010684 | (SEQ ID NO: 247) | (SEQ ID NO: 312) | (SEQ ID NO: 377) |
| AB-010685 | (SEQ ID NO: 248) | (SEQ ID NO: 313) | (SEQ ID NO: 378) |
| AB-010686 | (SEQ ID NO: 249) | (SEQ ID NO: 314) | (SEQ ID NO: 379) |
| AB-010687 | (SEQ ID NO: 250) | (SEQ ID NO: 315) | (SEQ ID NO: 380) |
| AB-010688 | (SEQ ID NO: 251) | (SEQ ID NO: 316) | (SEQ ID NO: 381) |
| AB-010689 | (SEQ ID NO: 252) | (SEQ ID NO: 317) | (SEQ ID NO: 382) |
| AB-010690 | (SEQ ID NO: 253) | (SEQ ID NO: 318) | (SEQ ID NO: 383) |
| AB-010691 | (SEQ ID NO: 254) | (SEQ ID NO: 319) | (SEQ ID NO: 384) |
| AB-010692 | (SEQ ID NO: 255) | (SEQ ID NO: 320) | (SEQ ID NO: 385) |
| AB-010693 | (SEQ ID NO: 256) | (SEQ ID NO: 321) | (SEQ ID NO: 386) |
| AB-010694 | (SEQ ID NO: 257) | (SEQ ID NO: 322) | (SEQ ID NO: 387) |
| AB-010695 | (SEQ ID NO: 258) | (SEQ ID NO: 323) | (SEQ ID NO: 388) |
| AB-010696 | (SEQ ID NO: 259) | (SEQ ID NO: 324) | (SEQ ID NO: 389) |
| AB-010697 | (SEQ ID NO: 260) | (SEQ ID NO: 325) | (SEQ ID NO: 390) |
| AB-010698 | (SEQ ID NO: 261) | (SEQ ID NO: 326) | (SEQ ID NO: 391) |
| AB-010699 | (SEQ ID NO: 262) | (SEQ ID NO: 327) | (SEQ ID NO: 392) |
| AB-010700 | (SEQ ID NO: 263) | (SEQ ID NO: 328) | (SEQ ID NO: 393) |
| AB-010701 | (SEQ ID NO: 264) | (SEQ ID NO: 329) | (SEQ ID NO: 394) |
| AB-010702 | (SEQ ID NO: 265) | (SEQ ID NO: 330) | (SEQ ID NO: 395) |

TABLE 7
Light chain CDR sequences

| Antibody ID | LCDR1 | LCDR2 | LCDR3 |
|---|---|---|---|
| AB-008873 | (SEQ ID NO: 34) | (SEQ ID NO: 45) | (SEQ ID NO: 56) |
| AB-009805 | (SEQ ID NO: 35) | (SEQ ID NO: 46) | (SEQ ID NO: 57) |
| AB-009806 | (SEQ ID NO: 36) | (SEQ ID NO: 47) | (SEQ ID NO: 58) |
| AB-009807 | (SEQ ID NO: 37) | (SEQ ID NO: 48) | (SEQ ID NO: 59) |
| AB-009808 | (SEQ ID NO: 38) | (SEQ ID NO: 49) | (SEQ ID NO: 60) |
| AB-009812 | (SEQ ID NO: 39) | (SEQ ID NO: 50) | (SEQ ID NO: 61) |
| AB-009813 | (SEQ ID NO: 40) | (SEQ ID NO: 51) | (SEQ ID NO: 62) |
| AB-009814 | (SEQ ID NO: 41) | (SEQ ID NO: 52) | (SEQ ID NO: 63) |
| AB-009815 | (SEQ ID NO: 42) | (SEQ ID NO: 53) | (SEQ ID NO: 64) |
| AB-009816 | (SEQ ID NO: 43) | (SEQ ID NO: 54) | (SEQ ID NO: 65) |
| AB-009817 | (SEQ ID NO: 44) | (SEQ ID NO: 55) | (SEQ ID NO: 66) |
| AB-010141 | (SEQ ID NO: 396) | (SEQ ID NO: 461) | (SEQ ID NO: 526) |
| AB-010142 | (SEQ ID NO: 397) | (SEQ ID NO: 462) | (SEQ ID NO: 527) |
| AB-010143 | (SEQ ID NO: 398) | (SEQ ID NO: 463) | (SEQ ID NO: 528) |
| AB-010144 | (SEQ ID NO: 399) | (SEQ ID NO: 464) | (SEQ ID NO: 529) |
| AB-010145 | (SEQ ID NO: 400) | (SEQ ID NO: 465) | (SEQ ID NO: 530) |
| AB-010146 | (SEQ ID NO: 401) | (SEQ ID NO: 466) | (SEQ ID NO: 531) |
| AB-010147 | (SEQ ID NO: 402) | (SEQ ID NO: 467) | (SEQ ID NO: 532) |
| AB-010148 | (SEQ ID NO: 403) | (SEQ ID NO: 468) | (SEQ ID NO: 533) |
| AB-010149 | (SEQ ID NO: 404) | (SEQ ID NO: 469) | (SEQ ID NO: 534) |
| AB-010150 | (SEQ ID NO: 405) | (SEQ ID NO: 470) | (SEQ ID NO: 535) |
| AB-010151 | (SEQ ID NO: 406) | (SEQ ID NO: 471) | (SEQ ID NO: 536) |
| AB-010152 | (SEQ ID NO: 407) | (SEQ ID NO: 472) | (SEQ ID NO: 537) |
| AB-010357 | (SEQ ID NO: 408) | (SEQ ID NO: 473) | (SEQ ID NO: 538) |
| AB-010358 | (SEQ ID NO: 409) | (SEQ ID NO: 474) | (SEQ ID NO: 539) |
| AB-010359 | (SEQ ID NO: 410) | (SEQ ID NO: 475) | (SEQ ID NO: 540) |
| AB-010360 | (SEQ ID NO: 411) | (SEQ ID NO: 476) | (SEQ ID NO: 541) |
| AB-010361 | (SEQ ID NO: 412) | (SEQ ID NO: 477) | (SEQ ID NO: 542) |
| AB-010362 | (SEQ ID NO: 413) | (SEQ ID NO: 478) | (SEQ ID NO: 543) |
| AB-010363 | (SEQ ID NO: 414) | (SEQ ID NO: 479) | (SEQ ID NO: 544) |
| AB-010364 | (SEQ ID NO: 415) | (SEQ ID NO: 480) | (SEQ ID NO: 545) |
| AB-010365 | (SEQ ID NO: 416) | (SEQ ID NO: 481) | (SEQ ID NO: 546) |
| AB-010366 | (SEQ ID NO: 417) | (SEQ ID NO: 482) | (SEQ ID NO: 547) |
| AB-010367 | (SEQ ID NO: 418) | (SEQ ID NO: 483) | (SEQ ID NO: 548) |
| AB-010661 | (SEQ ID NO: 419) | (SEQ ID NO: 484) | (SEQ ID NO: 549) |
| AB-010662 | (SEQ ID NO: 420) | (SEQ ID NO: 485) | (SEQ ID NO: 550) |
| AB-010663 | (SEQ ID NO: 421) | (SEQ ID NO: 486) | (SEQ ID NO: 551) |
| AB-010664 | (SEQ ID NO: 422) | (SEQ ID NO: 487) | (SEQ ID NO: 552) |
| AB-010665 | (SEQ ID NO: 423) | (SEQ ID NO: 488) | (SEQ ID NO: 553) |
| AB-010666 | (SEQ ID NO: 424) | (SEQ ID NO: 489) | (SEQ ID NO: 554) |
| AB-010667 | (SEQ ID NO: 425) | (SEQ ID NO: 490) | (SEQ ID NO: 555) |
| AB-010668 | (SEQ ID NO: 426) | (SEQ ID NO: 491) | (SEQ ID NO: 556) |
| AB-010669 | (SEQ ID NO: 427) | (SEQ ID NO: 492) | (SEQ ID NO: 557) |
| AB-010670 | (SEQ ID NO: 428) | (SEQ ID NO: 493) | (SEQ ID NO: 558) |
| AB-010671 | (SEQ ID NO: 429) | (SEQ ID NO: 494) | (SEQ ID NO: 559) |
| AB-010672 | (SEQ ID NO: 430) | (SEQ ID NO: 495) | (SEQ ID NO: 560) |
| AB-010673 | (SEQ ID NO: 431) | (SEQ ID NO: 496) | (SEQ ID NO: 561) |
| AB-010674 | (SEQ ID NO: 432) | (SEQ ID NO: 497) | (SEQ ID NO: 562) |
| AB-010675 | (SEQ ID NO: 433) | (SEQ ID NO: 498) | (SEQ ID NO: 563) |
| AB-010676 | (SEQ ID NO: 434) | (SEQ ID NO: 499) | (SEQ ID NO: 564) |
| AB-010677 | (SEQ ID NO: 435) | (SEQ ID NO: 500) | (SEQ ID NO: 565) |
| AB-010678 | (SEQ ID NO: 436) | (SEQ ID NO: 501) | (SEQ ID NO: 566) |
| AB-010679 | (SEQ ID NO: 437) | (SEQ ID NO: 502) | (SEQ ID NO: 567) |
| AB-010680 | (SEQ ID NO: 438) | (SEQ ID NO: 503) | (SEQ ID NO: 568) |
| AB-010681 | (SEQ ID NO: 439) | (SEQ ID NO: 504) | (SEQ ID NO: 569) |
| AB-010682 | (SEQ ID NO: 440) | (SEQ ID NO: 505) | (SEQ ID NO: 570) |
| AB-010683 | (SEQ ID NO: 441) | (SEQ ID NO: 506) | (SEQ ID NO: 571) |
| AB-010684 | (SEQ ID NO: 442) | (SEQ ID NO: 507) | (SEQ ID NO: 572) |
| AB-010685 | (SEQ ID NO: 443) | (SEQ ID NO: 508) | (SEQ ID NO: 573) |
| AB-010686 | (SEQ ID NO: 444) | (SEQ ID NO: 509) | (SEQ ID NO: 574) |
| AB-010687 | (SEQ ID NO: 445) | (SEQ ID NO: 510) | (SEQ ID NO: 575) |
| AB-010688 | (SEQ ID NO: 446) | (SEQ ID NO: 511) | (SEQ ID NO: 576) |
| AB-010689 | (SEQ ID NO: 447) | (SEQ ID NO: 512) | (SEQ ID NO: 577) |
| AB-010690 | (SEQ ID NO: 448) | (SEQ ID NO: 513) | (SEQ ID NO: 578) |
| AB-010691 | (SEQ ID NO: 449) | (SEQ ID NO: 514) | (SEQ ID NO: 579) |
| AB-010692 | (SEQ ID NO: 450) | (SEQ ID NO: 515) | (SEQ ID NO: 580) |
| AB-010693 | (SEQ ID NO: 451) | (SEQ ID NO: 516) | (SEQ ID NO: 581) |
| AB-010694 | (SEQ ID NO: 452) | (SEQ ID NO: 517) | (SEQ ID NO: 582) |
| AB-010695 | (SEQ ID NO: 453) | (SEQ ID NO: 518) | (SEQ ID NO: 583) |
| AB-010696 | (SEQ ID NO: 454) | (SEQ ID NO: 519) | (SEQ ID NO: 584) |
| AB-010697 | (SEQ ID NO: 455) | (SEQ ID NO: 520) | (SEQ ID NO: 585) |
| AB-010698 | (SEQ ID NO: 456) | (SEQ ID NO: 521) | (SEQ ID NO: 586) |
| AB-010699 | (SEQ ID NO: 457) | (SEQ ID NO: 522) | (SEQ ID NO: 587) |
| AB-010700 | (SEQ ID NO: 458) | (SEQ ID NO: 523) | (SEQ ID NO: 588) |
| AB-010701 | (SEQ ID NO: 459) | (SEQ ID NO: 524) | (SEQ ID NO: 589) |
| AB-010702 | (SEQ ID NO: 460) | (SEQ ID NO: 525) | (SEQ ID NO: 590) |

TABLE 8
Heavy and Light variable region sequences

| Antibody ID | V_{H} | V_{L} |
|---|---|---|
| AB-008873 | (SEQ ID NO: 67) | (SEQ ID NO: 78) |
| AB-009805 | (SEQ ID NO: 68) | (SEQ ID NO: 79) |
| AB-009806 | (SEQ ID NO: 69) | (SEQ ID NO: 80) |
| AB-009807 | (SEQ ID NO: 70) | (SEQ ID NO: 81) |
| AB-009808 | (SEQ ID NO: 71) | (SEQ ID NO: 82) |
| AB-009812 | (SEQ ID NO: 72) | (SEQ ID NO: 83) |
| AB-009813 | (SEQ ID NO: 73) | (SEQ ID NO: 84) |
| AB-009814 | (SEQ ID NO: 74) | (SEQ ID NO: 85) |
| AB-009815 | (SEQ ID NO: 75) | (SEQ ID NO: 86) |
| AB-009816 | (SEQ ID NO: 76) | (SEQ ID NO: 87) |
| AB-009817 | (SEQ ID NO: 77) | (SEQ ID NO: 88) |
| AB-010141 | (SEQ ID NO: 591) | (SEQ ID NO: 656) |
| AB-010142 | (SEQ ID NO: 592) | (SEQ ID NO: 657) |
| AB-010143 | (SEQ ID NO: 593) | (SEQ ID NO: 658) |
| AB-010144 | (SEQ ID NO: 594) | (SEQ ID NO: 659) |
| AB-010145 | (SEQ ID NO: 595) | (SEQ ID NO: 660) |
| AB-010146 | (SEQ ID NO: 596) | (SEQ ID NO: 661) |
| AB-010147 | (SEQ ID NO: 597) | (SEQ ID NO: 662) |
| AB-010148 | (SEQ ID NO: 598) | (SEQ ID NO: 663) |
| AB-010149 | (SEQ ID NO: 599) | (SEQ ID NO: 664) |
| AB-010150 | (SEQ ID NO: 600) | (SEQ ID NO: 665) |
| AB-010151 | (SEQ ID NO: 601) | (SEQ ID NO: 666) |
| AB-010152 | (SEQ ID NO: 602) | (SEQ ID NO: 667) |
| AB-010357 | (SEQ ID NO: 603) | (SEQ ID NO: 668) |
| AB-010358 | (SEQ ID NO: 604) | (SEQ ID NO: 669) |
| AB-010359 | (SEQ ID NO: 605) | (SEQ ID NO: 670) |
| AB-010360 | (SEQ ID NO: 606) | (SEQ ID NO: 671) |
| AB-010361 | (SEQ ID NO: 607) | (SEQ ID NO: 672) |
| AB-010362 | (SEQ ID NO: 608) | (SEQ ID NO: 673) |
| AB-010363 | (SEQ ID NO: 609) | (SEQ ID NO: 674) |
| AB-010364 | (SEQ ID NO: 610) | (SEQ ID NO: 675) |
| AB-010365 | (SEQ ID NO: 611) | (SEQ ID NO: 676) |
| AB-010366 | (SEQ ID NO: 612) | (SEQ ID NO: 677) |
| AB-010367 | (SEQ ID NO: 613) | (SEQ ID NO: 678) |
| AB-010661 | (SEQ ID NO: 614) | (SEQ ID NO: 679) |
| AB-010662 | (SEQ ID NO: 615) | (SEQ ID NO: 680) |
| AB-010663 | (SEQ ID NO: 616) | (SEQ ID NO: 681) |
| AB-010664 | (SEQ ID NO: 617) | (SEQ ID NO: 682) |
| AB-010665 | (SEQ ID NO: 618) | (SEQ ID NO: 683) |
| AB-010666 | (SEQ ID NO: 619) | (SEQ ID NO: 684) |
| AB-010667 | (SEQ ID NO: 620) | (SEQ ID NO: 685) |
| AB-010668 | (SEQ ID NO: 621) | (SEQ ID NO: 686) |
| AB-010669 | (SEQ ID NO: 622) | (SEQ ID NO: 687) |
| AB-010670 | (SEQ ID NO: 623) | (SEQ ID NO: 688) |
| AB-010671 | (SEQ ID NO: 624) | (SEQ ID NO: 689) |
| AB-010672 | (SEQ ID NO: 625) | (SEQ ID NO: 690) |
| AB-010673 | (SEQ ID NO: 626) | (SEQ ID NO: 691) |
| AB-010674 | (SEQ ID NO: 627) | (SEQ ID NO: 692) |
| AB-010675 | (SEQ ID NO: 628) | (SEQ ID NO: 693) |
| AB-010676 | (SEQ ID NO: 629) | (SEQ ID NO: 694) |
| AB-010677 | (SEQ ID NO: 630) | (SEQ ID NO: 695) |
| AB-010678 | (SEQ ID NO: 631) | (SEQ ID NO: 696) |
| AB-010679 | (SEQ ID NO: 632) | (SEQ ID NO: 697) |
| AB-010680 | (SEQ ID NO: 633) | (SEQ ID NO: 698) |
| AB-010681 | (SEQ ID NO: 634) | (SEQ ID NO: 699) |
| AB-010682 | (SEQ ID NO: 635) | (SEQ ID NO: 700) |
| AB-010683 | (SEQ ID NO: 636) | (SEQ ID NO: 701) |
| AB-010684 | (SEQ ID NO: 637) | (SEQ ID NO: 702) |
| AB-010685 | (SEQ ID NO: 638) | (SEQ ID NO: 703) |
| AB-010686 | (SEQ ID NO: 639) | (SEQ ID NO: 704) |
| AB-010687 | (SEQ ID NO: 640) | (SEQ ID NO: 705) |
| AB-010688 | (SEQ ID NO: 641) | (SEQ ID NO: 706) |
| AB-010689 | (SEQ ID NO: 642) | (SEQ ID NO: 707) |
| AB-010690 | (SEQ ID NO: 643) | (SEQ ID NO: 708) |
| AB-010691 | (SEQ ID NO: 644) | (SEQ ID NO: 709) |
| AB-010692 | (SEQ ID NO: 645) | (SEQ ID NO: 710) |
| AB-010693 | (SEQ ID NO: 646) | (SEQ ID NO: 711) |
| AB-010694 | (SEQ ID NO: 647) | (SEQ ID NO: 712) |
| AB-010695 | (SEQ ID NO: 648) | (SEQ ID NO: 713) |
| AB-010696 | (SEQ ID NO: 649) | (SEQ ID NO: 714) |
| AB-010697 | (SEQ ID NO: 650) | (SEQ ID NO: 715) |
| AB-010698 | (SEQ ID NO: 651) | (SEQ ID NO: 716) |
| AB-010699 | (SEQ ID NO: 652) | (SEQ ID NO: 717) |
| AB-010700 | (SEQ ID NO: 653) | (SEQ ID NO: 718) |
| AB-010701 | (SEQ ID NO: 654) | (SEQ ID NO: 719) |
| AB-010702 | (SEQ ID NO: 655) | (SEQ ID NO: 720) |

In some embodiments, the EphA2 antibody disclosed herein comprises a heavy chain variable region and a light chain variable region of an antibody designated as AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697. AB-010698, AB-010699, AB-010700, AB-010701, or AB-010702 or a variant thereof. The variant comprises a heavy chain variable region having a sequence that is at least 95% identical to that of the corresponding heavy chain variable region and a light chain variable region having a sequence that is at least 95% identical to the corresponding light chain variable region. For example, the variant may comprise a V_{H} that is at least 95% identical to that of AB-008873, AB-010148, AB-010363, or AB-010699 and/or a V_{L} that is at least 95% identical to that of AB-008873, AB-010148, AB-010363, or AB-010699.

In some embodiments, the EphA2 antibody disclosed herein comprises a heavy chain variable region and a light chain variable region of an antibody designated as AB-010361 or AB-010699.

In some embodiments, the EphA2 antibody disclosed herein comprises an HCDR1, HCDR2, HCDR3, LCDR1, LCDR2, and LCDR3 of an antibody designated as AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698, AB-010699, AB-010700, AB-010701, or AB-010702, or a variant thereof, and at least one, two, three, four, five, or all six of the CDRs of the variant contain 1 or 2 amino acid substitutions compared to the corresponding CDR. For example, at least one, two, three, four, five, or all six of the CDRs of the variant contain 1 or 2 amino acid substitutions compared to the CDRs of AB-008873, AB-010148, AB-010363, or AB-010699

In some embodiments, the EphA2 antibody disclosed herein comprises an HCDR1, HCDR2, HCDR3, LCDR1, LCDR2, and LCDR3 of an antibody designated as AB-010361 or AB-010699.

### Epitope

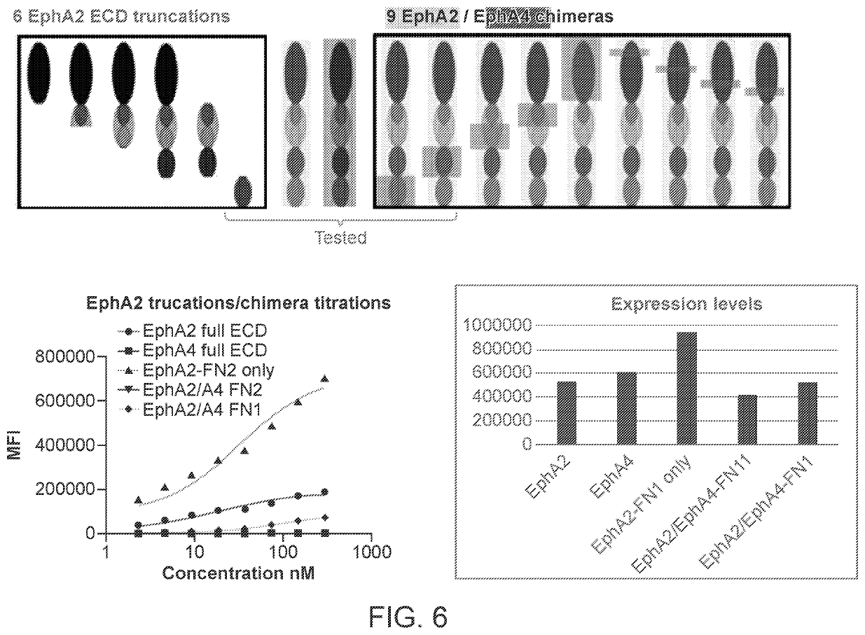

An EphA2 antibody disclosed herein binds to an epitope in the EphA2 protein (SEQ ID NO: 94). In some embodiments, an EphA2 antibody disclosed herein binds to an epitope in the FN2 domain of the EphA2 protein. In some embodiments, the binding can be detected using yeast display experiments illustrated in FIG. 6. The FN2 domain consists of residues 437-534 of SEQ ID NO: 94 and has a sequence of SEQ ID NO: 95 (TEPPKVRLEGRSTTSLSVSWSIPPPQQSRVWKYEVTYRKKGDSNSYNVRRTEGFSVTLDDLA PDTTYLVQVQALTQEGQGAGSKVHEFQTLSPEGSGN). In some embodiments, the epitope recognized by an EphA2 antibody disclosed herein overlaps with that of AB-010018.

Epitopes for the EphA2 antibodies disclosed herein can be determined by any method well known in the art, for example, by conventional immunoassays. In one example, the epitope to which the EphA2 antibody binds can be determined in a systematic screening by using overlapping peptides derived from the EphA2 sequence and determining binding by the antibody. In some cases, during the discovery process, the generation and characterization of antibodies may elucidate information about desirable epitopes. This information may allow screening for antibodies that compete for binding to the same epitope, e.g., an epitope in the FN2 domain of the EphA2 protein.

In some cases, an epitope binning method is performed, e.g., using Bio-layer interferometry (BLI) as described in Example 4. The underlying principle of epitope binning is that proteins with overlapping binding sites will sterically inhibit simultaneous binding. For each pair of antibodies, the antigen is pre-saturated with the 1^{st} monoclonal antibody (mAb), the 2^{nd} mAb is applied and signal from the binding of the 2^{nd} mAb to the antigen is detected and quantified. A significantly reduced signal from binding of 2^{nd} mAb to the antigen indicates that there are overlapping binding sites between the two antibodies. Pair-wise interactions of the antibodies are scored and used to bin the antibodies into sets defined by mutual competition. Epitope binning is useful for antibody characterization because antibodies in a bin may exhibit similar MOAs. Binning with antibodies with antibodies of known reactivity can inform epitope identification.

An EphA2 antibody disclosed herein binds to at least one, at least two, at least three, at least four, at least five residues selected from the group consisting of Pro439, Lys441, Arg443, Leu444, Arg447, Lys476, Gly477, Leu504, Gln506, Ser519, Lys520, Val521, His522, Glu523, Phe524, and Gln525, with the residue numbering referring to the EphA2 amino acid sequence of SEQ ID NO: 94. In one embodiment, the EphA2 antibody binds to at least one, at least two, at least three, at least four, at least five residues, at least six residues, at least seven residues, at least eight residues, or at least nine residues selected from the group consisting of Leu444, Arg447, Lys476, Gln506, Ser519, Lys520, Val521, Glu523, Phe524, and Gln525. In one embodiment, the EphA2 antibody binds to all the residues Leu444, Arg447, Lys476, Gln506, Ser519, Lys520, Val521, Glu523, Phe524, and Gln525.

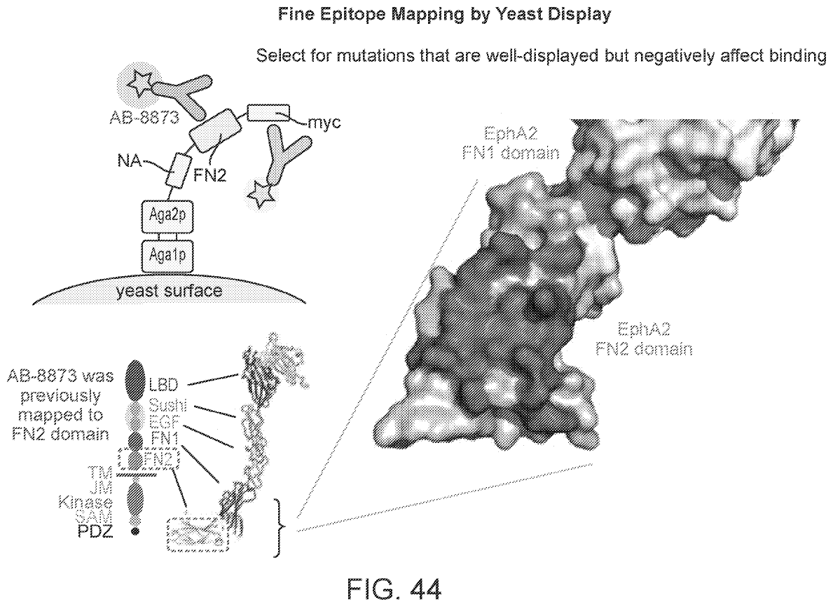

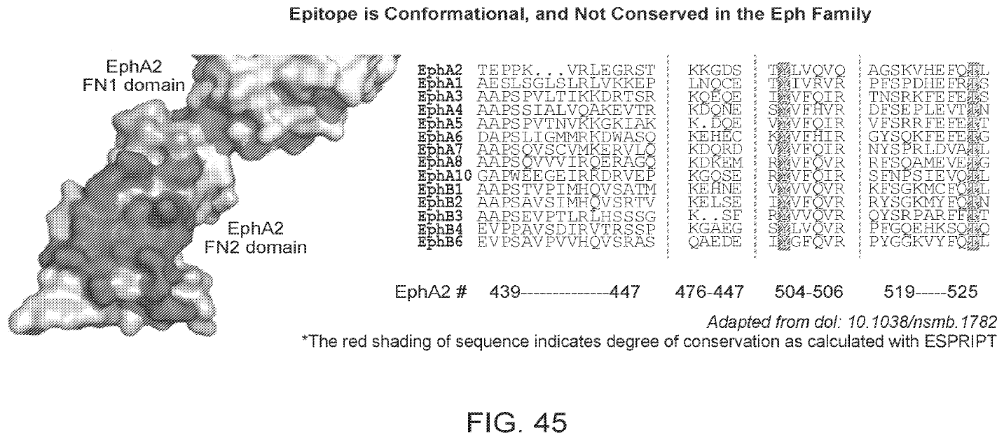

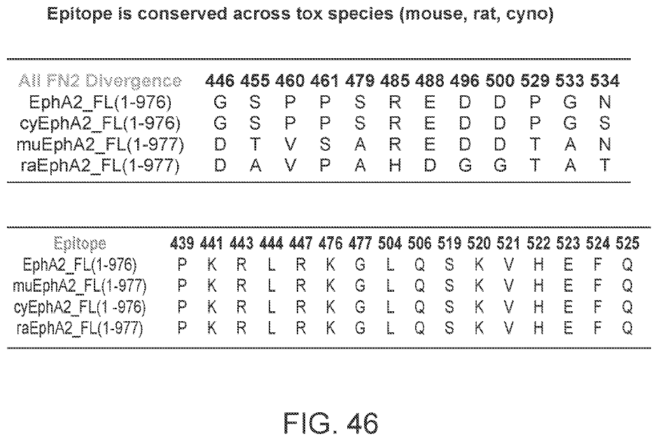

In some embodiments, the EphA2 antibody bind to a conformational epitope that is in one, two, three and/or four regions in the EphA2 protein: a first region consisting of amino acid residues 439-447, a second region consisting of amino acid residues 476-477, a third region consisting of amino acid residues 504-506, and a fourth region consisting of amino acid residues 519-525. In some embodiments, the epitope that the EphA2 antibody binds is conserved across species of cyno, rat, mouse and human, all of which are commonly used in toxicology studies. FIG. 46, FIGS. 44 and 45. Seiradake et al., Nat. Struct. Mol. Biol 17, 398-402 (2010).

### Tumor-Binding Activity

The activity of the EphA2 antibodies as described herein can be assessed for binding in binding assays. Nonlimiting examples of suitable assays include surface plasmon resonance analysis using a biosensor system such as a Biacore® system or a flow cytometry assay, which are further described in the EXAMPLES section.

In some embodiments, binding to EphA2 protein is assessed in a competitive assay format with a reference antibody AB-008873 or a reference antibody having the variable regions of AB-008873. In some embodiments, a variant EphA2 antibody in accordance with the present disclosure may block binding of the reference antibody in a competition assay by about 50% or more.

In some embodiments, binding assays to assess variant activity are performed on tumor tissues or tumor cells ex vivo, e.g., on tumor cells that were grown as a tumor graft in a syngeneic (immune-matched) mouse in vivo then harvested and processed within 24-48 hrs. Binding can be assessed by any number of means including flow cytometry.

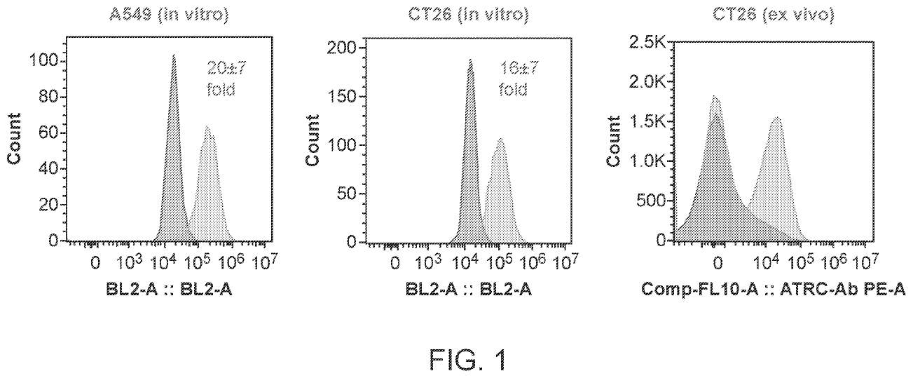

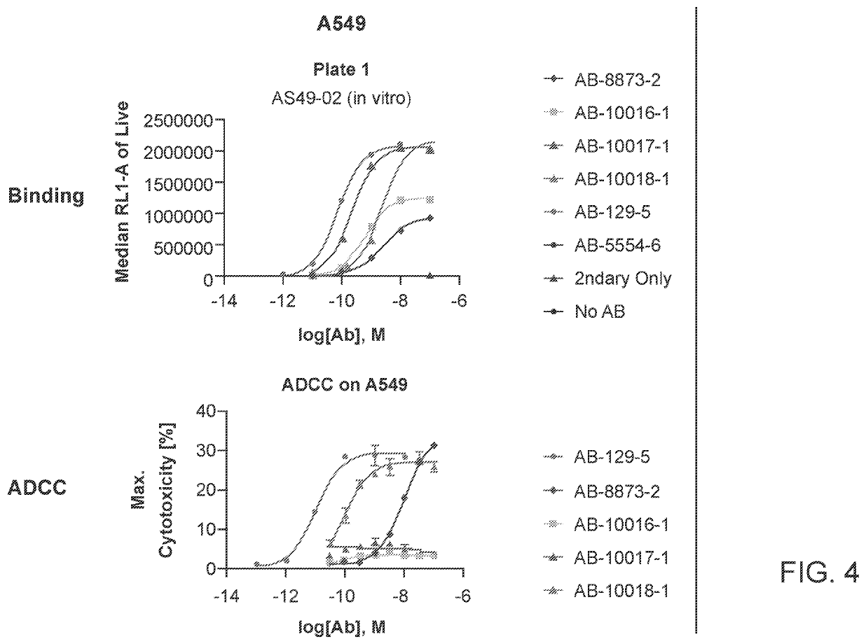

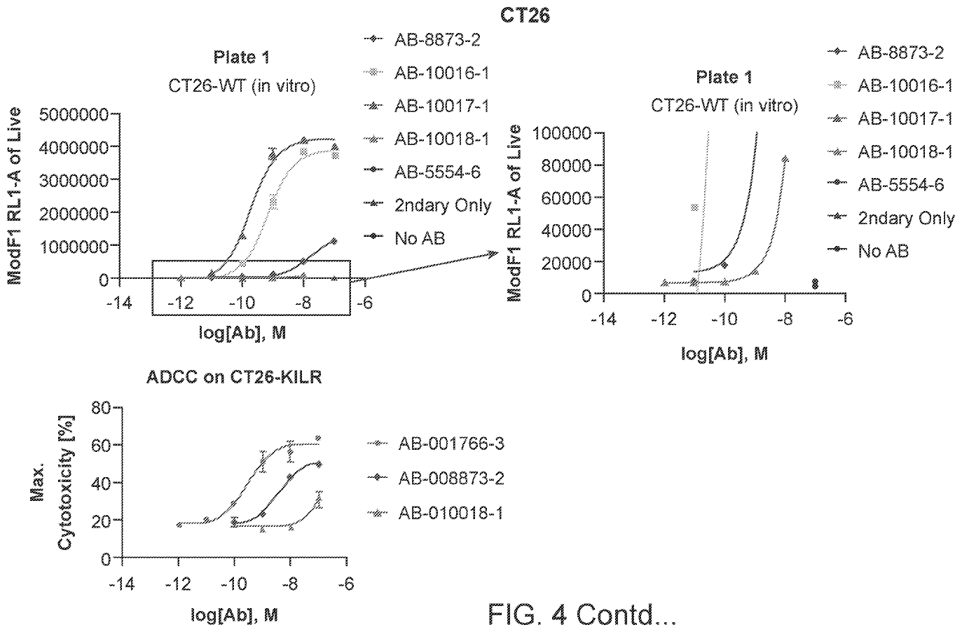

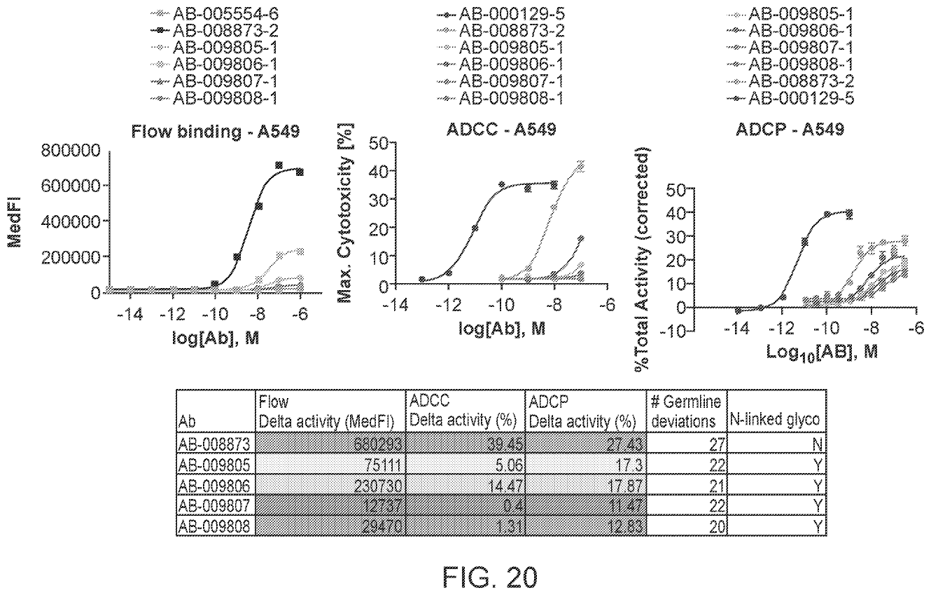

The antibodies disclosed herein bind specifically to tumor cells. In some embodiments the antibody is added to a cancer cell line and the binding is analyzed using a flow cytometry. AB-008873 showed a strong binding to 786-O, A375, A549, H522, LoVo, MDA-MB-231, PC3, RKO, SKOV3, SW1116, and CT26. Additionally, AB-008873 is also capable of binding to CT26 ex vivo cells. FIG. 1, Tables 23 and 24. Its sibling antibodies AB-009805, AB-009806, AB-009807, and AB-009808 also showed binding activity to A549 cells, but less than that of AB-008873. FIG. 20. Unlike AB-010018 (mouse monoclonal antibody precursor of humanized DS-8895a. See U.S. Pat. No. 9,150,657, Sequences 35 and 37 Daiichi Sankyo (IgG)), which showed binding and ADCC activity on A549 cells (human lung cancer cells) but did not on CT26 cells (murine colon cancer cells), AB-008873 demonstrated binding and ADCC activity on both A549 cells and CT26 cells. FIG. 4.

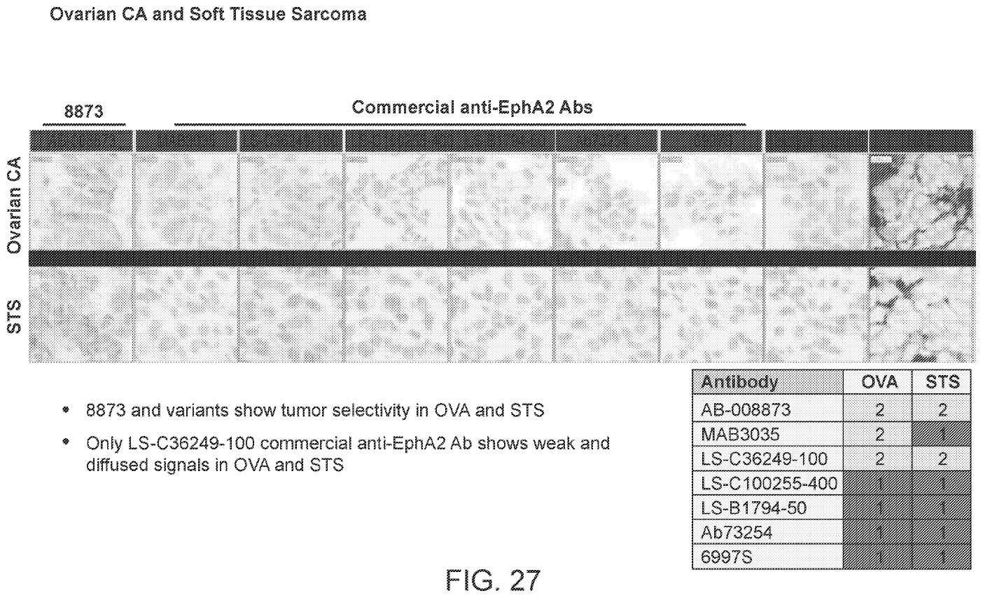

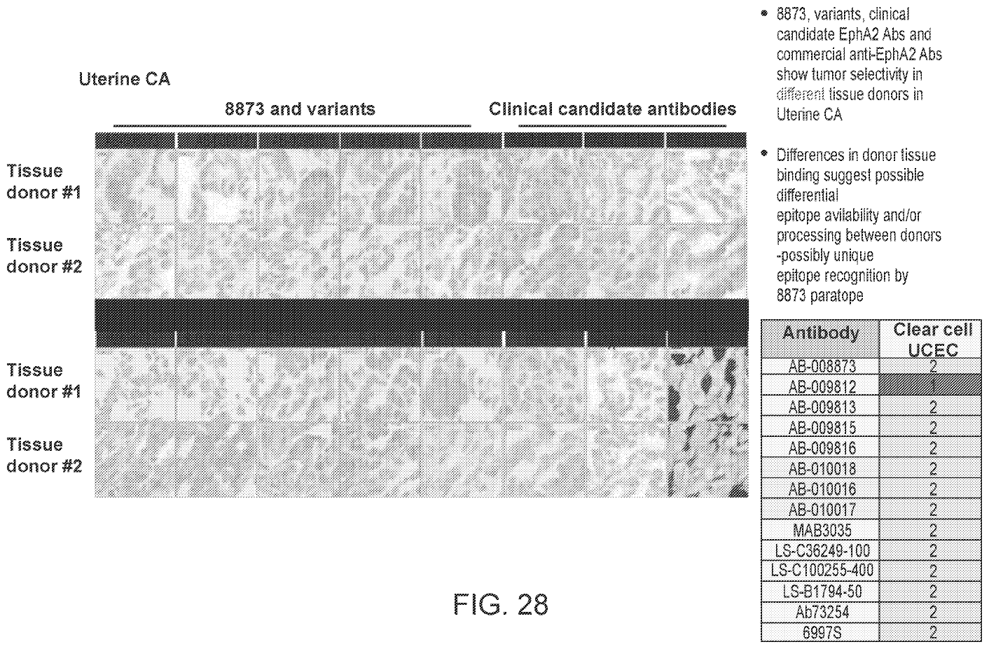

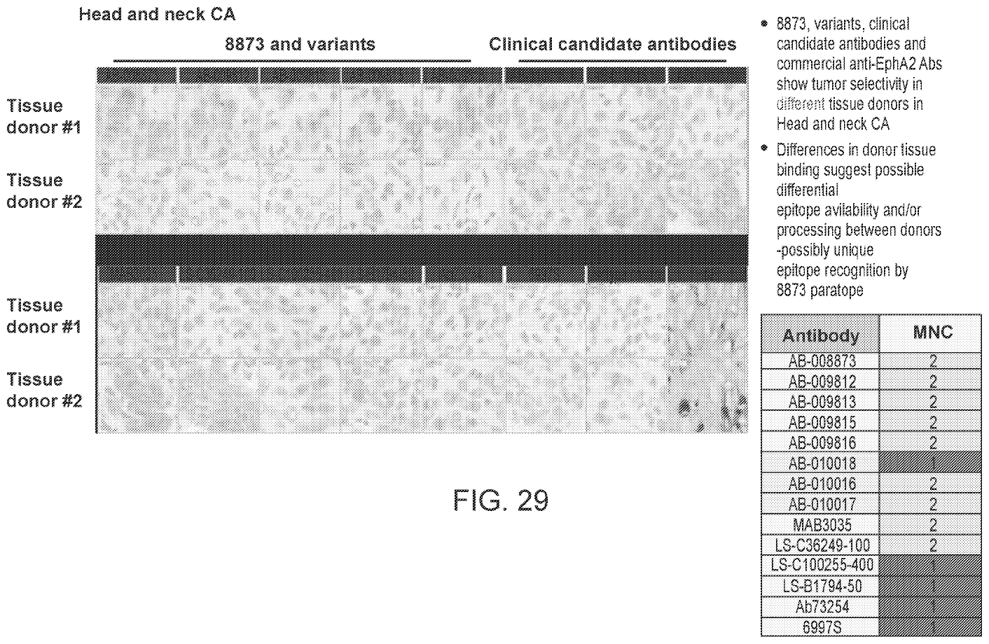

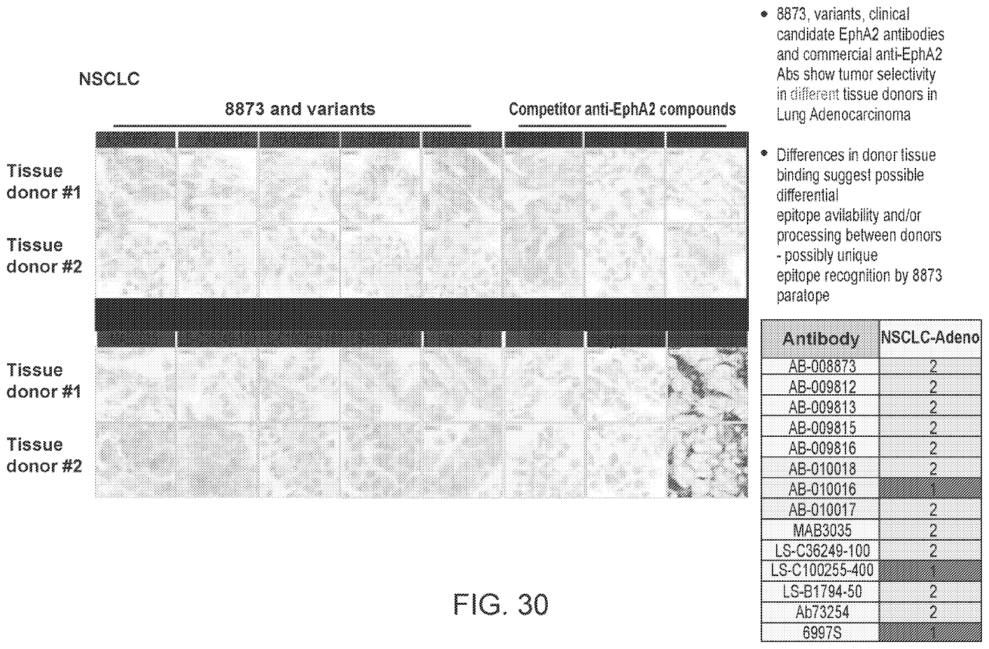

In some embodiments, the binding of the antibodies to bind to tumor cells are assessed by immunofluorescence methods, as described in the EXAMPLES. AB-008873 and its variants preferentially bind to various tumors but not to normal human tissues (FIGS. 27-34). As compared to AB-010018 and LS-C100255, AB-008873 advantageously showed less staining in normal tissues and a higher staining in tumor tissues. In one illustrative example, the AB-008873 showed preferential binding to ovarian cancer and soft tissue sarcoma (STS) than the respective tumor adjacent tissue (TATs); in contrast, all commercial anti-EphA2 antibodies tested, exception for LS-C 36249-100, did not show any detectable binding in these two tumors (FIG. 27). In some embodiments, for certain cancer types, such as uterine cancer, head and neck cancer, and NSCLS, an EphA2 antibody disclosed herein demonstrate tumor selectivity (i.e., preferentially bind to tumor tissue than TATs) in different donors. That is to say, the antibody shows tumor selectivity in some donors but not other donors. In one exemplary assay, AB-008873 and its variant antibodies, AB-009812, AB-009813, AB-009815, and AB-009816 showed tumor selectivity in different donors in uterine cancer, head and neck cancer, and NSCLS (FIG. 28-30). This suggests that there are possible differential epitope availability and/or processing between donors, or possibly a unique epitope recognition by the AB-008873 paratope.

In some embodiments, the antibody's binding activity of functional activity is assessed by determining EC₅₀ values, and in some embodiments additionally determining delta activity, i.e., the difference in specific activity between lower and upper plateaus of the activation curve expressed as percent of activity of a selected antibody having known in vitro activity. In typical embodiments, EC₅₀ values are compared to a reference antibody. For purposes of this disclosure, an antibody comprising the VH and VL regions of an EphA2 antibody disclosed herein and a mouse IgG2a Fc region when testing binding or functional activity using a tumor cell line, is employed as a reference antibody and included in an assay to assess variant activity relative to the reference antibody. The fold over EC₅₀ is calculated by dividing the EC₅₀ of the reference antibody by the EC₅₀ of the test antibody. Based on the resulting values, the antibodies were assigned to groups and given a ranking from 0-4 as follows: 0=(>500 nM); 1=<0.5; 2=0.5 to 2; 3=2 to 4; 4=>4.

### EphA2 Signaling Effect

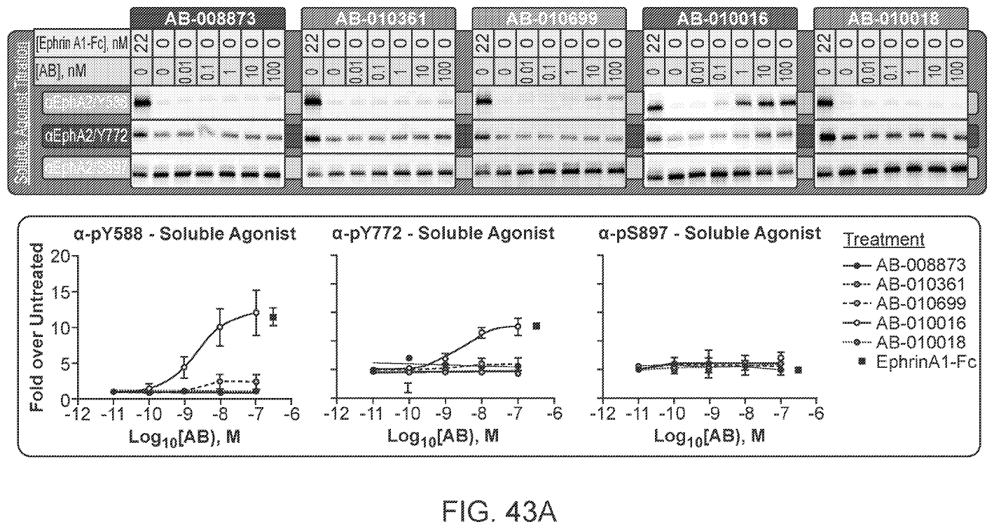

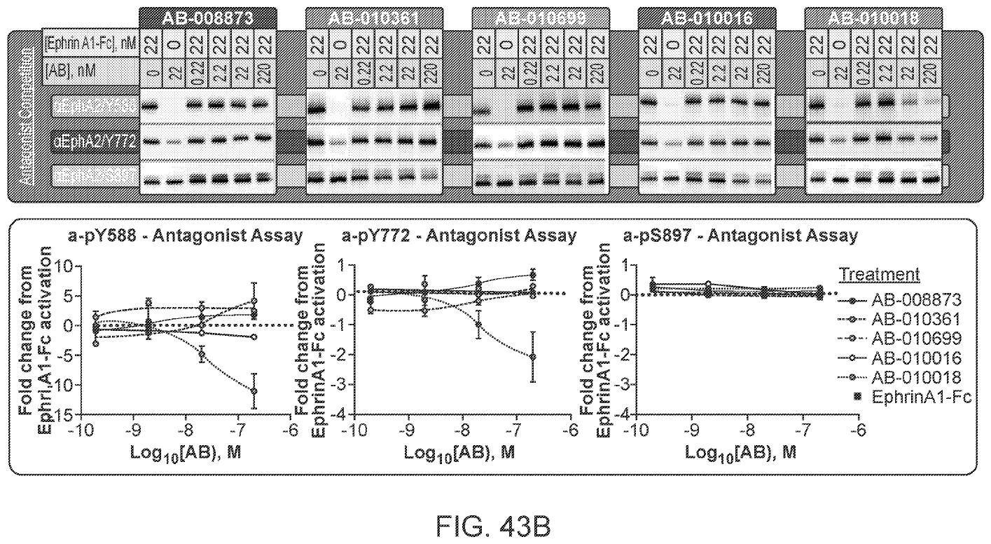

In some embodiments, an EphA2 antibody of the present disclosure exhibits no measurable agonistic effect on the ephrinA1-EphA2 signaling axis. In some embodiments, an EphA2 antibody of the present disclosure exhibits less agonistic effect on the ephrinA1-EphA2 signaling axis than the ephrin-A1 ligand. In some embodiments, an EphA2 antibody of the present disclosure activates the ephrin A1-EphA2 signaling axis at least 99%, 98% 95%, 90%, 85%, 80%, 75%, 70%, 65%, 60%, 55%, or 50% less than ephrin-A1 activates the ephrin A1-EphA2 signaling axis. In some embodiments, an EphA2 antibody of the present disclosure activates the ephrin A1-EphA2 signaling axis at least 99%, 98%, 95%, 90%, 85%, or 80% less than ephrin-A1 activates the ephrin A1-EphA2 signaling axis. FIG. 43A.

In some embodiments, an EphA2 antibody of the present disclosure shows weak agonistic effect on the ephrinA1-EphA2 signaling axis, with the effect occurring at apotency less than the potency of the ADCC of the antibody. In some embodiments, an EphA2 antibody of the present disclosure has an EC₅₀ for activation of the ephrinA1-EphA2 signaling axis that is at least 10-, 20-, 50-, 100-, 200-, 500-fold less potent than the ADCC EC₅₀ of the antibody.

In some embodiments, an EphA2 antibody of the present disclosure exhibits no antagonistic effect on the ephrinA1-EphA2 signaling axis. In some embodiments, an EphA2 antibody of the present disclosure exhibits less antagonistic effect on the ephrinA1-EphA2 signaling axis than AB-010018. FIG. 43B. Methods for measuring ephrinA1-EphA2 signaling axis and the effect of the EphA2 antibodies described herein are well known. In some embodiments, it can be accomplished by detecting the phosphorylation of EphA2 protein for example, one or more of the residues Y588, Y772, S897 of the EphaA2 protein. The degree of phosphorylation can be quantified using methods well known in the art, for example, Western Blot analysis using antibodies specific to described phosphorylation sites. One exemplary method of measuring ephrinA1-EphA2 signaling axis is disclosed in Example 5.

The limited effect on EphaA2 signaling provides a therapeutic advantage over other EphA2 antibodies that act as agonists or antagonists of the ephrin A1-EphA2 signaling axis by increasing the therapeutic index. For example, an EphaA2 antibody, or an immunoconjugate or bispecific version thereof, with limited effect on EphA2 signaling may be dosed at a level that provides both efficacy and safety while an EphaA2 antibody, or an immunoconjugate or bispecific version thereof, that agonizes the ephrin A1-EphA2 signaling axis could show a smaller, and potentially non-viable, therapeutic index.

### Fe Effector Function

In some embodiments, an EphA2 antibody of the present disclosure comprises an Fc region with effector function. Examples of effector functions include, but are not limited to, C1q binding and complement-dependent cytotoxicity (CDC), Fc receptor binding (e.g., FcγR binding), ADCC, antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, the Fc region of the EphA2 antibodies disclosed herein may be an Fc region engineered to alter one or more functional properties of the antibody, such as serum half-life, complement fixation, Fc receptor binding, and/or ADCC. Accordingly, an Fc region can comprise additional mutations to increase or decrease effector functions, i.e., the ability to induce certain biological functions upon binding to an Fc receptor expressed on an immune cell. Immune cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells. In some embodiments, an antibody of the present disclosure has enhanced ADCC and/or serum stability compared to antibody AB-008873 when the antibodies are assayed in a human IgG1 isotype format.

The EphA2 antibodies of the present disclosure may be evaluated in various assays for their ability to mediate FcR-dependent activity. In one assay, the binding activity of an EphA2 antibody is evaluated in an Fc receptor engagement assay. For purposes of testing variants, “engagement” of an Fc receptor occurs when a variant antibody binds to both a target tumor cell via its Fv region and an FcγR present on an immune cell via the antibody Fc region in such as manner to transduce a signal. If the Fc region is kept constant among variants that differ in their Fv regions, then the assay allows an evaluation of tumor binding activity across such variants in the context of potential signal transduction through a particular Fc region binding a particular Fc receptor. In some embodiments, binding of the antibody Fc region can result in clustering and/or internalization of the FcR, resulting in a luminescence signal in cells harboring a NFAT-RE-Luciferase reporter construct.

### ADCC Activity

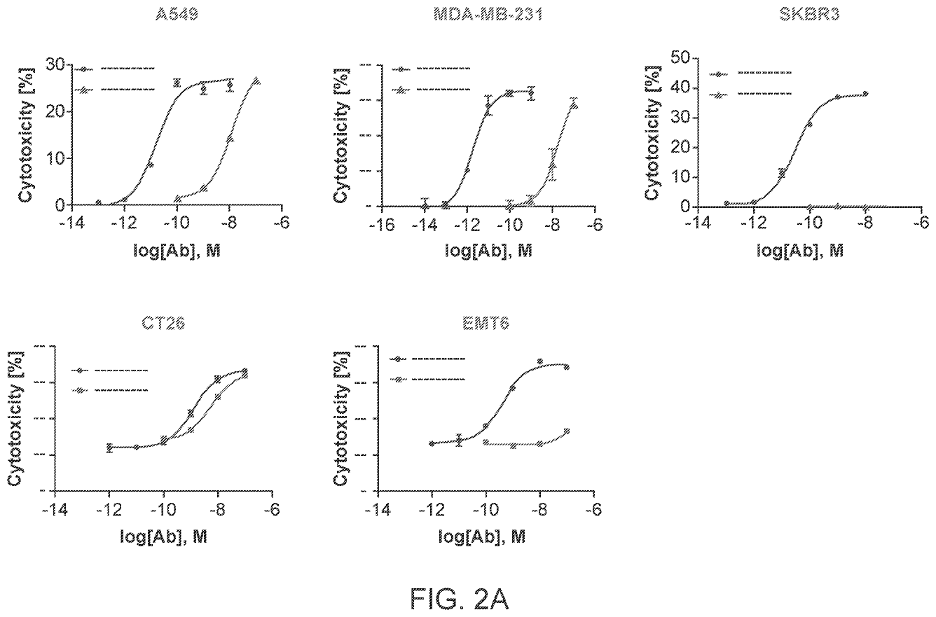

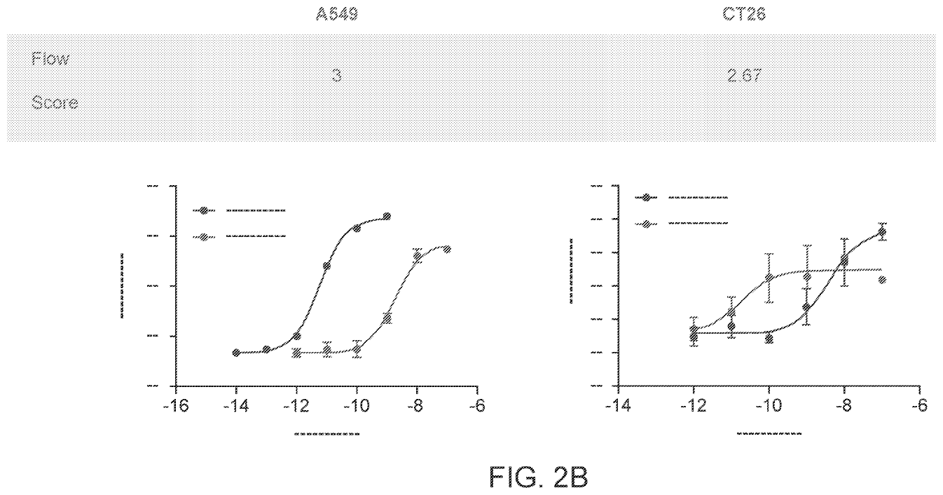

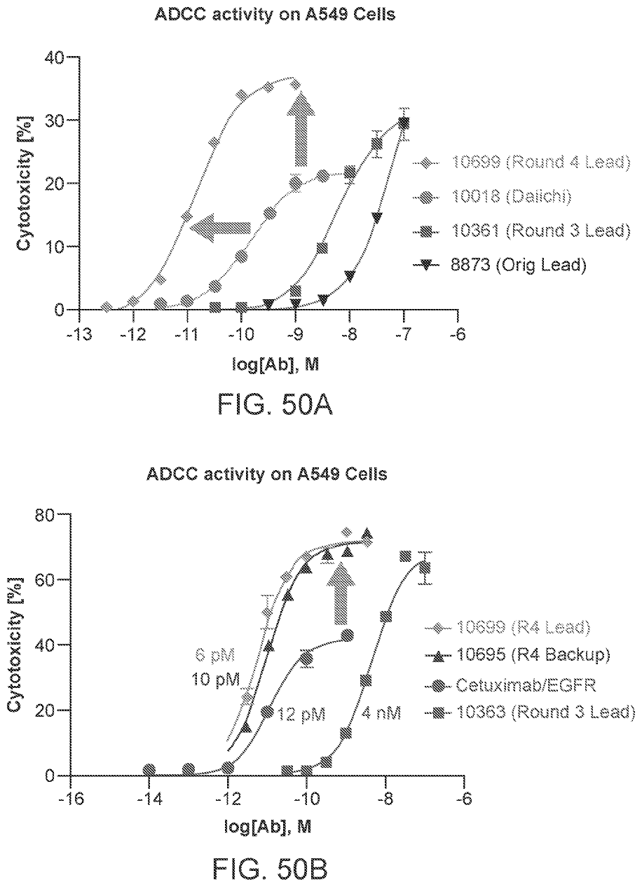

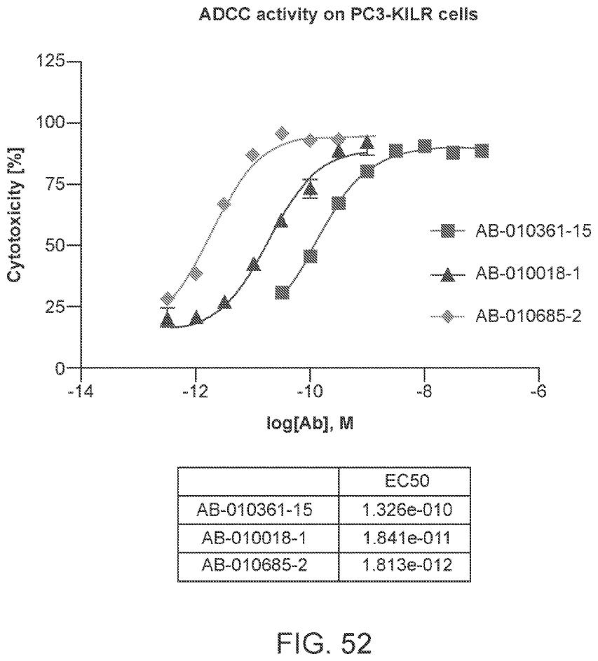

In some embodiments, an EphA2 antibody of the present disclosure has ADCC when the antibodies are assayed in a mouse IgG2a isotype format. In some embodiments, an EphA2 antibody of the present disclosure has ADCC activity that is comparable to that of AB-008873. The term “comparable activity,” refers to the ADCC activity of the EphA2 antibody in a range of 40% to 200% of the activity of a reference antibody that exhibits ADCC when evaluated under the same assay conditions. In one exemplary assay, AB-008873 showed dose dependent ADCC on A549, MDA-MB-231, CT26, and to a small extent EMT6. See FIG. 2A-2B and Table 25. Its sibling antibodies AB-009805, AB-009806, AB-009807, and AB-009808 also showed ADCC activity on A549 cells, but less than that of AB-008873. Certain variants exhibited improved ADCC activity, including AB-010699, AB-010361 and AB-010685. FIG. 50A-50B and FIG. 52.

### ADCP Activity

In some embodiments, ADCP activity of an EphA2 antibody (e.g., a variant of AB-008873) is assessed using fluorescently labeled, in vitro cultured tumor cells and Raw264.7 murine macrophages. In certain embodiments, opsonization of the tumor cell by the antibody leads to phagocytosis detected by flow cytometry. Variations of this assay have been described and can include co-labeling of tumor and effector cells or assessment of phagocytosis through FecyRIIa engagement (e.g., FcγRIIa-H ADCP Reporter Bioassay from Promega).

In one illustrative embodiment, The ADCP activity of an EphA2 antibody is evaluated using the method described above and in more detail in EXAMPLE 3. In one illustrative example, AB-008873 showed strong ADCP activity on A549 cells. FIG. 2B and FIG. 20. The sibling antibodies AB-009805, AB-009806, AB-009807, and AB-009808 also showed ADCP activity, but less than that of AB-008873. In one illustrative example, AB-008873 showed strong ADCP activity on A549 cells. FIG. 2A-2B, Table 25, and FIG. 20. The sibling antibodies AB-009805, AB-009806, AB-009807, and AB-009808 also showed ADCP activity, but less than that of AB-008873.

### ADC Activity

An EphA2 antibody is deemed to have ADC activity if, when the antibody is conjugated to a drug molecule (toxin) to form an antibody drug conjugate (ADC), said ADC can kill target cells. In some embodiments, the antibody is deemed to have ADC activity if the EC50 of the assay measuring the cell killing activity of the ADC is less than 1E-08. In one exemplary assay, the ADC activity of the EphA2 antibody is evaluated using a drug-conjugated secondary antibody. The antibody-drug conjugate assay involves tumor target cells, primary antibodies of interest (the EphA2 antibody to be tested), and a secondary antibody that is conjugated to a drug molecule, where the secondary antibody recognizes the primary antibody. Briefly, primary antibody dilutions are incubated with target cells at room temperature for a first period (for example, 10-30 minutes). The drug-conjugated secondary antibody is then added to the incubation mixture containing the target cells and the primary antibody. The mixture is then incubated for second period before measuring the extent of target cell lysis. In some embodiments, the assay generates a 100% cell lysis value by adding cell lysis buffer directly to target cell sample, which are not treated by the primary or drug-conjugated second antibody, and cell killing data from samples treated the antibody mixtures as disclosed above can be normalized to the value of 100% cell lysis. The results of the assay can be used to predict whether an ADC produced by conjugating a particular antibody and the drug molecule can kill target cells.

### In Vivo Activity

In some embodiments, activity of an EphA2 antibody variant is evaluated in vivo in a suitable animal tumor model. A reduction in tumor load reflects the anti-tumor function of an antibody.

In some embodiments, a variant of an antibody as described herein has at least 50%, or at least 60%, or 70%, or greater, of the anti-tumor activity of a reference antibody as shown in Tables 1-4 when evaluated under the same assay conditions to measure the anti-tumor activity in vivo. In some embodiments, an anti-tumor antibody exhibits improved activity, i.e., greater than 100% activity, compared to the reference antibody.

### Antibody Formats

In a further aspect of the invention, an EphA2 antibody in accordance with the disclosure may be an antibody fragment, e.g., a Fv, Fab, Fab′, scFv, diabody, or F(ab′)₂ fragment. In another embodiment, the antibody is a substantially full-length antibody, e.g., an IgG antibody or other antibody class or isotype as defined herein. For a review of certain antibody fragments, see Hudson et al. Nat. Med. 9: 129-134 (2003). Antibody fragments can be made by various techniques, including but not limited to proteolytic digestion of an intact antibody as well as production by recombinant host cells.

In some embodiments, an EphA2 antibody according to the present disclosure that is administered to a patient is an IgG of the IgG1 subclass. In some embodiments, such an antibody is an IgG of the IgG2, IgG3, or IgG4 subclass. In some embodiments, such an antibody is an IgM. In some embodiments, such an antibody has a lambda light chain constant region. In some embodiments, such an antibody has a kappa light chain constant region.

In some embodiments an EphA2 antibody in accordance with the present disclosure is in a monovalent format. In some embodiments, the tumor-targeting antibody is in a fragment format, e.g., a Fv, Fab, Fab′, scFv, diabody, or F(ab′)₂ fragment.

In some embodiments, EphA2 antibodies disclosed herein, including antibody fragments, of the present disclosure comprises an Fc region with effector function, e.g., exhibits antibody-dependent cellular cytotoxicity (ADCC), antibody-dependent cellular phagocytosis (ADCP), and/or complement-dependent cytotoxicity (CDC). In some embodiments, the Fc region may be an Fc region engineered to alter one or more functional properties of the antibody, such as serum half-life, complement fixation, Fc receptor binding, and/or ADCC. Accordingly, an Fc region can comprise additional mutations to increase or decrease effector functions, i.e., the ability to induce certain biological functions upon binding to an Fc receptor expressed on an immune cell. Immune cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells.

In some embodiments, an Fc region described herein can include additional modifications that modulate effector function. Examples of Fc region amino acid mutations that modulate an effector function include, but are not limited to, one or more substitutions at positions 228, 233, 234, 235, 236, 237, 238, 239, 243, 265, 269, 270, 297, 298, 318, 326, 327, 329, 330, 331, 332, 333, and 334 (EU numbering scheme) of an Fc region.

Illustrative substitutions that decrease effector functions include the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions that decrease effector functions include S228P, E233P, L235E, N297A, N297D, and P331S. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, to decrease effectors functions. Examples of substitutions that increase effector functions include, e.g., E333A, K326W/E333S, S239D/I332E/G236A, S239D/A330L/I332E, G236A/S239D/A330L/I332E, F243L, G236A, and S298A/E333A/K334A. In some embodiments, the Fc mutations include P329G, L234A, L235A, or a combination thereof. Descriptions of amino acid mutations in an Fc region that can increase or decrease effector functions can be found in, e.g., Wang et al., Protein Cell. 9(1): 63-73, 2018; Saunders, Front Immunol. June 7, eCollection, 2019; Kellner et al., Transfus Med Hemother. 44(5): 327-336, 2017; and Lo et al., J Biol Chem. 292(9):3900-3908, 2017.

In some embodiments, an Fc region may have one or more amino acid substitutions that modulate ADCC, e.g., substitutions at positions 298, 333, and/or 334 of the Fc region, according to the EU numbering scheme. Specifically, S298A, E333A, and K334A can be introduced to an Fc region to increase the affinity of the Fc region to FcγRIIIa and decrease the affinity of the Fc region to FcγRIIa and FcγRIIb.

An Fc region can also comprise additional mutations to increase serum half-life. Through enhanced binding to the neonatal Fc receptor (FcRn), such mutations in an Fc region can improve pharmacokinetics of the antibody. Examples of substitutions in an Fc region that increase the serum half-life of an antibody include, e.g., M252Y/S254T/T256E, T250Q/M428L, N434A, N434H, T307A/E380A/N434A, M428L/N434S. M252Y/M428L, D259I/V308F, N434S, V308W, V308Y, and V308F. Descriptions of amino acid mutations in an Fc region that can increase the serum half-life of an antibody can be found in, e.g., Dumet et al., MAbs. 26:1-10, 2019; Booth et al., MAbs. 10(7):1098-1110, 2018; and Dall'Acqua et al., J Biol Chem. 281(33):23514-24, 2006.

Furthermore, in some embodiments, an antibody of the disclosure may be chemically modified (e.g., one or more chemical moieties can be attached to the antibody) or be modified, e.g., produced in cell lines and/or in cell culture conditions to alter its glycosylation (e.g., hypofucosylation, afucosylation, or increased sialylation), to alter one or more functional properties of the antibody. For example, the antibody can be linked to one of a variety of polymers, for example, polyethylene glycol. In some embodiments, an antibody may comprise mutations to facilitate linkage to a chemical moiety and/or to alter residues that are subject to post-translational modifications, e.g., glycosylation.

In some embodiments, an EphA2 antibody described herein comprise an Fc region having altered glycosylation that increases the ability of the antibody to recruit NK cells and/or increase ADCC. In some embodiments, the Fc region comprises glycan containing no fucose (i.e., the Fc region is afucosylated). Afucosylated antibodies can be produced using cell lines that express a heterologous enzyme that depletes the fucose pool inside the cell (e.g., GlymaxX® by ProBioGen AG, Berlin, Germany). Non-fucosylated antibodies can also be produced using a host cell line in which the endogenous α-1,6-fucosyltransferase (FUT8) gene is deleted. See Satoh, M. et al., “Non-fucosylated therapeutic antibodies as next-generation therapeutic antibodies,” Expert Opinion on Biological Therapy, 6:11, 1161-1173, DOI: 10.1517/14712598.6.11.1161.

In some embodiments, an EphA2 antibody is constructed as a multivalent antibody. In some embodiments, an EphA2 antibody is constructed as a tetravalent molecule, comprising four EphA2 binding arms per molecule. Such constructs exhibit increased ADCC activity, as well as increased binding to tumor cells a measured by flow cytometry.

In some embodiments, an EphA2 antibody of the present disclosure is employed in a bispecific or multi-specific format, e.g., a tri-specific format. For example, in some embodiments, the antibody may be incorporated into a bispecific or multi-specific antibody that comprises a further binding domain that binds to the same or a different antigen.

There are a variety of possible formats that can be used in bispecific or multi-specific antibodies. The formats can vary elements such as the number of binding arms, the format of each binding arm (e.g., Fab, scFv, scFab, or VH-only), the number of antigen binding domains present on the binding arms, the connectivity and geometry of each arm with respect to each other, the presence or absence of an Fc domain, the Ig class (e.g., IgG or IgM), the Fc subclass (e.g., hIgG1, hIgG2, or hIgG4), and any mutations to the Fc (e.g., mutations to reduce or increase effector function or extend serum half-life). Also see Speiss et al., Alternative Molecular Formats and Therapeutic Applications for Bispecific Antibodies, Mol Immunol, 67, 95-106 (2015), in particular FIG. 1, for examples of bispecific and multispecific formats.

Illustrative antigens that can be targeted by a further binding domain in a bispecific or multi-specific antibody that comprises an antigen binding domain of an EphA2 antibody described herein, include, but are not limited to, antigens on T cells to enhance T cell engagement and/or activate T cells. Illustrative examples of such an antigen include, but are not limited to, CD3, CD2, CD4, CD5, CD6, CD8, CD28, CD40L, CD44, IL-15Rα, CD122, CD132, or CD25. In some embodiments, the antigen is CD3. In some embodiments, the antigen is in a T cell activating pathway, such as a 4-1BB/CD137, 4-1BBL/CD137L, OX40, OX40L, GITRL, GITR, CD27, CD70, CD28, ICOS, HVEM, or LIGHT antigen.

In some embodiments, an EphA2 antibody is incorporated into a bispecific or multi-specific antibody that comprises a binding domain that binds to a T-cell antigen. These bispecific antibodies or multi-specific antibodies can direct T cells to attach and lyse targeted tumor cells, i.e., the EphA2 expressing tumor cells. In some embodiments, the bispecific or multispecific antibody comprises a binding domain that binds to CD3. In some embodiments, the bispecific or multispecific antibody comprises a binding domain that binds to human CD3 comprising the anti-tumor antibodies described herein.

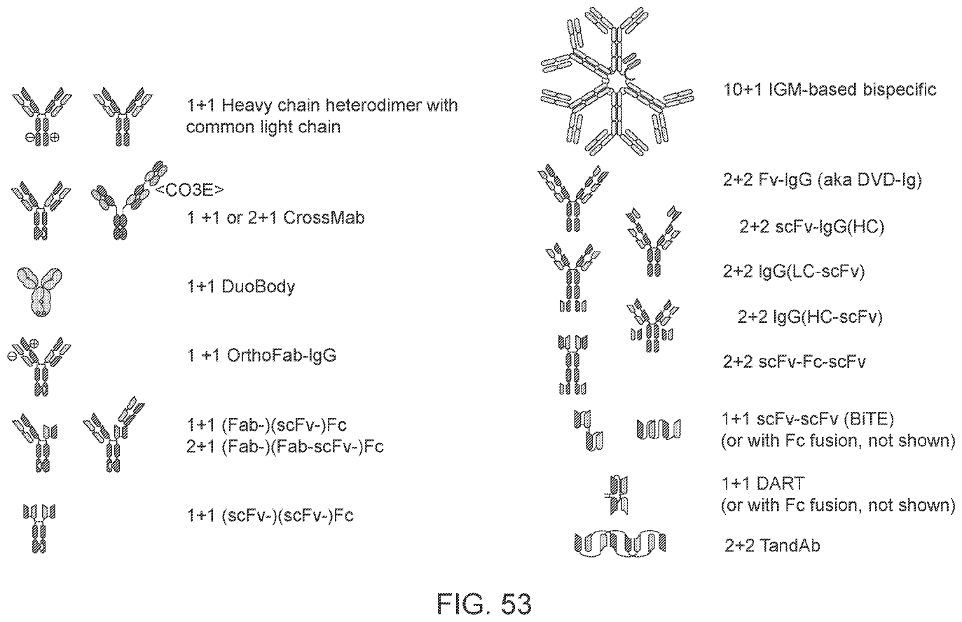

Table 9 provides specific examples of anti-CD3 binding arms that can be combined with any of the anti-EphA2 antibodies described herein. FIG. 53 provides examples of a variety of formats according to which anti-EphA2/anti-CD3 bispecific constructs that can be generated.

TABLE 9
CD3 heavy chain (VH) and light chain (VL) sequences.

| AB ID | VH | VL |
|---|---|---|
| AB-008703 | (SEQ ID NO: 133) | (SEQ ID NO: 134) |
| AB-008704 | (SEQ ID NO: 135) | (SEQ ID NO: 136) |
| AB-008705 | (SEQ ID NO: 137) | (SEQ ID NO: 138) |
| AB-008706 | (SEQ ID NO: 139) | (SEQ ID NO: 140) |
| AB-008707 | (SEQ ID NO: 141) | (SEQ ID NO: 142) |
| AB-008708 | (SEQ ID NO: 143) | (SEQ ID NO: 144) |
| AB-008709 | (SEQ ID NO: 145) | (SEQ ID NO: 146) |
| AB-008710 | (SEQ ID NO: 147) | (SEQ ID NO: 148) |
| AB-008711 | (SEQ ID NO: 149) | (SEQ ID NO: 150) |
| AB-008712 | (SEQ ID NO: 151) | (SEQ ID NO: 152) |
| AB-008713 | (SEQ ID NO: 153) | (SEQ ID NO: 154) |
| AB-008714 | (SEQ ID NO: 155) | (SEQ ID NO: 156) |
| AB-008715 | (SEQ ID NO: 157) | (SEQ ID NO: 158) |
| AB-008716 | (SEQ ID NO: 159) | (SEQ ID NO: 160) |
| AB-008717 | (SEQ ID NO: 161) | (SEQ ID NO: 162) |
| AB-008718 | (SEQ ID NO: 163) | (SEQ ID NO: 164) |
| AB-008719 | (SEQ ID NO: 165) | (SEQ ID NO: 166) |
| AB-008720 | (SEQ ID NO: 167) | (SEQ ID NO: 168) |
| AB-008721 | (SEQ ID NO: 169) | (SEQ ID NO: 170) |
| AB-008722 | (SEQ ID NO: 171) | (SEQ ID NO: 172) |
| AB-008723 | (SEQ ID NO: 173) | (SEQ ID NO: 174) |
| AB-008724 | (SEQ ID NO: 175) | (SEQ ID NO: 176) |
| AB-008725 | (SEQ ID NO: 177) | (SEQ ID NO: 178) |
| AB-008726 | (SEQ ID NO: 179) | (SEQ ID NO: 180) |
| AB-008727 | (SEQ ID NO: 181) | (SEQ ID NO: 182) |
| AB-008728 | (SEQ ID NO: 183) | (SEQ ID NO: 184) |
| AB-008729 | (SEQ ID NO: 185) | (SEQ ID NO: 186) |
| AB-008730 | (SEQ ID NO: 187) | (SEQ ID NO: 188) |
| AB-008731 | (SEQ ID NO: 189) | (SEQ ID NO: 190) |
| AB-008732 | (SEQ ID NO: 191) | (SEQ ID NO: 192) |
| AB-008733 | (SEQ ID NO: 193) | (SEQ ID NO: 194) |
| AB-008734 | (SEQ ID NO: 195) | (SEQ ID NO: 196) |
| AB-008735 | (SEQ ID NO: 197) | (SEQ ID NO: 198) |
| AB-008736 | (SEQ ID NO: 199) | (SEQ ID NO: 200) |

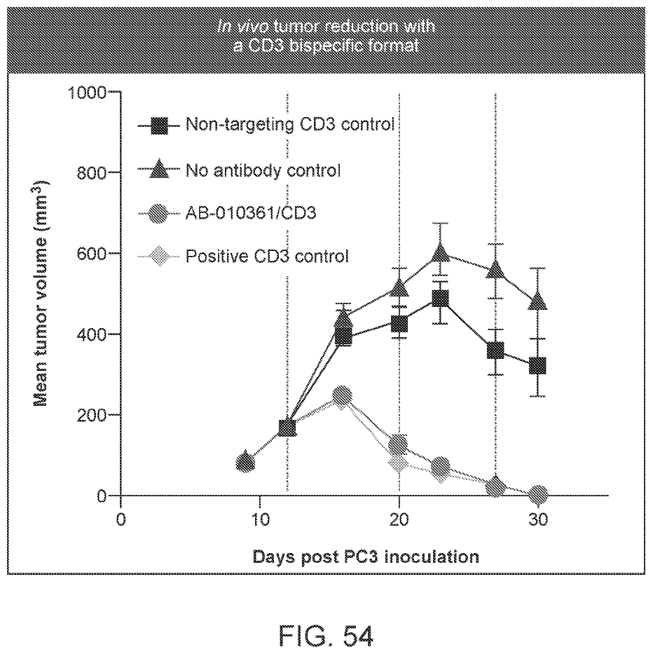

FIG. 54 shows treatment of mice carrying the tumors formed by PC3 tumor cells using an exemplary bispecific antibody led to tumor burden reduction and/or elimination of tumors in mice. Said bispecific antibody was generated using a 1+1 (Fab−)(scFv−)Fc format (shown in FIG. 53) and the CD3 arm of which comprises the VH/VL sequence of AB-008707.

Additional antigens that can be targeted by a further binding domain in a bispecific or multi-specific antibody that comprises an antigen binding domain of an EphA2 antibody described herein, include, but are not limited to, antigens on NK cells to activate or inhibit NK cell pathways.

Illustrative examples of such an antigen include activating NK cell receptors such as activating human Killer Immunoglobulin-like Receptor (KIR) family members, activating Ly49 family members, CD94-NKG2C/E/H heterodimeric receptors, NKG2D, SLAM family receptors including 2B4/CD244, CRACC/SLAMF7, NTB-A/SLAMF6, Fc gamma RIIIA/CD16a, CD27, CD100/Semaphorin 4D, CD160, natural cytotoxicity receptors, including NKp30, NKp44, and NKp46, DNAM-1/CD226, IL-2 receptor subunit beta (IL-2RB), IL-2 receptor subunit gamma (IL-2RG), 4-1BB/CD137, and CRTAM. Illustrative examples of such an antigen include inhibiting NK cell receptors such as inhibiting human KIR family members, inhibiting Ly49 family members, CD94/NKG2A, TIGIT and CD96, sialic acid-binding Siglecs (Siglec-3, -7, and -9), ILT2/LILRB1, KLRG1, LAIR-1, CD161/NKR-P1A, and CEACAM-1.

In some embodiments, an EphA2 antibody is incorporated into a bispecific or multi-specific antibody that comprises a binding domain from an agonist antibody that binds to 4-1BB.

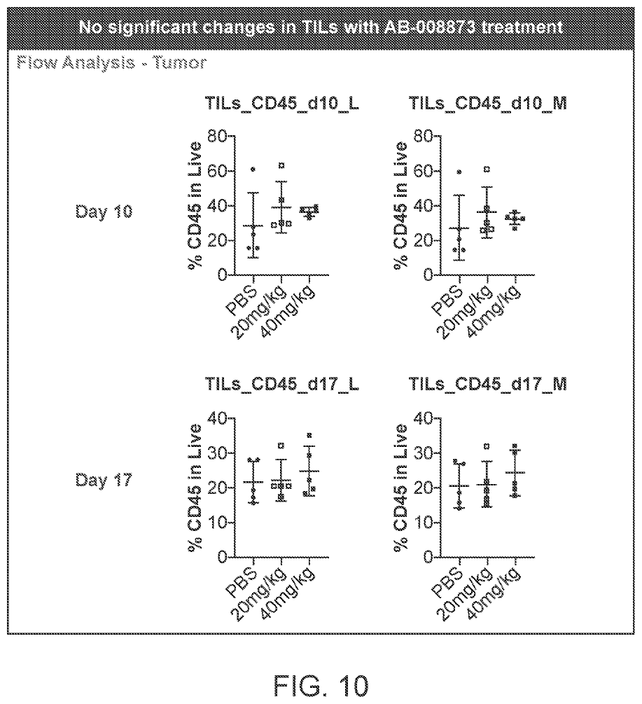

In one embodiment the 4-1 in agonist antibody is a bispecific antibody that is capable of binding to both EphA2 and 4-1BB. For purposes of this application, the term “4-1BB engager,” refers to the portion of a molecule (e.g., a bispecific antibody capable of binding to both 4-1BB and EphA2) that binds to 4-1BB. In some embodiments, the 4-1BB engager is an antibody or an antibody fragment (e.g., scFv) that binds to 4-1BB. In some embodiments, the 4-1BB engager is a multimeric 4-1BB ligand (“4-1BBL”), for example, a 4-1BBL trimer. In some embodiments, as further described below, the bispecific antibody comprises one or more scFv fragments of an anti-4-1BB antibody and an EphA2 antibody disclosed herein. In one embodiment, the 4-1BB agonist antibody is a trispecific antibody. Examples of bispecific and trispecific antibody constructs are described in US 20190010248, FIG. 1; WO2020025659, FIG. 1; Berezhnoy A. et al., Converting PD-L1-induced T-lymphocyte Inhibition into CD137-mediated Costimulation via PD-L1×CD137 Bispecific DART® Molecules. Poster presented at 30^{th} EORTC/AACR/NCI Symposium, Nov. 13-16, 2018, Dublin, Ireland, Compte, M. et al., A tumor-targeted trimeric 4-1BB-agonistic antibody induces potent tumor-targeting immunity without systemic toxicity. Nat Com 9, 4809 (2018), FIG. 1; U.S. Ser. No. 10/239,949, FIG. 10; and WO2019/092452, Example 2.

Non-limiting examples of the scFv of the anti 4-1BB antibodies that can be used in the fusion proteins are shown in Table 10. Exemplary linkers that can be used to connect the heavy chain or light chain of the EphA2 antibody and the 4-1BB ligand domains in the fusion are shown in Table 14. Various fusion proteins comprising the 4-1BBL and the EphA2 antibodies are shown in Table 14.

TABLE 10
scFv4-1BB sequences used in the fusion construct

| Part Name | Description | Part Sequence |
|---|---|---|
| SCFV-009036-01 | Anti-4-1BB SCFV | (SEQ ID NO: 111) |
| SCFV-009036-02 | Anti-4-1BB SCFV | (SEQ ID NO: 112) |
| SCFV-009365-01 | Anti-4-1BB SCFV | (SEQ ID NO: 113) |
| SCFV-009365-02 | Anti-4-1BB SCFV | (SEQ ID NO: 114) |
| SCFV-009366-01 | Anti-4-1BB SCFV | (SEQ ID NO: 115) |
| SCFV-009366-02 | Anti-4-1BB SCFV | (SEQ ID NO: 116) |
| SCFV-009037-01 | Anti-4-1BB SCFV | (SEQ ID NO: 117) |
| SCFV-009037-02 | Anti-4-1BB SCFV | (SEQ ID NO: 118) |
| SCFV-009809-01 | Anti-4-1BB SCFV | (SEQ ID NO: 119) |
| SCFV-009809-02 | Anti-4-1BB SCFV | (SEQ ID NO: 120) |
| SCFV-009810-01 | Anti-4-1BB SCFV | (SEQ ID NO: 121) |
| SCFV-009810-02 | Anti-4-1BB SCFV | (SEQ ID NO: 122) |

TABLE 11
Heavy Chain Sequences Used in 4-1BB Bispecific antibody

| | Sequence of | Sequence of |
|---|---|---|
| | CH123 fused to | CH123 not fused |
| IgG Identifier | 4-1BB engager | (if applicable) |
| hIgG1 | (SEQ ID NO: 123) | N/A |
| hIgG4_S228P_R409K | (SEQ ID NO: 124) | N/A |
| hIgG1_P329G_L234A_L235A | (SEQ ID NO: 125) | N/A |
| mIgG1 | (SEQ ID NO: 126) | N/A |
| hIgG1_kih | (SEQ ID NO: 127) | (SEQ ID NO: 128) |
| hIgG4_S228P_R409K_kih | (SEQ ID NO: 129) | (SEQ ID NO: 130) |
| hIgG1-Xmab | (SEQ ID NO: 131) | (SEQ ID NO: 132) |

Note: “CH123” refers to an IgG heavy chain constant region comprising CH1-CH2-hinge-CH3.

Additional 4-1BB antibody sequences suitable for use in generating bispecific constructs that are capable of binding to both EphA2 and 4-1BB are provided in Table 12.

TABLE 12
4-1BB antibody sequences suitable for
generating bispecific constructs

| 4-1BB antibody | V_{H} | V_{L} |
|---|---|---|
| hu106-1 | (SEQ ID NO: 721) | (SEQ ID NO: 743) |
| TABBY106 | (SEQ ID NO: 722) | (SEQ ID NO: 744) |
| TABBY107 | (SEQ ID NO: 723) | (SEQ ID NO: 745) |
| BBK4 | (SEQ ID NO: 724) | (SEQ ID NO: 746) |
| hu39E3G.4 | (SEQ ID NO: 725) | (SEQ ID NO: 747) |
| Urelumab | (SEQ ID NO: 726) | (SEQ ID NO: 748) |
| CTX-471 | (SEQ ID NO: 727) | (SEQ ID NO: 749) |
| CTX-471AF | (SEQ ID NO: 728) | (SEQ ID NO: 750) |
| MOR-6032 | (SEQ ID NO: 729) | (SEQ ID NO: 751) |
| MOR-7361 | (SEQ ID NO: 730) | (SEQ ID NO: 752) |
| MOR-7480 | (SEQ ID NO: 731) | (SEQ ID NO: 753) |
| MOR-7480.1/Utomilumab | (SEQ ID NO: 732) | (SEQ ID NO: 754) |
| MOR-7483 | (SEQ ID NO: 733) | (SEQ ID NO: 755) |
| Utomilumab | (SEQ ID NO: 734) | (SEQ ID NO: 756) |
| EU-101 | (SEQ ID NO: 735) | (SEQ ID NO: 757) |
| STA-551 | (SEQ ID NO: 736) | (SEQ ID NO: 758) |
| LVGN-6051 | (SEQ ID NO: 737) | (SEQ ID NO: 759) |
| AGEN-2373 | (SEQ ID NO: 738) | (SEQ ID NO: 760) |
| ADG-106/AG10131 | (SEQ ID NO: 739) | (SEQ ID NO: 761) |
| ATOR-1017 | (SEQ ID NO: 740) | (SEQ ID NO: 762) |
| Hz4B4-1 | (SEQ ID NO: 741) | (SEQ ID NO: 763) |
| Hz4B4-2 | (SEQ ID NO: 742) | (SEQ ID NO: 764) |

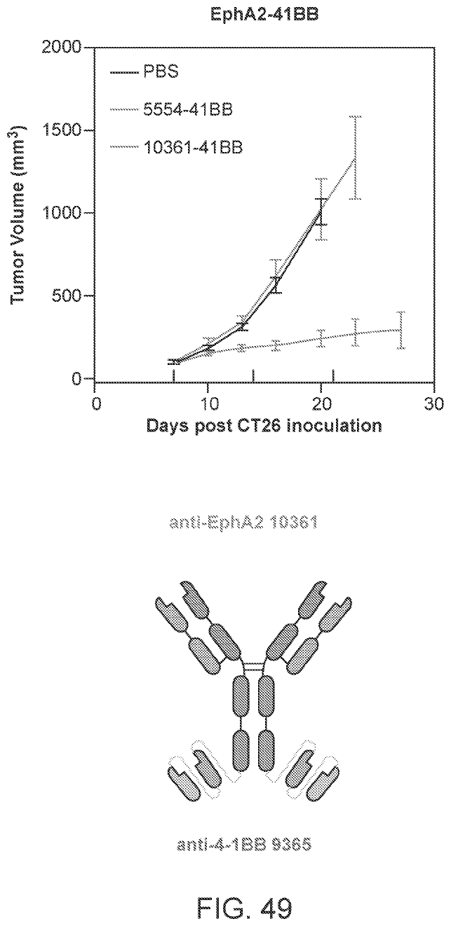

As shown in FIG. 49, one exemplary bispecific antibody that comprises two scFv fragments of anti-4-1BB antibody linked to the HC of the EphA2 antibody AB-010361 showed capability of reducing tumor growth in mice carrying tumors formed from CT26 cells.

In one embodiment, the fusion molecule comprises a silenced human IgG1 with three human 4-1BB ligand ectodomains attached via flexible linkers. See WO2019086499, FIGS. 1-3. Other 4-1BBL fusion molecules can be utilized with the tumor-targeting antibodies described herein, see Zhang et al., Targeted and Untargeted CD137L Fusion Proteins for the Immunotherapy of Experimental Solid Tumors, Clin Cancer Res., 2758-2767 (2007), FIG. 1; (Kermer et al., Combining Antibody-Directed Presentation of IL-15 and 4-1BBL in a Trifunctional Fusion Protein for Cancer Immunotherapy, Mol Cancer Ther, 112-121 (2014), FIG. 1.

In some embodiments, an EphA2 antibody is incorporated into a fusion molecule comprising one or more 4-1BB ligands (4-1BBL). In one embodiment, a trimer of 4-1BBL is C-terminally fused to either the light chain or heavy chain of an EphA2 antibody. In one embodiment, one or more individual 4-1BBL domains are connected via linkers, with one of the domains additionally fused to the EphA2 antibody via a linker. The 4-1BBL domains comprise entire ECD portion of the molecule or truncated forms that can still bind and activate 4-1BB. See WO2019086499, FIGS. 1-3. Non-limiting examples of the 4-1BBL domains that can be used in the fusion proteins are shown in Table 13. Exemplary linkers that can be used to connect the heavy chain or light chain of the EphA2 antibody and the 4-1BB ligand domains in the fusion are shown in Table 14. Various fusion proteins comprising the 4-1BBL and the EphA2 antibodies are shown in Table 14.

TABLE 13
4-1BBL domains used in the fusion constructs

| Part Name | Description | Part Sequence |
|---|---|---|
| h4-1BBL | Human 4-1BB ligand, residues | (SEQ ID NO: 106) |
| (THD) | 89-242 of ECD | |
| h4-1BBL | Human 4-1BB Ligand (89-242) | (SEQ ID NO: 107) |
| (THD)3-10 | Trimer with (GGSGG)2 linkers | |
| | (SEQ ID NO: 781) between | |
| | promoters | |
| h41BBL | Human 4-1BB Ligand (89-242) | (SEQ ID NO: 108) |
| (THD)3-2 | Trimer with GG linkers | |
| | between promoters | |
| h4-1BBL | Human 4-1BB ligand, residues | (SEQ ID NO: 109) |
| (71-248) | 71-248 of ECD | |
| h4-1BBL | Human 4-1BB Ligand (71-248) | (SEQ ID NO: 110) |
| (71-248)3-10 | Trimer with (GGSGG)2 linkers | |
| | between promoters | |

Note:
Part names are comprised of the 4-1BBL domain, number of domains, and length of linker between domains.

TABLE 14
4-1BB fusion constructs

| | | Valency | Fusion | Linker to 4-1BB- | |
|---|---|---|---|---|---|
| Molecule Name | IgG Identifier | (tumor:4-1BB) | Location | engaging Part | 4-1BB Engager* |
| h1-2p2-LC- | hIgG1 | 2:2 | LC-Cter | (SEQ ID NO: 105) | h41BBL(THD)3-2 |
| h41BBL(THD)3-2 | | | | | |
| h1-2p2-LC- | hIgG1 | 2:2 | LC-Cter | (SEQ ID NO: 105) | h41BBL(THD)3-10 |
| h41BBL(THD)3-10 | | | | | |
| h1-2p2-LC- | hIgG1 | 2:2 | LC-Cter | (SEQ ID NO: 105) | h41BBL(71-248)3-10 |
| h41BBL(71-248)3-10 | | | | | |
| h4-2p2-LC- | hIgG4_S228P_R409K | 2:2 | LC-Cter | (SEQ ID NO: 105) | h41BBL(THD)3-2 |
| h41BBL(THD)3-2 | | | | | |
| h4-2p2-LC- | hIgG4_S228P_R409K | 2:2 | LC-Cter | (SEQ ID NO: 105) | h41BBL(THD)3-10 |
| h41BBL(THD)3-10 | | | | | |
| h4-2p2-LC- | hIgG4_S228P_R409K | 2:2 | LC-Cter | (SEQ ID NO: 105) | h41BBL(71-248)3-10 |
| h41BBL(71-248)3-10 | | | | | |
| h1s-2p2-LC- | hIgG1_P329G_L234A_L235A | 2:2 | LC-Cter | (SEQ ID NO: 105) | h41BBL(THD)3-2 |
| h41BBL(THD)3-2 | | | | | |
| h1s-2p2-LC- | hIgG1_P329G_L234A_L235A | 2:2 | LC-Cter | (SEQ ID NO: 105) | h41BBL(THD)3-10 |
| h41BBL(THD)3-10 | | | | | |
| h1s-2p2-LC- | hIgG1_P329G_L234A_L235A | 2:2 | LC-Cter | (SEQ ID NO: 105) | h41BBL(71-248)3-10 |
| h41BBL(71-248)3-10 | | | | | |
| h4-2p2-HC- | hIgG4_S228P_R409K | 2:2 | HC-Cter | (SEQ ID NO: 91) | SCFV9365-02 |
| SCFV9365-02 | | | | | |
| m1-2p2-HC- | mIgG1 | 2:2 | HC-Cter | (SEQ ID NO: 91) | SCFV9365-02 |
| SCFV9365-02 | | | | | |
| h4-2p2-HC- | hIgG4_S228P_R409K | 2:2 | HC-Cter | (SEQ ID NO: 91) | SCFV9036-02 |
| SCFV9036-02 | | | | | |
| m1-2p2-HC- | mIgG1 | 2:2 | HC-Cter | (SEQ ID NO: 91) | SCFV9036-02 |
| SCFV9036-02 | | | | | |
| h4-2p2-HC- | hIgG4_S228P_R409K | 2:2 | HC-Cter | (SEQ ID NO: 91) | SCFV9809-01 |
| SCFV9809-01 | | | | | |
| m1-2p2-HC- | mIgG1 | 2:2 | HC-Cter | (SEQ ID NO: 91) | SCFV9809-01 |
| SCFV9809-01 | | | | | |
| h4-2p2-HC- | hIgG4_S228P_R409K | 2:2 | HC-Cter | (SEQ ID NO: 91) | SCFV9810-01 |
| SCFV9810-01 | | | | | |
| m1-2p2-HC- | mIgG1 | 2:2 | HC-Cter | (SEQ ID NO: 91) | SCFV9810-01 |
| SCFV9810-01 | | | | | |
| h1-2p1-HC- | hIgG1_kih | 2:1 | HC-Cter | (SEQ ID NO: 91) | h41BBL(THD)3-2 |
| h41BBL(THD)3-2 | | | | | |
| h1-2p1-HC- | hIgG1_kih | 2:1 | HC-Cter | (SEQ ID NO: 91) | h41BBL(THD)3-10 |
| h41BBL(THD)3-10 | | | | | |
| h1-2p1-HC- | hIgG1_kih | 2:1 | HC-Cter | (SEQ ID NO: 91) | h41BBL(71-248)3-10 |
| h41BBL(71-248)3-10 | | | | | |
| h4-2p1-HC- | hIgG4_S228P_R409K_kih | 2:1 | HC-Cter | (SEQ ID NO: 91) | h41BBL(THD)3-2 |
| h41BBL(THD)3-2 | | | | | |
| h4-2p1-HC- | hIgG4_S228P_R409K_kih | 2:1 | HC-Cter | (SEQ ID NO: 91) | h41BBL(THD)3-10 |
| h41BBL(THD)3-10 | | | | | |
| h4-2p1-HC- | hIgG4_S228P_R409K_kih | 2:1 | HC-Cter | (SEQ ID NO: 91) | h41BBL(71-248)3-10 |
| h41BBL(71-248)3-10 | | | | | |
| hl-Xmab-2p1-HC- | hIgG1-Xmab | 2:1 | HC-Cter | (SEQ ID NO: 91) | h41BBL(THD)3-2 |
| h41BBL(THD)3-2 | | | | | |
| h1-Xmab-2p1-HC- | hIgG1-Xmab | 2:1 | HC-Cter | (SEQ ID NO: 91) | h41BBL(THD)3-10 |
| h41BBL(THD)3-10 | | | | | |
| hl-Xmab-2p1-HC- | hIgG1-Xmab | 2:1 | HC-Cter | (SEQ ID NO: 91) | h41BBL(71-248)3-10 |
| h41BBL(71-248)3-10 | | | | | |

*Terminology is as follows, using “h42BBL(THD)3-2” as an illustrative example:
1. h stands for “human” and m stands for “mouse” (or m = mouse)
2. 41BBL stand for 4-1BB ligand (native sequence)
3. THD stand for “TNF homology domain” portion of 41BBL. In instances where a number range in parentheses after 41BBL, e.g., h41BBL(71-248)3-10, refers to that residue numbers 71-248 of the 4-1BB ligand were used.
4. “3” refers to three copies connected by polypeptide linker
5. “−2” typically refers to the length of the linker used to connect the ligands together. In one example, h41BBL(THD)3-2 represent h41BBL(THD) + GG + h41BBL(THD) + GG + h41BBL(THD); In another example, h41BBL(71-248)3-10 represents h41BBL(71-248) + (GGSGG)2 (SEQ ID NO: 781) + h41BBL(71-248) + (GGSGG)2 (SEQ ID NO: 781) + h41BBL(71-248)

In some embodiments, the tumor-targeting antibody is conjugated to one or more TLR agonists. In one embodiment, the tumor-targeting antibody conjugated to a TLR agonist is a bispecific or multispecific antibody. In some embodiments, the tumor-targeting antibody is a bispecific or multispecific antibody comprising an antigen binding domain of an antibody described herein that further comprises a TLR agonist.

In some embodiments, a bispecific or multispecific antibody comprising an antigen binding domain of an antibody described herein further comprises a binding domain that binds to a checkpoint antigen, PD1, PDL1, CTLA-4, ICOS, PDL2, IDO1, IDO2, B7-H3, B7-H4, BTLA, HVEM, TIM3, GAL9, GITR, HAVCR2, LAG3, KIR, LAIR1, LIGHT, MARCO, OX-40, SLAM, 2B4, CD2, CD27, CD28, CD30, CD40, CD70, CD80, CD86, CD137, CD160. CD39, VISTA, TIGIT, CGEN-15049, 2B4, CHK 1, CHK2, A2aR, or a B-7 family ligand or its receptor.

In some embodiments, a bispecific or multispecific antibody comprising an antigen binding domain of an antibody described herein further comprises a binding domain that targets a tumor-associated antigen. Illustrative tumor-associate antigens include, but are not limited to, EpCAM, HER2/neu, HER3/neu, G250, CEA, MAGE, proteoglycans. VEGF, VEGFR, EGFR, ErbB2, ErbB3, MET, IGF-1R, PSA, PSMA, EphA2, EphA3, EphA4, folate binding protein αVβ3-integrin, integrin α5β1 HLA, HLA-DR, ASC, CD1, CD2, CD4, CD6, CD7, CD8, CD11, CD13, CD14, CD19, CD20, CD21, CD22, CD23, CD24, CD30, CD33, CD37, CD40, CD41, CD47, CD52, c-erb-2, CALLA, MHCII, CD44v3, CD44v6, p97, ganglioside GM1, GM2, GM3, GD1 a, GD1 b, GD2, GD3, GT1 b, GT3, GQ1, NY-ESO-1, NFX2, SSX2, SSX4 Trp2, gp100, tyrosinase, Muc-1, telomerase, survivin, SLAMF7 EphG250, p53, CA125 MUC, Wue antigen, Lewis Y antigen, HSP-27, HSP-70, HSP-72, HSP-90, Pgp, PMEL17, MCSP, and cell surface targets GC182, GT468 or GT512.

In some embodiments, a binding domain of an antibody described herein may be incorporated into a chimeric antigen receptor (CAR-T) construct, an engineered TCR-T cell construct, or a combined CAR-T/TCR-T construct comprising the complete TCR complex (see Hardy et al. “Implications of T cell receptor biology on the development of new T cell therapies for cancer, Immunotherapy Vol. 12, No. 1. Published Online: 6 Jan. 2020 https://doi.org/10.2217/imt-2019-0046). Such constructs can be used to generate a modified immune cell such as a T-cell, NK-cell, or monocyte/macrophage comprising the binding domain of an antibody described herein. For example, in some embodiments, a first-generation CAR joins a single-chain variable region from the antibody to a CD3zeta (ζ) intracellular signaling domain of a CD3 T-cell receptor through hinge and transmembrane domains. In some embodiments, the CAR may contain co-stimulating domains, e.g., a second or third generation CAR may include an additional one or two-co-stimulating domains, such as 4-1BB, CD28, or OX-40). In additional embodiments, a CAR-containing cell, e.g., a CAR-T cell, may additionally be engineered to with an inducible expression component such as a cytokine, e.g., IL-12 or IL-15 to increase activation of CAR-T cells and activate innate immune cells.

In one embodiment of any of the above bispecific or multispecific antibody constructs, the tumor-targeting binding domain comprises all six CDRs (HCDR1, HCDR2, HCDR3, LCDR1, LCDR2, and LCDR3) sequences from the individual antibodies disclosed in Tables 6 and 7.

In one embodiment of any of the above bispecific or multispecific antibody constructs, the tumor-targeting binding domain comprises the V_{H} and V_{L} sequences from the individual antibodies disclosed in Table 8.

In one embodiment of any of the above bispecific or multispecific antibody constructs, the tumor-targeting binding domain comprises all six CDRs (HCDR1, HCDR2, HCDR3, LCDR1, LCDR2, and LCDR3) sequences from any one of antibodies AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698, AB-010699. AB-010700, AB-010701, or AB-010702. In some embodiments, in any of the above bispecific or multispecific antibody constructs, the tumor-targeting binding domain comprises all six CDRs (HCDR1, HCDR2, HCDR3, LCDR1. LCDR2, and LCDR3) sequences from AB-010361 or AB-010699.

In one embodiment of any of the above bispecific or multispecific antibody constructs, the tumor-targeting binding domain comprises the V_{H} and V_{L} sequences any one of AB-008873; AB-009805; AB-009806; AB-009807; AB-009808; AB-009812; AB-009813; AB-009814; AB-009815; AB-009816; and AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666. AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698. AB-010699, AB-010700, AB-010701, or AB-010702. In one embodiment of any of the above bispecific or multispecific antibody constructs, the tumor-targeting binding domain comprises the V_{H} and V_{L} sequences of AB-010361 or AB-010699.

### Generation of Antibodies

EphA2 antibodies as disclosed herein are commonly produced using vectors and recombinant methodology well known in the art (see, e.g., Sambrook & Russell, Molecular Cloning: A Laboratory Manual, Cold Spring Harbor Laboratory Press; Ausubel, Current Protocols in Molecular Biology). Reagents, cloning vectors, and kits for genetic manipulation are available from commercial vendors. Accordingly, in a further aspect of the invention, provided herein are isolated nucleic acids encoding a V_{H} and/or V_{L} region, or fragment thereof, of any of the tumor-targeting antibodies as described herein; vectors comprising such nucleic acids and host cells into which the nucleic acids are introduced that are used to replicate the antibody-encoding nucleic acids and/or to express the antibodies. Such nucleic acids may encode an amino acid sequence containing the V_{L} and/or an amino acid sequence containing the V_{H} of the tumor-targeting antibody (e.g., the light and/or heavy chains of the antibody). In some embodiments, the host cell contains (1) a vector containing a polynucleotide that encodes the V_{L} amino acid sequence and a polynucleotide that encodes the V_{H} amino acid sequence, or (2) a first vector containing a polynucleotide that encodes the V_{L} amino acid sequence and a second vector containing a polynucleotide that encodes the V_{H} amino acid sequence.

In a further aspect, the invention provides a method of making an EphA2 antibody as described herein. In some embodiments, the method includes culturing a host cell as described in the preceding paragraph under conditions suitable for expression of the antibody. In some embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected may vary according to the host cell intended to be used, useful cloning vectors generally can self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mpl8, mpl9, pBR322, pMB9, ColE1 plasmids, pCR1, RP4, phage DNAs, and shuttle vectors. These and many other cloning vectors are available from commercial vendors.

Expression vectors generally are replicable polynucleotide constructs that contain a nucleic acid of the present disclosure. The expression vector can be replicable in the host cells either as episomes or as an integral part of the chromosomal DNA. Suitable expression vectors include but are not limited to plasmids and viral vectors, including adenoviruses, adeno-associated viruses, retroviruses, and any other vector.

Suitable host cells for expressing an EphA2 antibody as described herein include both prokaryotic and eukaryotic cells. For example, an EphA2 antibody may be produced in bacteria, when glycosylation and Fc effector function are not needed. After expression, the antibody may be isolated from the bacterial cell paste in a soluble fraction and can be further purified. Alternatively, the host cell may be a eukaryotic host cell, including eukaryotic microorganisms, such as filamentous fungi or yeast, including fungi and yeast strains whose glycosylation pathways have been “humanized,” resulting in the production of an antibody with a partially or fully human glycosylation pattern, vertebrate, invertebrate, and plant cells. Examples of invertebrate cells include insect cells. Numerous baculoviral strains have been identified which may be used in conjunction with insect cells. Plant cell cultures can also be utilized as host cells.

In some embodiments, vertebrate host cells are used for producing an EphA2 antibody of the present disclosure. For example, mammalian cell lines such as a monkey kidney CV1 line transformed by SV40 (COS-7); human embryonic kidney line (293 or 293 cells as described, e.g., in Graham et al., J. Gen Virol. 36:59, 1977; baby hamster kidney cells (BHK); mouse sertoli cells (TM4 cells as described, e.g., in Mather, Biol. Reprod. 23:243-251, 1980 monkey kidney cells (CVI); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68, 1982; MRC 5 cells; and FS4 cells may be used to express an tumor-targeting antibodies. Other useful mammalian host cell lines include Chinese hamster ovary (CHO) cells, including DHFR− CHO cells (Urlaub et al., Proc. Natl. Acad. Sci. USA 77:4216, 1980); and myeloma cell lines such as Y0, NS0 and Sp2/0. Host cells of the present disclosure also include, without limitation, isolated cells, in vitro cultured cells, and ex vivo cultured cells. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268, 2003.

In some embodiments, an EphA2 antibody of the present disclosure is produced by a CHO cell line, e.g., the CHO-K1 cell line. One or more expression plasmids can be introduced that encode heavy and light chain sequences. For example, in one embodiment, an expression plasmid encoding a heavy chain disclosed herein, e.g., SEQ ID NO: 67, and an expression plasmid encoding a light chain disclosed herein, e.g., SEQ ID NO: 68 are transfected into host cells as linearized plasmids at a ratio of 1:1 in the CHO-K1 host cell line using reagents such as Freestyle Max reagent. Fluorescence-activated cell sorting (FACS) coupled with single cell imaging can be used as a cloning method to obtain a production cell line.

A host cell transfected with an expression vector encoding an EphA2 antibody of the present disclosure, or fragment thereof, can be cultured under appropriate conditions to allow expression of the polypeptide to occur. The polypeptides may be secreted and isolated from a mixture of cells and medium containing the polypeptides. Alternatively, the polypeptide may be retained in the cytoplasm or in a membrane fraction and the cells harvested, lysed, and the polypeptide isolated using a desired method.

In some embodiments, an EphA2 antibody of the present disclosure can be produced by in vitro synthesis (see, e.g., Sutro Biopharma biochemical protein synthesis platform).

In some embodiments, provided herein is a method of generating variants of an EphA2 antibody as disclosed herein. Thus, for example, a construct encoding a variant of a V_{H} CDR3 as described herein can be modified and the V_{H} region encoded by the modified construct can be tested for binding activity to CT26 cells and/or in vivo tumor-targeting activity in the context of a V_{H} region as described herein, that is paired with a V_{L} region or variant region as described herein. Similarly, a construct encoding a variant of a V_{L} CDR3 as described herein can be modified and the V_{L} region encoded by the modified construct can be tested for binding to CT26 cells, or other tumor cells, and/or in vivo tumor-targeting activity efficacy. Such an analysis can also be performed with other CDRs or framework regions and an antibody having the desired activity can then be selected.

### Tumor-Targeting Antibody Conjugates/Co-Stimulatory Agents

In a further aspect, an EphA2 antibody of the present disclosure may be conjugated or linked to therapeutic, imaging/detectable moieties, or enzymes. For example, the tumor-targeting antibody may be conjugated to a detectable marker, a cytotoxic agent, an immunomodulating agent, an imaging agent, a therapeutic agent, an oligonucleotide, or an enzyme. Methods for conjugating or linking antibodies to a desired molecule are well known in the art. The desired molecule may be linked to the antibody covalently or by non-covalent linkages.

In some embodiments, the antibody is conjugated, either directly or via a cleavable or non-cleavable linker, to a cytotoxic moiety or other moiety that exerts their effects on critical cellular processes required for survival (“payload”) to form an antibody-drug conjugate (“ADC”).

In some embodiments, the antibody is conjugated to an auristatin. In some embodiments, the ADC is an EphA2 antibody disclosed herein conjugated to a ZymeLink™ Auristatin (ZLA) payload. Such ADC constructs are referred to as the ZymeLink™ ADC constructs, as further disclosed below.

In some embodiments, ZymeLink™ ADCs of the present disclosure comprise an EphA2 antibody conjugated to a ZLA having structure (1) (also referred to herein as “Compound 1”) via a linker L.

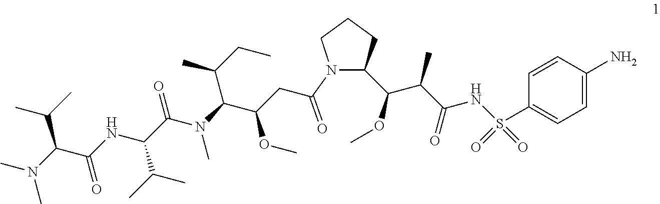 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C]

In certain embodiments, the ZymeLink™ ADCs have Formula (I):

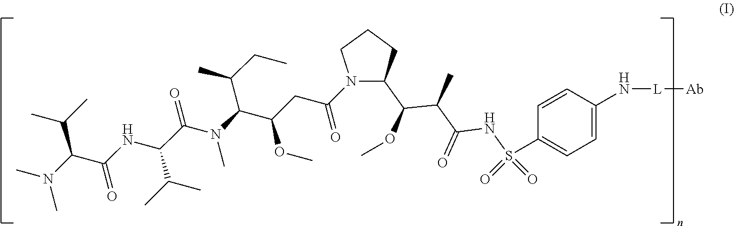

wherein:
L is a cleavable linker;
n is the drug-to-antibody ratio (DAR) and is an integer from 1 to 12, and
Ab is the antibody.

In some embodiments, the each of the n drugs in formula (I) is independently conjugated to the antibody Ab via one of n linkers L.

In some embodiments, in the ZymeLink™ ADCs of general Formula (I), linker L is a protease-cleavable linker.

In some embodiments, in the ZymeLink™ ADCs of Formula (I), linker L is a peptide-containing linker. In some embodiments, in the ADCs of Formula (I), linker L comprises a dipeptide selected from Val-Lys. Ala-Lys, Phe-Lys. Val-Cit, Phe-Cit, Leu-Cit, Ile-Cit and Trp-Cit.

In some embodiments, in the ZymeLink™ ADCs of Formula (I), linker L comprises a dipeptide and a stretcher.

In certain embodiments, linker L in ZymeLink™ ADCs of Formula (I) is conjugated to the antibody via a cysteine residue or a lysine residue on the antibody. In some embodiments, linker L in ADCs of Formula (I) is conjugated to the antibody via a lysine residue on the antibody.

In some embodiments, in the ZymcLink™ ADCs of Formula (I), n is an integer from 1 to 8. In some embodiments, in the ADCs of Formula (I), n has a value from 2 to 8, e.g., from 2 to 6, from 3 to 7, or from 4 to 8. In terms of upper limits, n can be less than 12, e.g., less than 11, less than 10, less than 9, less than 8, less than 7, less than 6, less than 5, less than 4, or less than 3. In terms of lower limits, n can be greater than 2, e.g., greater than 3, greater than 4, greater than 5, greater than 6, greater than 7, greater than 8, greater than 9, greater than 10, or greater than 11. Larger values of n, e.g., greater than 12, are also contemplated.

Combinations of any of the foregoing embodiments for ZymeLink™ ADCs of Formula (I) are also contemplated and each combination forms a separate embodiment for the purposes of the present disclosure.

In certain embodiments, the ZymeLink™ ADCs of the present disclosure have Formula (II):

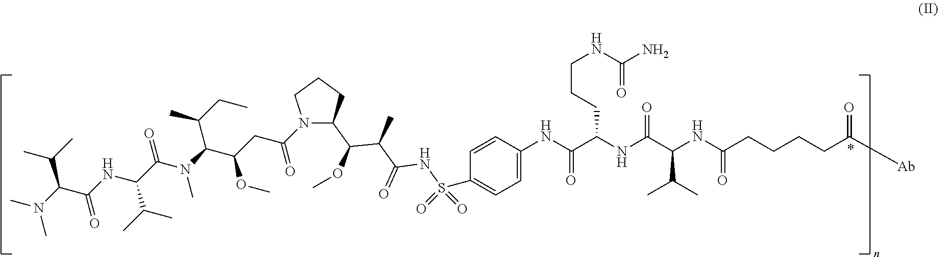

wherein:
n is the drug-to-antibody ratio (DAR) and is an integer from 1 to 12, and
Ab is the antibody.

In some embodiments, the each of the n drugs in formula (II) is independently conjugated to the antibody Ab via one of n linkers L.

In certain embodiments, the carbonyl group of Formula (II) marked with an asterisk forms a peptide bond with the side chain amine group of a lysine residue on the antibody (Ab).

In some embodiments, in the ZymeLink™ ADCs of general Formula (II), n is an integer from 1 to 8. In some embodiments, in the ADCs of general Formula (II), n has a value from 2 to 8, e.g., from 2 to 6, from 3 to 7, or from 4 to 8. In terms of upper limits, n can be less than 12, e.g., less than 11, less than 10, less than 9, less than 8, less than 7, less than 6, less than 5, less than 4, or less than 3. In terms of lower limits, n can be greater than 2, e.g., greater than 3, greater than 4, greater than 5, greater than 6, greater than 7, greater than 8, greater than 9, greater than 10, or greater than 11. Larger values of n, e.g., greater than 12, are also contemplated.

### Linker L

In the ADCs of the present disclosure, the antibody is linked to an auristatin analogue disclosed herein by a linker L. Linkers are bifunctional or multifunctional moieties capable of linking one or more drug molecules to an antibody. A linker may be bifunctional (or monovalent) such that it links a single drug to a single site on the antibody, or it may be multifunctional (or polyvalent) such that it links more than one drug molecule to a single site on the antibody. Linkers capable of linking one drug molecule to more than one site on the antibody may also be multifunctional.

Certain linkers useful in the present disclosure can be up to 30 carbon atoms in length. For example, the linkers can each independently be from 5 to 20 carbon atoms in length. The types of bonds used to link the linker to the cytotoxic agent and antibody of the present disclosure include, but are not limited to, amides, amines, esters, carbamates, ureas, thioethers, thiocarbamates, thiocarbonate and thioureas. One of skill in the art will appreciate that other types of bonds are useful in the provided ADCs.

Attachment of a linker to an antibody can be accomplished in a variety of ways, such as through surface lysines on the antibody, reductive coupling to oxidized carbohydrates on the antibody, or through cysteine residues on the antibody liberated by reducing interchain disulfide linkages. Alternatively, attachment of a linker to an antibody may be achieved by modification of the antibody to include additional cysteine residues (see, for example, U.S. Pat. Nos. 7,521,541; 8,455,622 and 9,000,130) or non-natural amino acids that provide reactive handles, such as selenomethionine, p-acetylphenylalanine, formylglycine or p-azidomethyl-L-phenylalanine (see, for example, Hofer et al., Biochemistry, 48:12047-12057 (2009); Axup et al., PNAS, 109:16101-16106 (2012); Wu et al., PNAS, 106:3000-3005 (2009); Zimmerman et al., Bioconj. Chem., 25:351-361 (2014)), to allow for site-specific conjugation.

Linkers include at least one functional group capable of reacting with the target group or groups on the antibody, and one or more functional groups capable of reacting with a target group on the drug. Suitable functional groups are known in the art and include those described, for example, in Bioconjugate Techniques (G. T. Hermanson, 2013, Academic Press).

Examples of target groups on the antibody to which a linker may be conjugated include the thiol groups of cysteine residues and the amine groups of lysine residues. Non-limiting examples of functional groups for reacting with free cysteines or thiols include maleimide, haloacetamide, haloacetyl, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Also useful in this context are “self-stabilizing” maleimides as described in Lyon et al., Nat. Biotechnol., 32:1059-1062 (2014). Non-limiting examples of functional groups for reacting with free amines include activated esters (such as N-hydroxysuccinamide (NHS) esters, sulfo-NHS esters, imido esters such as Traut's reagent, tetrafluorophenyl (TFP) esters and sulfodichlorophenyl esters), isothiocyanates, aldehydes and acid anhydrides (such as diethylenetriaminepentaacetic anhydride (DTPA)). Other examples include succinimido-1,1,3,3-tetra-methyluronium tetrafluoroborate (TSTU) and benzotriazol-1-yl-oxytripyrrolidinophosphonium hexafluorophosphate (PyBOP).

Other linkers include those having a functional group that allows for bridging of two interchain cysteines on the antibody, such as a ThioBridge® linker (Badescu et al., Bioconjug. Chem., 25:1124-1136 (2014)), a dithiomaleimide (DTM) linker (Behrens et al., Mol. Pharm., 12:3986-3998 (2015)), a dithioarylpyridazinedione-based linker (Lee et al., Chem. Sci., 7:799-802 (2016)), a dibromopyridazinedione-based linker (Maruani et al., Nat. Commun., 6:6645 (2015)) and others known in the art.

A linker may comprise one or more linker components. Typically, a linker will comprise two or more linker components. Exemplary linker components include functional groups for reaction with the antibody, functional groups for reaction with the drug, stretchers, peptide components, self-immolative groups, self-elimination groups, hydrophilic moieties, and the like. Various linker components are known in the art, some of which are described below.

Certain useful linker components can be obtained from various commercial sources, such as Pierce Biotechnology, Inc. (now Thermo Fisher Scientific Corporation, Waltham, MA) and Molecular Biosciences Inc. (Boulder, Colo.), or may be synthesized in accordance with procedures described in the art (see, for example, Toki et al., J. Org. Chem., 67:1866-1872 (2002); Dubowchik et al., Tetrahedron Letters, 38:5257-60 (1997); Walker, M. A., J. Org. Chem., 60:5352-5355 (1995); Frisch et al., Bioconjugate Chem., 7:180-186 (1996); U.S. Pat. Nos. 6,214,345 and 7,553,816, and International Patent Application Publication No. WO 02/088172).

Examples of linker components include, but are not limited to, N-(β-maleimidopropyloxy)-N-hydroxy succinimide ester (BMPS), N-(ε-maleimidocaproyloxy) succinimide ester (EMCS), N-[(R)-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (STAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(0-maleimidopropionamido)hexanoate](SMPH), iminothiolane (IT), sulfo-EMCS, sulfo-GMBS, sulfo-KMUS, sulfo-MBS, sulfo-SIAB, sulfo-SMCC, sulfo-SMPB and succinimidyl-(4-vinylsulfone)benzoate (SVSB).

Additional examples include bis-maleimide reagents such as dithiobismaleimidoethane (DTME), bis-maleimido-trioxyethylene glycol (BMPEO), 1,4-bismaleimidobutane (BMB), 1,4 bismaleimidyl-2,3-dihydroxybutane (BMDB), bismaleimidohexane (BMH), bismaleimidoethane (BMOE), BM(PEG)2 and BM(PEG)3; bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

Suitable linkers typically are more chemically stable to conditions outside the cell than to conditions inside the cell, although less stable linkers may be contemplated in certain situations, such as when the drug is selective or targeted and has a low toxicity to normal cells. Linkers may be “cleavable linkers” or “non-cleavable linkers.” A cleavable linker is typically susceptible to cleavage under intracellular conditions, for example, through lysosomal processes. Examples include linkers that are protease-sensitive, acid-sensitive, reduction-sensitive or photolabile. Non-cleavable linkers by contrast, rely on the degradation of the antibody in the cell, which typically results in the release of an amino acid-linker-toxin moiety.

In accordance with the present disclosure, linker L comprised by the ADCs of Formula (I) is a cleavable linker. Suitable cleavable linkers include, for example, linkers comprising a peptide component that includes two or more amino acids and is cleavable by an intracellular protease, such as lysosomal protease or an endosomal protease. A peptide component may comprise amino acid residues that occur naturally and/or minor amino acids and/or non-naturally occurring amino acid analogues, such as citrulline. Peptide components may be designed and optimized for enzymatic cleavage by a particular enzyme, for example, a tumor-associated protease, cathepsin B, C or D, or a plasmin protease.

In certain embodiments, linker L comprised by the ADCs of Formula (I) may be a peptide-containing linker. In some embodiments, linker L comprised by the ADCs may be a dipeptide-containing linker, such as a linker containing valine-citrulline (Val-Cit) or phenylalanine-lysine (Phe-Lys). Other examples of suitable dipeptides for inclusion in linker L include Val-Lys, Ala-Lys, Me-Val-Cit, Phe-homoLys, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me3Lys-Pro, PhenylGly-(D)Lys, Met-(D)Lys, Asn-(D)Lys, Pro-(D)Lys and Met-(D)Lys. Cleavable linkers may also include longer peptide components such as tripeptides, tetrapeptides or pentapeptides. Examples include, but are not limited to, the tripeptides Met-Cit-Val, Gly-Cit-Val, (D)Phe-Phe-Lys and (D)Ala-Phe-Lys, and the tetrapeptides Gly-Phe-Leu-Gly, Gly-Gly-Phe-Gly and Ala-Leu-Ala-Leu. In some embodiments, linker L comprised by the ADCs may be a peptide-containing linker, where the peptide is between two and five amino acids in length, for example, between two and four amino acids in length.

Additional examples of cleavable linkers include disulfide-containing linkers, such as, N-succinimydyl-4-(2-pyridyldithio) butanoate (SPBD) and N-succinimydyl-4-(2-pyridyldithio)-2-sulfo butanoate (sulfo-SPBD). Disulfide-containing linkers may optionally include additional groups to provide steric hindrance adjacent to the disulfide bond to improve the extracellular stability of the linker, for example, inclusion of a geminal dimethyl group. Other suitable linkers include linkers hydrolyzable at a specific pH or within a pH range, such as hydrazone linkers. Linkers comprising combinations of these functionalities may also be useful, for example, linkers comprising both a hydrazone and a disulfide are known in the art.

A further example of a cleavable linker is a linker comprising a β-glucuronide, which is cleavable by β-glucuronidase, an enzyme present in lysosomes and tumor interstitium (see, for example, De Graaf et al., Curr. Pharm. Des., 8:1391-1403 (2002)).

Cleavable linkers may optionally further comprise one or more additional components such as self-immolative and self-elimination groups, stretchers or hydrophilic moieties.

Self-immolative and self-elimination groups that find use in linkers include, for example, p-aminobenzyloxycarbonyl (PABC) and p-aminobenzyl ether (PABE) groups, and methylated ethylene diamine (MED). Other examples of self-immolative groups include, but are not limited to, aromatic compounds that are electronically similar to the PABC or PABE group such as heterocyclic derivatives, for example 2-aminoimidazol-5-methanol derivatives as described in U.S. Pat. No. 7,375,078. Other examples include groups that undergo cyclization upon amide bond hydrolysis, such as substituted and unsubstituted 4-aminobutyric acid amides (Rodrigues et al., Chemistry Biology, 2:223-227 (1995)) and 2-aminophenylpropionic acid amides (Amsberry et al., J. Org. Chem., 55:5867-5877 (1990)).

Stretchers that find use in linkers for ADCs include, for example, alkylene groups and stretchers based on aliphatic acids, diacids, amines or diamines, such as diglycolate, malonate, caproate and caproamide. Other stretchers include, for example, glycine-based stretchers, polyethylene glycol (PEG) stretchers and monomethoxy polyethylene glycol (mPEG) stretchers. PEG and mPEG stretchers also function as hydrophilic moieties.

Examples of components commonly found in cleavable linkers include, but are not limited to, SPBD, sulfo-SPBD, hydrazone, Val-Cit, maleidocaproyl (MC), MC-Val-Cit, MC-Val-Cit-PABC, Phe-Lys, MC-Phe-Lys, MC-Phe-Lys-PABC, maleimido triethylene glycolate (MT), MT-Val-Cit, MT-Phe-Lys, TFP and adipate (AD).

Selection of an appropriate linker for a given ADC may be readily made by the skilled person having knowledge of the art and taking into account relevant factors, such as the site of attachment to the antibody, any structural constraints of the payload drug and the hydrophobicity of the payload drug (see, for example, review in Nolting, Chapter 5, Antibody-Drug Conjugates: Methods in Molecular Biology, 2013, Ducry (Ed.), Springer).

In certain embodiments, linker L included in the ADCs of the present disclosure is a peptide-based linker having Formula (V):

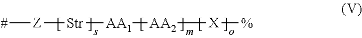

wherein:
Z is a linking group that joins the linker to a target group on the antibody;
Str is a stretcher;
AA₁ and AA₂ are each independently an amino acid, wherein AA₁-[AA₂]ₘ forms a protease cleavage site;
X is a self-immolative group;
s is 0 or 1;
m is 1, 2, 3 or 4;
o is 0, 1 or 2;
# is the point of attachment to the antibody, and
% is the point of attachment to the auristatin analogue.

In some embodiments, in Formula (V), m is 1, 2 or 3.

In some embodiments, in Formula (V), s is 1.

In some embodiments, in Formula (V), o is 0 (i.e., X is absent).

In some embodiments, in Formula (V):
Z is

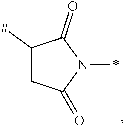

where # is the point of attachment to the antibody, and * is the point of attachment to the remainder of the linker.

In some embodiments, in Formula (V), Z is a carbonyl group (—C(O)—).

In some embodiments, in Formula (V), Str is selected from:

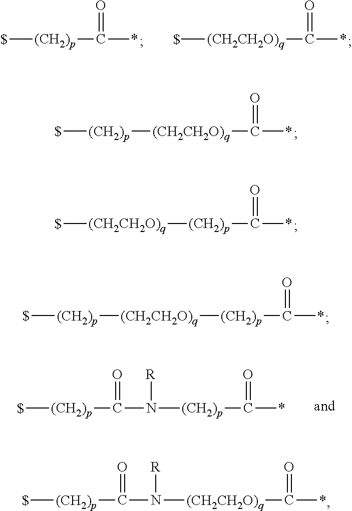

wherein:
each R is independently H or C₁-C₆ alkyl;
each p is independently an integer from 2 to 10;
each q is independently an integer from 1 to 10,
[symbol] is the point of attachment to Z, and
* is the point of attachment to the remainder of the linker.

In some embodiments, in Formula (V), Str is:

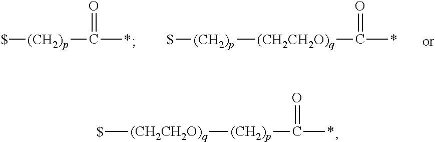

where p, q, [symbol] and * are as defined above.

In some embodiments, in Formula (V), Str is:

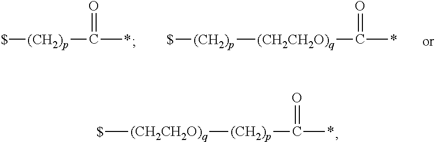

where [symbol] and * are as defined above, p is an integer from 2 to 6, and q is an integer from 2 to 8.

In some embodiments, in Formula (V), AA1-[AA2]m is selected from Val-Lys, Ala-Lys, Phc-Lys, Val-Cit, Phc-Cit, Leu-Cit, Ile-Cit, Trp-Cit, Phc-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me3Lys-Pro, PhenylGly-(D)Lys, Met-(D)Lys, Asn-(D)Lys, Pro-(D)Lys, Met-(D)Lys, Met-Cit-Val, Gly-Cit-Val, (D)Phe-Phe-Lys, (D)Ala-Phe-Lys, Gly-Phe-Leu-Gly and Ala-Leu-Ala-Leu.

In some embodiments, in Formula (V), m is 1 (i.e., AA1-[AA2]m is a dipeptide).

In some embodiments, in Formula (V), AA1-[AA2]m is a dipeptide selected from Val-Lys, Ala-Lys, Phe-Lys, Val-Cit, Phe-Cit, Leu-Cit, Ile-Cit and Trp-Cit.

In some embodiments, in Formula (V):
Z is a carbonyl group (—C(O)—);
Str is

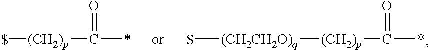

where [symbol] is the point of attachment to Z, * is the point of attachment to the remainder of the linker, p is an integer from 2 to 6, and q is an integer from 2 to 8;
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit;
s is 1; and
o is 0.

In some embodiments, in Formula (V):
Z is

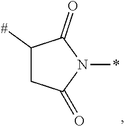

where # is the point of attachment to the antibody, and * is the point of attachment to the remainder of the linker;
Str is

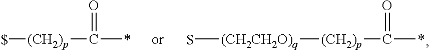

where [symbol] is the point of attachment to Z, * is the point of attachment to the remainder of the linker, p is an integer from 2 to 6, and q is an integer from 2 to 8;
m is 1 and AA₁-|AA₂|ₘ is a dipeptide selected from Val-Lys, Ala-Lys, Phe-Lys, Val-Cit, Phe-Cit, Leu-Cit, Ile-Cit and Trp-Cit;
s is 1; and
o is 0.

In certain embodiments, the linker included in the ZymeLink™ ADCs of the present disclosure has Formula (VI):

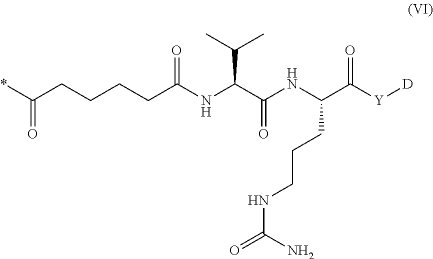

where:
* is the point of attachment to the antibody;
Y is one or more additional linker components, or is absent; and
D is the point of attachment to the auristatin analogue.

In some embodiments, in the linker of Formula (VI), Y is absent.

In certain embodiments, the linker included in the ZymeLink™ ADCs of the present disclosure has Formula (VII):

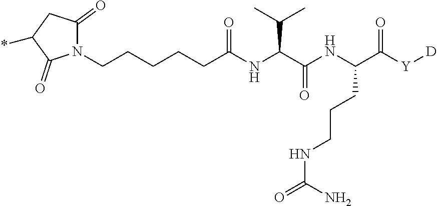

where:
* is the point of attachment to the antibody;
Y is one or more additional linker components, or is absent; and
D is the point of attachment to the auristatin analogue.

In some embodiments, in the linker of Formula (VII), Y is absent.

In certain embodiments, the linker included in the ZymeLink™ ADCs of the present disclosure has Formula (VIII):

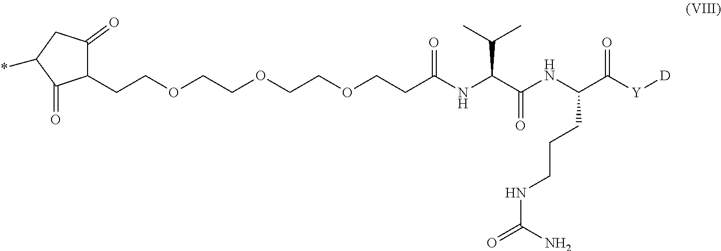

where:
* is the point of attachment to the antibody;
Y is one or more additional linker components, or is absent, and
D is the point of attachment to the auristatin analogue.

In some embodiments, in the linker of Formula (VIII), Y is absent.

In some embodiments, the antibody is conjugated to a microtubule inhibitor that induces apoptosis in cells undergoing mitosis by, for example, causing cell cycle arrest at G2/M. Nonlimiting examples of microtubule inhibitors that can be used include maytansine derivatives (DM1/DM4), or auristatins (MMAE/MMAF) and variants thereof, such as monomethyl auristatin D, PF-06380101, duostatin5, AS269, Tap18Hr1, AGD-0182, HPA-Auristatin F. In some embodiments, the payload is a tubulin-targeting agent, for example, hemiasterlin, tubulysin, or eribulin. In some embodiments, the payloads are DNA-damaging payloads, which include enediynes (calicheamicin), duocarmycin derivatives, pyrrolobenzodiazepine dimers (PBD dimers), and indolinobenzodiazepine pseudo-dimers.

In some embodiments, the antibody is conjugated to a cytotoxic agent including, but not limited to, e.g., ricin A chain, doxorubicin, daunorubicin, a maytansinoid, taxol, ethidium bromide, mitomycin, etoposide, tenoposide, vincristine, vinblastine, colchicine, dihydroxy anthracin dione, methotrexact, actinomycin, a diphtheria toxin, extotoxin A from Pseudomonas, Pseudomonas exotoxin40, abrin, abrin A chain, modeccin A chain, alpha sarcin, gelonin, mitogellin, restrictocin, cobran venom factor, a ribonuclease, engineered Shiga toxin, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, or a cisplatin. In some embodiments, the antibody may be linked to an agent such as an enzyme inhibitor, a proliferation inhibitor, a lytic agent, a DNA or RNA synthesis inhibitors, a membrane permeability modifier, a DNA metabolite, a dichloroethylsulfide derivative, a protein production inhibitor, a ribosome inhibitor, or an inducer of apoptosis. In some embodiments, the antibody is conjugated to a drug such as a topoisomeriase inhibitor, e.g., a topoisomeraise I inhibitor. Topoisomeraise I inhibitors include but are not limited to quinoline alkaloids (SN-38, DXd).

In some embodiments, the antibody is conjugated to a cancer chemotherapeutic agent, as described below.

In some embodiments, the antibody is conjugated to one or more of the cytotoxic and/or anti-mitotic compounds as disclosed in WO2014144871A1 and WO2016041082A1, the entire content of both applications are herein incorporated by reference.

In some embodiments, an EphA2 antibody as described herein is joined to a molecule that facilitates transport of the antibody across a biological membrane, e.g., by enhancing penetration of the membrane, facilitating protein translocation across membranes. Thus, for example, the antibody may be linked to a cell penetration agent, such as a cell-penetrating peptide. Examples of cell penetrating peptides include TAT, penetrating, polyarginine molecules, Kunitz domain-derived peptides, e.g., angiopep-2, SynB, buforin, transportan, amphiphathic peptides and others. In some embodiments, the antibody may be conjugated with a cationic molecule such as a polyamine. In some embodiments, the antibody may be conjugated to an agent that facilitates transport across the blood brain barrier, e.g., transcytosis. Thus, for example, the antibody may be conjugated to an agent that binds to endothelial cell receptors that are internalized, e.g., transferrin receptor, insulin receptor, insulin-like growth factor receptor, or a low-density lipoprotein receptor, and the like. In some embodiments, the antibody may be conjugated to a toxin facilitating entry of the antibody into the cytoplasm, e.g., Shiga toxin. In some embodiments, an EphA2 antibody as described herein can be conjugated to an engineered toxin body (ETBs) to facilitate internalization of the antibody into a cell.

In some embodiments, an EphA2 antibody described herein is conjugated or administered with a polypeptide immunomodulating agent, e.g., an adjuvant. Examples of immunomodulating agents include, but are not limited to, cytokines (e.g., transforming growth factor-β (TGFβ)), growth factors, lymphotoxins, tumor necrosis factor (TNF), hematopoietic factors, interleukins (e.g., interleukin-1 (IL-1), IL-2, IL-3, IL-6, IL-10, IL-12, IL-15, an IL-15/IL-15Rα, e.g., sushi domain, complex, IL-18, and IL-21), colony stimulating factors (e.g., granulocyte-colony stimulating factor (G-CSF) and granulocyte macrophage-colony stimulating factor (GM-CSF), interferons (e.g., interferon-α, -β or -γ, erythropoietin and thrombopoietin, or a combination thereof. In some embodiments, the antibody is linked or administered with a compound that stimulates the innate immune system, such as an adjuvant, a Toll-like receptor (TLR) agonist, a C-type lectin receptor (CLR) agonist, a retinoic acid-inducible gene I-like receptor (RLR) agonist, a saponin, a polysaccharide such as chitin, chitosan, β-glucan, an ISCOM, QS-21, a stimulator of interferon genes (STING) agonist, or another immunopotentiating agent.

In some embodiments, an EphA2 antibody described herein is conjugated to or administered with an IL-15 receptor agonist, such as an IL-15 fusion construct, an IL-15:IL-15Rα fusion construct or a single-chain IL-15:IL-15Rα (sushi) fusion construct. In one embodiment, the tumor-targeting antibody conjugated to an IL-15 receptor agonist is a bispecific or multispecific antibody. In some embodiments, the antibody is a bispecific or multispecific antibody comprising an antigen binding domain described herein that further comprises an IL-15 receptor agonist.

In one embodiment, an EphA2 antibody described herein is administered with a single-chain IL-15:IL-15Rα (sushi) fusion construct. In some embodiments, an EphA2 antibody is administered with a polymer-conjugated IL-15 construct, such as NKTR-255.

The IL-15:IL-15Rα single chain constructs can be administered to a subject comprising a therapeutically effective dose, for example in the range of less than 0.01 mg/kg body weight to about 25 mg/kg body weight, or 0.1-10 mg/kg, or in the range 1 mg-2 g per patient, or approximately 50 mg-1000 mg/patient.

In one embodiment, the single-chain IL-15 fusion construct comprises IL-15 joined to IL-15Rα (sushi) with a polypeptide linker. In one embodiment, the single-chain IL-15 fusion construct is joined via a polypeptide linker to another protein, such as an Fc for long half-life. See, for example, FIG. 9B in WO2018071919A1 (corresponding to U.S. patent Ser. No. 10/550,185). In one embodiment, the TL-15 is joined or fused to the N-terminus of the heavy chain of an Fc, and IL-15Rα(sushi) is joined or fused to the other Fc heavy chain N-terminus, using a heavy chain heterodimerization technology to form the desired hybrid Fc. See, for example, FIG. 9A in WO2018071919A1.

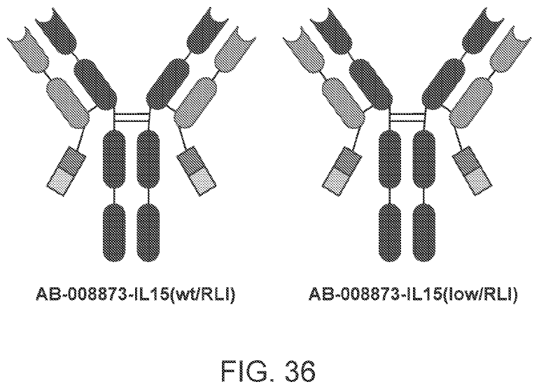

In one embodiment, the IL-15:IL-15Rα (sushi) single chain constructs are fused to the C-terminus of an antibody light chain, or the C-terminus of an antibody heavy chain, in both cases producing a molecule with two tumor-targeting binding sites (the Fab arms), and two IL15:IL15Ra units. Illustrative configurations of the fusion construct is shown in FIG. 36. In another embodiment, one copy of an IL15:IL15Rα fusion construct is fused to an EphA2 antibody, thereby producing an antibody molecule comprising two tumor-targeting binding sites (the Fab anns) and only one IL15:IL15Rα unit, for example using a knob-in-holes approach to heavy chain heterodimerization, or other heterodimerization technology.

In some embodiments, the IL-15:IL-15Rα (sushi) fusion constructs or the antibodies comprising the fusion constructs comprise a low affinity IL-15 variant having improved pharmacokinetics (PK). In some embodiments, the IL-15:IL-15Rα (sushi) fusion constructs comprise a high affinity IL-15 variant having increased agonist activity. In some embodiments, the high affinity IL-15 variant has an N72D mutation. In some embodiments, the high affinity variant is fused to a dimeric IL-15Rα sushi domain-IgG1 Fc fusion protein. In some embodiments, the IL-15:IL-15Rα (sushi) fusion construct is ALT-803. See Liu B, et al. (November 2016). “A Novel Fusion of ALT-803 (Interleukin (IL)-15 Superagonist) with an Antibody Demonstrates Antigen-specific Antitumor Responses”. The Journal of Biological Chemistry. 291 (46): 23869-23881. doi:10.1074/jbc.M116.733600. Sequences of the IL-15:IL-15Rα fusion constructs and linkers are provided in the Examples.

In some embodiments, antibodies comprising the IL15:IL15Rα fusion construct comprise one or more mutations in the Fc region described herein, for example E333A, K326W/E333S, S239D/I332E/G236A, S239D/A330L/I332E, G236A/S239D/A330L/I332E, F243L, G236A, and S298A/E333A/K334A. In some embodiments, antibodies comprising the IL15:IL15Rα fusion comprise one or more mutations in the Fc region that increase binding of the antibody to tumor cells, for example the mutations P329G, L234A, L235A, or a combination thereof.

In some embodiments, antibodies comprising the IL15:IL15Rα fusion construct comprises one or more sequences shown in Table 15. In some embodiments, the fusion protein comprises or consists of SEQ ID NO: 89, SEQ ID NO: 90, SEQ ID NO: 91, and SEQ ID NO: 92. In some embodiments, the fusion protein comprises or consists of SEQ ID NO: 89, SEQ ID NO: 90, SEQ ID NO: 91, and SEQ ID NO: 93.

TABLE 15
Sequences in antibodies comprising
the IL15:IL15Rα fusion construct

| Description | Sequence |
|---|---|
| AB-008873 mIgG2a Heavy Chain | (SEQ ID NO: 89) |
| AB-008873 mIgG2a Light Chain | (SEQ ID NO: 90) |
| Linker (G4S)3 | (SEQ ID NO: 91) |
| Linker TS(G4S)3 | (SEQ ID NO: 105) |
| IL15(wt/RLI) | (SEQ ID NO: 92) |
| IL15(low/RLI) | (SEQ ID NO: 93) |

In some embodiments, an EphA2 antibody described herein is conjugated to or administered with an IL-2 receptor agonist. In one embodiment, the tumor-targeting antibody that is conjugated to an IL-2 receptor agonist is a bispecific or multispecific antibody. In some embodiments, the antibody is a bispecific or multispecific antibody comprising an antigen binding domain of an antibody described herein and an IL-2 receptor agonist. In some embodiments, the IL-2 receptor agonist is pegylated IL-2.

In some embodiments, an EphA2 antibody described herein is conjugated to or administered with a construct that can act as a trap for transforming growth factor-β (TGFβ). In one embodiment, the TGFβ trap comprises the extracellular domain (ECD) of TGFβ. In one embodiment, the TGFβ trap comprises the extracellular domain (ECD) of TGFβRII. In one embodiment, the TGFβ trap is in the form of a bispecific antibody (see US2018/0118832A1, FIG. 1). The TGFβRII ECD can preferably trap TGFβ1, and its low affinity to TGFβ2 may mitigate potential cardiac toxicity.

Thus, in another aspect, an EphA2 antibody described herein comprises an extracellular domain (ECD) of the TGFβ Receptor fused to the C-terminus of the heavy chain or to the C-terminus of the light chain. In some embodiments, the TGFβ trap is a single trap construct. In some embodiments, the single TGFβ trap is a bispecific tumor-targeting TGFβ trap comprising a TGFβ RII ECD fused to any one of the antibodies disclosed herein via a flexible linker to the C-terminus of the heavy chain or to the C-terminus of the light chain.

In some embodiments, the TGFβ trap is a tandem trap construct. In some embodiments, the tandem TGFβ trap comprises an IgG fused to two TGFβRII ECDs. In some embodiments, the tandem TGFβ trap comprises two TGFβ2RII ECDs. In some embodiments, the two TGFβ2RII ECDs are fused in series and are linked by a short linker (for example L10 or L25). In some embodiments, the two TGFβ2RII ECDs are fused directly in series without a linker (L0). In some embodiments, the tandem TGFβRII ECDs are fused to the C-terminus of the heavy chain (HC-Cter), and the heavy chains were designed as an asymmetric pair such that the tandem trap is on only one heavy chain. In some embodiments, the asymmetric pair of heavy chains comprise knob-in-hole mutation that promote pairing of the heavy chains. For example, in some embodiments, one heavy chain comprises the amino acid substitutions T366S+L368A+Y407V (and optionally Y349C), and the other heavy chain comprises the amino acid substitution T336W (and optionally S354C). In some embodiments, the asymmetric single heavy chain C-ter fusion improves steric access of the Fc region to Fc gamma receptors and thereby improve function.

In some embodiments, the tandem TGFβ trap is fused to the C-terminus of the light chain (LC-Cter), such that both light chains comprise two TGFβRII ECDs. In these embodiments, the net molecule exhibits twice the TGFβ trapping capacity per molecule, and therefore may exhibit improved function.

In some embodiments, the bispecific TGFβ trap construct comprises human variable regions. In some embodiments, the bispecific TGFβ trap construct comprises a IgG1 or IgG2 constant region. In some embodiments, the bispecific TGFβ trap construct comprises a human IgG1 constant region. In some embodiments, the bispecific TGFβ trap construct comprises a mouse IgG2a constant region. In some embodiments, the variable regions of the TGFβ trap construct are fused in frame to the IgG constant regions.

Binding of the TGFβ trap construct can be determined using, for example, an ELISA assay, as described in the Examples. The ability of TGFβ trap constructs to bind to target tumor cells can be determined, for example, using flow-cytometry, as described in the Examples. The ability of TGFβ trap constructs to engage and stimulate Fc-gamma receptor in the presence of target tumor cells can be determined using a reporter bioassay, as described in the Examples. The ability of TGFβ trap constructs to inhibit tumor growth can be determined, for example, in a syngeneic mouse model, as described in the Examples.

In some embodiments, the antibody may be linked to a radionuclide, an iron-related compound, a dye, a fluorescent agent, or an imaging agent. In some embodiments, an antibody may be linked to agents, such as, but not limited to, metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores.

In one embodiment of any of the above constructs, the EphA2 antibody is any one of AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665. AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698, AB-010699, AB-010700, AB-010701, or AB-010702. In one embodiment of any of the above constructs, the tumor-targeting binding domain comprises the V_{H} and V_{L} sequences of AB-010361 or AB-010699.

### Methods of Inducing an Immune Response

In a further aspect, provided herein are methods of inducing an immune response by administering an EphA2 antibody as described herein to a subject that has a tumor. In some embodiments, the EphA2 antibody is an antibody set forth in Tables 6-8, or a variant thereof as described above. In some embodiments, the antibody or variant thereof comprises a modified Fc region comprising mutations described herein. For example, in some embodiments, the antibody comprises an Fc mutation that increases effect function selected from E333A, K326W/E333S, S239D/I332E/G236A, S239D/A330L/I332E, G236A/S239D/A330L/I332E, F243L, G236A, S298A/E333A/K334A, and P329G/L234A/L235A, or a combination thereof. In some embodiments, the antibody comprises a modified Fc region that is a-fucosylated. In some embodiments, the antibody is conjugated to or administered with an IL-15 receptor agonist, a TGFβ trap, a TLR agonist, or an agonist anti-4-1BB antibody. In some embodiments, the antibody is a bispecific or multispecific antibody described herein.

An immune response induced by administration of an antibody as described herein can be either an innate or adaptive immune response. In some embodiments, the antibody activates an immune response directly, e.g., via binding of the antibody to a target tumor cell and engagement with an Fc receptor on an effector cell such that the effector cell is activated. In some embodiments, the antibody indirectly activates an immune response by inducing immune responses that are initiated by antibody binding to the target cell and an effector cell with subsequent induction of downstream immune responses. In some embodiments, the antibody activates monocytes, myeloid cells, and/or NK cells, e.g., macrophages, neutrophils, dendritic cells, mast cells, basophils, eosinophile, and/or NK cells. In some embodiments, the antibody activates T lymphocytes and/or B cells.

### Treatment of Cancer

In a further aspect, an EphA2 antibody as provided herein, or a variant thereof as described herein, can be used and a therapeutic agent to treat cancer. In some embodiments, the antibody or variant thereof comprises a modified Fc region comprising mutations described herein. For example, in some embodiments, the antibody comprises an Fc mutation that increases effector function selected from E333A, K326W/E333S, S239D/I332E/G236A, S239D/A330L/I332E, G236A/S239D/A330L/I332E, F243L, G236A, S298A/E333A/K334A, and P329G/L234A/L235A, or a combination thereof. In some embodiments, the antibody comprises a modified Fc region that is afucosylated. In some embodiments, the antibody is conjugated to or administered with an IL-15 receptor agonist, a TGFβ trap, a TLR agonist, or an agonist anti-4-1BB antibody. In some embodiments, the antibody is a bispecific or multispecific antibody described herein.

In some aspects, the disclosure additionally provides methods of identifying subjects who are candidates for treatment with an EphA2 antibody having tumor-targeting effects. Thus, in one embodiment, the invention provides a method of identifying a patient who can benefit from treatment with an EphA2 antibody of the present disclosure. In one embodiment, the patient has tumor that expresses EphA2. In one embodiment, the patient has tumor that overexpresses EphA2. In some embodiments, the tumor sample is from a primary tumor. In alternative embodiments, the tumor sample is a metastatic lesion. Binding of antibody to tumor cells through a binding interaction with the EphA2 can be measured using any assay, such as immunohistochemistry or flow cytometry. In some embodiments, binding of antibody to at least 0.2%, 0.5%, or 1%, or at least 5% or 10%, or at least 20%, 30%, or 50%, of the tumor cells in a sample may be used as a selection criterion for determining a patient to be treated with an EphA2 antibody as described herein. In other embodiments, analysis of components of the blood, e.g., circulating exosomes and/or extracellular RNA-protein complex and/or extracellular protein, is used to identify a patient whose tumor cells are overexpressing EphA2.

An EphA2 antibody disclosed herein can be used to treat several different cancers. In some embodiments, a cancer patient who can benefit from the treatment of the EphA2 antibody has a cancer that expresses EphA2. In some embodiments, a cancer patient who can benefit from the treatment of the EphA2 antibody has a cancer overexpressing EphA2. In some embodiments, the cancer is a carcinoma or a sarcoma. In some embodiments, the cancer is a hematological cancer. In some embodiments, the cancer is breast cancer, prostate cancer, testicular cancer, renal cell cancer, bladder cancer, ovarian cancer, cervical cancer, endometrial cancer, lung cancer, colorectal cancer, anal cancer, pancreatic cancer, gastric cancer, esophageal cancer, hepatocellular cancer, head and neck cancer, a brain cancer, e.g., glioblastoma, melanoma, or a bone or soft tissue sarcoma. In one embodiment, the cancer is acral melanoma. In some embodiments, the cancer is acute lymphoblastic leukemia, acute myeloid leukemia, adrenocortical carcinoma, astrocytoma, basal-cell carcinoma, bile duct cancer, bone tumor, brainstem glioma, cerebellar astrocytoma, cerebral astrocytoma, ependymoma, medulloblastoma, supratentorial primitive neuroectodermal tumors, visual pathway and hypothalamic glioma, bronchial adenomas, Burkitt's lymphoma, central nervous system lymphoma, cerebellar astrocytoma, chondrosarcoma, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, epithelioid hemangioendothelioma (EHE), esophageal cancer, Ewing's sarcoma, extracranial germ cell tumor, extragonadal germ cell tumor, extrahepatic bile duct cancer, a cancer of the eye, intraocular melanoma, retinoblastoma, gallbladder cancer, gastrointestinal carcinoid tumor, gastrointestinal stromal tumor (GIST), germ cell tumor, gestational trophoblastic tumor, gastric carcinoma, hairy cell leukemia, hepatocellular carcinoma, Hodgkin lymphoma, hypopharyngeal cancer, hypothalamic and visual pathway glioma, childhood, intraocular melanoma, islet cell carcinoma, Kaposi sarcoma, kidney cancer, laryngeal cancer, leukemias, lip and oral cavity cancer, liposarcoma, liver cancer, non-small cell lung cancer, small-cell lung cancer, lymphomas, macroglobulinemia, male breast cancer, malignant fibrous histiocytoma of bone, medulloblastoma, Merkel cell cancer, mesothelioma, metastatic squamous neck cancer, mouth cancer, multiple endocrine neoplasia syndrome, multiple myeloma, mycosis fungoides, myelodysplastic syndromes, myelogenous leukemia, myeloid leukemia, adult acute, myeloproliferative disorders, chronic, myxoma, nasal cavity and paranasal sinus cancer, nasopharyngeal carcinoma, neuroblastoma, non-Hodgkin lymphoma, oligodendroglioma, oral cancer, oropharyngeal cancer, osteosarcoma, ovarian epithelial cancer, ovarian germ cell tumor, ovarian low malignant potential tumor, paranasal sinus and nasal cavity cancer, parathyroid cancer, penile cancer, pharyngeal cancer, pheochromocytoma, pineal astrocytoma, pineal germinoma, pineoblastoma, supratentorial primitive neuroectodermal tumors, pituitary adenoma, plasma cell neoplasia, pleuropulmonary blastoma, primary central nervous system lymphoma, rectal cancer, renal cell carcinoma, retinoblastoma, rhabdomyosarcoma, salivary gland cancer, Ewing sarcoma, Kaposi sarcoma, soft tissue sarcoma, uterine sarcoma, Sezary syndrome, non-melanoma skin cancer, melanoma, small intestine cancer, squamous cell carcinoma, squamous neck cancer, stomach cancer, cutaneous T-Cell lymphoma, throat cancer, thymoma, thyroid cancer, transitional cell cancer of the renal pelvis and ureter, trophoblastic tumor, gestational, urethral cancer, uterine cancer, vaginal cancer, vulvar cancer, Waldenström macroglobulinemia, or Wilms tumor.

In some embodiments, the cancer is lung cancer, e.g., non-small cell lung adenocarcinoma or squamous cell carcinoma; breast cancer, e.g., Triple⁻, ER/PR⁺Her2⁻, ER/PR⁻Her2⁺, or Triple⁻; colorectal cancer, e.g., adenocarcinoma, mucinous adenocarcinoma, or papillary adenocarcinoma; esophageal cancer; stomach cancer; kidney cancer, e.g., kidney clear cell cancer; ovarian cancer, e.g., ovarian endometrioid carcinoma, ovarian mucinous cystadenocarcinoma, or ovarian scrous cystadenocarcinoma; melanoma, e.g., acral melanoma, cutaneous melanoma, or mucosal melanoma; uterine or cervical cancer; liver cancer, e.g., hepatocellular carcinoma or bile duct carcinoma; bladder cancer, e.g., transitional or urothelial bladder cancer; or testicular cancer.

In some embodiments, an EphA2 antibody disclosed herein can be used to treat a gastric cancer, ovarian cancer, or soft tissue sarcoma. As discussed above, the EphA2 antibody, e.g., AB-008873, showed better tumor selectivity than several EphA2 antibodies derived from clinical candidates and commercial EphA2 antibodies as referenced in Table 34.

In one aspect, methods of the disclosure comprise administering an EphA2 antibody disclosed herein, or a variant thereof, as a pharmaceutical composition to a cancer patient in a therapeutically effective amount using a dosing regimen suitable for treatment of the cancer. The composition can be formulated for use in a variety of drug delivery systems. One or more physiologically acceptable excipients or carriers can also be included in the compositions for proper formulation. Suitable formulations for use in the present disclosure are found, e.g., in Remington: The Science and Practice of Pharmacy, 21st Edition, Philadelphia, PA. Lippincott Williams & Wilkins, 2005.

The tumor-targeting antibody is provided in a solution suitable for administration to the patient, such as a sterile isotonic aqueous solution for injection. The antibody is dissolved or suspended at a suitable concentration in an acceptable carrier. In some embodiments the carrier is aqueous, e.g., water, saline, phosphate buffered saline, and the like. The compositions may contain auxiliary pharmaceutical substances as required to approximate physiological conditions, such as pH adjusting and buffering agents, tonicity adjusting agents, and the like.

### Administration

The pharmaceutical compositions are administered to a patient in an amount sufficient to cure or at least partially arrest the disease or symptoms of the disease and its complications. An amount adequate to accomplish this is defined as a “therapeutically effective dose.” A therapeutically effective dose is determined by monitoring a patient's response to therapy. Typical benchmarks indicative of a therapeutically effective dose include the amelioration of symptoms of the disease in the patient. Amounts effective for this use will depend upon the severity of the disease and the general state of the patient's health, including other factors such as age, weight, gender, administration route, and the like Single or multiple administrations of the antibody may be administered depending on the dosage and frequency as required and tolerated by the patient. In any event, the methods provide a sufficient quantity of tumor-targeting antibody to effectively treat the patient.

An EphA2 antibody can be administered by any suitable means, including, for example, parenteral, intrapulmonary, and intranasal, administration, as well as local administration, such as intratumor administration. Parenteral infusions include intramuscular, intravenous, intraarterial, intraperitoneal, or subcutaneous administration. In some embodiments, the antibody may be administered by insufflation. In an illustrative embodiment, the antibody may be stored at 10 mg/ml in sterile isotonic aqueous saline solution for injection at 4° C. and is diluted in either 100 ml or 200 ml 0.9% sodium chloride for injection prior to administration to the patient. In some embodiments, the antibody is administered by intravenous infusion over the course of 1 hour at a dose of between 0.01 and 25 mg/kg. In other embodiments, the antibody is administered by intravenous infusion over a period of between 15 minutes and 2 hours. In still other embodiments, the administration procedure is via subcutaneous bolus injection.

The dose of antibody is chosen to provide effective therapy for the patient and is in the range of less than 0.01 mg/kg body weight to about 25 mg/kg body weight or in the range 1 mg-2 g per patient. Preferably the dose is in the range 0.1-10 mg/kg or approximately 50 mg-1000 mg/patient. The dose may be repeated at an appropriate frequency which may be in the range once per day to once every three months, or every six months, depending on the pharmacokinetics of the antibody (e.g., half-life of the antibody in the circulation) and the pharmacodynamic response (e.g., the duration of the therapeutic effect of the antibody). In some embodiments, the in vivo half-life of between about 7 and about 25 days and antibody dosing is repeated between once per week and once every 3 months or once every 6 months. In other embodiments, the antibody is administered approximately once per month.

In an illustrative embodiment, the antibody may be stored at 10 mg/ml or 20 mg/ml in a sterile isotonic aqueous solution. The solution can comprise agents such as buffering agents and stabilizing agents. For example, in some embodiments, a buffering agent such as histidine is included to maintain a formulation pH of about 5.5. Additional reagents such as sucrose or alternatives can be added to prevent aggregation and fragmentation in solution and during freezing and thawing. Agents such as polysorbate 80 or an alternative can be included to lower surface tension and stabilizes the antibody against agitation-induced denaturation and air-liquid and ice-liquid surface denaturation. In some embodiments, the solution for injection is stored at 4° C. and is diluted in either 100 ml or 200 ml 0.9% sodium chloride for injection prior to administration to the patient.

In some embodiments, antibody for IV administration is formulated at a target concentration of 20 mg/mL in 20 mM histidine buffer, 8% (w/v) sucrose and 0.02% (w/v) polysorbate 80, pH 5.5.

### Combination Therapy

An EphA2 antibody disclosed herein may be administered with one or more additional therapeutic agents, e.g., radiation therapy, chemotherapeutic agents and/or immunotherapeutic agents.

In some embodiments, an EphA2 antibody can be administered in conjunction with an agent that targets an immune checkpoint antigen. In one aspect, the agent is a biologic therapeutic or a small molecule. In another aspect, the agent is a monoclonal antibody, a humanized antibody, a human antibody, a fusion protein or a combination thereof. In certain embodiments, the agents inhibit, e.g., by blocking ligand binding to receptor, a checkpoint antigen that may be PD1, PDL1, CTLA-4, ICOS, PDL2, IDO1, IDO2, B7-H3, B7-H4, BTLA, HVEM, TIM3, GAL9, GITR, HAVCR2, LAG3, KIR, LAIR1, LIGHT, MARCO, OX-40, SLAM, 2B4, CD2, CD27, CD28, CD30, CD40, CD70, CD80, CD86, CD137 (4-1BB), CD160, CD39, VISTA, TIGIT, a SIGLEC, CGEN-15049, 2B4, CHK1, CHK2, A2aR, B-7 family ligands or their receptors, or a combination thereof. In some embodiments, the agent targets PD-1, e.g., an antibody that blocks PD-L1 binding to PD-1 or otherwise inhibits PD-1. In some embodiments, the agent targets CTLA-4. In some embodiments, the agent targets LAG3. In some embodiments, the agent targets TIM3. In some embodiments, the agents target ICOS.

In some embodiments, an EphA2 antibody can be administered in conjunction with a therapeutic antibody, such as an antibody that targets a tumor cell antigen. Examples of therapeutic antibodies include as rituximab, trastuzumab, tositumomab, ibritumomab, alemtuzumab, atezolizumab, avelumab, durvalumab, pidilizumab, AMP-224, AMP-514, PDR001, cemiplimab, BMS-936559, CK-301, epratuzumab, bevacizumab, elotuzumab, necitumumab, blinatumomab, brentuximab, cetuximab, daratumumab, denosumab, dinutuximab, gemtuzumab ibritumomab ipilimumab, nivolumab, obinutuzumab, ofatumumab, ado-trastuzumab, panitumumab, pembrolizumab, pertuzumab, ramucirumab, and ranibizumab. In some embodiments, an EphA2 antibody can be administered in conjunction with a therapeutic antibody that binds an extracellular RNA-protein complex comprising polyadenylated RNA, such as the antibody designated ATRC-101, see WO2020168231 incorporated herein in its entirety.

In some embodiments, an EphA2 antibody as described herein is administered with an agonist anti-4-1BB antibody such as urelumab (Bristol Meyers Squibb, BMS-663513, U.S. Pat. No. 7,288,638), utomilumab, (Pfizer, PF-05082566, WO2012145183), 1D8 and 5B9 (US20100279932), hu106-1, TABBY 101-TABBY 110 (WO2017205745, FIGS. 2A-2F), Hz4B4-1 (U.S. Pat. No. 6,458,934), BBK-1 and BBK-4 (U.S. Pat. No. 6,559,997), hu39E3.G4 (U.S. Pat. No. 6,887,673), CTX-471 and CTX-471AF (U.S. Ser. No. 10/279,038), MOR-6032, MOR-7361, MOR-7480 and MOR-7483 (U.S. Pat. No. 8,337,850), LVGN-6051 (US20210246218), AGEN-2373 (US 2021/0106693), ADG-106/AG10131 (US20200369776), ATOR-1017 (US20190352414).

In some embodiments, an EphA2 antibody is administered with a chemotherapeutic agent. Examples of cancer chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside; cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; docetaxel, platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylomithine (DMFO); retinoic acid derivatives such as bexarotene, alitretinoin; denilcukin diftitox; esperamicins; capecitabinc; and pharmaceutically acceptable salts, acids or derivatives of any of the above. Also included in this definition are anti-hormonal agents that act to regulate or inhibit honnone action on tumors such as anti-estrogens including for example tamoxifen, raloxifene, mifepristone, aromatase inhibiting 4(5)-imidazoles, 4-hydroxytamoxifen, trioxifene, keoxifene, LY 1 17018, onapristone, and toremifene (Fareston); and anti-androgens such as flutamide, nilutamide, bicalutamide, leuprolide, and goserelin; and pharmaceutically acceptable salts, acids or derivatives of any of the above. Further cancer therapeutic agents include sorafenib and other protein kinase inhibitors such as afatinib, axitinib, crizotinib, dasatinib, erlotinib, fostamatinib, gefitinib, imatinib, lapatinib, lenvatinib, mubritinib, nilotinib, pazopanib, pegaptanib, ruxolitinib, vandetanib, vemurafenib, and sunitinib; sirolimus (rapamycin), everolimus and other mTOR inhibitors. Examples of additional chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitoinycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea). Illustrative chemotherapeutic agents additionally include paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IxB kinase1 and other inhibitors of proteins or enzymes known to be upregulated, over-expressed or activated in cancers, the inhibition of which down regulates cell replication. Additional agents include asparaginase and a Bacillus Calmete-Guerin preparation.

In some embodiments, an EphA2 antibody as described herein is administered after, or at the same time, as a therapeutic agent, e.g., a chemotherapeutic agent, such as doxorubicin, that induces stress granules (“SG-inducing agent”). Increasing the amount of stress granules in cancer cells can promote targeting the tumor cells by the tumor-targeting antibody. Other exemplary therapeutic agents that can induce stress granules include pyrimidine analogs (e.g., 5-FU, under trade names of Adrucil®, Carac®, Efudex®, Efudix®); protease inhibitors (e.g., Bortezomib, under the trade name of Velcade®); kinase inhibitors (e.g, Sorafenib and Imatinib, under the trade names of Nexavar® and Gleevec®, respectively); Arsenic compounds (e.g., Arsenic trioxide, under the trade name of Trisenox®); Platinum-based compounds that induce DNA damage (e.g., Cisplatin and Oxaliplatin®, under the trade names of Platinol® and Eloxatin®, respectively); agents that disrupt microtubules (e.g., Vinblastin, under the trade name of Velban® or alkabban-AQ®; vincristin, under the trade name of Vincasar®, Margibo®, or Oncovin®; Vinorelbin, under the trade name of Navelbin®); topoisomerase II inhibitor (e.g., Etoposide, under the trade name of Etopophos, Toposar®, VePesid®); and agents that induce DNA damage, e.g., irradiation. Several exemplary therapeutic agents that can induce stress granules formation are disclosed in Mahboubi et al., Biochimica et Biophysica Acta 1863 (2017) 884-895.

Various combinations with the tumor-targeting antibody and the SG-inducing agent (or a combination of such agents) described herein may be employed to treat a cancer patient. By “combination therapy” or “in combination with”, it is not intended to imply that the therapeutic agents must be administered at the same time and/or formulated for delivery together, although these methods of delivery are within the scope described herein. The tumor-targeting antibody and the SG-inducing agent can be administered following the same or different dosing regimen. In some embodiments, the tumor-targeting antibody and the SG-inducing agent is administered sequentially in any order during the entire or portions of the treatment period. In some embodiments, the tumor-targeting antibody and the SG-inducing agent is administered simultaneously or approximately simultaneously (e.g., within about 1, 5, 10, 15, 20, or 30 minutes of each other). In still other embodiments, the SG-inducing agent may be administered 1, 2, 3, 4, 5, 6, 7, 8, 9, 10, or more days before administration of the tumor-targeting antibody. In some embodiments, the SG-inducing agent is administered from 1 to 4 weeks, or longer, before the tumor-targeting antibody is administered.

An EphA2 antibody may also be administered to a cancer patient in conjunction with a cell-based therapy, such as natural killer (NK) cell therapy or a cancer vaccine. In some instances, a cancer vaccine is a peptide-based vaccine, a nucleic acid-based vaccine, a cell-based vaccine, a virus-based or viral fragment-based vaccine or an antigen presenting cell (APC) based vaccine (e.g., dendritic cell based vaccine). Cancer vaccines include Gardasil®, Cervarix®, sipuleucel-T (Provenge®), NeuVax™ HER-2 ICD peptide-based vaccine, HER-2/neu peptide vaccine, AdHER2/neu dendritic cell vaccine, HER-2 pulsed DC1 vaccine, Ad-sig-hMUC-1/ecdCD40L fusion protein vaccine, MVX-ONCO-1, hTERT/survivin/CMV multipeptide vaccine, E39, J65, P10s-PADRE, rV-CEA-Tricom, GVAX®, Lucanix®, HER2 VRP, AVX901, ONT-10, ISAlOl, ADXSl 1-001, VGX-3100, INO-9012, GSK1437173A, BPX-501, AGS-003, IDC-G305, HyperAcute®-Renal (HAR) immunotherapy, Prevenarl3, MAGER-3.A1, NA17.A2, DCVax-Direct, latent membrane protein-2 (LMP2)-loaded dendritic cell vaccine (NCT02115126), HS410-101 (NCT02010203, Heat Biologies), EAU RF 2010-01 (NCT01435356, GSK), 140036 (NCT02015104, Rutgers Cancer Institute of New Jersey), 130016 (NCTO 1730118, National Cancer Institute), MVX-201101 (NCT02193503, Maxivax SA), ITL-007-ATCR-MBC (NCT01741038, Immunovative Therapies, Limited), CDR0000644921 (NCT00923143, Abramson cancer center of the University of Pennsylvania), SuMo-Sec-01 (NCT00108875, Julius Maximilians Universitaet Hospital), or MCC-15651 (NCT01176474, Medarex, Inc, BMS).

In some embodiments, an EphA2 antibody of the present disclosure may be administered with an agent, e.g., a corticosteroid, that mitigates side-effects resulting from stimulation of the immune system.

In the context of the present disclosure a therapeutic agent that is administered in conjunction with an EphA2 antibody of the present disclosure can be administered prior to administrations of the tumor-targeting antibody or after administration of the tumor-targeting antibody. In some embodiments, an EphA2 antibody may be administered at the same time as the additional therapeutic agent. In some embodiments, an EphA2 antibody and an additional therapeutic agent described above can be administered following the same or different dosing regimens. In some embodiments, the tumor-targeting antibody and the therapeutic agent are administered sequentially in any order during the entire treatment period or portions thereof. In some embodiments, the tumor-targeting antibody and the therapeutic agent are administered simultaneously or approximately simultaneously (e.g., within about 1, 5, 10, 15, 20, or 30 minutes of each other). In still other embodiments, the therapeutic agent may be administered 1, 2, 3, 4, 5, 6, 7, 8, 9, 10, or more days before the administration of the tumor-targeting antibody. In still other embodiments, the therapeutic agent may be administered 1, 2, 3, 4, 5, 6, 7, 8, 9, 10, or more days after the administration of the tumor-targeting antibody.

### Functional Assays

Also described herein are functional assays that can be used to determine the ability of the antibodies described herein to mediate FcR-dependent activity. In some embodiments, the assay measures antibody dependent cellular cytotoxicity (ADCC), antibody dependent cellular phagocytosis (ADCP), or complement-dependent cytoxicity (CDC).

### In Vivo Assays

In some embodiments, the activity of the antibodies is evaluated in vivo in an animal model that is known for specific human tumors. One exemplary model is the CT26 mouse model, as described in the EXAMPLES. Tumor-targeting activity of these antibodies in vivo may be assessed by using several assays, including but not limited to using flow cytometry to analyze the immune profiling of the blood and tumor, monitoring tumor growth, and performing immunofluorescence to semi-quantitative estimate tumor infiltration. In some embodiments, the effect of the antibody can be assessed using Survival, a normalized area above the curve metric (NAAC), and a normalized growth rate metric (NGRM), where NAAC and NGRM were both developed at Atreca. An “in vivo active” determination can be based on the in vivo activity was assessed by a p-value≤0.05 in at least one of the analyses of survival, NAAC, and NGRM, i.e., if an antibody exhibited a p-value of less than or equal to 0.05 for survival, NAAC, and/or NGRM (any one alone being sufficient), the antibody is considered “in vivo active”.

In one aspect, provided herein are antibodies that exhibit inhibitory effects on tumors, including decreasing rate of tumor growth, size, tumor invasion and/or metastasis. Such antibodies exhibit tumor-targeting effects in vivo, e.g., when administered to subjects that has a tumor expressing or overexpressing EphA2.

### Engineering Variants

In some embodiments, an antibody or variant thereof described herein is modified to have improved developability (i.e., reduced development liabilities), including but not limited to, decreased heterogeneity, increased yield, increased stability, improved net charges to improve pharmacokinetics, and or/reduced immunogenicity. In some embodiments, antibodies having improved developability can be obtained by introducing mutations to reduce or eliminate potential development liabilities. In some embodiments, antibodies having improved developability possess modifications as compared to a reference or control antibody in their amino acid sequence.

In some embodiments, the antibodies or variants thereof disclosed herein have improved developability while maintaining comparable or improved binding affinity to the target antigen as compared to a reference or control (unmodified) antibody. In some embodiments, the antibodies or variants thereof disclosed herein have improved developability while maintaining activities similar to a reference or control (unmodified) antibody.

In some embodiments, the antibodies or variants thereof have improved developability, e.g., as identified through various in vitro assays, such as aggregation assessment by HPLC or UPLC, hydrophobic interaction chromatography (HIC), polyspecificity assays (e.g., baculovirus particle binding), self-interaction nanoparticle spectroscopy (SINS), or mass spec analysis after incubation in an accelerated degradation condition such as high temperature, low pH, high pH, or oxidative H₂O₂. Mutations are successful if activity is maintained (or enhanced) while removing or reducing the severity of the liability.

Improved properties of antibodies or variants thereof as described herein include: (1) fits a standard platform (expression, purification, formulation); (2) high yield; (3) low heterogeneity (glycosylation, chemical modification, and the like); (4) consistent manufacturability (batch-to-batch, and small-to-large scale); (5) high stability (years in liquid formulation), e.g., minimal chemical degradation, fragmentation, and aggregation; and (6) long PK (in vivo half-life), e.g., no off-target binding, no impairment of FcRn recycling, and stable. Antibody liabilities are further described in Table 16.

TABLE 16
Description of potential development liabilities

| | | | |
|---|---|---|---|
| Free cysteine⁶ | Yield, heterogeneity, | Sequence comprises | |
| | stability, activity | an odd number of | |
| | | cysteines | |
| N-linked glycosylation | Yield, heterogeneity, | N(~P)(S, T)¹ | High |
| | activity | | |
| Abnormal net charge | Platform fit, PK | Sharma 2014² | High |
| Patches of hydrophobicity | Stability, PK | Sharma 2014 | High |
| Patches of same charge | Stability, PK | N/A (based on | Medium |
| | | structure) | |
| Proteolysis | Stability, PK | (K, R)(K, R)³ | Medium |
| Proteolysis | Stability, PK | DP | Medium |
| Asparagine deamidation | Heterogeneity, | NG; N(A, N, S, T)⁴ | Medium; |
| | stability, activity | | Low |
| Aspartate isomerization | Heterogeneity, | DG; D(A, D, S, T)⁵ | Medium; |
| | stability, activity | | Low |
| Lysine glycation | Heterogeneity, | K | Low |
| | stability, activity | | |
| Methionine oxidation | Heterogeneity, | M | Low |
| | stability, activity | | |
| Tryptophan oxidation | Heterogeneity, | W | Low |
| | stability, activity | | |

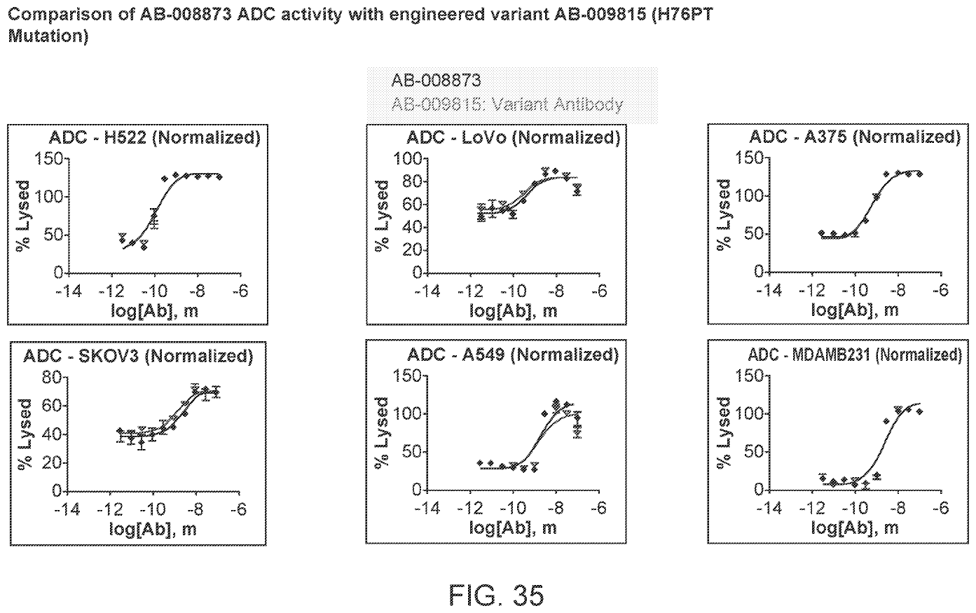

Note:
¹The N-linked glycosylation site is N-X-S/T, where X is any residue other than proline.
²Sharma et al., Proc. Natl. Acad. Sci. USA 111: 18601-18606, 2014
³This motif consists of a K or R, followed by a K or R. Stated differently, the motif can be KK, KR, RK, or RR.
⁴The dipeptide NG poses a medium risk of development liability. The dipeptides NA, NN, NS, and NT pose a low risk of development liability. N may also exhibit low risk of liability for other successor residues, e.g., D, H, or P. Stated differently, dipeptide ND, NH, or NP poses a low risk of development liability.
⁵Similarly to the above, the dipeptide DG poses a medium risk of development liability. The dipeptides DA, DD, DS, and DT pose a low risk of development liability. D may also exhibit low risk of development liability for other successor residues, e.g., N, H, or P.
⁶“Free cysteine” refers to a cysteine that does not form a disulfide bond with another cysteine and thus is left “free” as thiols. The presence of free cysteines in the antibody can be a potential development liability. Typically, an odd net number of cysteines in the protein shows a likelihood there is a free cysteine.
As one illustrative example, a variant was generated by introducing H76PT mutation to the heavy chain variable region of AB-009815 (SEQ ID NO: 75). This mutation removes a medium risk DP proteolytic cleavage site that could cause AB degradation during production, while in storage, or in the serum which decreases half-life after injection in mice/humans. The variant retains the ADC activity of AB-009815 in tumor cells, e.g., H522, LoVo, A375, SKOV3, A549, and MDAMB231 cells (FIG. 35)

Another goal for engineering variants is to reduce the risk of clinical immunogenicity: the generation of anti-drug antibodies against the therapeutic antibody. To reduce the risk, the antibody sequences are evaluated to identify residues that can be engineered to increase similarity to the intended population's native immunoglobulin variable region sequences.

The factors that drive clinical immunogenicity can be classified into two groups. First are factors that are intrinsic to the drug, such as: sequence; post-translational modifications; aggregates; degradation products; and contaminants. Second are factors related to how the drug is used, such as: dose level; dose frequency; route of administration; patient immune status; and patient HLA type.

One approach to engineering a variant to be as much like self as possible is to identify a close germline sequence and mutate as many mismatched positions (also known as “germline deviations”) to the germline residue type as possible. This approach applies for germline genes IGHV, IGHJ, IGKV, IGKJ, IGLV, and IGLJ, and accounts for all the variable heavy (V_{H}) and variable light (V_{L}) regions except for part of H-CDR3. Germline gene IGHD codes for part of the H-CDR3 region but typically exhibits too much variation in how it is recombined with IGHV and IGHJ (e.g., forward or reverse orientation, any of three translation frames, and 5′ and 3′ modifications and non-templated additions) to present a “self” sequence template from a population perspective.

Each germline gene can present as different alleles in the population. The least immunogenic drug candidate, in terms of minimizing the percent of patients with an immunogenic response, would likely be one which matches an allele commonly found in the patient population. Single nucleotide polymorphism (SNP) data from the human genome can be used to approximate the frequency of alleles in the population.

Another approach to engineering a lead for reduced immunogenicity risk is to use in silico predictions of immunogenicity, such as the prediction of T cell epitopes, or use in vitro assays of immunogenicity, such as ex vivo human T cell activation. For example, services such as those offered by Lonza, United Kingdom, are available that employ platforms for prediction of HLA binding and in vitro assessment to further identify potential epitopes.

Antibody variants can be designed to enhance the efficacy of the antibody. In some embodiments, design parameters can focus on CDRs, e.g., CDR3. Positions to be mutated can be identified based on structural analysis of antibody-antigen co-crystals (Oyen et al., Proc. Natl. Acad Sci. USA 114: E10438-E10445, 2017; Epub Nov. 14, 2017) and based on sequence information of other antibodies from the same lineage.

### Approaches to Mutation Design

Development liabilities can be removed or reduced by one or more mutations. Mutations are designed to preserve antibody structure and function while removing or reducing development liabilities and to improve function. In some embodiments, mutations to chemically similar residues can be identified that maintain size, shape, charge, and/or polarity. Illustrative mutations are described in Table 17.

TABLE 17
Preferred mutations to remove development liabilities

| | | | |
|---|---|---|---|
| Free cysteine | Odd # C | High | C→(A, S) |
| N-linked glycosylation | N(~P)(S, T) | High | N→(Q, D, S, A); (S, T)→(A, N) |
| Proteolytic cleavage | (K, R)(K, R) | Medium | K, R→(Q, S, A) |
| Proteolytic cleavage | DP | Medium | D→(E, S, A) |
| Asparagine deamidation | NG; N(A, N, S, T)* | Medium; Low | N→(Q, S, A); G→(A, S) |
| Aspartate isomerization | DG; D(A, D, S, T)* | Medium; Low | D→(E, S, A); G→(A, S) |
| Lysine glycation | K | Low | K→(R, Q, S, A) |
| Methionine oxidation | M | Low | M→(Q, L, S, A) |
| Tryptophan oxidation | W | Low | W→(Y, F) |

Note:
the last column of Table 17 shows preferred mutations. For example, C→(A, S) refers to C can be mutated to either A or S to remove development liabilities.

### Methods for Altering the Glycosylation of an Antibody

In another aspect, the antibodies described herein comprise an Fc region having altered glycosylation that increase the ability of the antibody to recruit NK cells and/or increase ADCC. In some embodiments, the Fc region comprises glycan containing no fucose (i.e., the Fc region is afucosylated). Fucosylated antibodies can be produced using cell lines that express a heterologous enzyme that depletes the fucose pool inside the cell (e.g., GlymaxX® by ProBioGen AG, Berlin, Germany). Non-fucosylated antibodies can also be produced using a host cell line in which the endogenous α-1,6-fucosyltransferase (FUT8) gene is deleted. See Satoh. M. et al., “Non-fucosylated therapeutic antibodies as next-generation therapeutic antibodies,” Expert Opinion on Biological Therapy, 6:11, 1161-1173, DOI: 10.1517/14712598.6.11.1161.

### Diagnosis of Cancer

The EphA2 antibodies disclosed herein can also be used for diagnosing a patient having a cancer suitable for treatment. In one aspect, the method comprises contacting a tumor sample from the patient with an antibody disclosed above and detecting binding of the antibody to the tumor sample. In some cases, the antibodies are conjugated to a detectable label that produces fluorescent, luminescent or colorimetric signals, and detecting the signal from the label indicates that tumor is suitable for treatment with an EphA2 antibody disclosed herein. In some cases, after the antibodies are contacted with the tumor sample, a labeled secondary antibody is added to the tumor sample that have been contacted with the antibody disclosed herein and detecting the signal from the secondary antibody indicates that the tumor expressing or overexpressing EphA2.

As discussed above, in some tumor types, e.g., uterine cancer, head and neck cancer, and NSCLC, an EphA2 antibody may display donor-specific tumor selectivity, i.e., the antibody may show tumor selectivity in some donors but not other donors. In these cases, it is desirable to determine the binding of the tumor to the EphA2 antibody before treating the patient.

### Screening Antibodies

Also disclosed herein is a method for selecting a tumor-targeting antibody. The method comprises contacting an antibody disclosed above with a polypeptide comprising SEQ ID NO: 94 or SEQ ID NO: 95 and contacting the antibody with a tumor cell and selecting the antibody if the antibody binds to the polypeptide and binds preferentially to the tumor cell as compared to normal cell.

The following examples are offered for illustrative purposes and are not intended to limit the invention. Those of skill in the art will readily recognize a variety of non-critical parameters that can be changed or modified to yield essentially the same results.

### EXAMPLES

### Example 1. AB-008873

AB-008873 was discovered in antibody repertoires generated by Immune Repertoire Capture® (IRC™) technology from plasmablast B cells isolated a non-small cell lung cancer patient with an active anti-tumor immune response after treatment with the anti-PD-1 antibody OPDIVO® (nivolumab) (Bristol Myers Squibb). AB-008873 was tested for cell surface binding to 6,000 human membrane proteins expressed on the surface of HEK293 cells by flow cytometry. Two membrane proteins showed binding over the background: EphA2 and FcGR1. No other ephrin type-A or type-B receptors had a binding signal above the background.

AB-008873 was further shown to bind the extracellular domain of EphA2 via an indirect and reverse indirect ELISA. The ELISAs also confirmed binding to mouse EphA2.

Next, AB-008873 was tested to determine if the previously observed the in vitro surface binding to A549 of the antibody was mediated by interaction with EphA2. 3 EphA2 guide RNAs were introduced into Cas9 overexpressing A549 cells. The EphA2 guide RNAs were designed by Synthego and Chop CRISPR tool. Exact sequences were selected based on low off-target score. The designed sgRNAs were synthesized and modified by Synthego. Electroporations of guide RNA was performed using the Neon™ Transfection System 10 μL Kit #MPK1096 according to the manufacturer's protocol. Final concentrations of the sgRNA were 100 μM (100 pmol/l). This resulted in a knockout of EphA2 gene in >90% of cells. The polyclonal EphA2 KO A549 cell population was then cultured for ˜4 days and assayed for cell surface binding by AB-008873. The fraction of cells in the AB-008873 positive gate was reduced from ˜98% (irrelevant guide RNA) to ˜10%, thus confirming EphA2 is the primary driver of AB-008873 A549 binding.

### Example 2. AB-008873 Variants

The sequence of AB-008873 was analyzed for potential liabilities. There are no high-risk liabilities. There is a medium-risk proteolytic cleavage site at H75D-H76P (i.e., heavy chain position 75 aspartate followed by heavy chain position 76 proline) and a medium-risk deamidation site at H108N-H109G. Low-risk liabilities include tryptophan oxidation at H34W and L90W; asparagine deamidation at H60N-H61N and L25N-L26N; lysine glycation at H67K, H100K, and L30K; and aspartate isomerization at H115D-H116A, L49D-L50D, L50D-L51S, and L91D-L92S.

AB-008873 was aligned to its closest human germline genes (IGHV4-34*02, IGHD2-8*01, IGHJ3*02, IGLV3-21*02, and IGLJ2*01), and to four of its known siblings (AB-009805, AB-009806, AB-009807, and AB-009808). Three variants were designed to explore differences between AB-008873 and its siblings. AB-009812 was designed with mutation H54RS to AB-008873 (heavy chain position 54 mutated from arginine to serine), to match H54S as found in the siblings and germline, and to create the N-linked glycosylation motif found in the siblings and germline (H52N-H53H-H54S). AB-009813 was designed with mutation H54RA to AB-008873, to remove the H54 arginine from AB-008873, but not create an N-linked glycosylation motif (ie, H52N-H53H-H54A is not an N-linked glycosylation motif). AB-009814 was designed with deletion of three residues, H61N-H62Y-H63N, to match the siblings and germline in that region. Note that AB-009814 could equivalently be described as a deletion of H60N-H61N-H62Y, H59Y-H60N-H61N, or H58N-H59Y-H60N and the resulting sequence is the same.

AB-009815 and AB-009816 were designed to remove the medium-risk proteolytic cleavage liability in AB-008873 at H75D-H76P. The variants make the mutation H76PT or H76PA, respectively. AB-009815 with the H76PT mutation also changes the sequence o germline.

AB-009817 was designed to remove the medium-risk deamidation site at H108N-H109G. The variant makes the mutation H109GA relative to AB-008873, to reduce the liability to a low risk deamidation site (i.e., NG is medium risk, but NA is low risk). H106C and H111C are predicted to form an intra-H3 disulfide bond, with H107-H110 in a 4-residue turn. Alternative approaches to removing the medium risk deamidation site include H108NS, H108NA, H108NQ, H108NL, or H108NY.

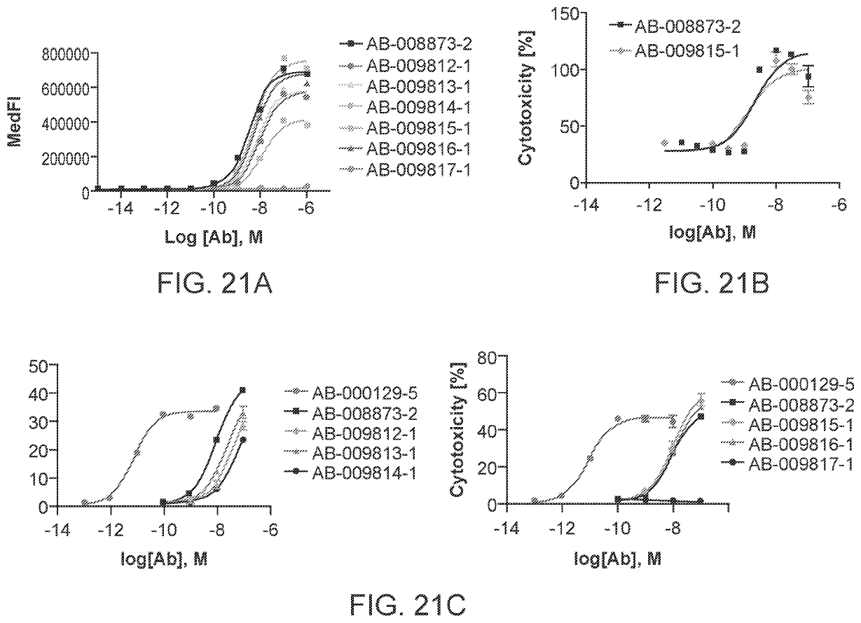

AB-008873, siblings AB-009805, AB-009806, AB-009807, AB-009808, and variants AB-009812, AB-009813, AB-009814, AB-009815, AB-009816, and AB-009817 were synthesized on a mouse IgG2a framework. All siblings show less binding, less ADCC, and less ADCP activity than AB-008873. See, FIG. 20. Variants AB-009815 and AB-009816, which remove the same medium-risk cleavage site, show similar levels of binding and activity as AB-008873. AB-009817 completely loses binding (tested up to 1 μM) and functional activity (tested up to 100 nM). Variants AB-009812, AB-009813, and AB-009814 show some binding and ADCC activity, though less than AB-008873. These results show that the N-linked glycosylation motif and the 3-aa insertion in H2 region affect binding, but they are not essential. The ADC (antibody-drug conjugate) activity of AB-009815 is the same as that of AB-008873. See, FIG. 21A-C and Table 18.

TABLE 18
Functional activity of engineered AB-008873 variants

| | | Flow | | ADCC | | |
|---|---|---|---|---|---|---|
| Reg ID | Mutation | EC₅₀ (nM) | Delta | EC₅₀ (nM) | Delta | Rationale |
| AB-008873 | | 3.6 | 680293 | 8.9 | 46.8 | |
| AB-009812 | H54RS | 9.6 | 567759 | 51.0 | 41.1 | Re-create NHS |
| | | | | | | glycosylation motif as |
| | | | | | | found in sibs and germline |
| AB-009813 | H54RA | 6.8 | 578049 | 26.0 | 39.9 | Keep glycosylation motif |
| | | | | | | out bt via alanine instead of |
| | | | | | | arginine |
| AB-009814 | H61NYNxxx | 11.5 | 407293 | 62.8 | 36 | Undo 3-residue insertion, |
| | | | | | | back to as found in sibs and |
| | | | | | | germline |
| AB-009815 | H76PT | 5.5 | 745456 | 10.4 | 59.8 | Remove medium-risk DP |
| | | | | | | proteolytic cleavage site |
| AB-009816 | H76PA | 5.5 | 670105 | 12.5 | 58 | Remove medium-risk DP |
| | | | | | | proteolytic cleavage site |
| AB-009817 | H109GA | inactive | 0 | inactive | 0 | Remove medium-risk NG |
| | | | | | | asparagine deamidation site |
| | | | | | | (leave as low-risk NA) |

The 12 variants AB-010141, AB-010142, AB-010143, AB-010144, AB-010145, AB-010146, AB-010147, AB-010148, AB-010149, AB-010150, AB-010151, and AB-010152 were designed to mutate AB-009815 closer to germline. Each variant exhibits 1 to 4 mutations at positions that differ from the closest germline. Some positions are framework; some are CDR. AB-010141 makes H31DG. AB-010142 makes L31NS. AB-010143 makes L97LV. AB-010144 makes H51VI. AB-010145 makes H86TS. AB-010146 makes H129AS. AB-010147 makes L60QR. AB-010148 combines H51VI and H86TS. AB-010149 combines H51VI and H129AS. AB-010150 combines H86TS and H129AS. AB-010151 combines H51VI, H86TS, and H129AS. AB-010152 combines H51VI, H86TS, H129AS, and L60QR. Further combinations of mutations to germline are of interest.

Anti-EphA2 Variants AB-010141, AB-010142, AB-010143, AB-010144, AB-010145, AB-010146, AB-010147, AB-010148, AB-010149, AB-010150, AB-010151, and AB-010152 were synthesized on a mouse IgG2a framework and assayed for ADCC, both by EC₅₀ and delta-activity as compared AB-008873 and melting temperature (Tm) measured using UNcle instrument. The results of these assays are shown in Table 19.

TABLE 19
Properties of anti-EphA2 antibodies

| | | | | ADCC | |
|---|---|---|---|---|---|
| AB regid | Parent | Mutations | Tm | EC₅₀ (nM) | dAct (%) |
| AB-008873 | N/A | N/A | 64.1 | 29.0 | 18.6 |
| AB-010141 | AB-009815 | H31DG | 63.1 | 4.6 | 24.2 |
| AB-010142 | AB-009815 | L31NS | 64.2 | | |
| AB-010143 | AB-009815 | L97LV | 65.4 | 25.1 | 21.6 |
| AB-010144 | AB-009815 | H51VI | 64.5 | 35.8 | 17.1 |
| AB-010145 | AB-009815 | H86TS | 64.1 | 48.9 | 19.2 |
| AB-010146 | AB-009815 | H129AS | 63.9 | 66.8 | 17.3 |
| AB-010147 | AB-009815 | L60QR | 66.5 | 79.4 | 15.8 |
| AB-010148 | AB-009815 | H51VI_H86TS | 64.4 | 45 | 17.4 |
| AB-010149 | AB-009815 | H51VI_H129AS | 64.6 | | |
| AB-010150 | AB-009815 | H86TS_H129AS | 64.4 | 65.9 | 17.1 |
| AB-010151 | AB-009815 | H51_H86_H129 | 64.6 | 33.3 | 17.5 |
| AB-010152 | AB-009815 | H51_H86_H129_L60 | 67.0 | | |

AB-010141 with the H31DG mutation exhibits improved ADCC EC₅₀ but decreases Tm by approximately 1° C. AB-010143 with L97LV exhibits improved Tm by approximately 1° C. AB-010147 with L60QR exhibits improved Tm by approximately 2° C. but might exhibit somewhat decreased ADCC activity. AB-010148 with H51VI and H86TS exhibits similar Tm and ADCC activity as compared to AB-008873.

Additional variants were generated based on AB-010148 and designated as AB-010357, AB-010358, AB-010359, AB-010360, AB-010361, AB-010362, AB-010363, AB-010364, AB-010365, AB-010366, and AB-010367.

AB-010148 was selected based on Tm and ADCC activity similar to that of AB-008873, while incorporating H76PT to remove the medium-risk DP proteolytic cleavage site and incorporating H51VI and H86TS to remove germline deviations to reduce potential immunogenicity. Combinations of L31DG, L97LV, and/or L60QR were added to AB-010148 to improve Tm, improve ADCC activity, and/or remove germline deviations. H31DG is predicted to remove an intramolecular salt bridge between H31D and H54R. To potentially compensate for this removal, H31DG was combined with either H54RA or H54RQ, along with L97LV and optionally L60QR, in variants AB-010364, AB-010365, AB-010366, and AB-010367.

Antibody variants AB-010357-AB-010367 were synthesized on a mouse IgG2a framework and assayed for ADCC, both by EC₅₀ and delta-activity (i.e., change in activity) as compared AB-008873 and melting temperature (Tm) measured using UNcle instrument. The results of these assays are shown in Table 20.

TABLE 20
Properties of Anti-EphA2 antibodies

| | | | | EC₅₀ | Fold- | dAct |
|---|---|---|---|---|---|---|
| AB regid | Parent | Mutations | Tm | (nM) | improved | (%) |
| AB-008873 | N/A | N/A | | 34.8 | 1.00 | 19.3 |
| AB-010357 | AB-010148 | L97LV | 65.3 | 11.6 | 3.00 | 23.1 |
| AB-010358 | AB-010148 | L60QR | 66.9 | 73.3 | 0.47 | 15.7 |
| AB-010359 | AB-010148 | L97LV_L60QR | 67.6 | 25.3 | 1.38 | 16.9 |
| AB-010360 | AB-010148 | H31DG | 64.0 | 4.3 | 8.09 | 26.1 |
| AB-010361 | AB-010148 | H31DG_L97LV | 65.0 | 3.1 | 11.23 | 24.1 |
| AB-010362 | AB-010148 | H31DG_L60QR | 65.9 | 12.8 | 2.72 | 19.6 |
| AB-010363 | AB-010148 | H31DG_L60QR_L97LV | 66.5 | 9.5 | 3.66 | 27.8 |
| AB-010364 | AB-010148 | H31DG_H54RA_L97LV | 64.5 | 14.5 | 2.40 | 26.3 |
| AB-010365 | AB-010148 | H31DG_H54RA_L97LV_L60QR | 66.7 | 34.6 | 1.01 | 21.3 |
| AB-010366 | AB-010148 | H31DG_H54RQ_L97LV | 64.7 | 28.9 | 1.20 | 25.9 |
| AB-010367 | AB-010148 | H31DG_H54RQ_L97LV_L60QR | 67.6 | 42.7 | 0.81 | 21.3 |

The results show that multiple variants exhibit improvements in both Tm and ADCC activity relative to AB-008873.

Antibodies AB-010357, AB-010361, and AB-010363 were then used as the basis for generation of further variants. AB-010357 was mutated to generate AB-010661, AB-010662, AB-010663, AB-010664, AB-010665, AB-010666, AB-010667, AB-010668, AB-010669, AB-010670, AB-010671. AB-010672, AB-010673, and AB-010674.

AB-010361 was mutated to generate AB-010675, AB-010676, AB-010677, AB-010678, AB-010679, AB-010680, AB-010681, AB-010682, AB-010683, AB-010684, AB-010685, AB-010686, AB-010687, and AB-010688.

AB-010363 was mutated to generate AB-010689, AB-010690, AB-010691, AB-010692, AB-010693, AB-010694, AB-010695, AB-010696, AB-010697, AB-010698, AB-010699, AB-010700, AB-010701, and AB-010702.

### Example 3. In Vitro Studies

### Immunophenotyping

Peripheral blood was blocked with 1:100 TruStain FcX PLUS Antibody (BioLegend 156604) for 10 min at 4° C. and stained for 30 min at 4° C. with a mastermix of antibodies for each panel. Next, 1.6 ml of 1× fix/lyse solution prepared according to manufacturer's protocol (BD 558049) was added to each sample and incubated for 10 min at RT. Samples were centrifuged at 500×g for 5 min, supernatant removed and transferred to a 96-well plate. Samples were centrifuged at 300×g for 5 min, washed with 200 μl of PBS, and resuspended in 100 μl of 1% FBS/5 mM EDTA/PBS for analysis on Beckman Coulter's Cytoflex flow cytometer. The results were analyzed using FlowJo_v10.7.1. Reagents for immunophenotyping are shown in Table 21.

TABLE 21
Reagents for immunophenotyping

| Lymphoid Panel | BioLegend | Myeloid Panel | BioLegend |
|---|---|---|---|
| CD45-PerCP | 103129 | CD45-PerCP | 103129 |
| CD3-APC | 152306 | Ly6C-APC | 128016 |
| CD8-AF700 | 100730 | F4/80-AF700 | 123130 |
| CD4-APCCy7 | 100414 | B220-APCCy7 | 103224 |
| CD25-PE | 101904 | Tim3-PE | 119704 |
| Tim3-PEDazzle594 | 134014 | CD11c-PEDazzle594 | 117348 |
| CD49b-PECy7 | 108922 | CD103-PECy7 | 121426 |
| CD62L-BV421 | 104436 | MHCII-BV421 | 107632 |
| PD1-BV510 | 135241 | CD24-BV510 | 101831 |
| CD44-BV605 | 103047 | Ly6G-BV605 | 127639 |
| CD19-BV650 | 115541 | CD11b-BV650 | 101259 |

Tumors were digested using Miltenyi's gentleMACS Octo Dissociator with heaters and Tumor Dissociation Kit, mouse (130-096-730), according to the manufacturer's instructions. The dissociated cells were cryopreserved in 10% FBS/DMSO. Dissociated cells were thawed using pre-warmed 2% FBS/RPMI dropwise, centrifuged at 300×g for 10 min, with supernatant decanted. Cells are blocked TStain FcX PLUS Antibody (BioLegend 156604) for 10 min at 4° C. and stained with a mastermix of antibodies for 30 min at 4° C. Next, 100 μl of PBS was added, cells were centrifuged at 300×g for 5 min supernatant decanted, and washed with another 200 μl PBS. Cells were resuspended in 100 μl of 1% FBS/5 mM EDTA/PBS with 1:500 Membrane integrity Dye (Intellicyt 90365) for analysis on Beckman Coulter's Cytoflex flow cytometer. Resulting data was analyzed using FlowJo_v10.7.1. Reagents for immunophenotyping in tumor cells are provided in Table 22.

TABLE 22
Reagents for immunophenotyping in tumor cells

| Lymphoid Panel | BioLegend | Myeloid Panel | BioLegend |
|---|---|---|---|
| CD3-APC | 152306 | Ly6C-APC | 128016 |
| CD8-AF700 | 100730 | F4/80-AF700 | 123130 |
| CD4-APCCy7 | 100414 | B220-APCCy7 | 103224 |
| CD25-PE | 101904 | CD45-PE | 103106 |
| NKG2D-PEDazzle594 | 130214 | CD11c-PEDazzle594 | 117348 |
| CD49b-PECy7 | 108922 | CD103-PECy7 | 121426 |
| PD1-BV421 | 135221 | MHCII-BV421 | 107632 |
| Gr1-BV510 | 108457 | CD24-BV510 | 101831 |
| CD45-BV605 | 103155 | Ly6G-BV605 | 127639 |
| CD19-BV650 | 115541 | CD11b-BV650 | 101259 |

### Immunophenotyping Results Summary:

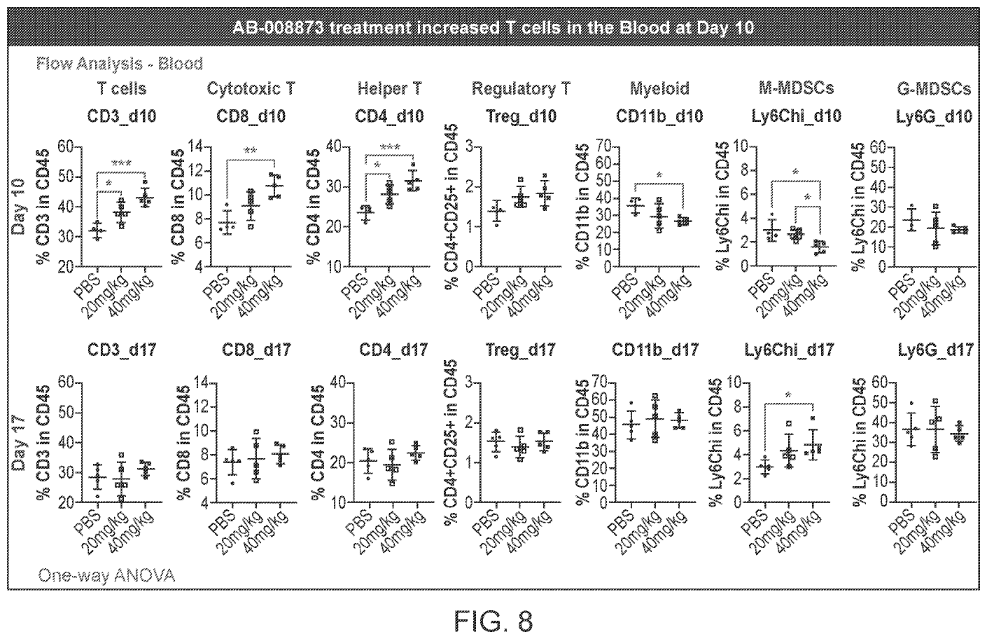

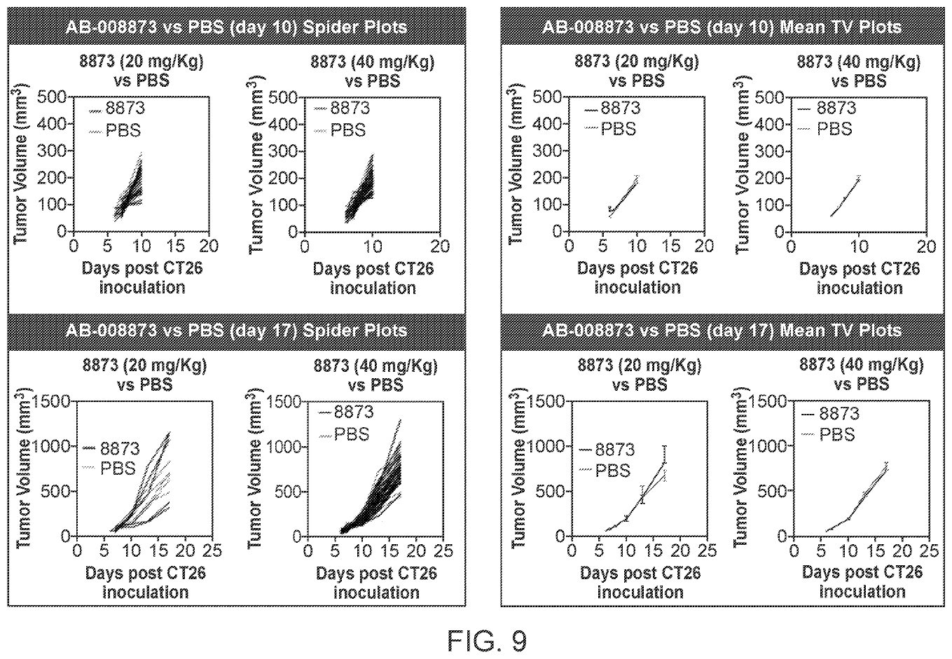

Early increases in total T cells, cytotoxic T cells, and helper T cells observed in the blood 2 days after the first dose. At the same time, decreases in overall myeloid, monocytes, or M-MDSCs were detected after treatment with 40 mg/kg AB-008873. These changes were no longer detected 9 days after the first dose and instead, an increase in CD11b+Ly6Chi monocytes or M-MDSCs was observed. FIG. 8

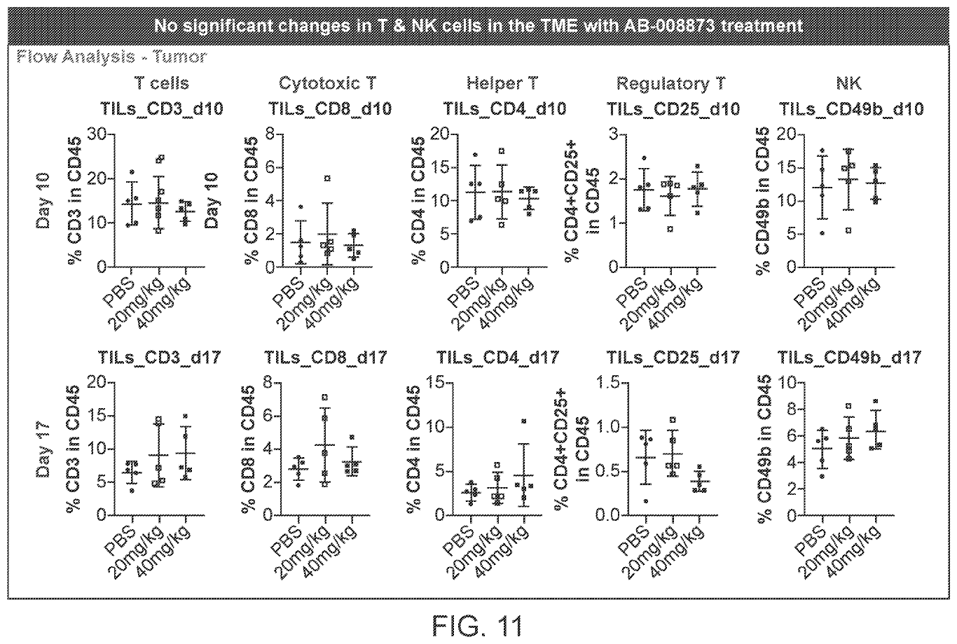

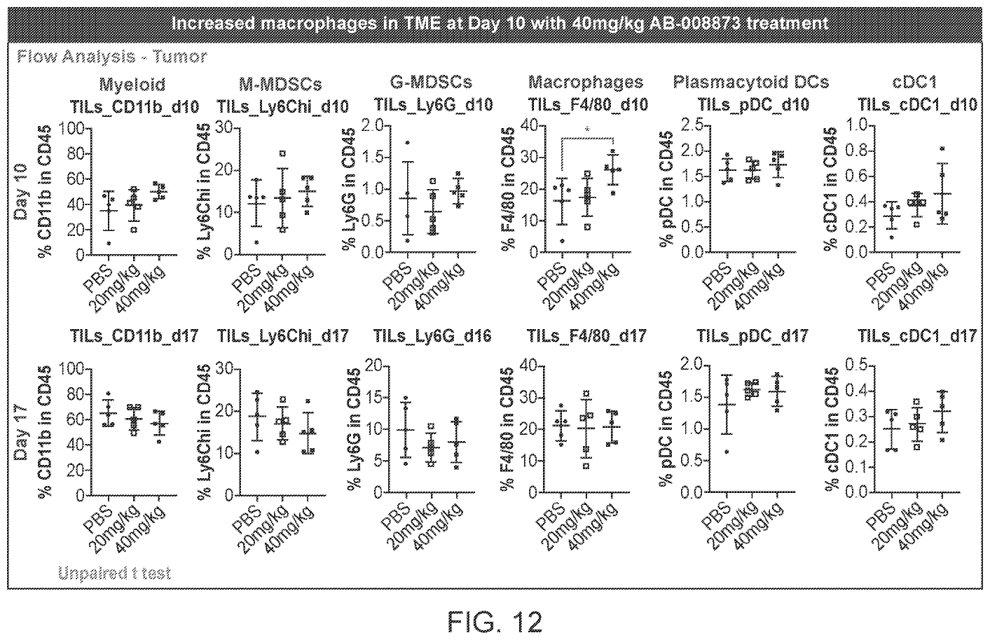

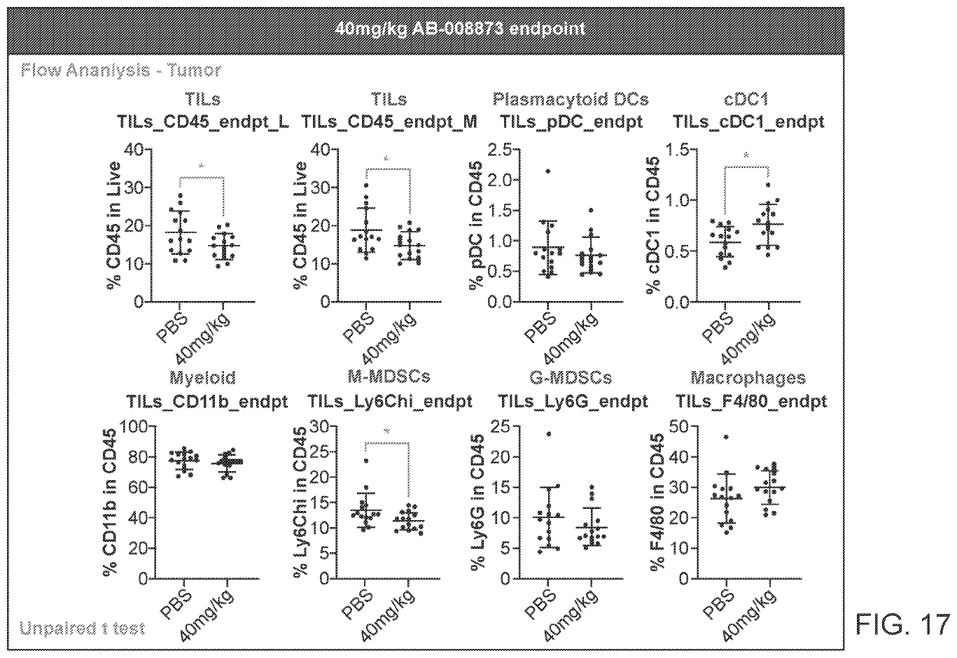

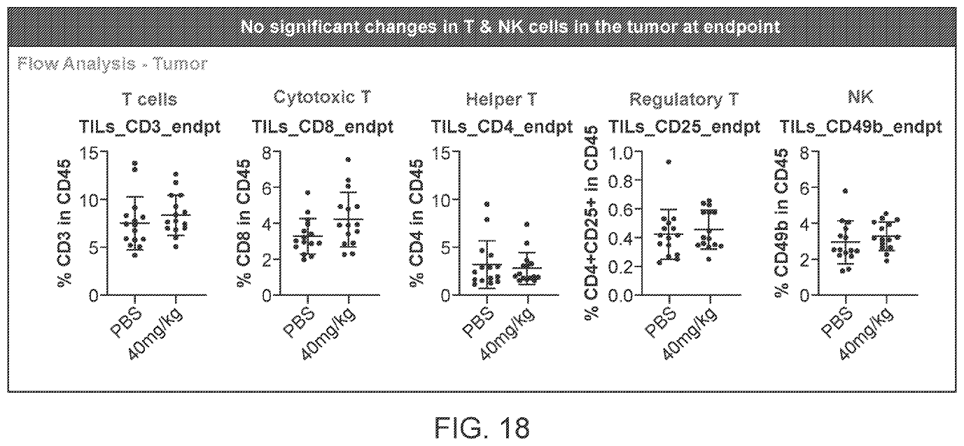

Tumors treated with AB-008873 showed no significant changes in the percentage of tumor infiltrating leukocytes (TILs) by flow cytometer at days 10 and 17 post inoculation compared to PBS treated controls. FIG. 10. Similarly, no significant changes in T cells, NK cells, monocytes/M-MDSCs, granulocytes/G-MDSCs were detected in the tumor at days 10 and 17 (FIG. 11 and FIG. 18) while total macrophages were increased at day 10 in mice treated with 40 mg/kg AB-008873. Tumors collected at endpoint and treated with 40 mg/kg AB-008873 showed evidence of decreased TILs, M-MDSCs, and slight increases in cDC1. FIG. 12 and FIG. 17.

### Binding Assays

### Flow Screen (In Vitro) Method:

Surface binding of the antibodies to in vitro grown cell lines was assessed by flow cytometry. Tumor cells were detached from their culture plate using Versene and counted. Cells were staining in BSA-containing buffer with primary antibody for 30 minutes at 4° C. with shaking. Following, cells were washed and stained with secondary PE-labeled antibody for 30 minutes at 4° C. with shaking. Before analysis on an Intellicyt iQue3 scanner, cells were counterstained with DAPI. MedFI values from live, single cells was expressed as fold over isotype control.

### Flow Screen (Ex Vivo) Method:

AB-008873 was conjugated using Thermo's SiteClick™ R-PE Antibody Labeling Kit for testing on dissociated CT26 ex vivo cells. Dissociated cells were thawed and cells were blocked with TruStain FcX™ (anti-mouse CD16/32) Antibody and stained with PE-conjugated antibodies and CD45-BV605 for 30 min at 4° C. Cells were washed 3 times with 200 l 1% FBS/1 mM EDTA/PBS and resuspended in assay buffer containing DAPI. Cells were analyzed using the Cytoflex and FlowJo_v10.7.1.

Surface binding of AB-008873 on several in vitro mouse and human tumor cell lines was assessed using flow cytometry. Among the cell lines screened, AB-008873 show binding on 786-0, A375, A549, H522, LoVo, MDA-MB-231, PC3, RKO, SKOV3, SW1116, and CT26. Additionally, AB-008873 bound CT26 ex vivo cells. FIG. 1, Tables 23 and 24.

TABLE 23
Surface binding profile of AB-008873 in human cell lines
Secondary Screens: In Vitro Cell Lines
Flow-Based Binding Assay

| 293T | 786O | A375 | A549 | BT474 | COLO205 | H522 | LoVo | MDAMB231 | PC3 | RKO | SKBR3 | SKMEL28 | SKOV3 | SW1116 | T84 |
|---|---|---|---|---|---|---|---|---|---|---|---|---|---|---|---|
| 2 | 3 | 2 | 3 | 0 | 0 | 3 | 3 | 3 | 4 | 4 | 0 | 0 | 3 | 2 | 1 |

TABLE 24
Surface binding profile of AB-008873 in mouse cell lines
Secondary Screens: In Vitro Cell Lines
Flow-Based Binding Assay

| CT26 | EMT6 | MC38 | RENCA |
|---|---|---|---|
| 2.66667 | 0.2 | 0 | 0 |

### ADCC

To detect Antibody-dependent cellular cytotoxicity (ADCC), KILR™-transfected target cells were opsonized with different concentrations of the antibody at room temperature for 20 minutes. Following, NK92 cells carrying a mouse extracellular-human intracellular CD16 chimera were added at a 5:1 ratio of effector to target cells. After a 4 h co-culture at 37° C. and 5% CO₂, pre-mixed KILR™ detection reagent was added to each well at equal volume and allowed to incubate for 30 min at room temperature in the dark before reading luminescence on a pate reader. Cytotoxicity was calculated after background (spontaneous release) subtraction and expressed as percent of a maximum lysis control.

ADCC activity of AB-008873 was assessed on several in vitro human and mouse tumor cell lines. Among the cell lines screened, AB-008873 showed dose-dependent ADCC on A549, MDA-MB-231, CT26, and to a small extent, on EMT6 cells. AB-008873 did not show any ADCC when the antibody was used up to 100 nM on SKBR3. FIG. 2A-2B and Table 25.

TABLE 25
Results of assessing the ADCC activity (FIG. 2A) and ADCP
activity (FIG. 2B) of AB-008873 in vitro functional assays.

| | | | Delta | |
|---|---|---|---|---|
| | n | EC₅₀ (nM) | Toxicity (%) | NAUC (%) |
| A549 | 15 | 15.3 ± 11.8 | 37.8 ± 19.2 | 30.7 ± 13.1 |
| MDAMB231 | 2 | 18.7 ± 1.4 | 53.2 ± 5.7 | 17.1 ± 1.7 |
| SKBR3 | 2 | >100 | 0 | 0 |
| CT26 | 4 | 10.2 ± 9.5 | 37.5 ± 5.9 | 52.6 ± 15.4 |
| EMT6 | 2 | >100 | 15.1 ± 10.6 | 7.9 ± 4.9 |

Note:
Table 25 summarizes the results in FIG. 2A. The values are shown as mean + SD. N = number of biological replicates; delta toxicity (%) = difference between lower and upper plateau of the sigmoidal dose response curve; NAUC (%) = Normalized Area Under the Curve, internal metric quantifying area under the curve of the sigmoidal dose response normalized to a known positive control.

Antibodies derived from the same lineage as AB-008873 (sibling antibodies) were tested for binding and for ADCC and ADCP activity on tumor cell line A549. Compared with AB-008873, all sibling antibodies showed less potent and less maximum binding and less ADCC and ADCP activity. Out of the sibling antibodies, AB-009806 showed the best binding and functional activity, followed by AB-009805. AB-009807 and AB-009808 showed little binding and ADCC activity but some ADCP activity. FIG. 20

Engineered variant antibodies of AB-0008873 were tested for binding and for ADCC activity on A549 tumor cells. Compared with AB-008873, AB-009815 and AB-009816 showed similar levels of binding and ADCC activity as AB-008873. AB-009812 and AB-009813 showed weaker binding and ADCC activity, followed by AB-009814. AB-009817 did not show any substantial binding or ADCC activity on A549 up to 100 nM. FIG. 21.

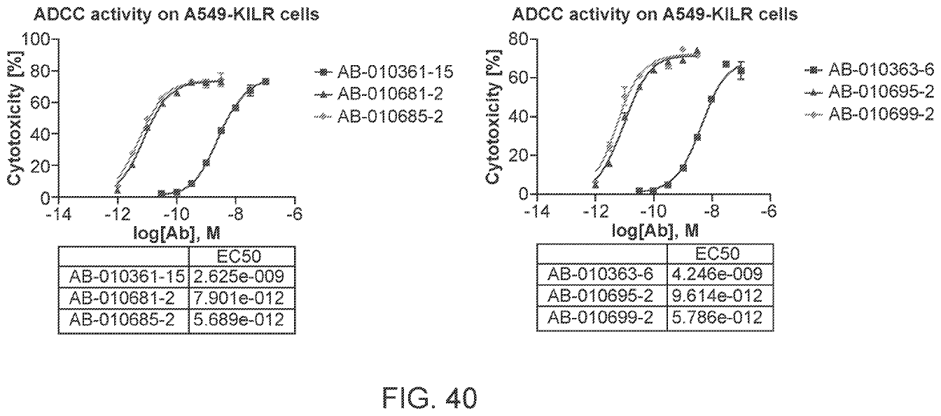

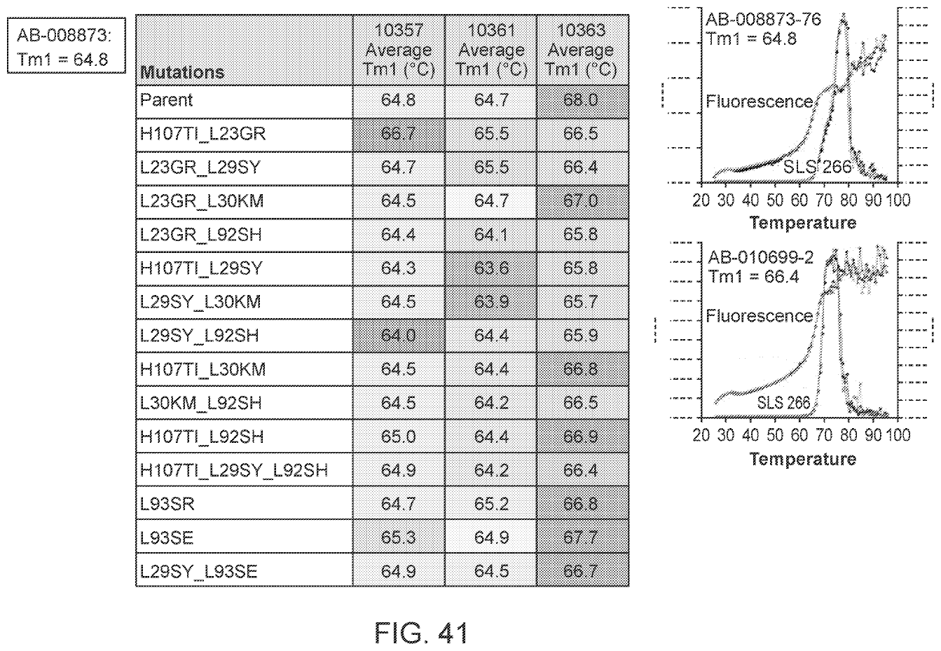

AB-010357, AB-010361, and AB-010363 and variants (Table 27) based on those antibodies exhibited ADCC activity greater than AB-008873 on the A549 tumor cells. All the variants tested exhibited ADCC activity at least as good as their parental antibody. Several variants showed a 10-to-100-fold increase in potency as compared to their parent, with some variants showed a greater than 700-fold increase. AB-010685. AB-010671, and AB-010699 had EC₅₀ values under these assay conditions of approximately 6 pM. AB-010681 and AB-010695 had EC₅₀ values of approximately 8 pM and 10 pM, respectively. FIG. 40 and Table 26. It is notable that both AB-010685 and AB-010699 have the same double mutations L29SY and L92SH. See Table 28.

TABLE 26
ADCC of anti-EphA2 antibodies

| | | | | ADCC - dAct | |
|---|---|---|---|---|---|
| | | ADCC - EC₅₀ (nM) | | rel. parent (%) | |
| AB ID | Mutations | Trial 1 | Trial 2 | Trial 1 | Trial 2 |
| AB-008873 | N/A | 24 | | | |
| AB-010357 | | 12.3 | 9.6 | 100.0 | 100.0 |
| AB-010661 | 10357_H107TI_L23GR | 5.7 | | 160.1 | |
| AB-010662 | 10357_L23GR_L29SY | 0.57 | | 166.4 | |
| AB-010663 | 10357_L23GR_L30KM | 4.7 | | 163.1 | |
| AB-010664 | 10357_L23GR_L92SH | 0.54 | | 167.7 | |
| AB-010665 | 10357_H107TI_L29SY | ~0.23 | 0.14 | 177.0 | 120.5 |
| AB-010666 | 10357_L29SY_L30KM | ~0.19 | 0.16 | 193.7 | 132.8 |
| AB-010667 | 10357_L29SY_L92SH | <0.03 | 0.012 | 154.8 | 108.9 |
| AB-010668 | 10357_H107TI_L30KM | 3.7 | | 162.9 | |
| AB-010669 | 10357_L30KM_L92SH | 0.44 | | 177.7 | |
| AB-010670 | 10357_H107TI_L92SH | 0.36 | | 178.2 | |
| AB-010671 | 10357_H107TI_L29SY_L92SH | <0.03 | 0.0064 | 157.1 | 106.7 |
| AB-010672 | 10357_L93SR | 12.4 | | 162.9 | |
| AB-010673 | 10357_L93SE | 3.9 | | 166.4 | |
| AB-010674 | 10357_L29SY_L93SE | ~0.13 | 0.10 | 171.2 | 97.2 |
| AB-010361 | | 4.0 | 2.2 | 100.0 | 100.0 |
| AB-010675 | 10361_H107TI_L23GR | 1.5 | | 122.5 | |
| AB-010676 | 10361_L23GR_L29SY | ~0.1 | 0.039 | 121.6 | 88.7 |
| AB-010677 | 10361_L23GR_L30KM | 1.6 | | 165.5 | |
| AB-010678 | 10361_L23GR_L92SH | ~0.14 | 0.047 | 154.2 | 93.9 |
| AB-010679 | 10361_H107TI_L29SY | ~0.15 | 0.040 | 171.5 | 102.9 |
| AB-010680 | 10361_L29SY_L30KM | ~0.081 | 0.031 | 161.1 | 106.1 |
| AB-010681 | 10361_L29SY_L92SH | <0.03 | 0.0079 | 152.1 | 104.6 |
| AB-010682 | 10361_H107TI_L30KM | 3.2 | | 147.9 | |
| AB-010683 | 10361_L30KM_L92SH | ~0.14 | 0.046 | 174.8 | 100.0 |
| AB-010684 | 10361_H107TI_L92SH | ~0.14 | 0.048 | 166.6 | 115.4 |
| AB-010685 | 10361_H107TI_L29SY_L92SH | <0.03 | 0.0057 | 161.1 | 106.4 |
| AB-010686 | 10361_L93SR | 6.4 | | 132.3 | |
| AB-010687 | 10361_L93SE | 2.8 | | 166.8 | |
| AB-010688 | 10361_L29SY_L93S | ~0.052 | 0.032 | 129.9 | 108.0 |
| AB-010363 | | 5.1 | 4.2 | 100.0 | 100.0 |
| AB-010689 | 10363_H107TI_L23R | 2.5 | | 88.0 | |
| AB-010690 | 10363_L23GR_L29S | 0.19 | | 112.4 | |
| AB-010691 | 10363_L23GR_L30K | 2.2 | | 85.3 | |
| AB-010692 | 10363_L23GR_L92S\ | 0.21 | | 105.0 | |
| AB-010693 | 10363_H107TI_L29 | 0.15 | | 129.0 | |
| AB-010694 | 10363_L29SY_L30K | 0.14 | | 131.3 | |
| AB-010695 | 10363_L29SY_L92SH | ~0.021 | 0.0096 | 148.3 | 118.8 |
| AB-010696 | 10363_H107TI_L30KM | 1.9 | | 114.7 | |
| AB-010697 | 10363_L30KM_L92SH | 0.30 | | 147.1 | |
| AB-010698 | 10363_H107TI_L92SH | 0.23 | | 142.1 | |
| AB-010699 | 10363_H107TI_L29SY_L92SH | ~0.014 | 0.0058 | 152.5 | 114.6 |
| AB-010700 | 10363_L93SR | 9.5 | | 75.7 | |
| AB-010701 | 10363_L93SE | 3.0 | | 99.2 | |
| AB-010702 | 10363_L29SY_L93SE | 0.065 | | 124.7 | |

TABLE 27
Sequence features of selected anti-EphA2 antibody variants

| | Variant based | Variant based | Variant based |
|---|---|---|---|
| | on AB-010357 | on AB-010361 | on AB-010363 |
| H107TI_L23GR | AB-010661 | AB-010675 | AB-010689 |
| L23GR_L29SY | AB-010662 | AB-010676 | AB-010690 |
| L23GR_L30KM | AB-010663 | AB-010677 | AB-010691 |
| L23GR_L92SH | AB-010664 | AB-010678 | AB-010692 |
| H107TI_L29SY | AB-010665 | AB-010679 | AB-010693 |
| L29SY_L30KM | AB-010666 | AB-010680 | AB-010694 |
| L29SY_L92SH | AB-010667 | AB-010681 | AB-010695 |
| H107TI_L30KM | AB-010668 | AB-010682 | AB-010696 |
| L30KM_L92SH | AB-010669 | AB-010683 | AB-010697 |
| H107TI_L92SH | AB-010670 | AB-010684 | AB-010698 |
| H107TI_L29SY_L92SH | AB-010671 | AB-010685 | AB-010699 |
| L93SR | AB-010672 | AB-010686 | AB-010700 |
| L93SE | AB-010673 | AB-010687 | AB-010701 |
| L29SY_L93SE | AB-010674 | AB-010688 | AB-010702 |

TABLE 28
Activities of anti-EphA2 antibodies

| | Variant based | Variant based | Variant based |
|---|---|---|---|
| | on AB-010357 | on AB-010361 | on AB-010363 |
| Mutations | EC₅₀ (nM) | EC₅₀ (nM) | EC₅₀ (nM) |
| Parent (AB-010357, | 10.95 | 3.1 | 4.65 |
| AB-010361, or | | | |
| AB-010363) | | | |
| H107TI_L23GR | 5.7 | 1.5 | 2.5 |
| L23GR_L29SY | 0.57 | 0.039 | 0.19 |
| L23GR_L30KM | 4.7 | 1.6 | 2.2 |
| L23GR_L92SH | 0.54 | 0.047 | 0.21 |
| H107TI_L29SY | 0.14 | 0.04 | 0.15 |
| L29SY_L30KM | 0.16 | 0.031 | 0.14 |
| L29SY_L92SH | 0.012 | 0.0079 | 0.0096 |
| H107TI_L30KM | 3.7 | 3.2 | 1.9 |
| L30KM_L92SH | 0.44 | 0.046 | 0.30 |
| H107TI_L92SH | 0.36 | 0.048 | 0.23 |
| H107TI_L29SY_L92SH | 0.0064 | 0.0057 | 0.0058 |
| L93SR | 12.4 | 6.4 | 9.5 |
| L93SE | 3.9 | 2.8 | 3.0 |
| L29SY_L93SE | 0.10 | 0.032 | 0.065 |

AB-010699 and AB-010361 exhibited greater ADCC activity on A549 cells than AB-010018, while both AB-010699 and AB-010695 were more toxic than cetuximab on A549 cells. FIGS. 50A and 50B. AB-010699 showed several thousand-fold higher ADCC activity on A549 cells and higher thermostability (as reflected in a 2.0° C. higher Tm1) as compared to AB-008873. AB-010685 was also highly potent on PC3-KILR cells and exhibited a 10-fold improvement in ADCC as compared to AB-010018. FIG. 52.

### ADCP

For antibody-dependent cellular phagocytosis (ADCP), the cell membranes of target cells were labeled with green fluorescent membrane dye preceding opsonization with different concentrations of antibody at room temperature for 30 minutes. RAW264.7 mouse macrophage (effector) cells were then added at a 1:1 ratio with the target cells and co-cultured at 37° C. and 5% CO₂ for 2 hours. After incubation, the cell mixture was dissociated with 2 mM ethylenediaminetetraacetic acid (EDTA, pH 7.4). The cell suspension was then washed several times in an isotonic salt solution and probed with a commercially available anti-CD11b antibody, conjugated with Allophycocyanin (APC). After a 30-minute incubation at 4° C. with this antibody, the cell suspension was washed again before being analyzed by flow cytometry. Cytotoxicity was calculated as the fraction of effector cells recorded that also displayed signal for green fluorescent dye and was expressed as a percentage of the total effector cell population.

The activity of AB-008873 was tested on A549 and CT26 cells. The results, as shown in FIG. 2B, demonstrated dose-dependent activity in both models.

AB-008873 was tested for ADCP activity on A549 cells alongside its siblings. All siblings showed reduced activity compared to AB-008873. FIG. 20.

### ADC

AB-008873 was tested for its antibody-drug conjugate activity using a secondary, toxin-conjugated antibody. In brief, target cells were detached from the culture plate and cell concentration was adjusted to 31,250 cells/mL in assay media. 2,500 cells were added to each well of a 96 well plate and incubated with different concentrations of primary antibody for 15 min at room temperature. Following, secondary Fab anti-mouse IgG Fc conjugated to Duocannycin with a cleavable linker (Moradec, #AM-202-DD) was added at a final concentration of 250 ng/mL. Cells were incubated for 72 h at 37° C. and 5% CO₂. At the end of the incubation period, 100 μl CellTiter-Glo® was added to each well and allowed to incubate for 5-10 min at room temperature before reading luminescence in a BMG ClarioSTAR plate reader. Data was then normalized to a maximum lysis control and plotted using graph pad prism.

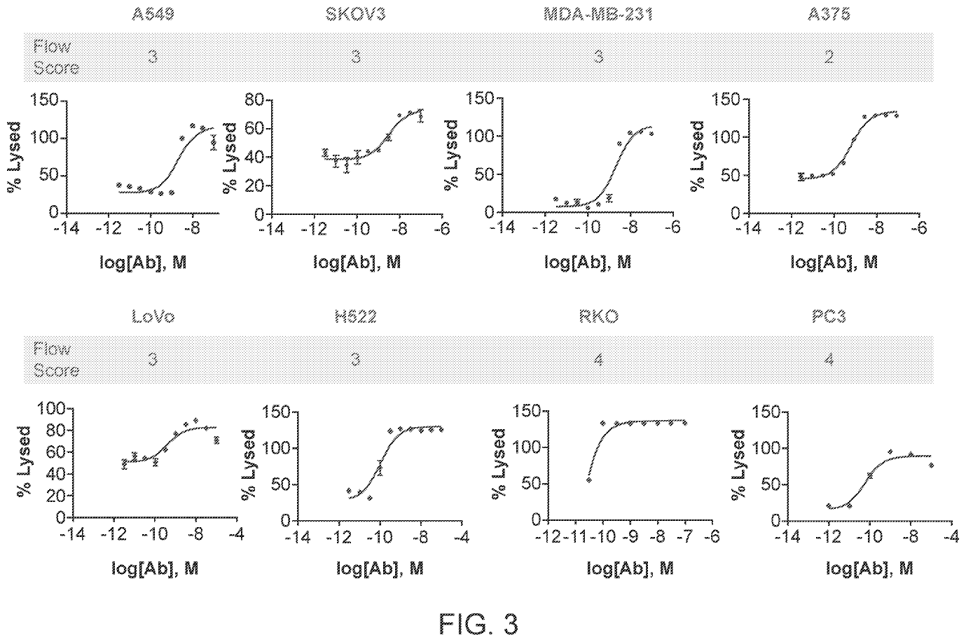

The ADC activity of AB-008873 was assessed on several in vitro human and mouse tumor cell lines. Among the cell lines screened, AB-008873 showed dose dependent ADC on A549, SKOV3, MDA-MB-231, A375, LoVo, H522, RKO, and PC3. As shown in FIG. 3, AB-008873 demonstrated ADC activity on these tumor cells.

The ADC activity of AB-008873 was compared to the engineered variant AB-009815 (H76PT mutation) on H522, LoVo, A375, SKOV3, A549, and MDA-MB-231 cells. AB-009815 showed similar activity compared to AB-008873 in all cell lines tested. As shown in FIG. 35 show that the ADC activity of AB-009815 is substantially identical to AB-008873 on these tumor cells.

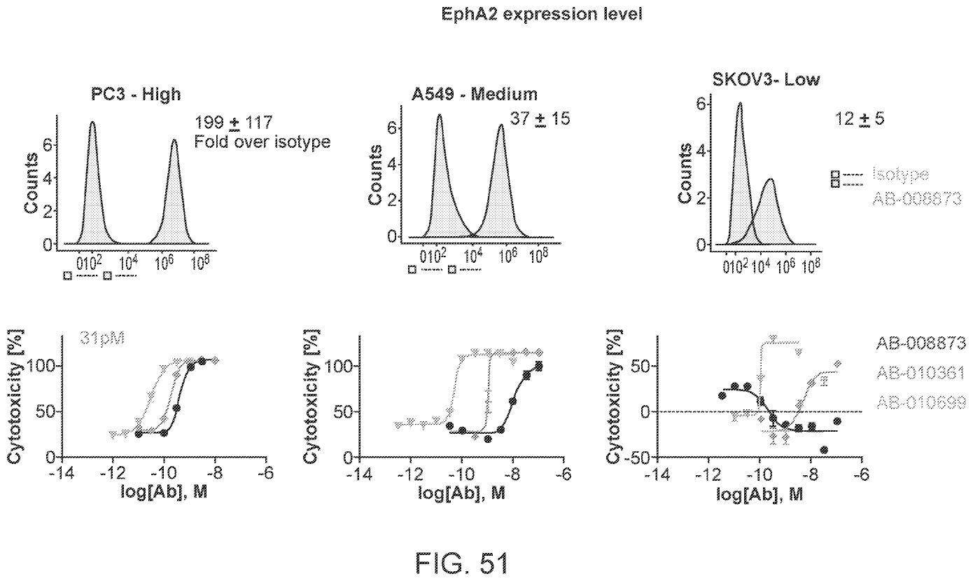

The ADC activity of AB-010361 and AB-010699 was assayed as described above and was compared to that of AB-008873, with the results shown in FIG. 51. Both AB-010361 and AB-010699 had similar ADC activity as AB-008873 in cells expressing high levels of EphA2 and exhibited enhanced ADC activity in cells expressing low levels of EphA2.

### Thermostability

AB-010357, AB-010361, and AB-010363 and variants based on those antibodies were assayed for thermostability using an Unchained Labs UNcle instrument. The concentration of purified antibodies was adjusted as needed to between 0.2 mg/ml and 0.5 mg/ml in PBS immediately prior to analysis. To determine melting temperature (Tm) intrinsic protein fluorescence was measured at 473 nm every 1.1° C. as temperature was increased linearly from 25° C. to 95° C. at a rate of 0.3° C./min. The UNcle Analysis software (version 4.01) was used to find the Tm as the first derivative of the barycentric mean (BCM). All antibodies tested were at least as stable as AB-008873, with AB-010363 and variants thereof exhibiting improved thermostability as indicated by the increased Tm1. Table 29 provides thermostability of anti-EphA2 antibodies.

TABLE 29
Thermostability of anti-EphA2 antibodies

| | Variant based | Variant based | Variant based |
|---|---|---|---|
| | on AB-010357 | on AB-010361 | on AB-010363 |
| | Average Tm1 | Average Tm1 | Average Tm1 |
| Mutations | (° C.) | (° C.) | (° C.) |
| Parent (AB-010357, | 64.8 | 64.7 | 68.0 |
| AB-010361, or | | | |
| AB-010363) | | | |
| H107TI_L23GR | 66.7 | 65.5 | 66.5 |
| L23GR_L29SY | 64.7 | 65.5 | 66.4 |
| L23GR_L30KM | 64.5 | 64.7 | 67.0 |
| L23GR_L92SH | 64.4 | 64.1 | 65.8 |
| H107TI_L29SY | 64.3 | 63.6 | 65.8 |
| L29SY_L30KM | 64.5 | 63.9 | 65.7 |
| L29SY_L92SH | 64.0 | 64.4 | 65.9 |
| H107TI_L30KM | 64.5 | 64.4 | 66.8 |
| L30KM_L92SH | 64.5 | 64.2 | 66.5 |
| H107TI_L92SH | 65.0 | 64.4 | 66.9 |
| H107TI_L29SY_L92SH | 64.9 | 64.2 | 66.4 |
| L93SR | 64.7 | 65.2 | 66.8 |
| L93SE | 65.3 | 64.9 | 67.7 |
| L29SY_L93SE | 64.9 | 64.5 | 66.7 |

### Example 4. In Vivo Studies

### Tumor Cell Implantation/Inoculation

Mouse CT26 tumor cells were propagated in culture by passaging cells every 2 to 3 days (1:10 subcultures) for 6 passages. On the day of inoculation, cells were collected, counted, and diluted to 5×10⁶ cells/mL in RPMI medium without supplements. Cell viability was recorded as 86.8% at lime of inoculation.

Female, 6-week-old BALB/c mice were inoculated in the right hind flank by subcutaneous injection with 1×10⁶ CT26 cells/mouse in 0.2 mL Waymouth's media without supplements. The day of cell inoculation was designated as Study Day 0.

### Randomization

Mice were inoculated with CT26 tumor cells on Study Day 0. To achieve study groups with consistent and homogenous tumor volumes an overage of approximately 50% was included. Mouse tumors routinely became visible and palpable five days after cell inoculation. Tumor volumes were measured twice prior to randomization. On Study Day 9, mice were randomized to ensure homogenous tumor volumes using the ‘matched distribution’ randomization function of the StudyLog lab animal management software. Mice with pre-ulcerated tumors, irregular shaped tumors, or multiple tumors were excluded from randomization. Animal IDs were assigned randomly within each treatment group on the day of randomization.

### Test Article Dosing

Test article AB-008873 was administered starting on Day 8 by intraperitoneal (IP) injection twice weekly at 20 or 40 mg/kg based on group mean body weight. Mice received either 1 or 3 doses before they were removed from study for analysis two days after dosing (Day 10 or Day 17, respectively).

Mice in the vehicle control groups were dosed at 10 mL/kg DPBS based on group mean body weight using the same dosing schedule as the test article.

### Termination

Mice were euthanized by (1) carbon dioxide asphyxiation followed by cervical dislocation or (2) isoflurane inhalation and cardiac puncture followed by cervical dislocation at predetermined time points (i.e., Day 10 or Day 17).

Several assays were performed to analyze the effect of the antibody. Flow cytometry was used to analyze the immune profiling of the blood and tumor. Tumor volumes were measured. Semi-quantitative estimates of tumor infiltrates were generated using immunofluorescence staining.

### Tissue Harvest for Immunofluorescence

Mice were euthanized by carbon dioxide asphyxiation followed by cervical dislocation. Tumors were excised from the mice, weighed, and placed in pre-filled tubes containing 10% neutral buffered formalin (Caplugs). Tumors were fixed for 24 hours at room temperature and then transferred to 80% ethanol prior to slicing and embedding in paraffin.

### Immunofluorescence Analysis

### Tumor Processing for Immunofluorescence Analysis

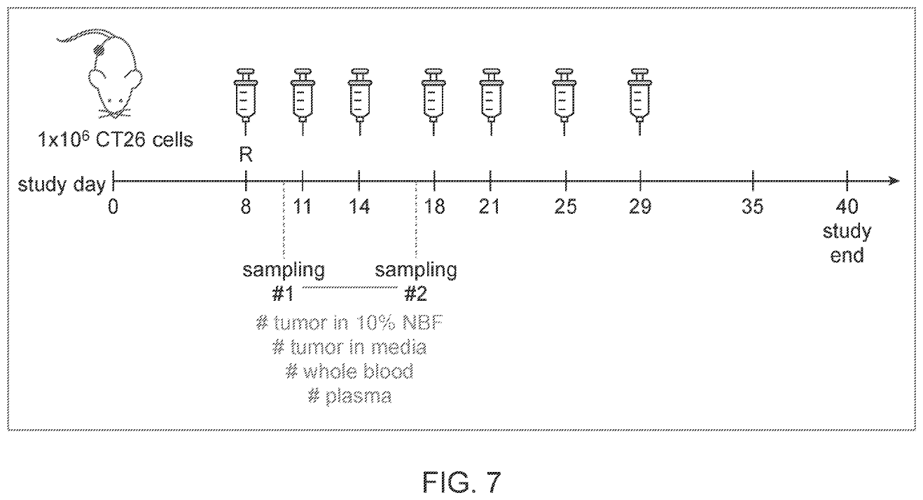

Each fixed tumor was dissected using a slicer matrix with 2.0 or 3.0 mm section slide intervals (Zivic 5526 or 5527), to allow for assessment of tumor microenvironment at 3 to 6 evenly spaced locations to account for tumor heterogeneity. The resultant tissue slices from each tumor were placed in tissue cassettes (up to three tumors in one cassette; including a piece of mouse spleen as a landmark for orientation) and subsequently processed in graded alcohols and xylene and embedded in paraffin per manufacturer's instructions using a Tissue Tek VIP® 6 A1 Vacuum Infiltration Processor (Sakura) and Tissue-Tek® TEC™ 5 embedding console system (Sakura). Blocks containing tumors were sectioned at 5 μm using a Accu-Cut® SRM™ 200 Rotary Microtome (Sakura). Sections were mounted to slide and dried. In one exemplary procedure, samples (e.g., tumor samples) were sectioned by longitudinal slicing into 2 or 3 mm thick slices using a mold. Sections are then embedded face down for examination. This approach is ideal for assessing infiltrates across the tumor and fitting multiple tumors per block. FIG. 7 and Table 30 show the procedures and reagents used in an in vivo study to assess the effect of the AB-008873 treatment to mice.

TABLE 30
Reagents and dosing information

| | | | dose level | Dose | # of | | |
|---|---|---|---|---|---|---|---|
| n▪ | Test Art. | Lot# | (mg/kg) | Freq. | Doses | Route | MODEL |
| 15 | AB-008873 | 2 | 40 | 2/Wk | 7 | i.p. | CT26 |
| 15 | PBS | 12620008 | 0 | 2/Wk | 7 | i.p. | CT26 |
| 5 | AB-008873 | 2 | 20 | 2/Wk | 1 | i.p. | CT26 |
| 5 | AB-008873 | 2 | 40 | 2/Wk | 1 | i.p. | CT26 |
| 5 | PBS | 12020008 | 0 | 2/Wk | 1 | i.p. | CT26 |
| 5 | AB-008873 | 2 | 20 | 2/Wk | 3 | i.p. | CT26 |
| 5 | AB-008873 | 2 | 40 | 2/Wk | 3 | i.p. | CT26 |
| 5 | PBS | 12620008 | 0 | 2/Wk | 3 | i.p. | CT26 |

### Immunofluorescence Staining

Immunofluorescence staining was performed on 5 μm formalin-fixed paraffin-embedded tissue sections of mouse CT26 tumors following standard immunostaining protocols. The primary antibodies were utilized with species-specific secondary antibodies and detected using standard methodology.

The tissue sections were baked at 65° C. for 30 minutes, dewaxed in xylene and rehydrated. Antigen retrieval was performed under high pressure either at 95° C. for 20 minutes or 110° C. for 15 minutes using Target Retrieval Solution (Dako) in Decloaking Chamber NxGen (Biocare Medical). The sections were blocked for 15 minutes at room temperature with Bloxall (Vector Labs).

### T Cell Marker Protocol

Dual labeling for cytotoxic T cells (“T_{cyt} cells”, which are CD3+CD8+) and regulatory T cells (T_{reg} cells, i.e., CD4+/forkhead-box protein P3; FoxP3+) populations was performed as follows. Following antigen retrieval and blocking with Bloxall, tissue sections were blocked in blocking buffer (3% bovine serum albumin with 3% normal donkey serum in 1× phosphate-buffered saline) for 30 minutes or 1 hour at room temperature. After blocking, the slides were incubated with primary antibodies against CD8 for 1 hour at room temperature, FoxP3 overnight at 4° C., or with species-appropriate isotype controls at corresponding times and temperatures. The slides were subsequently washed with Wash Buffer (Thermo-Fisher Scientific), incubated with species-specific secondary PowerVision Poly-HRP (Leica) for 30 minutes at room temperature followed by incubation with CF Tyrainide 647 (for CD8) or 488 (for FoxP3) Working Solution (Biotium) for 3 minutes at room temperature, respectively.

To provide signal in distinct channels for the second primary antibody, elution immunofluorescence was utilized through pressure and heat application. After washing and block steps, the second primary antibody against CD3 and CD4 or species-appropriate isotype control was applied to sections as describe in the previous paragraph. The slides were subsequently washed with Wash Buffer (Thermo-Fisher Scientific), incubated with species-specific secondary PowerVision Poly-HRP (Leica) for 30 minutes at room temperature followed by incubation with CF Tyramide 488 (for CD3) or 647 (for CD4) Working Solution (Biotium) for 3 minutes at room temperature, respectively.

All tissue slides were counterstained with Hoechst dye, mounted in PermaFluor Aqueous Mounting Medium (ThermoFisher Scientific), and coverslipped. Images were acquired using an AxioScan whole slide scanner (Carl Zeiss Microscopy).

### Dendritic Cell Marker Protocol

Dual labeling for dendritic cells (DC cells, i.e., CD3− CD103+) populations was performed as follows. Following antigen retrieval and blocking with Bloxall, tissue sections were blocked in blocking buffer (3% bovine serum albumin with 3% normal donkey serum in 1× phosphate-buffered saline) for 30 minutes or 1 hour at room temperature. After blocking, the slides were incubated with the primary antibody against CD103 for 1 hour at room temperature or with the species-appropriate isotype control at corresponding time and temperature. The slides were subsequently washed with Wash Buffer (Thermo-Fisher Scientific), incubated with species-specific secondary PowerVision Poly-HRP (Leica) for 30 minutes at room temperature followed by incubation with CF Tyramide 488 (for CD103) Working Solution (Biotium) for 3 minutes at room temperature.

To provide signal in distinct channels for the second primary antibody, elution immunofluorescence was utilized through pressure and heat application. After washing and block steps, the second primary antibody against CD3 or species-appropriate isotype control was applied to sections and incubated overnight at 4° C. The slides were subsequently washed with Wash Buffer (Thermo-Fisher Scientific), incubated with species-specific secondary PowerVision Poly-HRP (Leica) for 30 minutes at room temperature followed by incubation with CF Tyramide 647 (for CD3) Working Solution (Biotium) for 3 minutes at room temperature.

All tissue slides were counterstained with Hoechst dye, mounted in PermaFluor Aqueous Mounting Medium (ThermoFisher Scientific), and coverslipped. Images were acquired using an AxioScan whole slide scanner (Carl Zeiss Microscopy).

### Macrophage Marker Protocol

Following antigen retrieval and blocking with Bloxall, tissue sections were successively blocked for 15 minutes with Avidin block (Biocare), Biotin block (Biocare), and blocking buffer (3% bovine serum albumin with 3% normal donkey serum in 1×PBS) for 1 hour at room temperature. After blocking, the slides were incubated with primary antibodies against F4/80 and inducible nitric oxide synthase (iNOS), F4/80 and Arginase-1 (Arg-1), or species-appropriate isotype controls overnight at 4° C. The next day, slides were washed with Wash Buffer (Thermo-Fisher Scientific), incubated for 30 minutes at room temperature with species-specific (for F4/80 and IgG) secondary antibody conjugated to biotin and then 5 minutes at room temperature with Streptavidin Conjugate CF® 647 Working Solution (Biotium). The slides were subsequently washed twice with Wash Buffer, incubated with species-specific (for iNOS/Arg-1 and IgG) secondary PowerVision Poly-HRP for 30 minutes at room temperature followed by incubation with CF Tyramide 488 Working Solution (Biotium) for 3 minutes at room temperature.

All tissue slides were counterstained with Hoechst dye, mounted in PermaFluor Aqueous Mounting Medium (ThermoFisher Scientific), and coverslipped. Images were acquired using an AxioScan whole slide scanner (Carl Zeiss Microscopy).

### Algorithm-Based Digital Image Analysis

Positive immunoreactivity was assessed by analyzing whole slide images (Zeiss AxioScan) using Indica Labs HALO software through application of the HighPlex FL v3.2.1 algorithm to identify the double positive cell populations (Table 31). For each of the two to six tumor tissue sections per tumor, regions of interest were manually annotated, while peripheral tissues such as skin, adipose, and connective tissues as well as tissue folds and other artifacts were excluded. Individual cells were detected based on HOECHST nuclear stain. Detection thresholds for fluorescent signals were adjusted based on antibody-specific signal strength and nonspecific background signal for each experiment. Cell count estimates were determined for each tumor and normalized to cell counts observed in the corresponding IgG antibody control-stained tumor.

TABLE 31
Marker Phenotype for Immune Cell Subsets

| Cell Type | Marker Phenotype |
|---|---|
| T_{cyt} cells | CD3⁺ CD8⁺ |
| T_{reg} cells | CD4⁺ FoxP3⁺ |
| Dendritic cells | CD3− CD103+ |
| M1 macrophages | F4/80⁺ iNOS⁺ |
| M2 macrophages | F4/80⁺ Arg-1⁺ |

Arg-1 = arginase-1;
CD = cluster of differentiation;
FoxP3 = forkhead-box protein P3;
iNOS = inducible nitric oxide synthase;
M1 = M1-type (classically activated) macrophage;
M2 = M2-type (alternatively activated) macrophage;
T_{cyt} cell = cytotoxic T cell;
T_{reg} cell = regulatory T cell

### Results

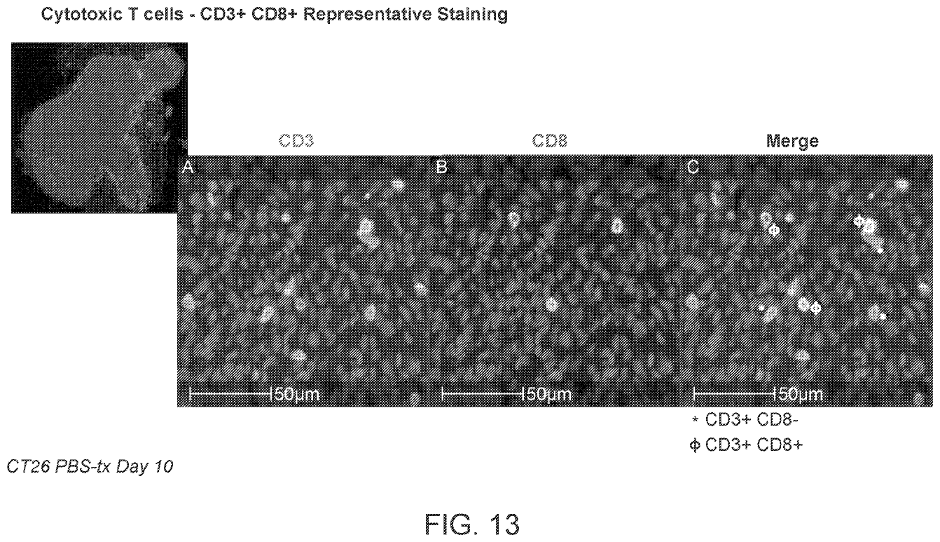

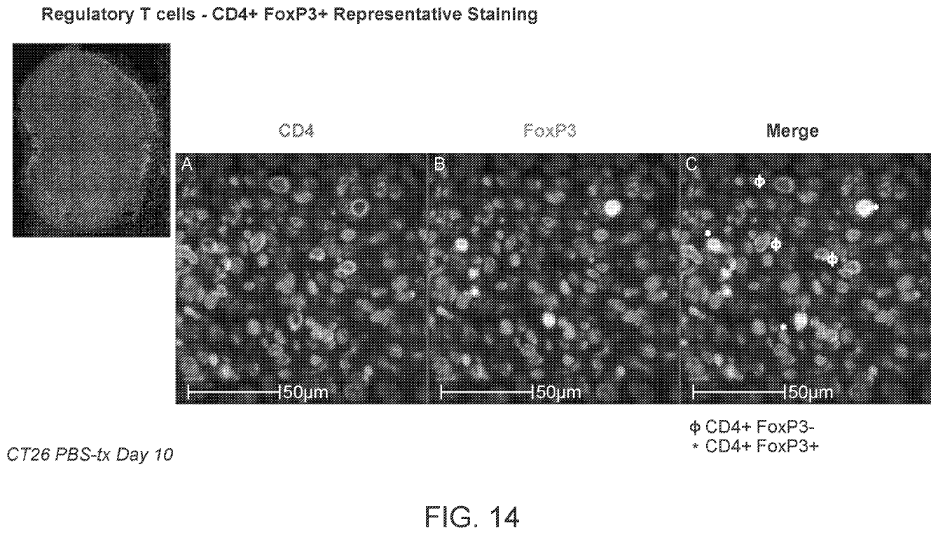

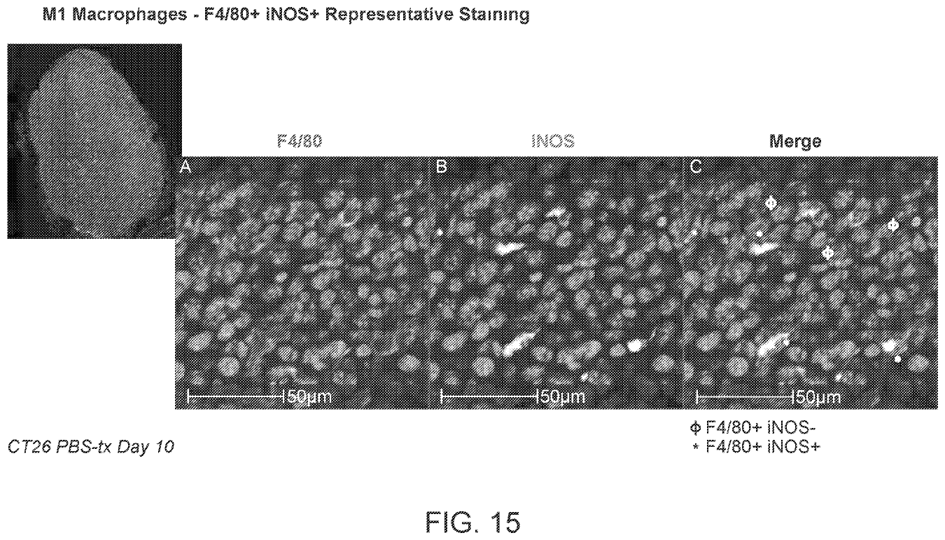

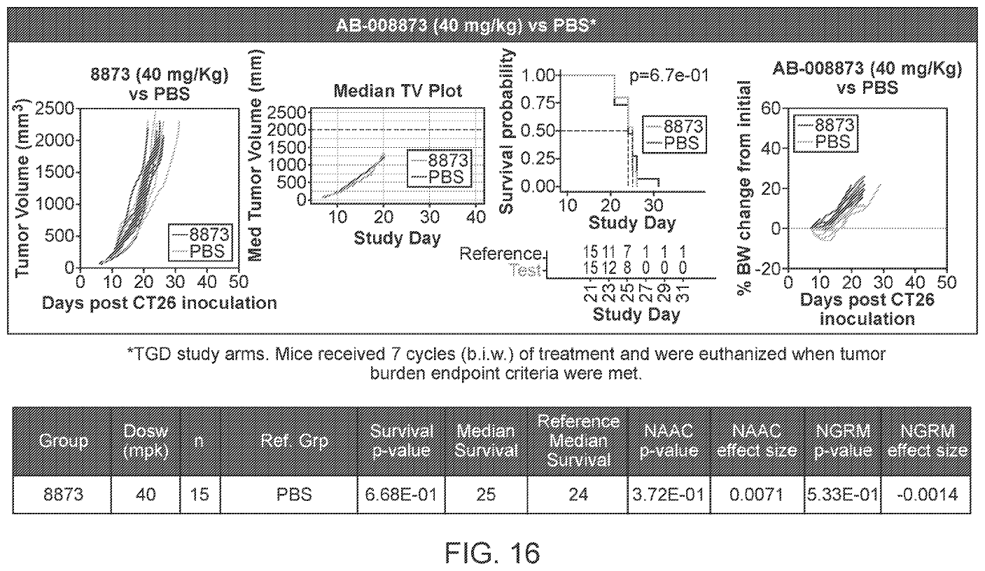

A significant increase in CD3+ and CD4+ cell populations with a concomitant decrease in Treg cells was observed in CT26 tumors from mice treated with 40 mg/kg AB-008873 vs PBS by 10 days after administration of the antibody. FIGS. 13-15. By Day 17, a significant increase in cytotoxic T cells and M1 macrophages (i.e., pro-inflammatory macrophages) were noted in CT26 tumors from mice treated with AB-008873 vs. PBS. The results are summarized in Tables 32 and 33. AB-008873 did not demonstrate significant anti-tumor activity vs PBS at either 20 mg/kg or 40 mg/kg. The results also show that the AB-008873 treatment showed early transient changes in blood and that the AB-008873 treatment induced TME changes associated with anti-tumorigenic, pro-inflammatory changes vs PBS in the CT26 model. FIG. 9 and FIG. 16.

TABLE 32
Changes in % of CD45 by Flow cytometry with AB-008873 treatment

| | | | | | | | M- | G- | | | |
|---|---|---|---|---|---|---|---|---|---|---|---|
| 40 mg/kg | Day | TILs | T_{cyt} | Tₕₑₗₚ | T_{reg} | NK | MDSCs | MDSCs | MΦ | pDC | cDC1 |
| Blood | 10 | — | ↑* | ↑* | ns | — | ↓* | ns | — | — | — |
| | 17 | — | ns | ns | ns | — | ↑* | ns | — | — | — |
| Tumor | 10 | ns | ns | ns | ns | ns | ns | ns | ↑* | ns | ns |
| | 17 | ns | ns | ns | ns | ns | ns | ns | ns | ns | ns |
| | end | ↓* | ns | ns | ns | ns | ↓* | ns | ns | ns | ↑* |

TABLE 33
Changes by IF with AB-008873 treatment

| | Day | CD3+ | T_{cyt} | CD4+ | T_{reg} | DC | MΦ | M1 | M2 |
|---|---|---|---|---|---|---|---|---|---|
| AB-008873 | 10 | ↑* | ns | ↑* | ↓* | ns | ns | ns | ns |
| 40 mg/kg | 17 | ns | ↑* | ns | ns | ns | ns | ↑* | ns |
| AB-008873 | 10 | ns | ns | ns | ns | ns | ns | ns | ns |
| 20 mg/kg | 17 | ns | ns | ns | ns | ns | ns | ↑* | ns |

### Tissue Reactivity

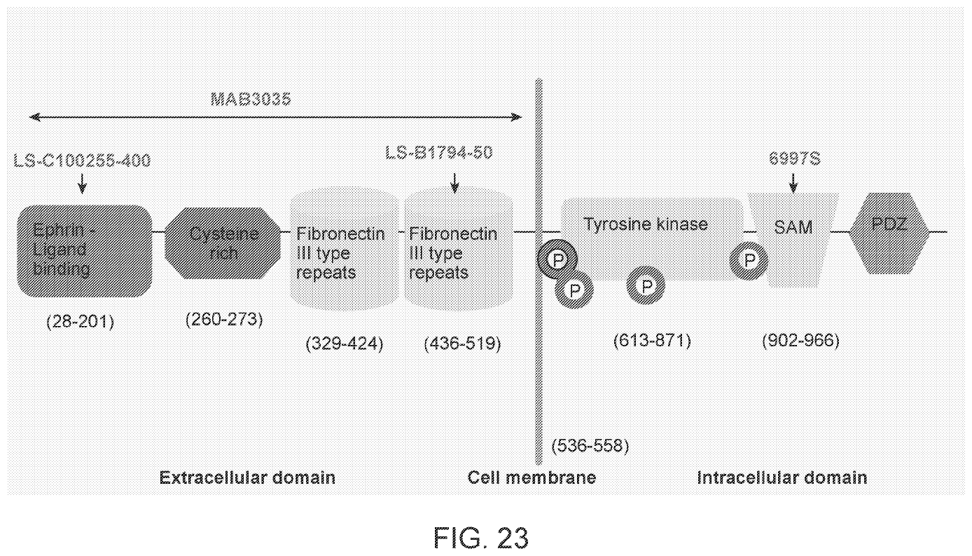

AB-008873 and its variants derived against distinct EphA2 epitopes, including AB-010016, AB-010017, and AB-010018 and MAB 3035, LS-C36249-100, LS-C100255-400, LS-B1794-50, and AB-0073254 were tested on human tumor and tumor adjacent tissues (TAT) (Table 34). FIG. 23 depicts binding sites of commercial anti-EphA2 antibodies. Typically, commercial anti-EphA2 antibodies bind to the extracellular domain or intracellular domain of Eph2A. For instance, MAB3035 (a.a. Gln25-Asn534), LS-C36249-100 (extracellular domain), LS-C100255-400 (a.a. 30-60), LS-B1794-50 (a.a. 450-500), and Ab73254 (extracellular domain) bind to sites in the extracellular domain, while 6997S (around a.a. Arg907) binds to sites in the intracellular domain. Briefly, fixed tissues were washed with 2.5% donkey serum to block non-specific binding. Sections were incubated with a primary antibody in 2.5% donkey serum diluent. Sections were incubated with Cy5 conjugated secondary antibody and counterstained with Hoechst, mounted in anti-fade mountant and stained using H&E. Tumor types and their related TAT (n=2-6 per indication) used include non-small cell lung cancer (NSCLC)-squamous cell carcinoma, NSCLC-adenocarcinoma, renal cell carcinoma (i.e., clear cell and papillary subtypes), melanoma, esophageal cancer, breast cancer (i.e., triple negative, hormone receptor-positive, and HER2-positive molecular phenotypes), colorectal cancer, soft tissue sarcoma, and pancreatic, prostate, and urothelial cancers.

TABLE 34
Antibodies tested on human tumor and tumor adjacent
tissues in immunofluorescence studies

| Category | Name | |
|---|---|---|
| 8873 and variants | AB-008873 | |
| | AB-009812 | |
| | AB-009813 | |
| | AB-009815 | |
| | AB-009816 | |
| EphA2 antibodies | AB-010016 | |
| derived from | AB-010017 | |
| clinical candidates | AB-010018 | |
| Commercial | Extracellular | MAB3035 (Gln25-Asn534) |
| anti-EphA2 Abs | | LS-C36249-100 |
| | | (extracellular domain) |
| | | LS-C100255-400 (AA 30-60) |
| | | LS-B1794-50 (AA 450-500) |
| | | Ab73254 (extracellular domain) |
| | Cytoplasmic | 6997S (around Arg907) |

In brief, commercially obtained frozen resected tumor samples and tumor adjacent tissue from non-autologous patients were sectioned to slide and lightly fixed using 4% paraformaldehyde. Following a buffer wash, the slides were incubated in a blocking reagent containing 2.5% normal donkey serum in phosphate-buffered saline (PBS), pH 7.0, at room temperature and then incubated overnight at 4° C. in primary antibody diluted in 2.5% donkey serum in PBS (concentration range of 0.1-30 μg/mL). The primary antibody is a chimeric sequence comprised of a human Fv derived from the Atreca library and a mouse IgG2a Fc sequence. After the primary antibody and subsequent wash in buffer solution, the slide is incubated in donkey anti-mouse secondary antibody conjugated to AlexaFluor 647 at room temperature. Following a wash step, the tissue is counterstained with Hoescht dye which ubiquitously labels cell nuclei, washed again, and coverslipped.

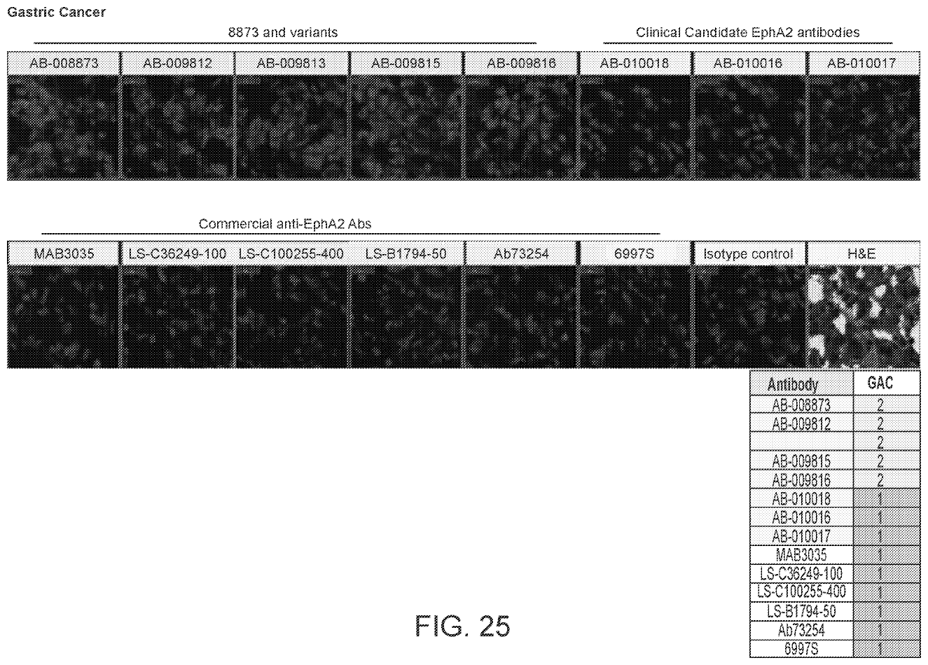

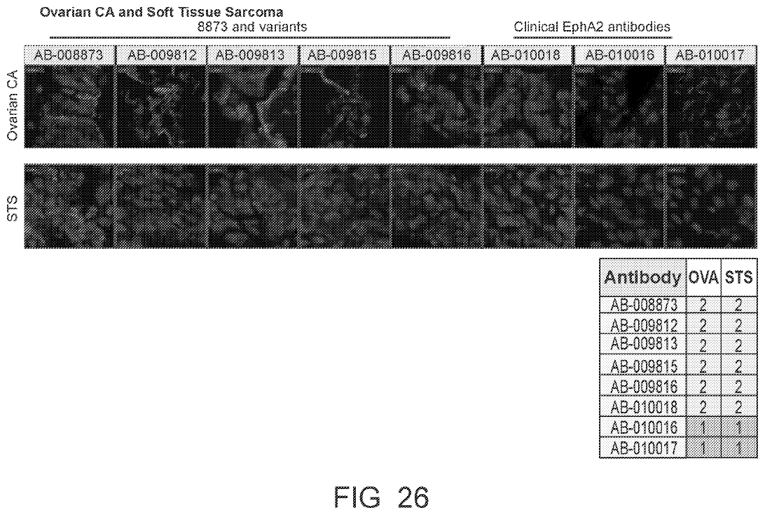

AB-008873 immunoreactivity was detected in NSCLC adenocarcinoma, melanoma, esophageal cancer, ovarian cancer, HER2+ breast cancer, soft tissue sarcoma, anal cancer, gastric cancer, uterine cancer, and head and neck cancer. FIGS. 25-30. Nearly identical profiles were detected for its engineered variant antibodies. FIGS. 25-27. AB-008873 showed approximately 80% overlap of immunoreactive tumor cores with the AB-010018 and 90% with the commercial antibody derived against the EphA2 extracellular domain. Table 35. Interestingly, in these experiments, AB-008873 signal, and that of all engineered variants, was detectable in gastric cancer whereas no reactivity was noted with anti-EphA2 compounds (i.e., antibodies AB-010018 (mouse monoclonal antibody precursor of clinical candidate humanized DS-8895a. See U.S. Pat. No. 9,150,657, SEQ ID NO: 35 and 37), AB-010016 (derived from MedImmune's clinical candidate Medi-547), and AB-010017 (derived from Merrimack's clinical candidate MM-310)) nor the commercial anti-EphA2s. FIG. 25. Furthermore, AB-008873 and its engineered variants demonstrated a punctate binding pattern across multiple cancer types including ovarian and uterine cancers and soft tissue sarcoma while any labeling by antibodies AB-010018, AB-010016, and AB-010017 or commercial antibodies was diffused with cytoplasmic signal predominant. FIGS. 26-28.

TABLE 35
IHC score of AB-008873 and its variants on 21 types of fresh frozen human tumor and TAT

| | | | IHC score | | | | | | | | | | |
|---|---|---|---|---|---|---|---|---|---|---|---|---|---|
| | | | | | RCC- | RCC- | | | | | | | |
| | | | NSCLC- | NSCLC- | clear | papil- | | | | | ER+ | Her2+ | |
| Ab Info | | | SQ | Adeno | cell | lary | MELA | ESO | OVA | TNBC | BRC | BRC | CRC |
| 8873 and | AB-008873 | | 1 | 2 | 1 | 1 | 2 | 2 | 2 | 1 | 1 | 2 | 1 |
| variants | AB-009812 | | 1 | 2 | 1 | 1 | 2 | 2 | 2 | 1 | 1 | 2 | 1 |
| | AB-009813 | | 1 | 2 | 1 | 1 | 2 | 2 | 2 | 1 | 1 | 1 | 1 |
| | AB-009815 | | 1 | 2 | 1 | 1 | 2 | 2 | 2 | 1 | 1 | 2 | 1 |
| | AB-009816 | | 1 | 2 | 1 | 1 | 2 | 2 | 2 | 1 | 1 | 2 | 1 |
| EphA2 | AB-010018 | | 2 | 2 | 1 | 1 | 3 | 2 | 2 | 2 | 2 | 2 | 1 |
| antibodies | AB-010016 | | 1 | 1 | 1 | 1 | 1.5 | 1 | 1 | 1 | 1 | 1 | 1 |
| derived from | AB-010017 | | 1 | 2 | 1 | 1 | 2 | 1 | 1 | 1 | 1 | 1 | 2 |
| clinical | | | | | | | | | | | | | |
| candidates | | | | | | | | | | | | | |
| Commercial | Extra- | MAB3035 | 2 | 2 | 1 | 1 | 3 | 2 | 2 | 1 | 1 | 1 | 1 |
| anti-EphA2 | cellular | (Gln25- | | | | | | | | | | | |
| Abs | | Asn534) | | | | | | | | | | | |
| | | LS- | 2 | 2 | 1 | 1 | 3 | 2 | 2 | 1 | 1 | 2 | 1 |
| | | C36249-100 | | | | | | | | | | | |
| | | (extra- | | | | | | | | | | | |
| | | cellular) | | | | | | | | | | | |
| | | LS- | 1 | 1 | 1 | 1 | 2 | 1 | 1 | 1 | 1 | 1 | 1 |
| | | C100255-400 | | | | | | | | | | | |
| | | (AA | | | | | | | | | | | |
| | | 30-60) | | | | | | | | | | | |
| | | LS- | 1 | 2 | 1 | 2 | 2 | 1 | 1 | 1 | 1 | 1 | 1 |
| | | B1794- | | | | | | | | | | | |
| | | 50 (AA | | | | | | | | | | | |
| | | 450- | | | | | | | | | | | |
| | | 500) | | | | | | | | | | | |
| | | Ab73254 | 2 | 2 | 1 | 1 | 2 | 1 | 1 | 1 | 1 | 1 | 1 |
| | | (extra- | | | | | | | | | | | |
| | | cellular) | | | | | | | | | | | |
| | Cytoplasmic | 6997S | 1 | 1 | 1 | 1 | 2 | 2 | 1 | 1 | 1 | 1 | 1 |
| | | (around | | | | | | | | | | | |
| | | Arg907) | | | | | | | | | | | |

| | | | IHC score | | | | | | | | | | | |
|---|---|---|---|---|---|---|---|---|---|---|---|---|---|---|
| | | | | | | | | | | | UCEC- | UCEC- | | |
| | | | | | | | | | | UCEC- | clear | papil- | | |
| Ab Info | | | PANC | PROC | STS | URO | ANC | HNC | GAC | endometrioid | cell | lary | * | ** |
| 8873 and | AB-008873 | | 1 | 1 | 2 | 1 | 2 | 2 | 2 | 1 | 2 | 2 | | 69% |
| variants | AB-009812 | | 1 | 1 | 2 | 1 | 2 | 2 | 2 | 1 | 1 | 2 | 91% | 62% |
| | AB-009813 | | 1 | 1 | 2 | 1 | 2 | 2 | 2 | 1 | 2 | 2 | 91% | 62% |
| | AB-009815 | | 1 | 1 | 2 | 1 | 2 | 2 | 2 | 1 | 2 | 2 | 100% | 69% |
| | AB-009816 | | 1 | 1 | 2 | 1 | 2 | 2 | 2 | 1 | 2 | 2 | 100% | 69% |
| EphA2 | AB-010018 | | 1 | 1 | 2 | 1 | 2 | 1 | 1 | 2 | 2 | 2 | 82% | |
| antibodies | AB-010016 | | 1 | 1 | 1 | 1 | 2 | 2 | 1 | 1 | 2 | 2 | 45% | 31% |
| derived from | AB-010017 | | 1 | 1 | 1 | 1 | 2 | 2 | 1 | 2 | 2 | 3 | 55% | 46% |
| clinical | | | | | | | | | | | | | | |
| candidates | | | | | | | | | | | | | | |
| Commercial | Extra- | MAB3035 | 2 | 1 | 1 | 1 | 2 | 2 | 1 | 2 | 2 | 2 | 73% | 69% |
| anti-EphA2 | cellular | (Gln25- | | | | | | | | | | | | |
| Abs | | Asn534) | | | | | | | | | | | | |
| | | LS- | 1 | 1 | 2 | 1 | 2 | 2 | 1 | 2 | 2 | 3 | 91% | 85% |
| | | C36249-100 | | | | | | | | | | | | |
| | | (extra- | | | | | | | | | | | | |
| | | cellular) | | | | | | | | | | | | |
| | | LS- | 1 | 1 | 1 | 1 | 1 | 1 | 1 | 1 | 2 | 2 | 27% | 23% |
| | | C100255-400 | | | | | | | | | | | | |
| | | (AA | | | | | | | | | | | | |
| | | 30-60) | | | | | | | | | | | | |
| | | LS- | 1 | 1 | 1 | 1 | 1 | 1 | 1 | 1 | 2 | 2 | 36% | 31% |
| | | B1794- | | | | | | | | | | | | |
| | | 50 (AA | | | | | | | | | | | | |
| | | 450- | | | | | | | | | | | | |
| | | 500) | | | | | | | | | | | | |
| | | Ab73254 | 1 | 1 | 1 | 1 | 1 | 1 | 1 | 1 | 2 | 1 | 27% | 31% |
| | | (extra- | | | | | | | | | | | | |
| | | cellular) | | | | | | | | | | | | |
| | Cytoplasmic | 6997S | 1 | 1 | 1 | 1 | 2 | 1 | 1 | 1 | 2 | 2 | 45% | 38% |
| | | (around | | | | | | | | | | | | |
| | | Arg907) | | | | | | | | | | | | |

*refers to % overlap of positive cancer types between Ab-008873 and other antibodies.
**refers to % overlap of positive cancer types between DS-8895 and other antibodies.

AB-010361 and AB-010363 were assayed as above. Both variants retained similar reactivity as AB-008873 to most of the tested cancer samples. However, there were some notable differences observed for reactivity to melanoma, soft tissue sarcoma, endometrioid uterine cancer, esophageal cancer, triple negative breast cancer, ovarian cancer, and urothelial cancer. AB-010361 and AB-010363 show higher binding affinity than AB-008873 on melanoma, soft tissue sarcoma, endometrioid uterine cancer. AB-010361 shows higher binding affinity than AB-008873 on esophageal cancer and triple negative breast cancer. AB-010363 shows higher binding affinity than AB-008873 on ovarian cancer, and urothelial cancer. Furthermore, AB-010361 shows enhanced reactivity in triple negative breast cancer as compared to AB-010363, but diminished reactivity as compared to AB-010363 in ovarian and urologic cancer. AB-010363 shows enhanced reactivity in urothelial cancer compared to AB-010361, AB-010018, AB-010016, and AB-010017, but diminished tumor-selective binding in esophageal cancer as compared to AB-008873. Table 36 illustrates reactivity of anti-EphA2 antibodies to cancers.

TABLE 36
Reactivity of anti-EphA2 antibodies to cancers

| | AB-008873 | | | | | AB-010361 | | | | | AB-010363 | | | | |
|---|---|---|---|---|---|---|---|---|---|---|---|---|---|---|---|
| | | | 3 | 1 | | | | 3 | 1 | | | | 3 | 1 | |
| | 30 | 10 | μg/ | μg/ | 0.3 | 30 | 10 | μg/ | μg/ | 0.3 | 30 | 10 | μg/ | μg/ | 0.3 |
| Indication | μg/ml | μg/ml | [symbol] | [symbol] | μg/ml | μg/ml | μg/ml | [symbol] | [symbol] | μg/ml | μg/ml | μg/ml | [symbol] | [symbol] | μg/ml |
| MELA | 2 | 2 | 1 | 1 | 1 | 2 | 2 | 2 | 1 | 1 | 2 | 2 | 2 | 1 | 1 |
| STS | 2 | 2 | 2 | 2 | 1 | 2 | 2 | 2 | 2 | 2 | 2 | 2 | 2 | 2 | 2 |
| UCEC- | 2 | 2 | 1 | 1 | 1 | 2 | 2 | 2 | 1 | 1 | 2 | 2 | 2 | 1 | 1 |
| [symbol] | | | | | | | | | | | | | | | |
| ESO | 2 | 2 | 1 | 1 | 1 | 2 | 2 | 2 | 2 | 1 | 1 | 1 | 1 | 1 | 1 |
| OVA | 2 | 1 | 1 | 1 | 1 | 1 | 1 | 1 | 1 | 1 | 2 | 2 | 2 | 2 | 1 |
| TNBC | 1 | 1 | 1 | 1 | 1 | 2 | 2 | 1 | 1 | 1 | 1 | 1 | 1 | 1 | 1 |
| URO | 1 | 1 | 1 | 1 | 1 | 1 | 1 | 1 | 1 | 1 | 2 | 2 | 2 | 1 | 1 |
| UCEC-clear | 2 | 2 | 2 | 2 | 2 | 2 | 2 | 2 | 2 | 2 | 2 | 2 | 2 | 2 | 2 |
| [symbol] | | | | | | | | | | | | | | | |
| UCEC- | 2 | 2 | 2 | 2 | 1 | 2 | 2 | 2 | 2 | 1 | 2 | 2 | 2 | 2 | 1 |
| [symbol] | | | | | | | | | | | | | | | |
| NSCLC-SQ | 1 | 1 | 1 | 1 | 1 | 1 | 1 | 1 | 1 | 1 | 1 | 1 | 1 | 1 | 1 |
| NSCLC- | 1 | 1 | 1 | 1 | 1 | 1 | 1 | 1 | 1 | 1 | 1 | 1 | 1 | 1 | 1 |
| [symbol] | | | | | | | | | | | | | | | |
| RCC-clear | 1 | 1 | 1 | 1 | 1 | 1 | 1 | 1 | 1 | 1 | 1 | 1 | 1 | 1 | 1 |
| [symbol] | | | | | | | | | | | | | | | |
| RCC- | 1 | 1 | 1 | 1 | 1 | 1 | 1 | 1 | 1 | 1 | 1 | 1 | 1 | 1 | 1 |
| [symbol] | | | | | | | | | | | | | | | |
| ER+ BRC | 1 | 1 | 1 | 1 | 1 | 1 | 1 | 1 | 1 | 1 | 1 | 1 | 1 | 1 | 1 |
| Her2+ BRC | 1 | 1 | 1 | 1 | 1 | 1 | 1 | 1 | 1 | 1 | 1 | 1 | 1 | 1 | 1 |
| CRC | 1 | 1 | 1 | 1 | 1 | 1 | 1 | 1 | 1 | 1 | 1 | 1 | 1 | 1 | 1 |
| PANC | 1 | 1 | 1 | 1 | 1 | 1 | 1 | 1 | 1 | 1 | 1 | 1 | 1 | 1 | 1 |
| PROC | 1 | 1 | 1 | 1 | 1 | 1 | 1 | 1 | 1 | 1 | 1 | 1 | 1 | 1 | 1 |

| | AB-010016 | | | | | AB-010018 | | | | |
|---|---|---|---|---|---|---|---|---|---|---|
| | 30 | 10 | 3 | 1 | 0.3 | 30 | 10 | 3 | 1 | 0.3 |
| Indication | μg/ml | μg/ml | μg/ml | μg/ml | μg/ml | μg/ml | μg/ml | μg/ml | μg/ml | μg/ml |
| MELA | 1 | 1 | 1 | 1 | 1 | 2 | 2 | 2 | 2 | 2 |
| STS | 2 | 1 | 1 | 1 | 1 | 2 | 2 | 2 | 2 | 2 |
| UCEC- | 1 | 1 | 1 | 1 | 1 | 2 | 2 | 2 | 2 | 2 |
| [symbol] | | | | | | | | | | |
| ESO | 1 | 1 | 1 | 1 | 1 | 2 | 2 | 2 | 2 | 2 |
| OVA | 1 | 1 | 1 | 1 | 1 | 1 | 1 | 1 | 1 | 1 |
| TNBC | 1 | 1 | 1 | 1 | 1 | 1 | 1 | 1 | 1 | 1 |
| URO | 2 | 1 | 1 | 1 | 1 | 1 | 1 | 1 | 1 | 1 |
| UCEC-clear | 2 | 1 | 1 | 1 | 1 | 2 | 2 | 2 | 2 | 2 |
| [symbol] | | | | | | | | | | |
| UCEC- | 1 | 1 | 1 | 1 | 1 | 2 | 2 | 2 | 2 | 2 |
| [symbol] | | | | | | | | | | |
| NSCLC-SQ | 1 | 1 | 1 | 1 | 1 | 1 | 1 | 1 | 1 | 1 |
| NSCLC- | 1 | 1 | 1 | 1 | 1 | 1 | 1 | 1 | 1 | 1 |
| [symbol] | | | | | | | | | | |
| RCC-clear | 1 | 1 | 1 | 1 | 1 | 1 | 1 | 1 | 1 | 1 |
| [symbol] | | | | | | | | | | |
| RCC- | 1 | 1 | 1 | 1 | 1 | 1 | 1 | 1 | 1 | 1 |
| [symbol] | | | | | | | | | | |
| ER+ BRC | 1 | 1 | 1 | 1 | 1 | 1 | 1 | 1 | 1 | 1 |
| Her2+ BRC | 1 | 1 | 1 | 1 | 1 | 1 | 1 | 1 | 1 | 1 |
| CRC | 1 | 1 | 1 | 1 | 1 | 1 | 1 | 1 | 1 | 1 |
| PANC | 1 | 1 | 1 | 1 | 1 | 1 | 1 | 1 | 1 | 1 |
| PROC | 1 | 1 | 1 | 1 | 1 | 1 | 1 | 1 | 1 | 1 |

Score of 1 indicates no signal above TAT or isotype control. [symbol]
indicates data missing or illegible when filed

TABLE 36
(Continued)

| Indication | AB-010016 | | | | | AB-010018 | | | | |
|---|---|---|---|---|---|---|---|---|---|---|
| | 30 | 10 | 3 | 1 | 0.3 | 30 | 10 | 3 | 1 | 0.3 |
| | ug/ml | ug/ml | ug/ml | ug/ml | ug/ml | ug/ml | ug/ml | ug/ml | ug/ml | ug/ml |
| MELA | 1 | 1 | 1 | 1 | 1 | 2 | 2 | 2 | 2 | 2 |
| STS | 2 | 1 | 1 | 1 | 1 | 2 | 2 | 2 | 2 | 2 |
| UCEC- | 1 | 1 | 1 | 1 | 1 | 2 | 2 | 2 | 2 | 2 |
| ESO | 1 | 1 | 1 | 1 | 1 | 2 | 2 | 2 | 2 | 2 |
| OVA | 1 | 1 | 1 | 1 | 1 | 1 | 1 | 1 | 1 | 1 |
| TNBC | 1 | 1 | 1 | 1 | 1 | 1 | 1 | 1 | 1 | 1 |
| URO | 2 | 1 | 1 | 1 | 1 | 1 | 1 | 1 | 1 | 1 |
| UCEC-clear cell | 2 | 1 | 1 | 1 | 1 | 2 | 2 | 2 | 2 | 2 |
| UCEC-papillary | 1 | 1 | 1 | 1 | 1 | 2 | 2 | 2 | 2 | 2 |
| NSCLC-SQ | 1 | 1 | 1 | 1 | 1 | 1 | 1 | 1 | 1 | 1 |
| NSCLC-Adeno | 1 | 1 | 1 | 1 | 1 | 1 | 1 | 1 | 1 | 1 |
| RCC-clear cell | 1 | 1 | 1 | 1 | 1 | 1 | 1 | 1 | 1 | 1 |
| RCC-papillary | 1 | 1 | 1 | 1 | 1 | 1 | 1 | 1 | 1 | 1 |
| ER+ BRC | 1 | 1 | 1 | 1 | 1 | 1 | 1 | 1 | 1 | 1 |
| Her2+ BRC | 1 | 1 | 1 | 1 | 1 | 1 | 1 | 1 | 1 | 1 |
| CRC | 1 | 1 | 1 | 1 | 1 | 1 | 1 | 1 | 1 | 1 |
| PANC | 1 | 1 | 1 | 1 | 1 | 1 | 1 | 1 | 1 | 1 |
| PROC | 1 | 1 | 1 | 1 | 1 | 1 | 1 | 1 | 1 | 1 |

Score of 1 indicates no signal above TAT or isotype control.

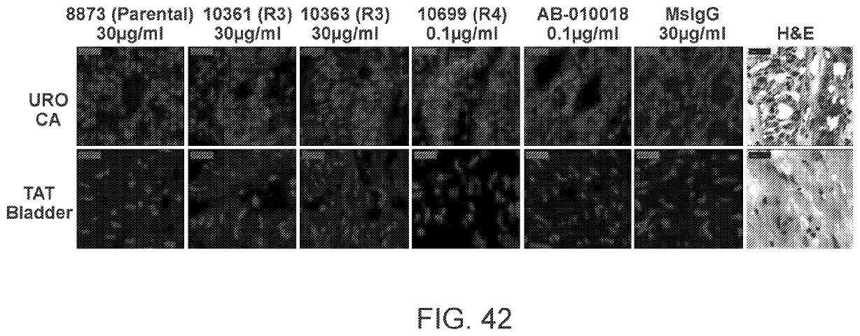

AB-010699 showed increased potency in many tumor types as compared to AB-010018 and AB-00873 and AB-010361. However, AB-010699 is less potent than its parent AB-010361 against soft tissue carcinoma in this assay. FIG. 42; Table 37, 38, and 39.

TABLE 37
Binding to EphA2 epitopes across cancer types by various EphA2 antibodies

| | AB-008873 (μg/ml) | | | | | | AB-010361 (μg/ml) | | | | | | AB-010699 (μg/ml) | | | | | | AB-010018 (μg/ml) | | | | | |
|---|---|---|---|---|---|---|---|---|---|---|---|---|---|---|---|---|---|---|---|---|---|---|---|---|
| Indication | 30 | 10 | 3 | 1 | 0.3 | 0.1 | 30 | 10 | 3 | 1 | 0.3 | 0.1 | 30 | 10 | 3 | 1 | 0.3 | 0.1 | 30 | 10 | 3 | 1 | 0.3 | 0.1 |
| NSCLC-SQ | 2 | 2 | 1 | 1 | 1 | 1 | 2 | 1 | 1 | 1 | 1 | 1 | 2 | 2 | 2 | 2 | 2 | 2 | 2 | 1 | 1 | 1 | 1 | 1 |
| NSCLC- | 2 | 1 | 1 | 1 | 1 | 1 | 2 | 2 | 1 | 1 | 1 | 1 | 2 | 2 | 1 | 1 | 1 | 1 | 1 | 1 | 1 | 1 | 1 | 1 |
| Adeno | | | | | | | | | | | | | | | | | | | | | | | | |
| RCC- | 1 | 1 | 1 | 1 | 1 | 1 | 1 | 1 | 1 | 1 | 1 | 1 | 1 | 1 | 1 | 1 | 1 | 1 | 2 | 2 | 2 | 1 | 1 | 1 |
| papillary | | | | | | | | | | | | | | | | | | | | | | | | |
| MELA | 2 | 2 | 1 | 1 | 1 | 1 | 2 | 2 | 2 | 1 | 1 | 1 | 2 | 2 | 2 | 2 | 2 | 1 | 2 | 2 | 2 | 2 | 2 | 1 |
| ESO | 2 | 1 | 1 | 1 | 1 | 1 | 2 | 1 | 1 | 1 | 1 | 1 | 2 | 2 | 1 | 1 | 1 | 1 | 2 | 1 | 1 | 1 | 1 | 1 |
| PANC | 1 | 1 | 1 | 1 | 1 | 1 | 1 | 1 | 1 | 1 | 1 | 1 | 1 | 1 | 1 | 1 | 1 | 1 | 2 | 2 | 2 | 2 | 2 | 2 |
| STS | 2 | 2 | 2 | 1 | 1 | 1 | 2 | 2 | 2 | 2 | 1 | 1 | 1 | 1 | 1 | 1 | 1 | 1 | 2 | 1 | 1 | 1 | 1 | 1 |
| URO | 1 | 1 | 1 | 1 | 1 | 1 | 1 | 1 | 1 | 1 | 1 | 1 | 2 | 2 | 2 | 2 | 2 | 2 | 2 | 2 | 2 | 2 | 2 | 2 |
| Uterine | 2 | 1 | 1 | 1 | 1 | 1 | 2 | 2 | 1 | 1 | 1 | 1 | 2 | 2 | 2 | 2 | 1 | 1 | 2 | 2 | 2 | 2 | 2 | 1 |

Score of 1 indicates no signal above TAT or isotype control.

TABLE 38
Binding to EphA2 epitopes across cancer types by various EphA2 antibodies

| | | | | | | | | | | | | | stage | | | | | | | | | | | |
|---|---|---|---|---|---|---|---|---|---|---|---|---|---|---|---|---|---|---|---|---|---|---|---|---|
| | AB-008873 (μg/ml) | | | | | | AB-010361 (μg/ml) | | | | | | AB-010699 (μg/ml) | | | | | | AB-010018 (μg/ml) | | | | | |
| Indication | 30 | 10 | 3 | 1 | 0.3 | 0.1 | 30 | 10 | 3 | 1 | 0.3 | 0.1 | 30 | 10 | 3 | 1 | 0.3 | 0.1 | 30 | 10 | 3 | 1 | 0.3 | 0.1 |
| NSCLC-SQ | 2 | 2 | 1 | 1 | 1 | 1 | 2 | 1 | 1 | 1 | 1 | 1 | 2 | 2 | 2 | 2 | 2 | 2 | 2 | 1 | 1 | 1 | 1 | 1 |
| NSCLC- | 2 | 1 | 1 | 1 | 1 | 1 | 2 | 2 | 1 | 1 | 1 | 1 | 2 | 2 | 1 | 1 | 1 | 1 | 1 | 1 | 1 | 1 | 1 | 1 |
| Adeno | | | | | | | | | | | | | | | | | | | | | | | | |
| RCC- | 1 | 1 | 1 | 1 | 1 | 1 | 1 | 1 | 1 | 1 | 1 | 1 | 1 | 1 | 1 | 1 | 1 | 1 | 2 | 2 | 2 | 1 | 1 | 1 |
| papillary | | | | | | | | | | | | | | | | | | | | | | | | |
| MELA | 2 | 2 | 1 | 1 | 1 | 1 | 2 | 2 | 2 | 1 | 1 | 1 | 2 | 2 | 2 | 2 | 2 | 1 | 2 | 2 | 2 | 2 | 2 | 1 |
| ESO | 2 | 1 | 1 | 1 | 1 | 1 | 2 | 1 | 1 | 1 | 1 | 1 | 2 | 2 | 1 | 1 | 1 | 1 | 2 | 1 | 1 | 1 | 1 | 1 |
| PANC | 1 | 1 | 1 | 1 | 1 | 1 | 1 | 1 | 1 | 1 | 1 | 1 | 1 | 1 | 1 | 1 | 1 | 1 | 2 | 2 | 2 | 2 | 2 | 2 |
| STS | 2 | 2 | 2 | 1 | 1 | 1 | 2 | 2 | 2 | 2 | 1 | 1 | 1 | 1 | 1 | 1 | 1 | 1 | 2 | 1 | 1 | 1 | 1 | 1 |
| URO | 1 | 1 | 1 | 1 | 1 | 1 | 1 | 1 | 1 | 1 | 1 | 1 | 2 | 2 | 2 | 2 | 2 | 2 | 2 | 2 | 2 | 2 | 2 | 2 |
| Uterine | 2 | 1 | 1 | 1 | 1 | 1 | 2 | 2 | 1 | 1 | 1 | 1 | 2 | 2 | 2 | 2 | 1 | 1 | 2 | 2 | 2 | 2 | 2 | 1 |

Score of 1 indicates no signal above TAT or isotype control.

TABLE 39
Binding to EphA2 epitopes across cancer types by various EphA2 antibodies

| | AB-008873 | | | | | | | | |
|---|---|---|---|---|---|---|---|---|---|
| | (parental) | | | AB-010361 | | | AB-010699 | | |
| Cancer | Reactive | Total | Reactivity | Reactive | Total | Reactivity | Reactive | Total | Reactivity |
| Indication | # | # | % | # | # | % | # | # | % |
| Esophageal | 0 | 10 | 0% | 3 | 10 | 30% | 8 | 10 | 80% |
| (Squamous) | | | | | | | | | |
| Lung | 5 | 20 | 25% | 8 | 20 | 40% | | | |
| (Squamous) | | | | | | | | | |
| Breast | 2 | 32 | 6% | 10 | 32 | 31% | | | |
| Renal | 1 | 34 | 3% | 10 | 34 | 29% | | | |
| Ovarian | 2 | 36 | 6% | 6 | 36 | 17% | | | |

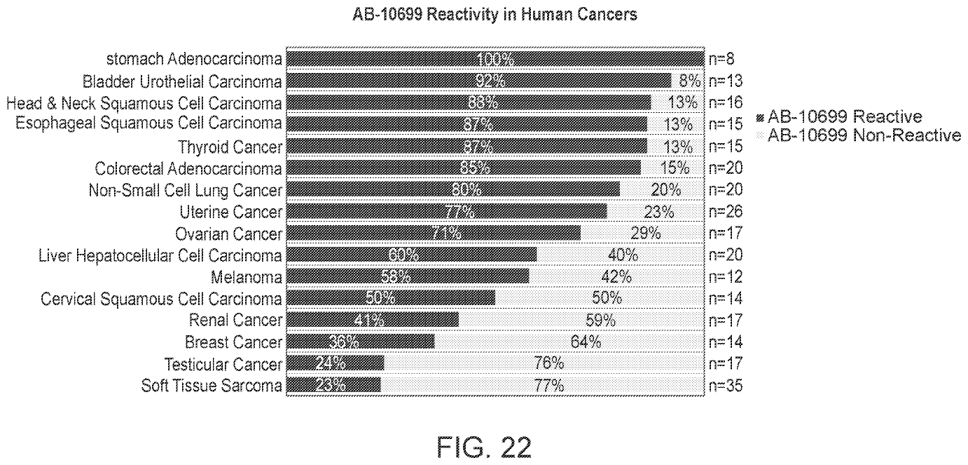

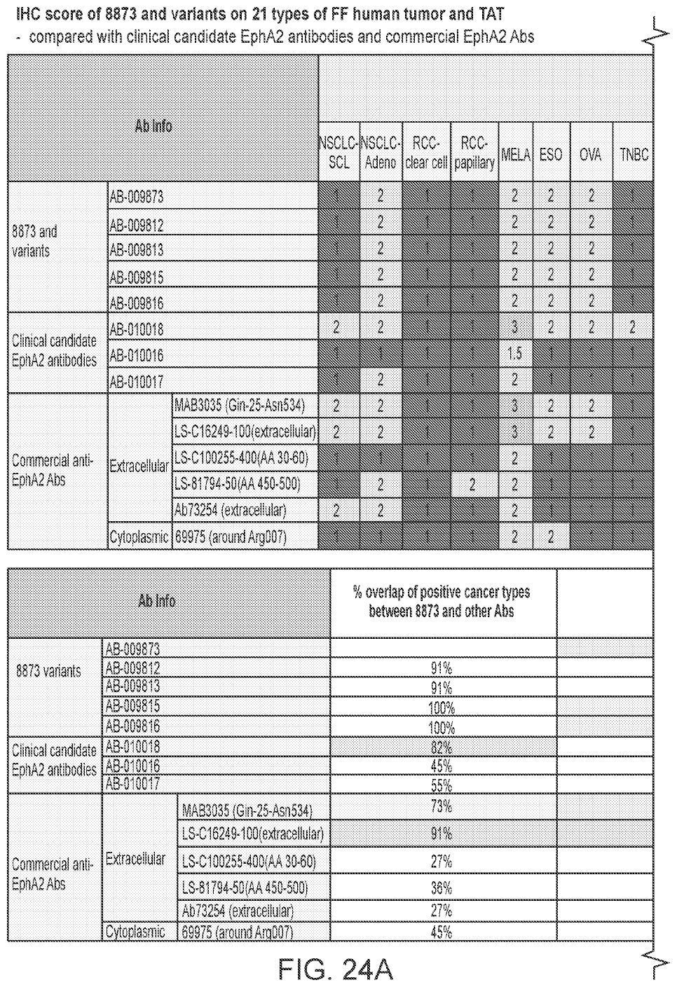

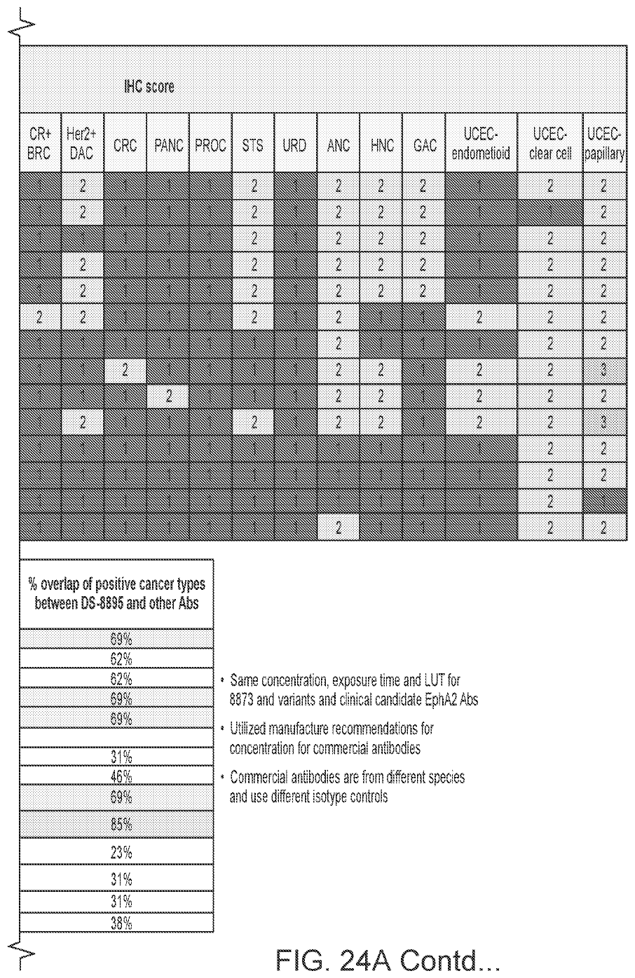

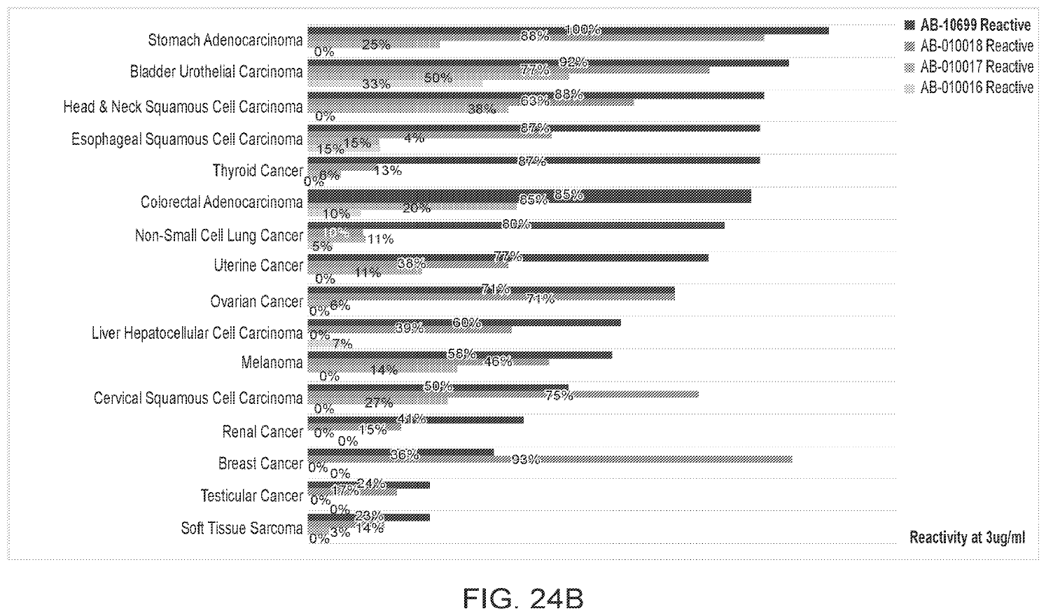

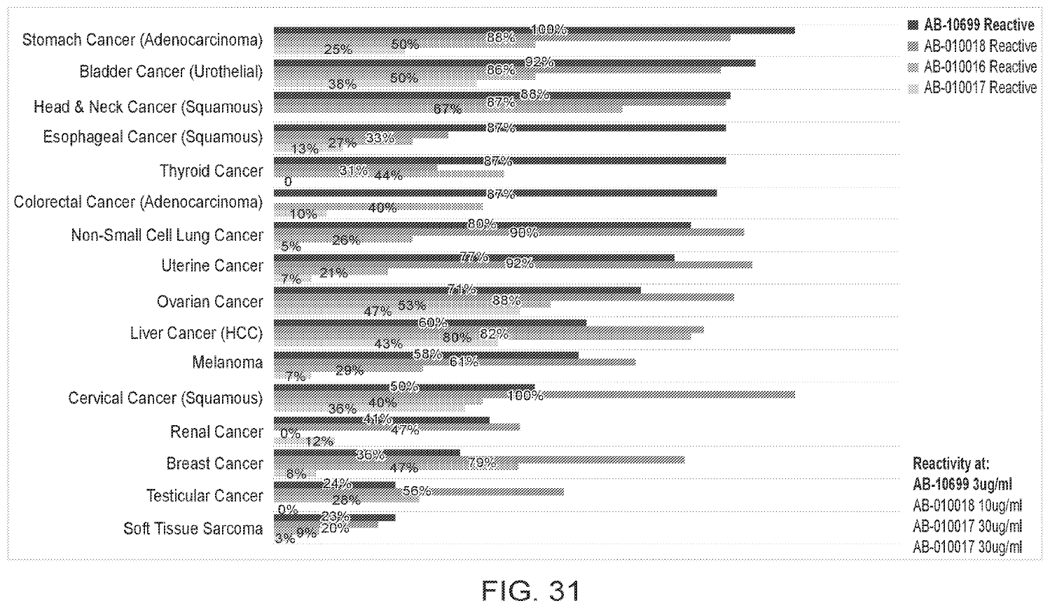

The reactivity of AB-010699 at 3 μg/ml in human cancers was tested across a panel of human cancer samples. As shown in FIG. 22, AB-010699 showed greater than 20% reactivity across all cancers tested, with results ranging from 100% reactivity in stomach adenocarcinoma (number of samples tested=8) to 23% reactivity in soft tissue sarcoma (number of samples tested −35). AB-010016 and AB-010017 (that bind to the ligand binding domain of EphA2) and AB-010018 were tested for reactivity over the same set of tissue samples as AB-010699, all at a concentration of 3 ug/ml. As shown in FIG. 24, the signal of these three comparator antibodies was very low for many of the samples tested. The assay was repeated with increased concentrations, with AB-010016 and AB-010017 being tested at 30 μg/ml and AB-010018 being tested at 10 μg/ml while AB-010699 was maintained at 3 μg/ml. AB-010699 showed a differentiated reactivity over all three comparator antibodies tested. FIG. 31.

Immunohistochemistry staining was performed on fresh frozen TMAs of select normal human tissues (i.e., liver, kidney, stomach, heart, pancreas, lung; n=3 per tissue type) with human uterine cancer as the positive control, following the manufacturer's instructions of Vector Laboratory's standard MOM protocol. In short, the frozen slides were dried at room temperature for 15 minutes and fixed with 4% PFA at room temperature for 15 minutes. After fixation, the slides were washed with wash buffer and incubated in MOM blocking reagent. After blocking the slides were incubated with AB-008873, its engineered variants, antibody AB-010018, or mouse IgG2a, the isotype control, at 3 μg/mL, overnight at 4° C. Commercial antibodies were also tested at 10 μg/mL. Next, the slides were washed with Wash Buffer and incubated with a secondary for 40 minutes at room temperature. The slides were washed and then the chromogenic reaction was developed by incubating them in Betazoid DAB for 2 minutes. After the color development the slides were counter stained with Hematoxylin QS for 15 seconds, dehydrated, mounted and then imaged in AxioScan whole slide scanner. Antibodies being tested include AB-008873 and engineered variants include AB-009812, AB-009813, AB-009815, AB-009816. AB-010018, and commercial anti-Eph2A antibody (LC-C100255-400, LS-B174-50).

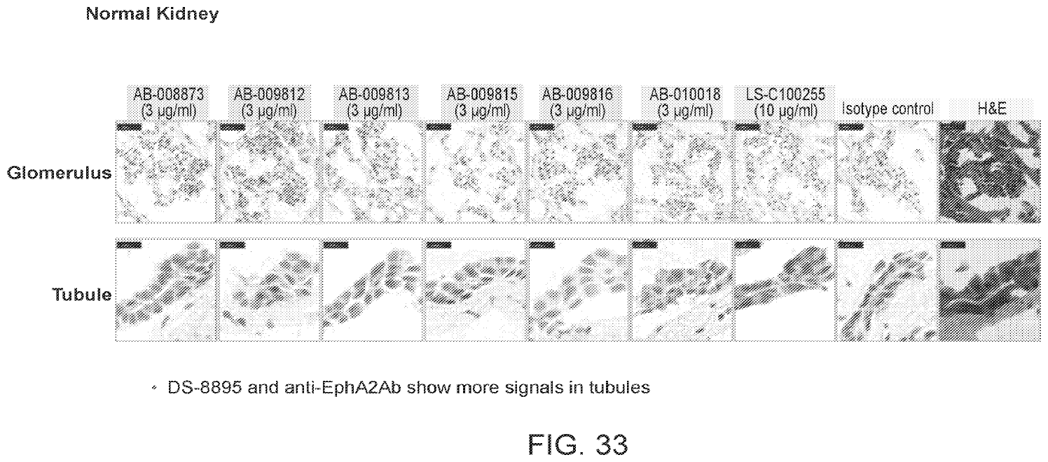

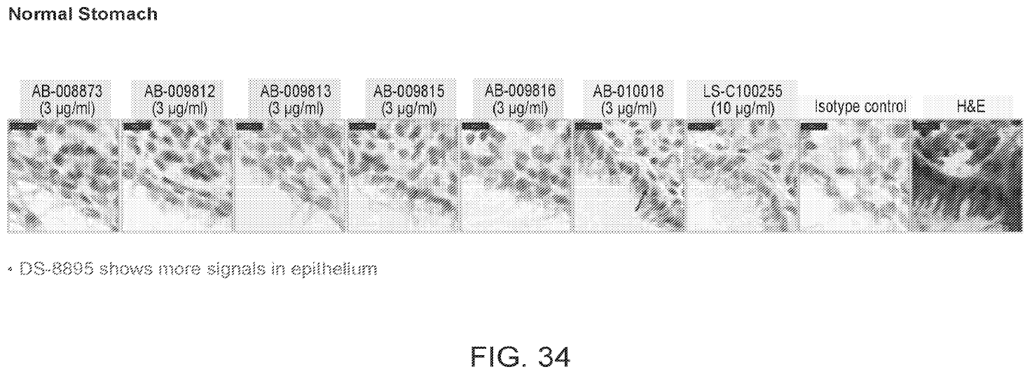

The results show that AB-008873 and its engineered variants demonstrated no detectable to faint cytoplasmic signal in all normal human tissues tested. Table 40. Unlike AB-008873, the antibody AB-010018 revealed enhanced labeling of epithelium in stomach as well as moderate signal with renal tubules. Similar normal tissue staining profiles were noted with the commercial antibodies tested. FIG. 33-34.

TABLE 40
Results of the binding of anti-EphA2 antibodies to normal human tissue

| | | Normal Human Tissue IHC Screen | | | | | |
|---|---|---|---|---|---|---|---|
| Ab Info | | Liver | Kidney | Stomach | Heart | Pancreas | Lung |
| 8873 and variants | AB-008873 | Neg to faint | Faint | Faint | Faint | Faint | Neg to faint |
| | AB-009812 | Neg to faint | Faint | Faint | Faint | Faint | Faint |
| | AB-009813 | Neg to faint | Faint | Faint | Faint | Faint | Neg to faint |
| | AB-009815 | Neg to faint | Faint | Faint | Faint | Faint | Faint |
| | AB-009816 | Neg to faint | Faint | Faint | Faint | Faint | Neg to faint |
| EphA2 Abs derived | AB-010018 | Neg to faint | Moderate | Faint to | Neg to | Neg to | Neg to faint |
| from clinical | | | | mod | faint | faint | |
| candidates | | | | | | | |
| Commercial EphA2 | LS- | Faint | Moderate | Faint to | Faint | Faint | Faint |
| Ab | C100255-400 | | | mod | | | |
| | LS-B1794-50 | Faint | Moderate | Faint to | Faint | Faint | Faint |
| | | | | mod | | | |

Note all antibodies were used at 3 μg/ml, except for LS-C100255-400 and LS-B1794-50, which were used at 10 μg/ml.

### Example 5. EPHA2 Signaling

The effect of AB-008873 and variants AB-010361 and AB-010699 on EphA2 signaling was tested along with antibodies AB-010016 and AB-010018. To assess EphA2 signaling, antibodies specific to described phosphorylation sites in the intracellular domain were used. In brief, MDA-MB-231 cells were seeded in 6 well plates and allowed to attach overnight. Next day, cells were serum-starved for 24 h. Following, antibodies and ephrin-A1 Fc were added at the respective concentrations and allowed to incubate for 15 min at 37° C. and 5% CO₂. Cells were immediately lysed and processed for Western Blot analysis. Samples were loaded on a gel and run for 70 min at 200V. After transfer to PVDF membrane, phospho-specific antibodies were used quantify receptor activation. In parallel, total EphA2 and GAPDH were detected and quantified and used for normalization post image analysis. To assess agonistic effects of the antibodies on the ephrinA1-EphA2 signaling axis, MDA-MB-231 cells were incubated with different concentrations of antibody for 15 min followed by ephrinA1-FC at 1 μg/mL. Cells were subsequently processed for Western Blot analysis as described above.

For all phosphorylation sites tested, weak or no agonistic activation and no interference in ephrinA1-EphA2 signaling axis were detected for AB-008873 and AB-010361. AB-010699 exhibited agonistic activity but only at 20% the activation induced by the natural ligand ephrin A1 (in Fc format). FIG. A.

In contrast AB-010016 and AB-010018 significantly interfere with EphA2 signaling, with AB-010016 acting as a strong agonist and AB-010018 acting as an antagonist of the signaling activity. FIGS. 43 A and B. AB-010016 activated signaling at comparable levels to the ephrin A1 ligand. FIG. 43A

### Example 6. Epitope Binning by Bio-Layer Interferometry

Epitope binning was performed using an Octet Red 96E instrument (Sartorius) using the in-tandem format. In this format, antigen is captured on a biosensor tip, a first antibody (or other binding partner) is bound to saturation, and subsequently the ability of a second antibody (or binding partner) known to recognize the antigen is assessed. If substantial binding by the second species is no longer observed, it is supportive that simultaneous binding is sterically hindered (i.e., the second species is “blocked” by pre-saturation of the 1^{st}) and that the two may bind overlapping epitopes. If, however, substantial binding of the second species is observed, despite saturation of the first, it is supportive that the two bind non-overlapping epitopes. Groups of antibodies that block one another can be considered to populate the same epitope bin.

In this experiment four antibodies and one natural ligand that recognize EphA2 were tested. The Fv regions for three of the antibodies were derived from therapeutic candidates that entered human clinical trials: AB-010016 derived from MedImmune's MEDI-547, disclosed in Peng et al., J. Mol. Biol. 2011 Oct. 21; 413(2): 390-405. doi: 10.1016/j.jmb.2011.08.018; AB-010017 derived from Merrimack's MM-310, disclosed in Geddie et al., MAbs. 2017 January; 9(1): 58-67. Doi: 10.1080/19420862.2016.125904; Kamnoun et al., Nat Biomed Eng. 2019 April; 3(4):264-280. doi: 10.1038/s41551-019-0385-4. Epub 2019 Apr. 5; and AB-010018 mouse monoclonal antibody precursor (U.S. Pat. No. 9,150,657, Sequences 35 and 37) of Daiichi-Sankyo's DS-8895a, disclosed in Hasegawa J. et al., Cancer Biol Ther. 2016 November; 17(11):1158-1167. doi: 10.1080/15384047.2016.1235663. Epub 2016 Sep. 21; Shitara K. et al., J Immunother Cancer, 2019 Aug. 14; 7(1):219. doi: 10.1186/s40425-019-0679-9, US844988; U.S. Pat. No. 9,150,657.

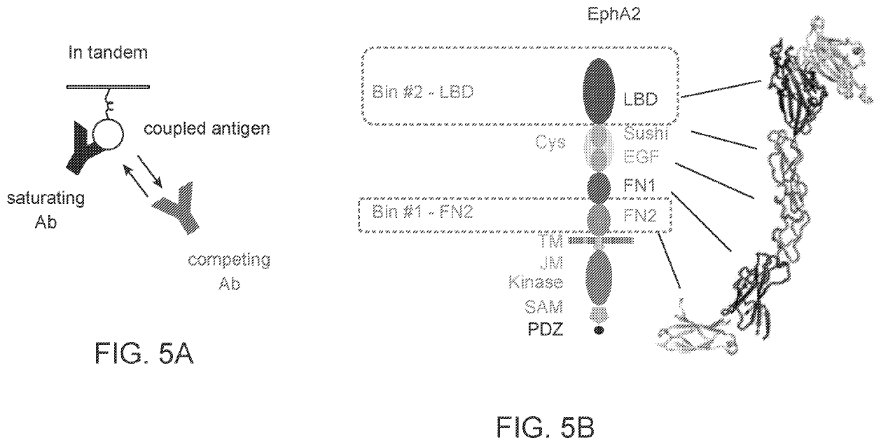

The fourth antibody Fv region (AB-008873) was discovered via Atreca's Immune Repertoire Capture (IRC™). Antibodies were produced with mouse IgG2a constant regions. The natural ligand tested was EphrinA1 fused to Fc (Acro, EF1-H5251). Antigen immobilized on the biosensor was the extracellular domain of human EphA2 (25-534) with a C-terminal HIS tag (R&D Systems, 3035-A2) and captured using Ni-NTA tips (Sartorius, 18-5102). Protein solutions were made in 1× Kinetics buffer (Satorius, 18-1092) supplemented with 2.5 mM Imidazole to reduce non-specific binding. Each pairwise interaction was assessed in both orders of addition, and controls for binding in the absence of antigen or with antigen but without a first binding species included. Binning indicated the five molecules populated only two Bins. The first bin contains molecules AB-008873 and AB-010018 and was interpreted to represent epitopes on the membrane proximal fibronectin domain of EphA2 based on the reported epitope location of DS-8895a (Hasegawa, J. et al., Novel anti-EPHA2 antibody, DS-8895a for cancer treatment. Cancer Biology and Therapy, 17(11), 1158-1167 (2016)). The second bin contains molecules AB-010017. AB-010016, and EphrinA1-Fc and was interpreted to represent epitopes on the ligand binding domain (Peng, L. et al., Structural and functional characterization of an agonistic anti-human EphA2 monoclonal antibody. Journal of Molecular Biology, 413(2), 390-405 (2011); Geddie, M. L. et al., Improving the developability of an anti-EphA2 single-chain variable fragment for nanoparticle targeting. MAbs, 9(1), 58-67 (2017)). FIG. 5, Tables 41-42. Referring to FIG. 5, the FN2 location in the first bin is inferred from domain deletion data reported in Hasegawa et al., 2016, FIG. 1, noting that the reported MMP cleavage sites are (385/395/432/435).

TABLE 41
Results of Octet in tandem binning for epitope mapping

| Analyte 1 | Analyte 2 | Notes/Scoring | TIP | Ligand |
|---|---|---|---|---|
| 8873-02 | 8873-02 | Blocks (self) despite | NiNTA | R&D ECD-06 |
| | | faster dissoc. | | |
| 10016-01 | 8873-02 | Binding observed (curve | NiNTA | R&D ECD-06 |
| | | behind light green) | | |
| 10017-01 | 8873-02 | Binding observed | NiNTA | R&D ECD-06 |
| 10018-01 | 8873-02 | Blocks | NiNTA | R&D ECD-06 |
| Ephrin-A1 | 8873-02 | Binding observed | NiNTA | R&D ECD-06 |
| Buffer | 8873-02 | Positive control OK - | NiNTA | R&D ECD-06 |
| | | EphA2 + competitor | | |
| Buffer | 8873-02 | Background control OK - | NiNTA | Buffer |
| | | competitor alone | | |

TABLE 42
Epitope recognized by AB-008873
overlaps with that of AB-010018

| | Bin #1 - FN2 | | Bin #2 - LBD | | |
|---|---|---|---|---|---|
| | 8873-2 | 10018-01 | 10017-01 | 10016-01 | Ephrin-A1 |
| 8873-2 | blocked | blocked | binds | binds | binds |
| 100018-01 | blocked | blocked | binds | binds | binds |
| 10017-01 | binds | binds | blocked | blocked | blocked |
| 10016-01 | binds | binds | blocked | blocked | blocked |
| Ephrin-A1 | binds | binds | blocked | blocked | blocked |

### Epitope Domain Investigation by Yeast Surface Display

To further validate AB-008873 epitope domain mapping, subdomains of human EphA2 and chimeras of human EphA2 and human EphA4 were displayed on the surface of yeast. AB-008873 had shown binding to EphA2 but not homologous receptor EphA4 in other assays (screening at Integral Molecular). 5 constructs (shown in Table 43) were selected for display on the surface of yeast and staining via AB-008873:

TABLE 43
EphA2 subdomains

| | | |
|---|---|---|
| EphA2 | Full ECD | QGKEVVLLDFAAAGGELGWLTHPYGKGWDLMQNIMNDMPI |
| (aa 25-534) | | YMYSVCNVMSGDQDNWLRTNWVYRGEAERIFIELKFTVRDC |
| | | NSFPGGASSCKETFNLYYAESDLDYGTNFQKRLFTKIDTIAPD |
| | | EITVSSDFEARHVKLNVEERSVGPLTRKGFYLAFQDIGACVAL |
| | | LSVRVYYKKCPELLQGLAHFPETIAGSDAPSLATVAGTCVDH |
| | | AVVPPGGEEPRMHCAVDGEWLVPIGQCLCQAGYEKVEDACQ |
| | | ACSPGFFKFEASESPCLECPEHTLPSPEGATSCECEEGFFRAPQ |
| | | DPASMPCTRPPSAPHYLTAVGMGAKVELRWTPPQDSGGREDI |
| | | VYSVTCEQCWPESGECGPCEASVRYSEPPHGLTRTSVTVSDLE |
| | | PHMNYTFTVEARNGVSGLVTSRSFRTASVSINQTEPPKVRLEG |
| | | RSTTSLSVSWSIPPPQQSRVWKYEVTYRKKGDSNSYNVRRTE |
| | | GFSVTLDDLAPDTTYLVQVQALTQEGQGAGSKVHEFQTLSPE |
| | | GSGN (SEQ ID NO: 101) |
| EphA2 | FN2 only | TEPPKVRLEGRSTTSLSVSWSIPPPQQSRVWKYEVTYRKKGDS |
| (aa 437-534) | | NSYNVRRTEGFSVTLDDLAPDTTYLVQVQALTQEGQGAGSK |
| | | VHEFQTLSPEGSGN (SEQ ID NO: 95) |
| EphA4_ECD | Full ECD | SRVYPANEVTLLDSRSVQGELGWIASPLEGGWEEVSIMDEKN |
| (aa 23-546) | | TPIRTYQVCNVMEPSQNNWLRTDWITREGAQRVYIEIKFTLRD |
| | | CNSLPGVMGTCKETFNLYYYESDNDKERFIRENQFVKIDTIAA |
| | | DESFTQVDIGDRIMKLNTEIRDVGPLSKKGFYLAFQDVGACIA |
| | | LVSVRVFYKKCPLTVRNLAQFPDTITGADTSSLVEVRGSCVNN |
| | | SEEKDVPKMYCGADGEWLVPIGNCLCNAGHEERSGECQACKI |
| | | GYYKALSTDATCAKCPPHSYSVWEGATSCTCDRGFFRADND |
| | | AASMPCTRPPSAPLNLISNVNETSVNLEWSSPQNTGGRQDISY |
| | | NVVCKKCGAGDPSKCRPCGSGVHYTPQQNGLKTTKVSITDLL |
| | | AHTNYTFEIWAVNGVSKYNPNPDQSVSVTVTTNQAAPSSIAL |
| | | VQAKEVTRYSVALAWLEPDRPNGVILEYEVKYYEKDQNERS |
| | | YRIVRTAARNTDIKGLNPLTSYVFHVRARTAAGYGDFSEPLEV |
| | | TTNTVPSRIIGDGANS (SEQ ID NO: 102) |
| EphA2_A4in | EphA2 with | QGKEVVLLDFAAAGGELGWLTHPYGKGWDLMQNIMNDMPI |
| FN1 | EphA4 FN1 | YMYSVCNVMSGDQDNWLRTNWVYRGEAERIFIELKFTVRDC |
| | domain | NSFPGGASSCKETFNLYYAESDLDYGTNFQKRLFTKIDTIAPD |
| | | EITVSSDFEARHVKLNVEERSVGPLTRKGFYLAFQDIGACVAL |
| | | LSVRVYYKKCPELLQGLAHFPETIAGSDAPSLATVAGTCVDH |
| | | AVVPPGGEEPRMHCAVDGEWLVPIGQCLCQAGYEKVEDACQ |
| | | ACSPGFFKFEASESPCLECPEHTLPSPEGATSCECEEGFFRAPQ |
| | | DPASMPCTRPPSAPLNLISNVNETSVNLEWSSPQNTGGRQDIS |
| | | YNVVCKKCGAGDPSKCRPCGSGVHYTPQQNGLKTTKVSITDL |
| | | LAHTNYTFEIWAVNGVSKYNPNPDQSVSVTVTTNQTEPPKVR |
| | | LEGRSTTSLSVSWSIPPPQQSRVWKYEVTYRKKGDSNSYNVR |
| | | RTEGFSVTLDDLAPDTTYLVQVQALTQEGQGAGSKVHEFQTL |
| | | SPEGSGN (SEQ ID NO: 103) |
| EphA2_A4in | EphA2 with | QGKEVVLLDFAAAGGELGWLTHPYGKGWDLMQNIMNDMPI |
| FN2 | EphA4 FN2 | YMYSVCNVMSGDQDNWLRTNWVYRGEAERIFIELKFTVRDC |
| | domain | NSFPGGASSCKETFNLYYAESDLDYGTNFQKRLFTKIDTIAPD |
| | | EITVSSDFEARHVKLNVEERSVGPLTRKGFYLAFQDIGACVAL |
| | | LSVRVYYKKCPELLQGLAHFPETIAGSDAPSLATVAGTCVDH |
| | | AVVPPGGEEPRMHCAVDGEWLVPIGQCLCQAGYEKVEDACQ |
| | | ACSPGFFKFEASESPCLECPEHTLPSPEGATSCECEEGFFRAPQ |
| | | DPASMPCTRPPSAPHYLTAVGMGAKVELRWTPPQDSGGREDI |
| | | VYSVTCEQCWPESGECGPCEASVRYSEPPHGLTRTSVTVSDLE |
| | | PHMNYTFTVEARNGVSGLVTSRSFRTASVSINQAAPSSIALVQ |
| | | AKEVTRYSVALAWLEPDRPNGVILEYEVKYYEKDQNERSYRI |
| | | VRTAARNTDIKGLNPLTSYVFHVRARTAAGYGDFSEPLEVTT |
| | | NTVPSRIIGDGANS (SEQ ID NO: 104) |

Constructs were expressed as Aga2 fusions with an internal HA tag between Aga2 and the displayed protein as well as a C-terminal Myc tag (Boder, E. T., & Wittrup, K. D., Yeast surface display for screening combinatorial polypeptide libraries. Nature Biotechnology, 15(6), 553-557 (1997)). Yeast cells were cultured in SDCAA (Teknova, S0543) and induced with SGCAA (Teknova, S0542). Cells were stained with pairs of primary and secondary antibodies. AB-008873 Lot 2 (Atum, mIgG2a) and Donkey anti-Mouse IgG AF647 (JacksonImmunoResearch 715-605-150) were used for 10-point titrations to assess 8873 binding. Chicken IgY anti-cMyc (Life technologies A21281) and Goat anti Chicken IgY FITC (Life technologies A11039) were used to assess expression levels. The analysis was run on the iQue3 flow cytometer. Staining demonstrates binding of AB-008873 to the full extracellular domains EphA2, but not EphA4. The FN2 domain of EphA2 alone is sufficient to mediate binding and expresses at high levels. When the FN2 domain of EphA4 is swapped into EphA2, the binding to AB-008873 was lost. When the FN1 domain of EphA4 was swapped into in EphA2, the binding to AB-008873 was still observed. FIG. 6. As shown in FIG. 6, EphA4 chimeras offer stability advantages and can inform the finer mapping at the domain-level by expressing EphA2 truncations. Referring to FIG. 6 lower panel, FN2 domain (EphA2-FN2) alone shows strong binding signal (triangle). FN2 domain alone also has high relative expression. EphA2 full ECD shows a binding signal (circle) relatively lower than the binding signal driven by the FN2 domain alone. EphA4 full ECD shows no binding signal (square). Swapping the FN1 domain from EphA4 into EphA2 enables binding of the epitope (diamond). Binding is lost when the FN2 domain from EphA4 is swapped into EphA2. These results indicate that the epitope of AB-008873 is located at the membrane-proximal FN2 domain of EphA2.

Finer, residue-level resolution for AB-008873's epitope was determined by generating a library of FN2 mutants and testing their binding to AB-008873 via yeast display. The results of the testing indicated that AB-008873 binds a conformational epitope, spanning four stretches of primary sequence of EphA2, comprising the following residues: Pro439, Lys441, Arg443, Leu444, Arg447, Lys476, Gly477, Leu504, Gln506, Ser519, Lys520, Val521, His522, Glu523, Phe524, and Gln525. FIG. 44. The residues contained in the epitope are not conserved within the EphA2 family. FIG. 45. However, the epitope residues are conserved across species commonly used in toxicology studies. FIG. 46. The epitope of AB-008873 is located on the opposite side of the FN2 domain from the reported LBD-FN2 site. See Nikolov. D. B et. al. (2014) Cell Adh Migr 8, 360-365.

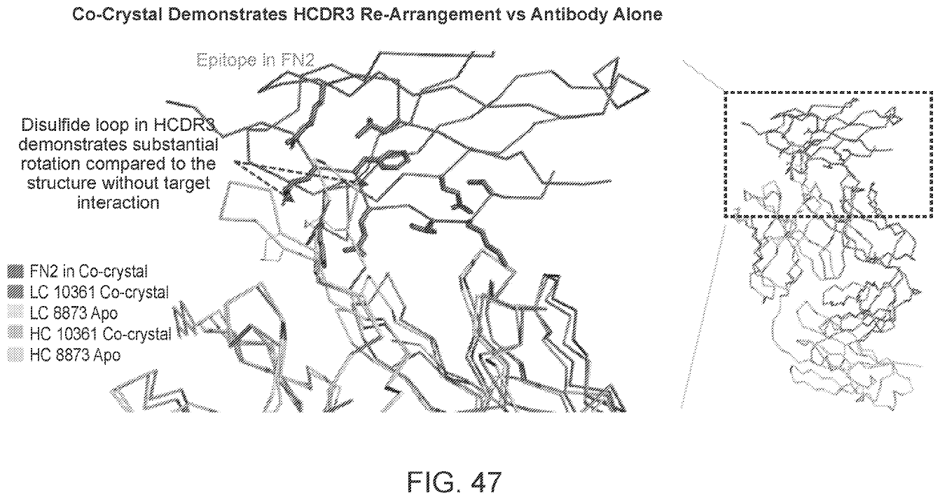

Co-crystallization studies showed that the disulfide loop in the HCDR3 of AB-008873 demonstrated substantial rotation as compared to the structure without target interaction. FIG. 47. Furthermore, the co-crystal and yeast display data aligned and provided additional details on the epitope. Combining these data indicated epitope residues that are not in direct contact with the CDRs of AB-008873 (as defined by a 4 Angstrom distance constraint) but can substantially reduce or abrogate binding when mutated in yeast display: Pro439, Lys441, Arg443, Gly477, Leu504, and His522.

Additional yeast display assays indicated that the epitopes of AB-008873 and AB-010018 overlap but are distinct. The epitope of AB-010018 is a protrusion on an adjacent face of FN2 relative to the AB-008873 epitope, with two residues shared between epitopes. AB-010018 was shown to bind to Thr472, Arg474, Asp478, Ser479, Asn480, Gly477, and Leu504 in this assay.

### Example 7. Binding Affinity

### Steady State KD

Binding of antibodies to the FN2 domain of hEphA2 was measured using the Biacore® T200 SPR system at 25° C. using HBS-EP+ buffer (0.01 M HEPES pH 7.4, 0.15 M NaCl, 3 mM EDTA, 0.005% v/v Surfactant P20). Antibodies in mIgG2a format were captured at levels between 475-715 RU via an anti-mouse Fc antibody immobilized to a CM5 chip. Binding was assessed using a titration of C-terminally His-tagged hEphA2-FN2 (Thr437-Asn534+His tag) associated for 30 s or 75 s at 30 μL/min. Data was double-reference subtracted using both a surface without captured antibody and from injections of HBS-EP+ buffer. The equilibrium response was measured at 4 seconds before the end of association with a window of 5 seconds and fit using the Biacore T200 Evaluation Software 3.1 with the Steady State Affinity Model. The results are shown in Table 44.

TABLE 44
The binding kinetics of the EphA2 antibodies
to the FN2 domain of hEphA2

| | Steady-State | | | |
|---|---|---|---|---|
| | Monovalent KD | Standard | | |
| Antibody | (M) | Error of KD | Chi2 (RU2) | n |
| AB-008873 | 2.26E−06 | 5.9E−08 | 0.134 | 2 |
| AB-010361 | 1.11E−06 | 5.5E−08 | 0.363 | 3 |
| AB-010363 | 1.31E−06 | 2.6E−08 | 0.068 | 3 |
| AB-010699 | 1.89E−08 | 2.9E−10 | 0.286 | 3 |

Monovalent affinity to hEphA2-FN2 increased from ˜2 M from parental antibody AB-008873 to ˜20 nM for the engineered variant AB-010699.

Binding of EphA2 Antibodies to the FN2 Domain of hEphA2

Binding of EphA2 antibodies to the FN2 domain of hEphA2 was measured using the Biacore® T200 SPR system. Biacore® assays were conducted at 25° C. using HBS-EP+ buffer (0.01 M HEPES pH 7.4, 0.15 M NaCl, 3 mM EDTA, 0.005% v/v Surfactant P20) using the FN2 domain as the analyte with the antibodies in mIgG2a format as the immobilized ligand. Data was analyzed using 1:1 Langmuir binding model and steady-state model in Biacore Evaluation software.

C-terminally His-tagged human EphA2 FN2 domain (Thr437-Asn534+His tag) was used as the analyte. The analyte was associated for 120 seconds and dissociated for 100 seconds or 300 seconds at 30 μL/min. Titration of 0, 11.1 nM, 33.3 nM, 100 nM, 300 nM, and 900 nM run for AB-010699 and AB-010018 as mIgG2a. The results are shown in Table 45.

TABLE 45
Measurement results of binding of EphA2
antibodies to the FN2 domain of hEphA2

| Antibody | ka (1/Ms) | kd (1/s) | KD (M) | Rmax (RU) |
|---|---|---|---|---|
| AB-010699 | 3.9E+6 | 0.075 | 1.9E−08 | 110.2 |
| AB-010018 | 4.9E+6 | 0.0021 | 4.3E−09 | 74.9 |

### Binding of EphA2 Antibodies to the FN2 Domain, and Fragments of the FN1-FN2 Domains of the EphA2

Additional Biacore® assays were conducted to determine binding of AB-010699 human Fab fragment and AB-010018 human Fab fragment to the ECD, FN1-FN2 domains, FN2 domain, and fragments of the FN1-FN2 domains of the EphA2 protein. These assays were conducted using the EphA2 proteins fused to the N-terminal side of a human Fe (hEphA2-Fc fusion) as the immobilized ligand and AB-010699 and AB-010018 in Fab format as the analyte. The hEphA2-Fc fusions were captured with an anti-human Fe immobilized to a CM5 chip.

Biacore® assays were conducted at 25° C. using HBS-EP+ buffer (0.01 M HEPES pH 7.4, 0.15 M NaCl, 3 mM EDTA, 0.005% v/v Surfactant P20). Data was double-reference subtracted and data was analyzed using 1:1 Langmuir binding model and steady-state model in Biacore Evaluation software.

The analyte was associated for 120 seconds and dissociated for 100 seconds for AB-010699 or 300 seconds for AB-010018 at 30 L/min. Binding to hEphA2-ECD-Fc tested using 100, 300, and 900 nM analyte. Binding to all other ligands tested using 33.3, 100, and 300 nM analyte. The results are shown in Tables 46-47.

TABLE 46
Binding kinetics of AB-010699

| Ligand | ka (1/Ms) | kd (1/s) | KD (M) | Rmax (RU) |
|---|---|---|---|---|
| hEphA2-FN12- | 5.0E+6 | 4.3E−2 | 8.6E−9 | 149.9 |
| (327-534) - Fc | | | | |
| hEphA2-FN2- | 1.0E+7 | 8.5E−2 | 8.6E−9 | 59.2 |
| (437-534) - Fc | | | | |
| hEphA2-clv385-534-Fc | 6.05E+6 | 7.2E−2 | 1.2E−8 | 136.4 |
| hEphA2-clv395-534-Fc | 5.72E+6 | 7.0E−2 | 1.2E−8 | 155.5 |
| hEphA2-clv432-534-Fc | 7.78E+6 | 7.5E−2 | 9.6E−9 | 145.9 |
| hEphA2-clv435-534-Fc | 8.42E+6 | 7.8E−2 | 9.3E−9 | 150.1 |

TABLE 47
Binding kinetics of for AB-010018

| Ligand | ka (1/Ms) | kd (1/s) | KD (M) | Rmax (RU) |
|---|---|---|---|---|
| hEphA2-ECD(25-534) | 3.2E+5 | 2.0E−3 | 6.3E−9 | 120.2 |
| hEphA2-FN12- | 1.8E+5 | 1.9E−3 | 1.1E−8 | 115.7 |
| (327-534) - Fc | | | | |
| hEphA2-FN2- | 4.3E+5 | 2.7E−3 | 6.1E−9 | 52.4 |
| (437-534) - Fc | | | | |
| hEphA2-clv385-534-Fc | 2.6E+5 | 2.7E−3 | 1.0E−8 | 111.9 |
| hEphA2-clv395-534-Fc | 2.6E+5 | 2.6E−3 | 1.0E−8 | 138.4 |
| hEphA2-clv432-534-Fc | 4.0E+5 | 3.0E−3 | 7.5E−9 | 112.3 |
| hEphA2-clv435-534-Fc | 4.3E+5 | 3.1E−3 | 7.3E−9 | 114.5 |

The calculated monovalent KD of AB-010699 is similar across all hEphA2 variants (ranging from 9 to 12 nM) and there appears to be no differentiated binding to different hEphA2 recombinant fragment representing cleavage forms (ranging from 9 to 12 nM).

The calculated monovalent KD of AB-010018 is also similar across all hEphA2 variants (ranging from 6 to 11 nM) and there also appears to be no differentiated binding to different hEphA2 cleavage forms (ranging from 7 to 10 nM).

### Binding of EphA2 Antibodies as SCFV

Single-chain variable fragments (SCFVs) of 10699 were expressed with C-terminal His-tags in mammalian cells and purified by Nickel Sepharose affinity chromatography. SCFVs were assayed in triplicate for thermostability using an Unchained Labs UNcle instrument. The concentration of purified antibodies was adjusted to 0.5 mg/ml in PBS immediately prior to analysis. To determine melting temperature (Tm) intrinsic protein fluorescence was measured at 473 nm every 1.1° C. as temperature was increased linearly from 25° C. to 95° C. at a rate of 0.3° C./min. The UNcle Analysis software (version 4.01) was used to find the Tm as the first derivative of the barycentric mean (BCM). Binding of the SCFVs to the FN2 domain of hEphA2 was measured in triplicate using the Biacore T200 SPR system at 25° C. using HBS-EP+ buffer (0.01 M HEPES pH 7.4, 0.15 M NaCl, 3 mM EDTA, 0.005% v/v Surfactant P20). SCFVs were captured at levels between 200-300 RU via an anti-His antibody immobilized to a CM5 chip. Binding was assessed using a titration of FLAG-tagged hEphA2-FN2 (300 nM, 100 nM, 33 nM, 11 nM, 3.7 nM) associated for 120 s and dissociated for 30 s at 30 μL/min. Data was double-reference subtracted using both a surface without captured SCFV and from injections of HBS-EP+ buffer. The 1:1 Langmuir binding model was used to fit each titration and the average of three replicates is reported. Table 48 shows amino acid sequences in SCFV constructs, and the results are shown in Table 49.

TABLE 48
SCFV Constructs

| Construct | Orientation | Linker | Sequence |
|---|---|---|---|
| SCFV- | VH-VL | (G4S)4 (SEQ | (SEQ ID NO: 765) |
| 010699-01 | | ID NO: 782) | |
| SCFV- | VL-VH | (G4S)4 (SEQ | (SEQ ID NO: 766) |
| 010699-02 | | ID NO: 782) | |
| SCFV- | VH-VL | (G4S)3 (SEQ | (SEQ ID NO: 767) |
| 010699-05 | | ID NO: 91) | |
| SCFV- | VL-VH | (G4S)3 (SEQ | (SEQ ID NO: 768) |
| 010699-06 | | ID NO: 91) | |

TABLE 49
Binding kinetics of EphA2 scFvs scFV construct

| | | Average | | | |
|---|---|---|---|---|---|
| | | Tm (° C.) | Average KD (M) | | |
| | mg/L | [Standard | to FN2_FLAG | Average ka | Average kd |
| | yield | Deviation] | [% CV] | (1/Ms) [% CV] | (1/s) [% CV] |
| SCFV-010699-01 | 160 | 66.3 [0.6] | 2.4E−08 [0.6%] | 4.2E+06 [1.4%] | 0.10 [1.0%] |
| SCFV-010699-02 | 82 | 65.0 [0.3] | 3.0E−08 [0.9%] | 5.2E+06 [0.7%] | 0.16 [1.6%] |
| SCFV-010699-05 | 97 | 65.0 [0.6] | 2.7E−08 [1.1%] | 3.9E+06 [3.2%] | 0.11 [2.4%] |
| SCFV-010699-06 | 179 | 63.7 [0.7] | 3.1E−08 [1.2%] | 4.4E+06 [1.4%] | 0.13 [0.7%] |

All the SCFV constructs exhibited an average KD (24-31 nM) that is comparable to that of the native AB-010669 monovalent KD.

### Example 8. Multivalent Formats

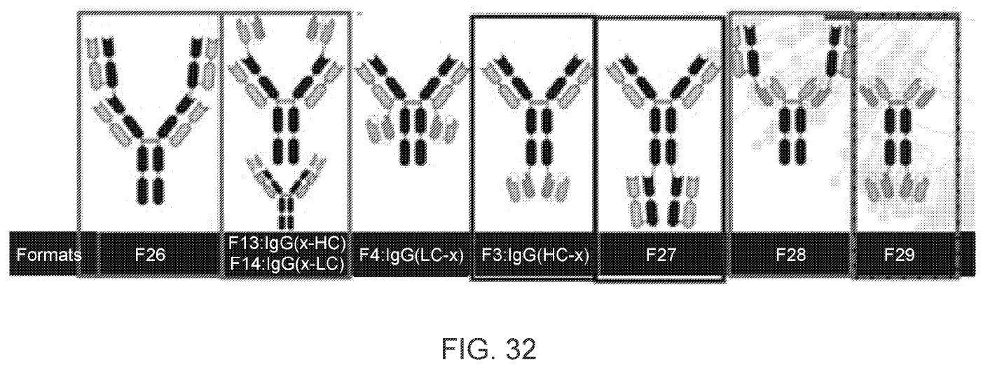

AB-008873 was used to construct tetravalent molecules that had four EphA2 binding arms per molecule. FIG. 32 shows schematics of the formats, including the format of the Fc regions. The constructs were tested for ADCC, and cell binding activity as described above. Certain of the tetravalent constructs tested exhibited improved ADCC and cell binding over the bivalent format. FIG. 32 (RR) and Table 50. Those constructs that included components fused to the C-terminus of Fc exhibited substantial reduction of ADCC activity.

TABLE 50
Tetravalent constructs

| | | | | | Flow Binding - EC₅₀ | | Flow Binding - dAct | |
|---|---|---|---|---|---|---|---|---|
| | | Fold rel | | Fold rel | | Fold rel | | Fold rel |
| | ADCC - EC₅₀ | AB-8873 | ADCC - dAct | AB-8873 | | AB-8873 | | AB-8873 |
| Molecule | M | IgG | % | IgG | M | IgG | MedFl | IgG |
| A B-008873-hlgG1 | 1.6E−08 | 1.0 | 30.0 | 1.0 | 6.1E−09 | 1.0 | 609705 | 1.0 |
| SCFV-8873-01_hFc1_F2 | 1.8E−08 | 0.9 | 28.3 | 0.9 | 1.4E−08 | 0.4 | 223880 | 0.4 |
| Fab8873-(GGSGG)3- | 1.5E−09 | 10.5 | 32.8 | 1.1 | 1.3E−09 | 4.6 | 2230775 | 3.7 |
| HC-AB-008873-h1- | | | | | | | | |
| 4p0_F26 | | | | | | | | |
| Fab8873-(GGSPG)3- | 1.2E−09 | 13.3 | 31.0 | 1.0 | 4.8E−10 | 17.6 | 1556556 | 3.4 |
| HC-AB-008873-h1- | | | | | | | | |
| 4p0_F26 | | | | | | | | |
| SCFV8873-01- | 1.9E−09 | 8.3 | 27.6 | 0.9 | 4.4E−10 | 13.5 | 1835412 | 3.1 |
| (GGSGG)3-HC-AB- | | | | | | | | |
| 008873-h1-2p2_F13 | | | | | | | | |
| SCFV8873-01- | 1.4E−09 | 11.9 | 34.4 | 1.1 | 1.8E−09 | 3.8 | 2658851 | 4.5 |
| (GGSPG)3-HC-AB- | | | | | | | | |
| 008873-h1-2p2_F13 | | | | | | | | |
| SCFV8873-01- | 1.1E−09 | 15.0 | 29.8 | 1.0 | 2.1E−09 | 3.0 | 2558140 | 4.4 |
| (GGSGG)3-LC-AB- | | | | | | | | |
| 008873-h1-2p2_F14 | | | | | | | | |
| SCFV8873-01- | 3.1E−10 | 52.3 | 36.9 | 1.2 | 4.4E−10 | 13.6 | 2873733 | 5.0 |
| (GGSPG)3-LC-AB- | | | | | | | | |
| 008873-h1-2p2_F14 | | | | | | | | |
| AB-8873-h1-2p2-LC- | 1.2E−08 | 1.3 | 30.9 | 1.0 | 7.5E−10 | 7.8 | 1307497 | 2.2 |
| SCFV-008873-01_F4 | | | | | | | | |
| Ab8873-h1-2p2-HC- | 1.0E−07 | 0.2 | 3.3 | 0.1 | 6.7E−10 | 10.2 | 737080 | 1.2 |
| (GGSGG)3-SCFV8873- | | | | | | | | |
| 01_F3 | | | | | | | | |
| Ab8873-h1-2p2-HC- | 1.0E−07 | 0.2 | 3.6 | 0.1 | 3.0E−09 | 1.9 | 913380 | 1.6 |
| (GGSPG)3-SCFV8873- | | | | | | | | |
| 01_F3 | | | | | | | | |
| Ab8873-h1-4p0-HC- | 1.0E−07 | 0.2 | 1.6 | 0.1 | 1.3E−09 | 4.5 | 1042829 | 1.8 |
| (GGSGG)3- | | | | | | | | |
| Fab8873_F27 | | | | | | | | |
| Ab8873-h1-4p0-HC- | 1.0E−07 | 0.2 | 8.9 | 0.3 | 1.7E−09 | 5.0 | 615030 | 1.3 |
| (GGSPG)3- | | | | | | | | |
| Fab8873_F27 | | | | | | | | |
| Fab8873-SCFV8873- | 2.9E−09 | 5.5 | 33.8 | 1.1 | 9.9E−10 | 5.9 | 1442867 | 2.4 |
| 01_h1-2p2_F28 | | | | | | | | |
| SCFV8873-01-h1-2p2- | 1.0E−07 | 0.2 | 3.5 | 0.1 | 1.3E−09 | 4.6 | 1109331 | 2.0 |
| HC-(GGSGG)3- | | | | | | | | |
| SCFV8873-01_F29 | | | | | | | | |
| SCFV8873-01-h1-2p2- | 1.7E−09 | 9.7 | 7.6 | 0.3 | 3.4E−09 | 2.2 | 1393777 | 2.3 |
| HC-(GGSPG)3- | | | | | | | | |
| SCFV8873-01_F29 | | | | | | | | |

### Example 9. AB-008873-IL15 Fusions

Receptor-linked interleukin-15 (RLI) is made by covalently joining the IL-15 receptor alpha sushi domain to IL-15 via a glycine-serine linker. RLI enables trans presentation of IL-15 to the IL-15 receptor complex (IL-15 receptor beta and IL-15 common gamma chain) on T and NK immune cells, and this trans presentation encourages immune responses such as proliferation and survival of naïve CD8+ T cells, development of memory CD8+ T cells, improved survival and upregulation of lytic molecules and CD16a on NK cells, etc.

Bispecific antibodies, referred to as IL-15 MultiMabs, that couple RLI to the anti-tumor antibody AB-008873 were developed. In one example of an IL-15 MultiMab, a 2-plus-2 molecule (2p2) that links an RLI domain to the C-terminus of each light chain of an IgG molecule via a glycine-serine linker was created. These RLI domains can be either wildtype (“wt/RLI”) or a mutated variant expected to have lower affinity for the IL-15 receptor complex (“low/RLI”). The two molecules described are shown here as FIG. 36, and sequences of the component parts are provided in Table 15 above.

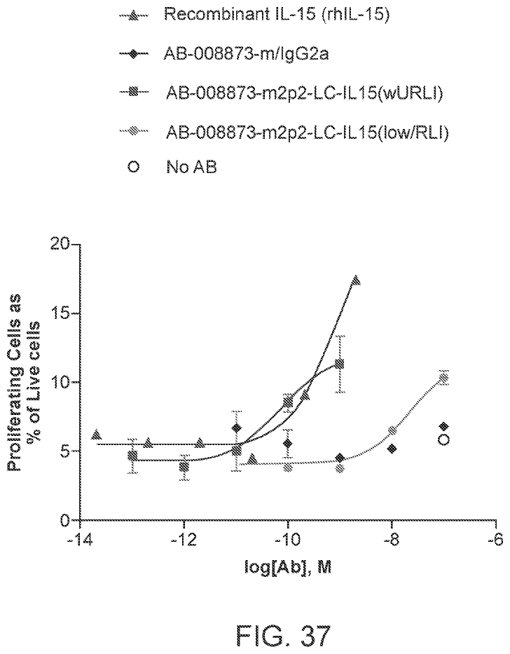

These IL-15 MultiMabs have been assessed for their ability to induce proliferation of primary NK cells. Primary human NK cells were labeled with CellTrace CFSE cell proliferation dye (Invitrogen Cat. C4554) and mixed with AB-008873 IL-15 MultiMabs. After 3 days of treatment, cells were analyzed with a flow cytometer and gated for cells that lost signal from proliferation dye relative to untreated cells. See FIG. 37.

The AB-008873 IL-15 MultiMabs showed dose-dependent increases in human NK cell proliferation. Antibodies fused with wild-type RLI (“wt/RLI”) were more potent than those fused with low affinity RLI (“low/RLI”), and unmodified antibodies (“AB-008873-mIgG2a”) did not show a measurable response up to 100 nM.

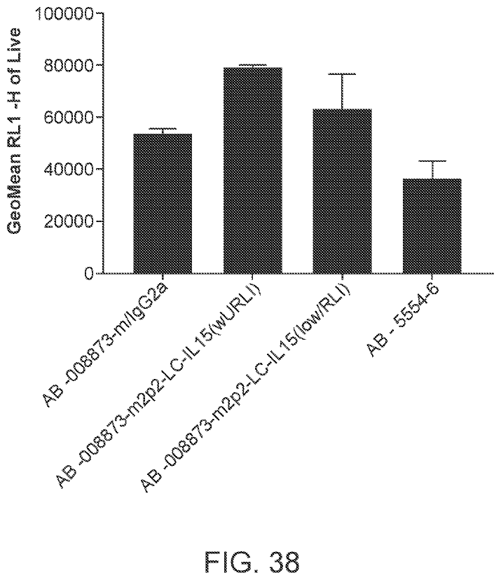

AB-008873 IL15 MultiMabs were assessed for their ability to bind to target tumor cells using flow cytometry. Parental antibody or IL-15 MultiMabs were added to CT26 ex vivo cells at 100 nM and incubated on ice, followed by washing. Secondary Alexa Fluor 647-conjugated anti-mouse antibody and cell membrane integrity dye was added and incubated on ice, followed by washing. Stained cells were analyzed by flow cytometry, gated for viable cells, and quantified for Alexa Fluor 647 detection. The data is shown in FIG. 38. The results indicate that addition of the RLI domain to AB-008873 did not negatively impact binding of the antibody to tumor target cells.

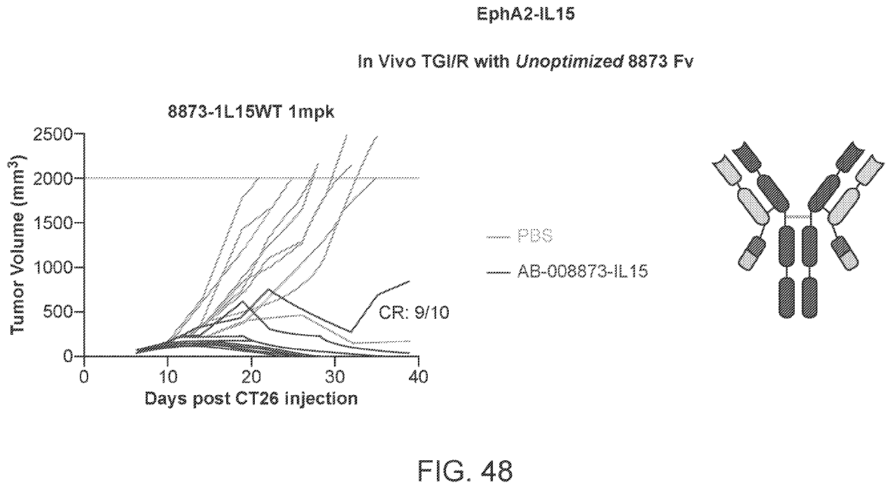

As shown in FIG. 48, AB-008873-m2p2-LC-IL15(wt/RLI) was active in tumor growth inhibition and regression (TGI/R) studies in syngeneic mouse models using CT26 tumor cells.

### Example 10. 4-1BB and 4-1BBL Constructs

4-1BB fusion proteins were tested for their tumor targeting activities. These fusion proteins comprise (i) at least the antigen binding domains of the EphA2 antibodies and 4-1BB ligand domains (Table 13) or (ii) at least the antigen binding domains of the EphA2 antibodies and the ScFv portions of the anti-4-1BB antibodies (Table 10).

Bispecific antibodies that comprise two scFv fragments of anti-4-1BB antibody linked to the HC of the EphA2 antibodies AB-008873 or AB-010361 (EphA2-4-1BB bispecific antibodies) were constructed. Anti-hen egg lysozyme targeting anti-4-1BB antibody (5554-41BB) was also constructed and used as a negative control.

After confirming that they retained the ability to bind to PC3 cells, the EphA2-4-1BB bispecific antibodies were assayed for their ability to activate human 4-1BB using an inducible reporter cell system (Promega cat JA2351). 4-1n reporter cells were either cultured alone with the EphA2-4-1BB bispecific antibodies or co-cultured with A549 tumor cells that had been treated with the EphA2-4-1BB bispecific antibodies. The 4-1BB antibody Urelumab (AB-009811) was included as a non-tumor targeting control. After 5 hours of culture, 4-1BB activation was determined from the reporter cells by measuring bioluminescence output.

Results from this assay indicated that the EphA2-4-1BB bispecific antibodies only exhibited 4-1BB activation in the presence of tumor cells while the Urelumab control activated 4-1BB with us without the presence of tumor cells.

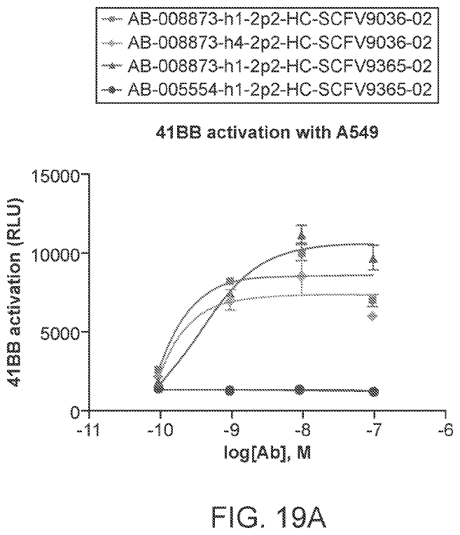

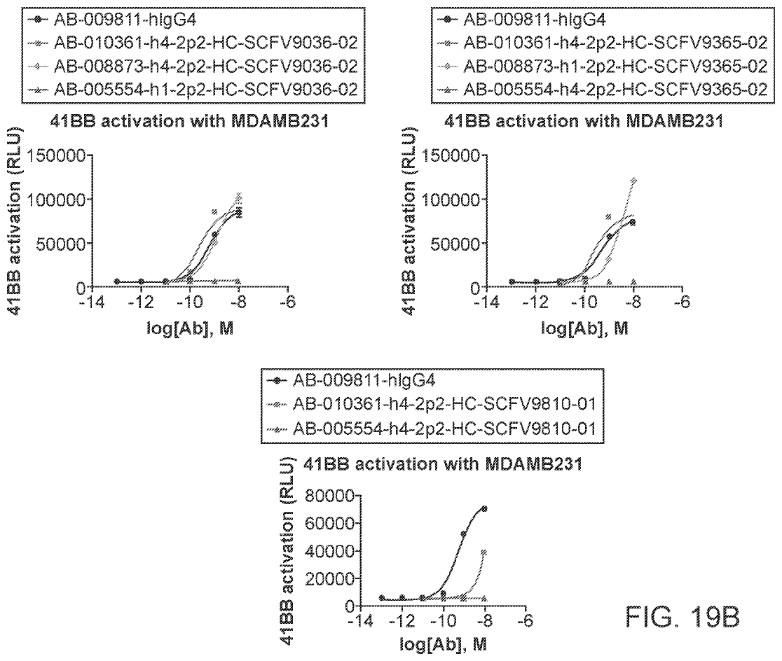

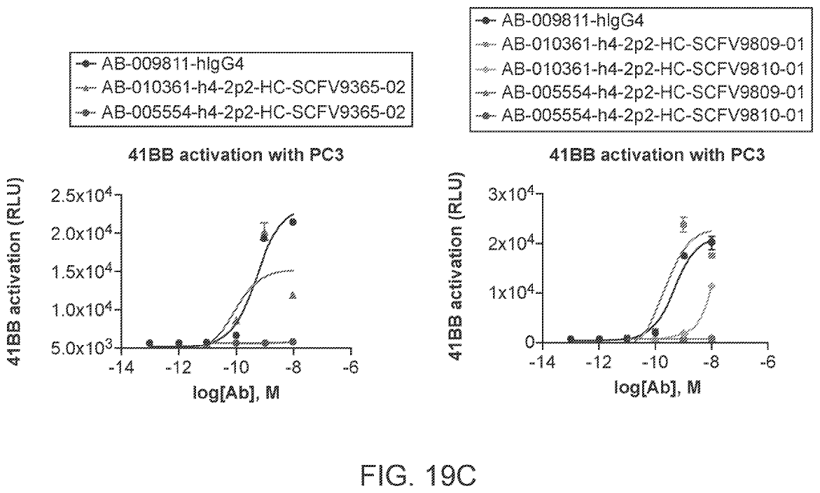

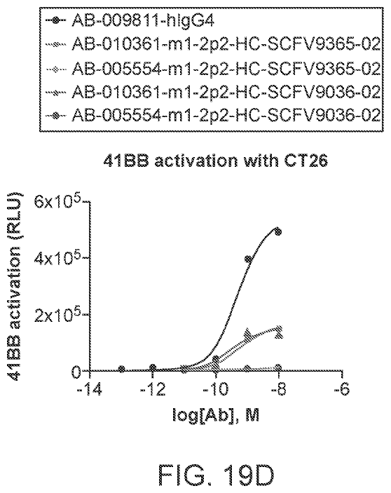

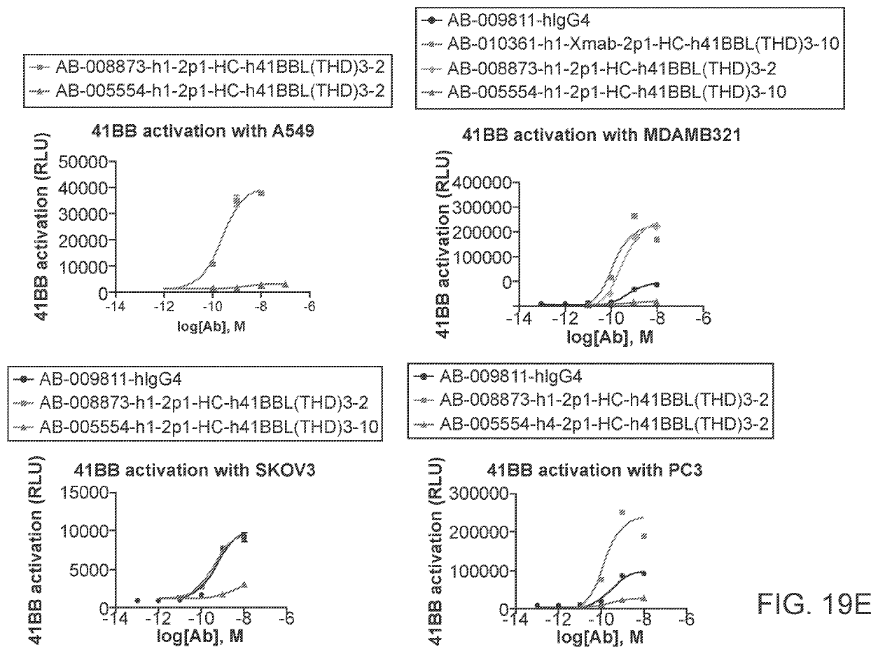

The activation assay was repeated using MDA-MB-231, CT26, or PC3 tumor cells. The EphA2-4-1BB bispecific antibodies showed activation of 4-1BB on all tumor cell lines tested. FIG. 19A-D.

The bispecific antibody that comprises two scFv fragments of anti-4-1BB antibody linked to the HC of the EphA2 antibody AB-010361 was tested in BALB/c mice that developed tumors after they have been inoculated with CT26 cells. Mice were dosed intraperitoneally with test article once a week for four weeks (7-, 14-, 21-, and 28-days post CT26 implantation) at a dose level of 10 mg/kg. Vehicle control, PBS, was dosed at 10 mL/kg. As compared to the group that were treated with PBS and the group of mice that were treated with an anti-hen egg lysozyme targeting anti-4-1BB antibody (“5554-41BB”), mice treated with the EphA2-4-1BB bispecific antibody showed reduced tumor growth during the treatment period. FIG. 49.

The mice treated with the EphA2-4-1BB bispecific antibodies as above were assayed for the presence of liver toxicity, a known side effect of certain 4-1BB agonist-based therapeutics, including anti-4-1BB antibody Urelumab. Liver toxicity was determined by measuring ALT (alanine aminotransferase) and AST (aspartate aminotransferase) which are released into the bloodstream when liver damage occurs. The ALT was measured using the MAK052 assay kit (Sigma-Aldrich) which measures the amount of pyruvate generated and AST was measured using the MAK055 assay kits (Sigma-Aldrich) which measures the amount of glutamate generated.

Results of these assays showed that mice treated with the AB-01361-4-1BB bispecific antibody and the AB-5554-41BB negative control do not show increase over vehicle of ALT & AST levels while the antibody 3H3 at 10 mg/kg (mouse surrogate for Urelumab) shows increased serum ALT & AST levels (ALT ˜8-fold increase, p<0.0001).

Furthermore, livers from mice treated with the AB-01361-4-1BB bispecific antibody showed no visible signs of liver inflammation as determined by tissue staining using hematoxylin and eosin.

Portal vein infiltration and expansion to parenchyma was detected with 10 mpk 3H3 but not AB-010361-41BB.

Constructs that comprise AB-008873 fused with h41BBL (THD)3-2 were generated and assayed for the ability to activate 4-1BB for tumor cell lines A549, MDA-MB231, SKOV3, and PC3, as described above for the EphA2-4-1BB bispecific antibodies. AB-009811 was included as a positive control and 5554-41BBL (THD) 3-10 or 5554-41BBL (THD) 3-2 was used as a negative control. The AB-008873-h41BBL construct activated 4-1BB in all cell lines tested. FIG. 19E.

### Example 11. CD3 Bispecific Constructs

This example describes CD3 bispecific antibodies comprising the anti-tumor antibodies described herein. Table 9 provides specific examples of anti-CD3 binding arms that can be combined with any of the anti-EphA2 antibodies described herein. FIG. 53 provides examples of anti-EphA2/anti-CD3 bispecific constructs that can be generated.

Bispecific constructs generated using a 1+1 format with the CD3 arm comprising the VH/VL sequence of AB-008707 were assayed for in vivo activity in a mouse model as follows. NSG-DKO mice were inoculated subcutaneously in the flank with 5e6 PC3 tumor cells in 50% matrigel on Day 0. Human PBMCs from three individual donors were engrafted via IV tail vein injection on day I following inoculation (10e6 cells/mouse). Mice were randomized into 5 mice per group per donor based on tumor volume and treated with either vehicle, 5 mg/kg anti-hen egg lysozyme non-targeting control 5554/CD3, 5 mg/kg AB-010361/CD3, or 1 mg/kg cetuximab/CD3 positive control intraperitoneally 1×/week for 3 weeks (indicated by the dotted vertical lines). Tumor volumes were measured twice per week until mice were euthanized. The results show that treatment with AB-010361/CD3 (“AB-010361/CD3”) and cetuximab/CD3 positive control (“Positive CD3 control”), but not AB-05554/CD3 negative control (“Non-targeting CD3 control”), led to a decrease in tumor burden, effectively eliminating tumors. FIG. 54. Averaging across the 3 hPBMC donors, comparing 10361-CD3 vs 5554-CD3 is significant with p=3e-7 for NAAC. Positive control 129-CD3 is significant vs 5554-CD3 with p=2e-7 for NAAC.

### Example 12. ADC Constructs

### ADC In Vitro

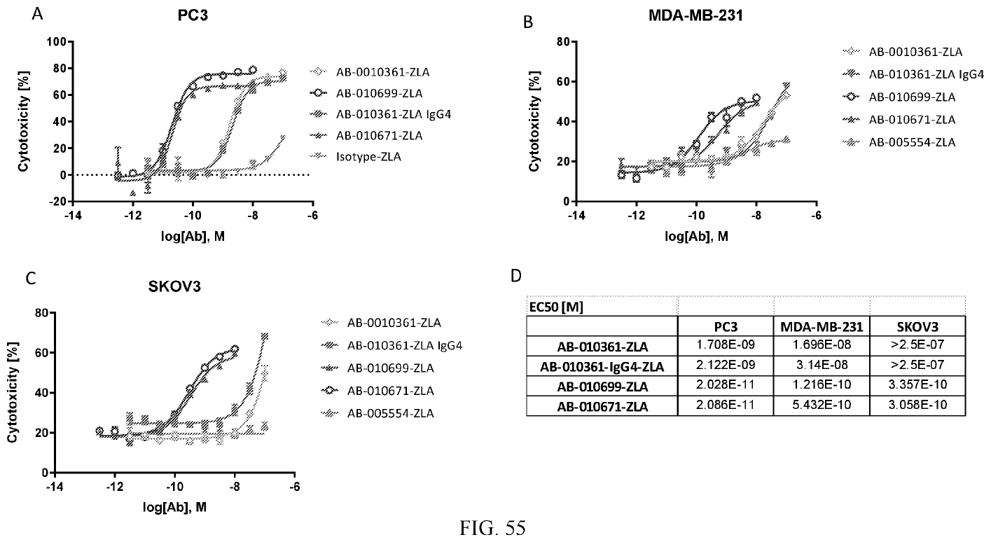

This experiment tested the cytotoxicity of antibody-drug conjugates (“conjugates”) each comprising an EphA2 antibody directly conjugated via a lysine to a ZymeLink™ Auristatin (ZLA) payload having structure (1) as disclosed above on SKOV3, MDAMB231, and PC3 tumor cells. Each of the ADCs has Formula (I). The average drug to antibody ratio (DAR) for each molecule ranges from 2.9 and 3.6 as determined by Mass Spectrometry. The EphA2 antibodies assessed include AB-010361 (IgG1), AB-10699 (IgG1), AB-010361 (IgG4), and AB-010671 (IgG1) and their conjugates are shown as “AB-0010361-ZLA,” “AB-010699-ZLA,” “AB-010361-ZLA-IgG4,” and “AB-010671-ZLA” in FIG. 55A-D. Target tumor cells were detached from the culture plate and cell concentration was adjusted to 31,250 cells/mL in assay media. 2,500 cells were added to each well of a 96 well plate and incubated with different concentrations of directly conjugated primary antibody for 15 min at room temperature. Cells were then incubated for 72 h at 37° C. and 5% CO2. At the end of the incubation period, 100 μl CellTiter-Glo® was added to each well and allowed to incubate for 5-10 min at room temperature before reading luminescence in a BMG ClarioSTAR plate reader. Data were then normalized to a maximum lysis control and plotted using graph pad prism software.

All ZLA-conjugated EphA2 antibodies tested showed dose-dependent cytotoxic activity over isotype-ZLA control in vitro on PC3, MDA-MB-231, and SKOV3 cells. FIG. 55A-C. EC₅₀ values are listed in FIG. 55D. The results show that AB-010699-ZLA and AB-010671 had higher potency but similar maximum cytotoxicity compared to AB-010361-ZLA and AB-010361-IgG4-ZLA. AB-010361-ZLA and AB-010361-IgG4-ZLA showed similar activity.

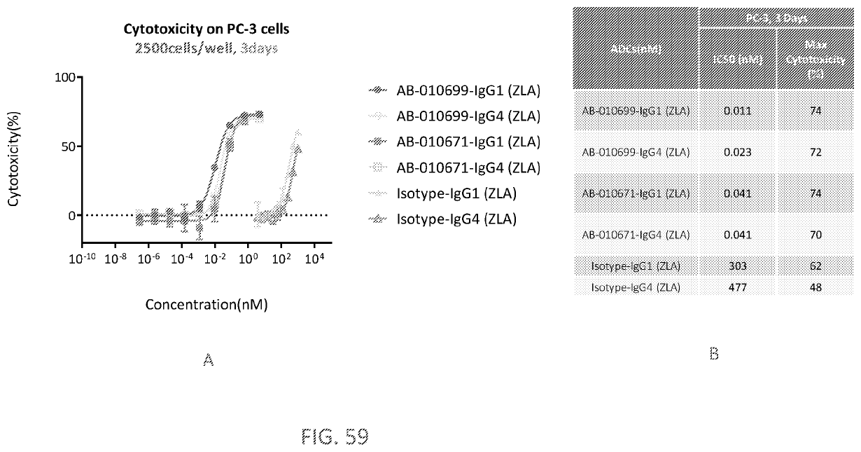

The assay was repeated in PC3 cells with AB-010699 and AB-010671 in both IgG1 and IgG4 formats. FIG. 59A. ADC constructs comprising AB-010699 Fvs exhibited slightly higher potency as compared to constructs comprising AB-010671 Fvs. No appreciable differences in potency was observed between the IgG1 and IgG4 of the same Fv. FIG. 59B.

### In Vivo PC3 Model

This experiment assessed the activity of ZLA-Conjugated EphA2 antibodies AB-010671-ZLA and AB-010699-ZLA in vivo. 2×10⁶ PC3 cells were inoculated in 0.2 mL of 1:1 DPBS with Matrigel subcutaneously into each of the male NPG mice. Tumors were allowed to establish and then randomized at an average size of 150 mm³ with 10 mice per treatment cohort. Treatment started at the day of randomization (Day 0) with weekly intravenous injections of the conjugates at the indicated doses. Body weight as well as tumor sizes were monitored twice weekly. Study was ended at day 35 after 5 doses. At endpoint, mice were euthanized, and tumors excised and weighted.

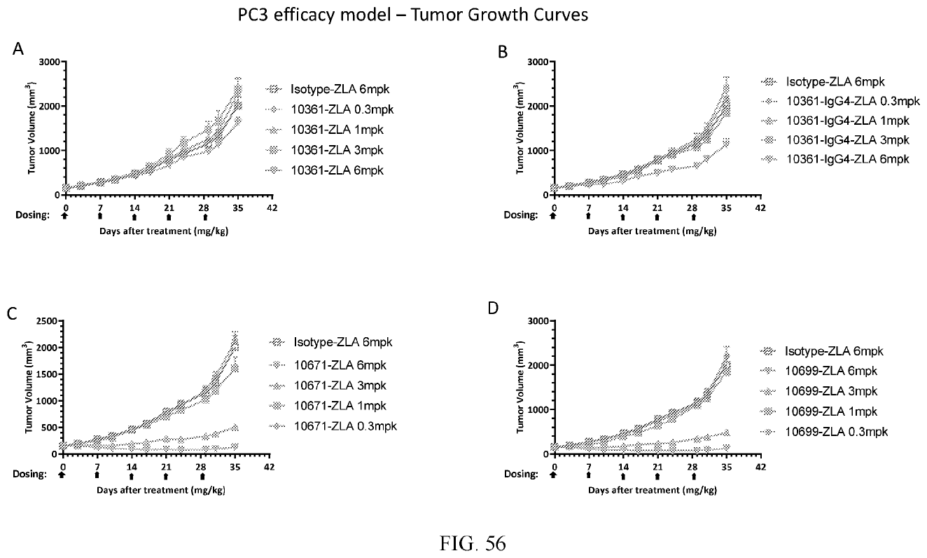

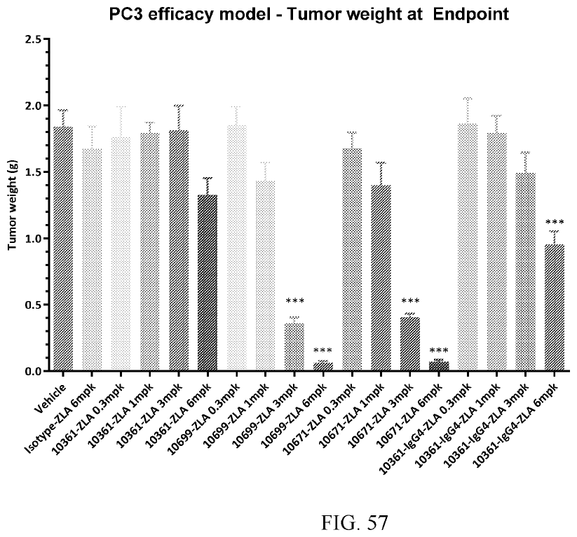

The results showed that AB-010671-ZLA (FIG. 56C and AB-010699-ZLA (FIG. 56D) exhibited dose dependent anti-tumor activity with significant tumor reduction over isotype-ZLA observed at 3 and 6 mg/kg. AB-010361-ZLA and AB-010361-IgG4-ZLA showed weak activity at the highest dose tested (FIGS. 56A and 56B) with AB-010361-IgG4-ZLA being more efficacious as AB-010361-ZLA. This tumor size observation is confirmed by endpoint tumor weight analysis (FIG. 57). AB-016699-ZLA and AB-010671-ZLA showed significantly smaller tumors in mice dosed with the conjugates at 3 mg/kg and 6 mg/kg whereas AB-010361-IgG4-ZLA showed significantly smaller tumors at 6 mg/kg.

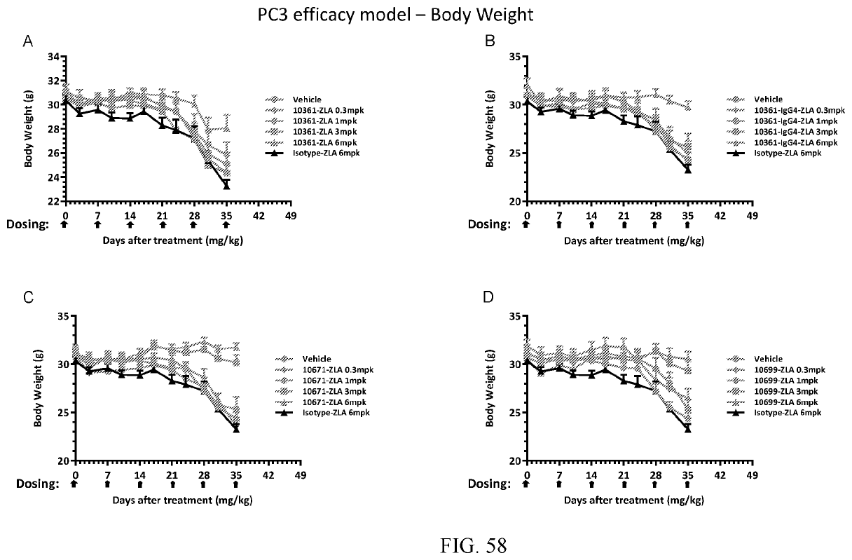

Body weights were measured throughout the study and inversely correlated with tumor burden (FIG. 58). Weight loss occurred in mice towards the end of the study starting from day 28 and was most prominent for vehicle and isotype control groups as well as tumors who didn't respond to therapy as outlined above. Mice in 3 and 6 mg/kg cohorts of AB-010699-ZLA and AB-010671 ZLA showed no loss in body weight as did mice in the 6 mg/kg cohort of AB-010361-IgG4-ZLA.

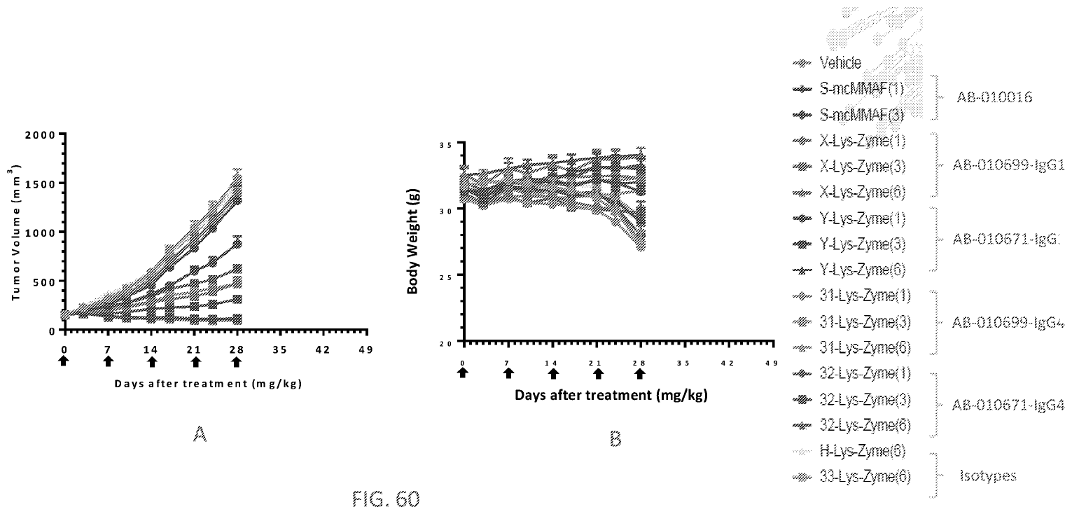

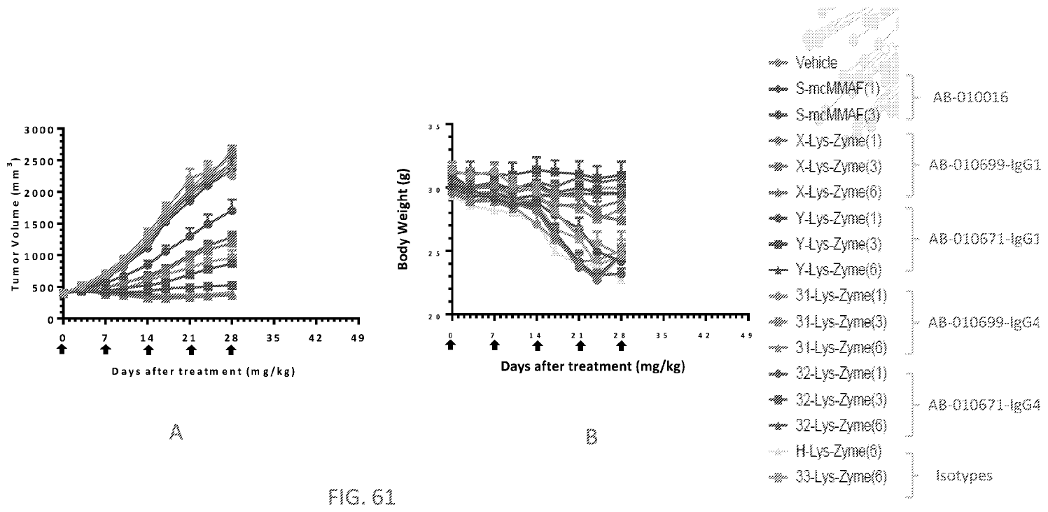

The assay was repeated with AB-010699 and AB-010671 in both IgG1 and IgG4 formats at 1, 3, and 6 mg/kg, AB-010016, and vehicle and isotype controls in mice carrying PC3 tumor cells measuring ˜150 mm³ or ˜400 mm³. AB-010699 and AB-010671 in both IgG1 and IgG4 formats showed anti-tumor activity at 3 and 6 mg/kg in both the ˜150 mm³ (FIG. 60A) and the larger more established tumor models (FIG. 61A), with AB-010699-IgG4 exhibiting slightly better efficacy compared to AB-010671-IgG4. FIG. 60B and FIG. 61B show the body weight of the mice over the course of the studies.

### INCORPORATION BY REFERENCE

Each and every publication and patent document referred to in this disclosure is incorporated herein by reference in its entirety for all purposes to the same extent as if each such publication or document was specifically and individually indicated to be incorporated herein by reference.

While the invention has been described with reference to the specific examples and illustrations, changes can be made and equivalents can be substituted to adapt to a particular context or intended use as a matter of routine development and optimization and within the purview of one of ordinary skill in the art, thereby achieving benefits of the invention without departing from the scope of what is claimed and their equivalents.

What is claimed is:

1. An immunoconjugate comprising an antibody that binds to ephrin receptor A2 (EphA2) and a cytotoxic agent,
wherein the cytotoxic agent is an auristatin analogue, and
wherein the antibody is conjugated to the auristatin analogue.

2. The immunoconjugate of claim 1, wherein the auristatin analogue has a formula of:

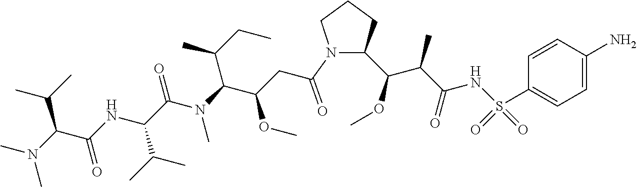 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C]

3. The immunoconjugate of claim 2, wherein the linker has a formula of 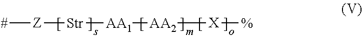
wherein:
Z is a linking group that joins the linker to a target group on the antibody;
Str is a stretcher:
AA₁ and AA₂ are each independently an amino acid, wherein AA₁-[AA₂]ᵣ forms a protease cleavage site;
X is a self-immolative group;
s is 0 or 1;
m is 1, 2, 3 or 4;
o is 0, 1 or 2;
# is the point of attachment to the antibody, and
% is the point of attachment to the auristatin analogue.

4. The immunoconjugate of claim 1, wherein the immunoconjugate has a formula of: 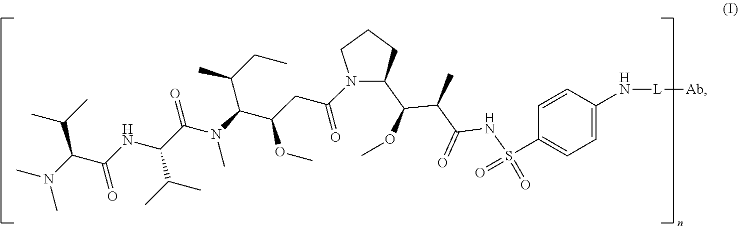
wherein:
L is a cleavable linker;
n is the drug-to-antibody ratio (DAR) and is an integer from 1 to 12, and
Ab is the antibody.

5. The immunoconjugate of claim 1, wherein the immunoconjugate has a formula of: 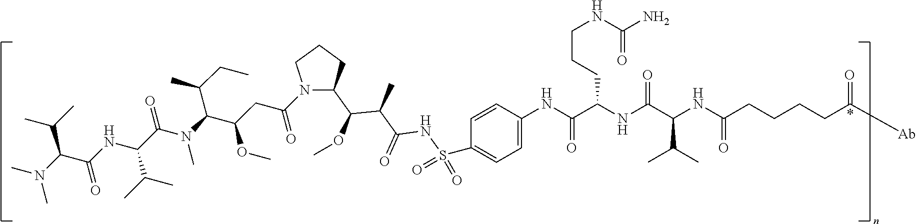
wherein n is the drug-to-antibody ratio (DAR) and is an integer from 1 to 12, and
Ab is the antibody.

6. The immunoconjugate of any one of claims 1-5, wherein the antibody of the immunoconjugate binds to an epitope of EphA2 located in the FN2 domain of EphA2, wherein the FN2 domain has a sequence of
(SEQ ID NO: 95)
TEPPKVRLEGRSTTSLSVSWSIPPPQQSRVWKYEVTYRKKGDSNSYNVR
RTEGFSVTLDDLAPDTTYLVQVQALTQEGQGAGSKVHEFQTLSPEGSG
N.

7. The immunoconjugate of claim 6 wherein the epitope comprises at least one, at least two, at least three, at least four, at least five, or at least six, at least seven, at least eight, or at least nine, or all ten of amino acid residues Leu444, Arg447, Lys476, Gln506, Ser519, Lys520, Val521, Glu523, Phe524, and Gln525 of SEQ ID NO: 94.

8. The immunoconjugate of any of claims 1-7, wherein the antibody of the immunoconjugate has one or more properties selected from the group consisting of:
a) activates the ephrin A1-EphA2 signaling axis at least 95%, 90%, 85%, or 80% less than ephrin-A1 activates the ephrin A1-EphA2 signaling axis,
b) binds preferentially to a tumor tissue than normal tissue,
c) has an EC₅₀ for activation of the ephrinA1-EphA2 signaling axis that is at least 10-, 20-, 50-, 100-, 200-, 500-fold less potent than the EC₅₀ of the antibody
d) has ADCP activity, and
e) has ADCC activity.

9. The immunoconjugate of any one of claims 1-8, wherein the antibody comprises:
a heavy chain variable region comprising:
an HCDR1 comprising a sequence (SEQ ID NO:1), or a variant HCDR1 in which 1, 2, 3, 4, or 5 amino acids are substituted relative to the sequence;
an HCDR2 comprising a sequence (SEQ ID NO:12), or a variant HCDR2 in which 1, 2, 3, 4, or 5 amino acid is substituted relative to the sequence; and
an HCDR3 comprising a sequence (SEQ ID NO:23), or a variant HCDR3 in which 1, 2, 3, 4, or 5 amino acids are substituted relative to the sequence;
a light chain variable region comprising:
an LCDR1 comprising a sequence (SEQ ID NO:34), or a variant LCDR1 in which 1, 2, 3, 4, or 5 amino acid is substituted relative to the sequence;
an LCDR2 comprising a sequence (SEQ ID NO:45), or variant LCDR2 in which 1, 2, or 3 amino acid is substituted relative to the sequence; and
an LCDR3 comprising a sequence (SEQ ID NO:56), or a variant LCDR3 in which 1, 2, 3, 4, or 5 amino acid is substituted relative to the sequence.

10. The immunoconjugate of any one of claims 1-8, wherein the antibody comprises:
a heavy chain variable region comprising:
an HCDR1 sequence set forth in any one of SEQ ID NOS:1-11 and 201-265 or a variant thereof in which 1, 2, 3, 4, or 5 amino acids are substituted relative to the sequence;
an HCDR2 sequence set forth in any one of SEQ ID NOS:12-22 and 266-330 or a variant thereof in which 1, 2, 3, 4, or 5 amino acid is substituted relative to the sequence; and
an HCDR3 sequence set forth in any one of SEQ ID NOS:23-33 and 331-395 or a variant thereof in which 1, 2, 3, 4, or 5 amino acids are substituted relative to the sequence;
a light chain variable region comprising:
an LCDR1 sequence set forth in any one of SEQ ID NOS:34-44 and 396-460 or a variant thereof in which 1, 2, 3, 4, or 5 amino acid is substituted relative to the sequence;
an LCDR2 sequence set forth in any one of SEQ ID NOS:45-55 and 461-525 or a variant thereof in which 1, 2, or 3 amino acid is substituted relative to the sequence; and
an LCDR3 sequence set forth in any one of SEQ ID NOS:56-66 and 526-590 or a variant thereof in which 1, 2, 3, 4, or 5 amino acid is substituted relative to the sequence.

11. The immunoconjugate of any one of claims 1-8, wherein the antibody comprises:
(a) an HCDR1 sequence of GGSX₁X₂ X₃YX₄WS where X₁ is F or L, X₂ is S or N, and X₃ is D or G, and X₄ is Y or H (SEQ ID NO: 769);
(b) an HCDR2 sequence of EX₁NHX₂GSX₃X₄YNNYNPSLKS, where X₁ is I or V, X₂ is A, Q, R or S, X₃ is I or T, and X₄ is N or S (SEQ ID NO: 770);
(c) an HCDR3 sequence of AKPX₁RPHC X₂NGVCX₃SGDAFDI, where X₁ is L or F, X₂ is I or T; and X₃ is Y or S (SEQ ID NO: 771);
(d) an LCDR1 sequence of X₁GNNIGX₂ X₃ X₄VH, where X₁ is G or R, X₂ is S, T, or Y, X₃ is K or M, and X₄ is N or S (SEQ ID NO: 772); (e) an LCDR2 sequence of DDSDRPS (SEQ ID NO: 45); and
(f) an LCDR3 sequence of QVWDX1 X2SDHX3V, where X1 is H or S, X2 is E, R, or S, and X3 is L or V (SEQ ID NO: 773).

12. The immunoconjugate of claim any one of claims 1-8, wherein the antibody comprises:
(a) an HCDR1 sequence of GGSX₁X₂ X₃YX₄WS where X₁ is F or L, X₂ is S or N, and X₃ is D or G, and X₄ is Y or H (SEQ ID NO: 769);
(b) an HCDR2 sequence of EX₁NHX₂GSX₃X₄YNPSLKS, where X₁ is I or V, X₂ is A, Q, R or S, X₃ is I or T, and X₄ is N or S (SEQ ID NO: 774);
(c) an HCDR3 sequence of AKPX₁RPHCX₂NGVCX₃SGDAFDI, where X₁ is L or F, X₂ is I or T; and X₃ is Y or S (SEQ ID NO: 771);
(d) an LCDR1 sequence of X₁GNNIGX₂ X₃ X₄VH, where X₁ is G or R, X₂ is S, T, or Y, X₃ is K or M, and X₄ is N or S (SEQ ID NO: 772);
(e) an LCDR2 sequence of DDSDRPS (SEQ ID NO: 45); and
(f) an LCDR3 sequence of QVWDX₁ X₂SDHX₃V, where X₁ is H or S, X₂ is E, R, or S, and X₃ is L or V (SEQ ID NO: 773).

13. The immunoconjugate of any one of claims 1-8, wherein the antibody comprises:
(a) an HCDR1 sequence of GGSX₁X₂DYX₃WS where X₁ is F or L, X₂ is S or N, and X₃ is Y or H (SEQ ID NO: 775);
(b) an HCDR2 sequence of EX₁NHX₂ GS X₃ X₄YNPSLKS, where X₁ is I or V, X₂ is R or S, X₃ is I or T, and X₄ is N or S (SEQ ID NO: 776); or an HCDR2 sequence of EX₁NHX₂ GS X₃ X₄YNNYNPSLKS, where X₁ is I or V, X₂ is R or S, X₃ is I or T, and X₄ is N or S (SEQ ID NO: 777); and
(c) an HCDR3 sequence of AKP X₁RPHCTNGVCX₂SGDAFDI, where X₁ is L or F and X₂ is Y or S (SEQ ID NO: 778);
(d) an LCDR1 sequence of GGNNIGX₁KNVH, where X₁ is S or T (SEQ ID NO: 779);
(e) an LCDR2 sequence DDSDRPS (SEQ ID NO: 45); and
(f) an LCDR3 sequence QVWDSSSDHLV (SEQ ID NO: 56).

14. The immunoconjugate of any of claims 1-13, wherein the V_{H} of the antibody comprises an amino acid sequence having at least 95% identity to (SEQ ID NO: 67); and the V_{L} comprises an amino sequence having at 95% identity to (SEQ ID NO: 78).

15. The immunoconjugate of any of claims 2-13, wherein the V_{H} of the antibody comprises an amino acid sequence having at least 95% identity to a V_{H} of any one of SEQ ID NOS:67-77 and 591-655; and the V_{L} comprises an amino sequence having at 95% identity to a V_{L} of any one of SEQ ID NOS:78-88 and 656-720.

16. The immunoconjugate of any of claims 1-15, wherein the antibody comprises:
a V_{H} region comprising amino acid sequence SEQ ID NO:67 and a V_{L} region comprising amino acid sequence SEQ ID NO:78;
a V_{H} region comprising amino acid sequence SEQ ID NO:68 and a V_{L} region comprising amino acid sequence SEQ ID NO:79;
a V_{H} region comprising amino acid sequence SEQ ID NO:69 and a V_{L} region comprising amino acid sequence SEQ ID NO:80;
a V_{H} region comprising amino acid sequence SEQ ID NO:70 and a V_{L} region comprising amino acid sequence SEQ TD NO:81;
a V_{H} region comprising amino acid sequence SEQ ID NO:71 and a V_{L} region comprising amino acid sequence SEQ ID NO:82;
a V_{H} region comprising amino acid sequence SEQ ID NO:72 and a V_{L} region comprising amino acid sequence SEQ ID NO:83;
a V_{H} region comprising amino acid sequence SEQ ID NO:73 and a V_{L} region comprising amino acid sequence SEQ ID NO:84;
a V_{H} region comprising amino acid sequence SEQ ID NO:74 and a V_{L} region comprising amino acid sequence SEQ ID NO:85;
a V_{H} region comprising amino acid sequence SEQ ID NO:75 and a V_{L} region comprising amino acid sequence SEQ ID NO:86;
a V_{H} region comprising amino acid sequence SEQ ID NO:76 and a V_{L} region comprising amino acid sequence SEQ ID NO:87;
a V_{H} region comprising amino acid sequence SEQ ID NO:77 and a V_{L} region comprising amino acid sequence SEQ ID NO:88,
a V_{H} region comprising amino acid sequence SEQ ID NO:624 and a V_{L} region comprising amino acid sequence SEQ ID NO:689,
a V_{H} region comprising amino acid sequence SEQ ID NO:652 and a V_{L} region comprising amino acid sequence SEQ ID NO:717, or
a V_{H} region comprising amino acid sequence SEQ ID NO:607 and a V_{L} region comprising amino acid sequence SEQ ID NO:672.

17. The immunoconjugate of any one of claims 1-8, wherein the antibody comprises:
(1) a heavy chain variable (V_{H}) region and a light chain variable (V_{L}) region, wherein:
(a) the V_{H} region has at least 70% identity to SEQ ID NO:67; and comprises a CDR1 of SEQ ID NO:1, or the CDR1 of SEQ ID NO:1 in which 1, 2, 3, 4, or 5 amino acids are substituted; a CDR2 of SEQ ID NO:12, or the CDR2 of SEQ ID NO:12 in which 1, 2, 3, 4, or 5 amino acids are substituted; a CDR3 of SEQ ID NO:23 or the CDR3 of SEQ ID NO:23 in which 1, 2, 3, 4, or 5 are substituted; and
(b) the V_{L} region has at least 70% identity to SEQ ID NO: 78, and comprises a CDR1 of SEQ ID NO:34 or the CDR1 of SEQ ID NO:34 in which 1, 2, 3, 4, or 5 amino acids are substituted; a CDR2 of SEQ ID NO:45, or the CDR2 of SEQ ID NO:45 in which 1, 2, 3, 4, or 5 amino acids are substituted; a CDR3 of SEQ ID NO:56 or the CDR3 of SEQ ID NO:56 in which 1, 2, 3, 4, or 5 are substituted.
(2) a heavy chain variable (V_{H}) region and a light chain variable (V_{L}) region, wherein:
(a) the V_{H} region has at least 70% identity to SEQ ID NO:652; and comprises a CDR1 of SEQ ID NO:262, or the CDR1 of SEQ ID NO:262 in which 1, 2, 3, 4, or 5 amino acids are substituted; a CDR2 of SEQ ID NO:327, or the CDR2 of SEQ ID NO:327 in which 1, 2, 3, 4, or 5 amino acids are substituted; a CDR3 of SEQ ID NO:392 or the CDR3 of SEQ ID NO:392 in which 1, 2, 3, 4, or 5 are substituted; and
(b) the V_{L} region has at least 70% identity to SEQ ID NO: 717, and comprises a CDR1 of SEQ ID NO:457 or the CDR1 of SEQ ID NO:457 in which 1, 2, 3, 4, or 5 amino acids are substituted; a CDR2 of SEQ ID NO:522, or the CDR2 of SEQ ID NO:522 in which 1, 2, 3, 4, or 5 amino acids are substituted; a CDR3 of SEQ ID NO:587 or the CDR3 of SEQ ID NO:587 in which 1, 2, 3, 4, or 5 are substituted; or
(3) a heavy chain variable (V_{H}) region and a light chain variable (V_{L}) region, wherein:
(a) the V_{H} region has at least 70% identity to SEQ ID NO:607; and comprises a CDR1 of SEQ ID NO:217, or the CDR1 of SEQ ID NO:217 in which 1, 2, 3, 4, or 5 amino acids are substituted; a CDR2 of SEQ ID NO:282, or the CDR2 of SEQ ID NO:282 in which 1, 2, 3, 4, or 5 amino acids are substituted; a CDR3 of SEQ ID NO:347 or the CDR3 of SEQ ID NO:347 in which 1, 2, 3, 4, or 5 are substituted; and
(b) the V_{L} region has at least 70% identity to SEQ ID NO: 672, and comprises a CDR1 of SEQ ID NO:412 or the CDR1 of SEQ ID NO:412 in which 1, 2, 3, 4, or 5 amino acids are substituted; a CDR2 of SEQ ID NO:477, or the CDR2 of SEQ ID NO:477 in which 1, 2, 3, 4, or 5 amino acids are substituted; a CDR3 of SEQ ID NO:542 or the CDR3 of SEQ ID NO:542 in which 1, 2, 3, 4, or 5 are substituted

18. The immunoconjugate of any one of claims 1-8, wherein the antibody comprises:
a heavy chain variable (V_{H}) region and a light chain variable (V_{L}) region,
(i) wherein the V_{H} region has at least 70% identity to SEQ ID NO:67 and comprises
(a) an HCDR1 sequence of GGSX₁X₂DYX₃WS where X₁ is F or L, X₂ is S or N, and X₃ is Y or H (SEQ ID NO: 775);
(b) an HCDR2 sequence of EX₁NHX₂ GS X₃ X₄YNPSLKS, where X₁ is I or V, X₂ is R or S, X₃ is I or T, and X₄ is N or S (SEQ ID NO: 776); or an HCDR2 sequence of EX₁NHX₂ GS X₃ X₄YNNYNPSLKS, where X₁ is I or V, X₂ is R or S, X₃ is I or T, and X₄ N or S (SEQ ID NO: 777); and
(c) an HCDR3 sequence of AKP X₁RPHCTNGVCX₂SGDAFDI, where X₁ is L or F and X₂ is Y or S (SEQ ID NO: 778); and
(ii) wherein the V_{L} region has at least 70% identity to SEQ ID NO:78 and comprises
(d) an LCDR1 sequence of GGNNIGX₁KNVH, where X₁ is S or T (SEQ ID NO: 779);
(e) an LCDR2 sequence DDSDRPS (SEQ ID NO: 45); and
(f) an LCDR3 sequence QVWDSSSDHLV (SEQ ID NO: 56).

19. The immunoconjugate of any one of claim 1-18, wherein the antibody comprises an HCDR1, HCDR2, HCDR3, LCDR1, LCDR2, and LCDR3 of an antibody designated as AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698, AB-010699, AB-010700, AB-010701, or AB-010702, or a variant thereof in which at least one, two, three, four, five, or all six of the CDRs contain 1 or 2 amino acid substitutions compared to the corresponding CDR.

20. The immunoconjugate of any one of claims 1-18, wherein the antibody comprises a heavy chain variable region and a light chain variable region of an antibody designated as AB-008873; AB-009805; AB-009806; AB-009807; AB-009808; AB-009812; AB-009813; AB-009814; AB-009815; AB-009816; AB-009817, AB-010141, AB-010142, AB-010143, AB-010144, AB-010145, AB-010146, AB-010147, AB-010148, AB-010149, AB-010150, AB-010151, AB-010152, AB-010357, AB-010358, AB-010359, AB-010360, AB-010361, AB-010362, AB-010363, AB-010364, AB-010365, AB-010366, AB-010367, AB-010661, AB-010662, AB-010663, AB-010664, AB-010665, AB-010666, AB-010667, AB-010668, AB-010669, AB-010670, AB-010671, AB-010672, AB-010673, AB-010674, AB-010675, AB-010676, AB-010677, AB-010678, AB-010679, AB-010680, AB-010681, AB-010682, AB-010683, AB-010684, AB-010685, AB-010686, AB-010687, AB-010688, AB-010689, AB-010690, AB-010691, AB-010692, AB-010693, AB-010694, AB-010695, AB-010696, AB-010697, AB-010698, AB-010699, AB-010700, AB-010701, or AB-010702, or a variant thereof, wherein the variant comprises a heavy chain variable region having a sequence that is at least 95% identical to that of the corresponding heavy chain variable region and a light chain variable region having a sequence that is at least 95% identical to the corresponding light chain variable region.

21. The immunoconjugate of any one of claims above, wherein at least 1 or 2 of the substitutions in the antibody are conservative substitutions; at least 50% of the substitutions are conservative substitutions; or all of the substitutions are conservative substitutions.

22. The immunoconjugate of any one of claims 1-8, wherein the antibody comprises a V_{H} region of any one of SEQ ID NO 67-77 and 591-655, and/or a V_{L} region of any one of SEQ ID NO: 78-88 and 656-720, or an antibody comprising a V_{H} region with at least 80% identity to any one of SEQ ID NO 67-77 and 591-655 and a V_{L} region having at least 80% identity to any one of SEQ ID NO: 78-88 and 656-720, with variations to the corresponding V_{H} or V_{L} regions present only in Framework regions.

23. The immunoconjugate of any one of claims 1-8, wherein the antibody comprises an HCDR1 of any one of SEQ ID NOS:1-11, and 201-265, an HCDR2 of any one of SEQ ID NOS:12-22 and 266-330, an HCDR3 of any one of SEQ ID NOS:23-33 and 331-395, an LCDR1 of any one of SEQ ID NOS:34-44 and 396-460, an LCDR2 of any one of SEQ ID NOS:45-55 and 461-525, an LCDR3 of any one of SEQ ID NOS:56-66 and 526-590,
wherein the FW regions in the V_{H} region of the antibody are at least 80% identical to the FW regions present in the V_{H} region of SEQ ID NO 67-77 and 591-655, and
wherein the FW regions in the V_{L} region of the antibody are at least 80% identical to the FW regions present in the V_{L} region of SEQ ID NO: 78-88 and 656-720.

24. The immunoconjugate of any one of claims 1-23, wherein the antibody is a non-natural antibody.

25. The immunoconjugate of any one of claims 1-21, wherein the antibody is a bispecific antibody comprising two antigen binding fragments, one binding to EphA2, and the other binds to a different antigen.

26. The immunoconjugate of claim 25, wherein the different antigen is 4-1bb or CD3.

27. A pharmaceutical composition comprising an immunoconjugate of any one of claims 1 to 26 and a pharmaceutically acceptable carrier.

28. A method of inducing an immune response and/or treating cancer, the method comprising administering an immunoconjugate of any one of claims 1 to 26.

29. The method of claim 28, wherein the immune response comprises an antibody-dependent cellular cytotoxicity (ADCC) and/or antibody dependent cellular phagocytosis (ADCP).

30. The method of claim 28 or 29, wherein the immunoconjugate is administered intravenously.

31. A method of treating a cancer expresses EphA2, the method comprising administering the immunoconjugate of any one of claims 1-26 to a patient having the cancer.

32. The method of claim 31, wherein the cancer is a gastric cancer, ovarian cancer, soft tissue sarcoma, lung cancer, head and neck cancer, uterine cancer, breast cancer, colorectal cancer, esophageal cancer, stomach cancer, kidney cancer, melanoma, liver cancer, bladder cancer, or testicular cancer.

33. The method of claim 32, wherein the cancer is non-small cell lung cancer, triple negative breast cancer, colorectal cancer, ovarian cancer or melanoma.

34. The method of any one of claims 31-33, wherein the immunoconjugate is administered intravenously.

35. The method of any one of claims 31 to 34, further comprising administering chemotherapy and/or radiation therapy.

36. The method of any one of claims 31 to 35, further comprising administering an agent that targets an immunological checkpoint antigen.

37. The method of claim 36, wherein the agent is a monoclonal antibody.

38. The method of claim 37, wherein the monoclonal antibody blocks PD-1 ligand binding to PD-1.

39. The method of claim 38, wherein the monoclonal antibody is an anti-PD-1 antibody.
